(19) United States
(12) Patent Application Publication
(10) Pub. No.: US 2024/0166598 A1
(43) Pub. Date: May 23, 2024

(54) COMPOUNDS, COMPOSITIONS, AND METHODS OF USING THE SAME

(71) Applicants: Arizona Board of Regents on Behalf of the University of Arizona, Tucson, AZ (US); The Board of Trustees of the University of Illinois, Urbana, IL (US)

(72) Inventors: Gregory R. Thatcher, Tucson, AZ (US); Zhengnan Shen, Tucson, AZ (US); Rui Xiong, Chicago, IL (US); Kiira Ratia, Chicago, IL (US); Lijun Rong, Westmont, IL (US); Laura Cooper, Chicago, IL (US)

(21) Appl. No.: 18/264,109

(22) PCT Filed: Feb. 2, 2022
(86) PCT No.: PCT/US2022/014965

§ 371 Date: Aug. 3, 2023

Related U.S. Application Data
(60) Provisional application No. 63/146,871, filed on Feb. 8, 2021.
(60) Provisional application No. 63/243,635, filed on Sep. 13, 2021.

(51) Int. Cl. C07D 205/04; A61K 39/215; A61K 39/39; A61K 45/06; A61P 31/14; C07C 211/49; C07C 237/30; C07D 209/08; C07D 209/14; C07D 211/58; C07D 211/62; C07D 213/81; C07D 215/12; C07D 333/20; C07D 333/58; C07D 401/12; C07D 403/12; C07D 409/12; C07D 409/14; C07D 417/12; C07H 19/067

(52) U.S. Cl. CPC C07D 205/04; A61K 39/215; A61K 39/39; A61K 45/06; A61P 31/14; C07C 211/49; C07C 237/30; C07D 209/08; C07D 209/14; C07D 211/58; C07D 211/62; C07D 213/81; C07D 215/12; C07D 333/20; C07D 333/58; C07D 401/12; C07D 403/12; C07D 409/12; C07D 409/14; C07D 417/12; C07H 19/067

(57) ABSTRACT

Provided herein are PLpro inhibitors and methods of treating and/or preventing an infection caused by a coronavirus by administration of one or more of said PLpro inhibitors to a subject in need thereof, as well as pharmaceutical formulations and kits for use in these methods.

### PRIORITY CLAIM

This application claims priority to U.S. Provisional Application No. 63/146,871, filed Feb. 8, 2021, and U.S. Provisional Application No. 63/243,635, filed Sep. 13, 2021, the contents of which are hereby incorporated by reference in their entireties.

### STATEMENT REGARDING FEDERALLY SPONSORED RESEARCH

This invention was made with government support under Grant No. TR002003, awarded by National Institutes of Health. The government has certain rights in the invention.

### TECHNICAL FIELD

The present disclosure relates generally to SARS-COV-2 PLpro inhibitors and methods of using the same for the treatment and/or prevention of infections, diseases, and symptoms thereof caused by coronaviruses, including SARS-CoV-2.

### BACKGROUND

The COVID-19 pandemic (SARS-CoV-2) is caused by severe acute respiratory syndrome coronavirus 2 (SARS-CoV-2) and has had a profound socioeconomic effect on humankind. The early sequencing of the SARS-CoV-2 genome has allowed comparisons with other coronaviruses including the Middle East Respiratory Syndrome CoV (MERS-CoV) and the earlier SARS-CoV, which like SARS-CoV-2 uses the angiotensin-converting enzyme 2 (ACE2) receptor to recognize host cells.^{1,2,3} SARS-CoV-2 shares 82% overall amino acid sequence identity with SARS-CoV and ˜50% with MERS-CoV.⁴ The high homology of SARS-CoV-2 to other coronaviruses has allowed the rapid understanding of viral biology, from particle attachment, entry, replication and primary translation (polyprotein processing), assembly, maturation, to release and shedding.^{5,6} The SARS-CoV-2 spike protein recognizes and attaches to ACE2, or the cell surface serine protease TMPRSS2, promoting viral entry. ^{1,2,3,7} Following entry, viral RNA is translated by the host ribosome to yield two large overlapping open reading frames (ORFs), ORF1a and ORF1b. Two viral cysteine proteases, the coronavirus main protease (3CLpro; nsp5) and the papain-like protease (PLpro; nsp3), proteolytically process these two viral polyproteins to yield individual non-structural proteins (nsps) that then assemble in complexes with host membrane components.⁸ The RNA-dependent RNA polymerase encoded by nsp12, a proteolytic product of 3CLpro, is a molecular target of FDA-approved COVID19 treatment, remdesivir.⁹ PLpro, recognizes the P4-P1 sequence LxGG and cleaves at three sites to release nsps 1-3. Nsp3 (1922aa, 215 kDa) incorporates PLpro itself (residues 1602-1855) and is the largest component of the replication and transcription complex.^{10,11} The catalytic activity of 3CLpro and PLpro is essential for viral replication and survival, making inhibition of these enzymes a compelling strategy for antiviral therapy.

### SUMMARY

The present disclosure provides SARS-COV-2 PLpro inhibitors and methods of using the same to treat and/or prevent infections and diseases caused by coronaviruses.

In some aspects, the present disclosure provides compounds of Formula I:

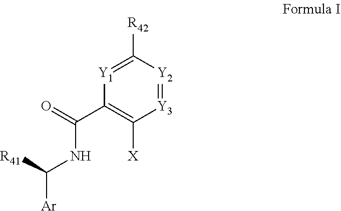

or pharmaceutically acceptable salt thereof, wherein
X is -Me, -Et, —OMe, or —CH═CH₂;
Y₁-Y₃ are independently selected from —N and —CH;
Ar is selected from aryl, heteroaryl, heterocyclyl, cycloalkyl, and cycloalkenyl, wherein each Ar group can be substituted with 1, 2, 3, 4, or 5 Rd groups;
R₄₁ and R₄₂ are independently selected from —C₁-C₆ alkyl, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, —(C₁-C₆ alkylenyl)R^{c}R^{c′}, and —C₁-C₆ alkyl;
R^{a} and R^{b} are independently selected at each occurrence from —H, —C₁-C₆ alkenyl, —C₁-C₆ alkynyl, —C₁-C₆ haloalkyl, —R^{c}, and —C₁-C₆ alkyl, wherein the —C₁-C₆ alkyl can be substituted with a substituent selected from —OR^{e}, —NR^{e}R^{f}, —C(O)OR^{e}, —C(O)NR^{e}R^{f}, —S(O)₂R^{e}, —S(O)₂NR^{e}R^{f}, and
—R^{c}; R^{c} and R^{c′} are independently selected at each occurrence from aryl, heteroaryl, heterocyclyl, cycloalkyl, and cycloalkenyl, wherein each R^{c} group can be substituted with 1, 2, 3, 4, or 5 R^{d} groups; and R^{d} is independently selected at each occurrence from —C₁-C₆ alkyl, —C₂-C₆ alkenyl, —C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f},
   wherein R^{e} and R^{f}, are independently selected at each occurrence from —H, —C₁-C₆ alkyl, —C₁-C₆ cycloalkyl, aryl, heteroaryl and —C₁-C₆ haloalkyl.

In some embodiments, X is -Me. In another embodiment, each of Y₁-Y₃ are —CH. In some embodiments, R₄₁ is a —C₁-C₆ alkyl. In certain embodiments, Ar is an aryl.

In certain embodiments, the present disclosure provides compounds of Formula II:

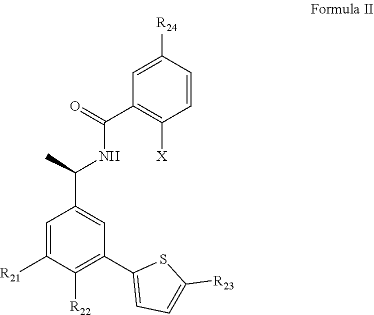

or pharmaceutically acceptable salt thereof, wherein
X is -Me, -Et, —OMe, or —CH═CH₂;
R₂₁, R₂₂, R₂₃ and R₂₄ are independently selected from —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′}; and
R^{a} and R^{b} are independently selected at each occurrence from —H, —C₁-C₆ alkenyl, —C₁-C₆ alkynyl, —C₁-C₆ haloalkyl, R^{c}, and —C₁-C₆ alkyl, wherein the —C₁-C₆ alkyl can be substituted with —OR^{e}, —NR^{e}R^{f}, —C(O)OR^{e}, —C(O)NR^{e}R^{f}, —S(O)₂R^{e}, —S(O)₂NR^{e}R^{f}, or R^{c};
R^{c} and R^{c′} are independently selected at each occurrence from aryl, heteroaryl, heterocyclyl, cycloalkyl, or cycloalkenyl, wherein each R^{c} group can be substituted with 1, 2, 3, 4, or 5 R^{d} groups; and R^{d} is independently selected at each occurrence from —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, wherein R^{e} and R^{f} are independently selected at each occurrence from —H, —C₁-C₆ alkyl, —C₁-C₆ cycloalkyl, aryl, heteroaryl and —C₁-C₆ haloalkyl.

In some embodiments, W₁ is O. In another embodiment, W₁ is N. In another embodiment, m₁, m₂, n₁ and n₂ are 1.

In yet another embodiment, a compound of Formula II is:

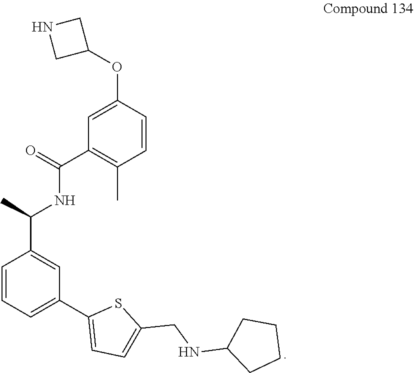 [SMILES: Cc1ccc(OC2CNC2)cc1C(=O)N[C@H](C)c1cccc(-c2ccc(CNC3CCCC3)s2)c1]

In certain embodiments, the present disclosure provides compounds for Formula XI:

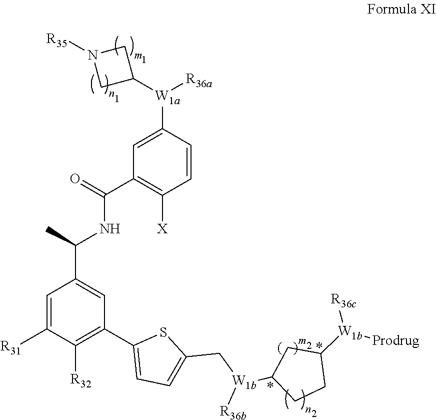

or a pharmaceutically acceptable salt thereof, wherein
X is -Me, -Et, —OMe, —CH═CH₂,
W₁ₐ and W_{1b} is —N, —O or —C; wherein if W₁ₐ═O, R₃₆ₐ does not exist and/or if W_{1b}═O, R_{36b} does not exist;
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

In some embodiments, the prodrug is selected from the group consisting of hydroxyl, carboxyl, amine, phosphate, phosphonate, amidine, guanine and/or carbohydrate.

In certain embodiments, the present disclosure provides compounds of Formula XII:

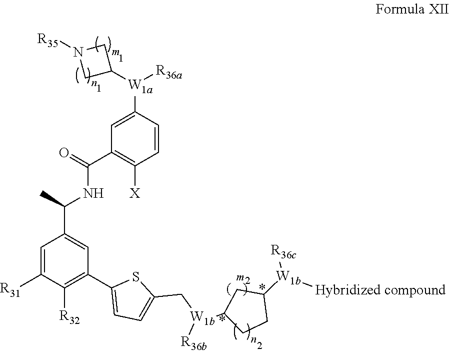

or a pharmaceutically acceptable salt thereof, wherein
X is -Me, -Et, —OMe, —CH═CH₂;
W₁ₐ and W_{1b} is —N, —O or —C; wherein if W₁═O, R₃₆ does not exist,
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₃, R₃₄, R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

In certain embodiments, the present disclosure provides compounds of Formula XIII:

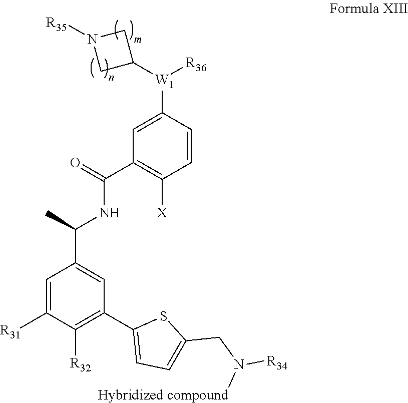

or a pharmaceutically acceptable salt thereof, wherein
X is -Me, -Et, —OMe, or —CH═CH₂;
W₁ is —N or —O; wherein if W₁═O, R₃₆ does not exist;
R₃₁ and R₃₂ are independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₄, R₃₅, and R₃₆ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
m and n=1-3.

In some embodiments, the hybridized compound is selected from the group consisting of kinase inhibitors, NAMPT inhibitors, coronavirus inhibitors and virus inhibitors.

In yet another embodiment, a compound of Formula XII or XIII is selected from:

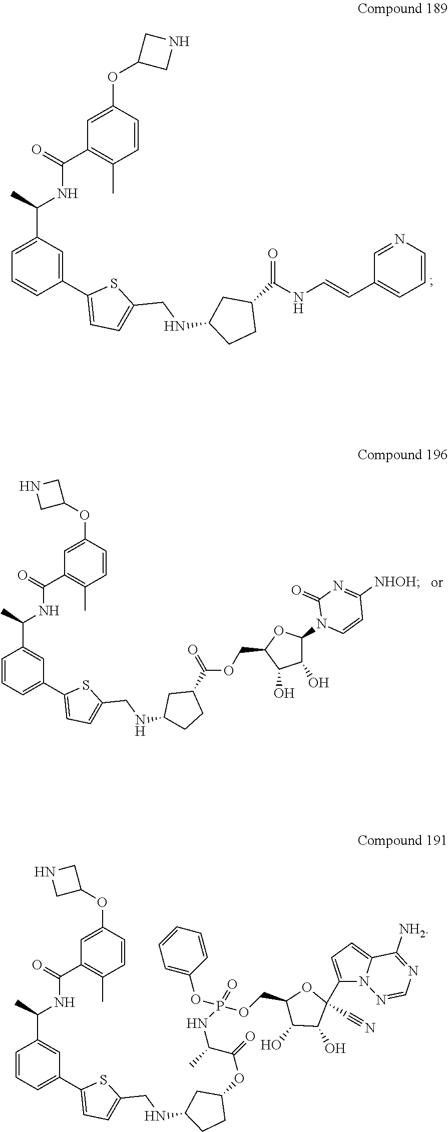

In yet another embodiment, the present disclosure provides compounds of Formula XIV:

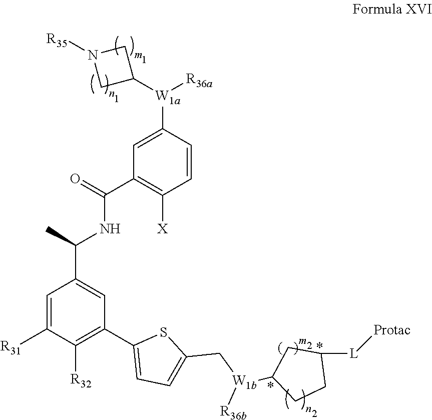

or a pharmaceutically acceptable salt thereof, wherein
X is -Me, -Et, —OMe, —CH═CH₂;
W₁ₐ and W_{1b} is —N, —O or —C; wherein if W₁═O, R₃₆ does not exist
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl;
m₁, m₂, n₁ and n₂=1-3; and
L is a (poly)ethyleneglycol or substituted alkyl groups optionally interdispersed with O, N, S, P or Si atoms.

In some embodiments, the Protac is a hetero bifunctional molecule that connects a POI ligand to an E3 ubiquitin ligase (E3) (VHL, CRBN, IAPs, and MDM2) recruiting ligand selected from the group consisting of thalidomide, pomalidomide, lenalidomide, and VHL.

In some embodiments, a compound of Formula XVI is:

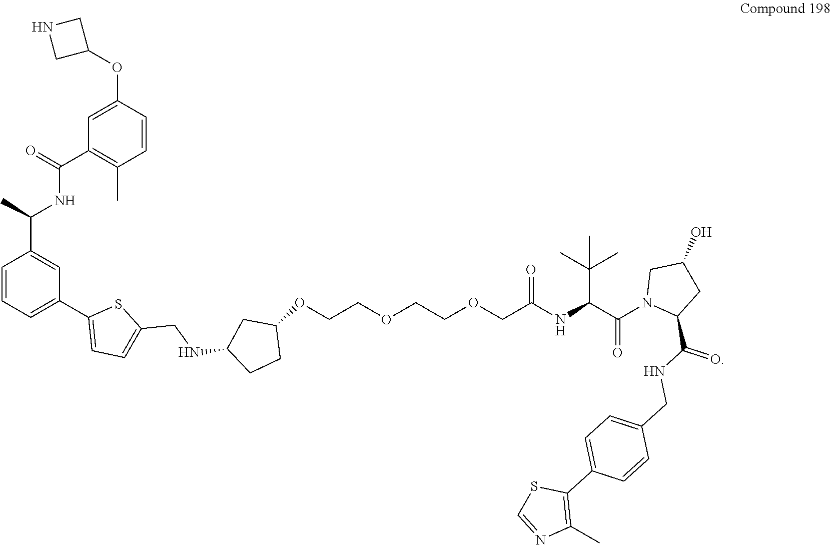 [SMILES: Cc1ccc(OC2CNC2)cc1C(=O)N[C@H](C)c1cccc(-c2ccc(CN[C@H]3CC[C@@H](OCCOCCOCC(=O)N[C@H](C(=O)N4C[C@H](O)C[C@H]4C(=O)NCc4ccc(-c5scnc5C)cc4)C(C)(C)C)C3)s2)c1]

In some aspects, the present disclosure provides a pharmaceutical composition comprising one or more PLpro inhibitors described herein. In some embodiments, the pharmaceutical composition is packaged in a packaging material and identified in print, in or on said packaging material, for use in treating and/or preventing an infection caused by a coronavirus.

In some aspects, the present disclosure provides a method of treating and/or preventing an infection caused by a coronavirus in a subject in need thereof, comprising administered to the subject the pharmaceutical composition comprising one or more PLpro inhibitors described herein.

In some embodiments, the coronavirus is SARS-CoV-2. In some embodiments, the subject is 65 years or older. In yet another embodiment, the subject has one or more underlying medical conditions selected from the group consisting of cancer, chronic kidney disease, chronic obstructive pulmonary disease (COPD), immunocompromised state, obesity, serious heart conditions, sickle cell disease, Type 2 diabetes mellitus, asthma, cerebrovascular disease, cystic fibrosis, hypertension or high blood pressure, neurologic conditions, liver disease, pregnancy, pulmonary fibrosis, smoking, thalassemia, and Type 1 diabetes mellitus.

In some embodiments, the methods further comprise administering to the subject one or more antiviral agents. In some embodiments, the one or more antiviral agents is selected from the group consisting of remdesivir, favipiravir, lopinavir, ritonavir, nitazoxanide, danoprevir, ASC-09, umifenovir, nafamostat, brequinar, AT-527, ABX464, merimepodib, molnupiravir, opaganib, ivermectin, and hydroxychloroquine. In yet another embodiment, the one or more antiviral agents is a vaccine. In some embodiments, the vaccine is selected from the group consisting of BNT162b2 (Pfizer/BioNTech), mRNA-1273 (Moderna), AZD1222/ChAdOxl (AstraZeneca/Oxford Univ), Ad5-vectored COVID-19 vaccine (CanSino Biologies), CoronaVac (Sinovac), and/or NVX-CoV2373 (Novavax).

### BRIEF DESCRIPTION OF THE DRAWINGS

FIG. 1A is a representative schematic of a high-throughput (HTS) screening method to identify SARS-COV-2 PLpro inhibitors in accordance with embodiments of the present disclosure. FIG. 1B-1D illustrate high-throughput screen and counterscreen for SARS-CoV-2 PLpro inhibitors. High-throughput screening was performed against a TargetMol Bioactives Library that contains 1,283 FDA-approved drugs and 761 drugs approved by regulatory bodies in other countries (FIG. 1B) and a 10,000-compound SMART library subset from ChemDiv (FIG. 1C). Compounds producing >40% inhibition of PLpro enzymatic activity were selected for follow-up studies. FIG. 1D shows counterscreen of selected compounds against human deubiquitinating enzyme, USP7

FIG. 2A shows structures of the exemplary PLpro inhibitors avasimibe, candesartan cilexetil, CPI-169, MK-3903, pyrantel pamoate, and GRL0617 and representative graphs of the dose dependent inhibition of those inhibitors in accordance with embodiments of the present disclosure. FIG. 2B is a representative graph of an overlay of surface plasmon resonance (SPR) sensograms of the single-cycle kinetics for HTS hits (0.08 μM to 50 μM, 2.5-fold dilutions) of the PLpro inhibitors of FIG. 2A in accordance with embodiments of the present disclosure. FIG. 2C shows representative graphs of the binding of GRL0617 and CPI-169 to SARS-CoV-2 PLpro as measured by SPR in accordance with embodiments of the present disclosure.

FIG. 3A shows a representative image of an overall structure and domain organization of PLpro and the PLpro-ubiquitin complex where GRL0167 is shown in cyan in accordance with embodiments of the present disclosure. FIG. 3B shows a representative image of the twisting the BL2 loop induced by GRL0617 binding where conformation of the ubiquitin-bound BL2 loop is shown in pale orange and the GRL0167-bound loop is shown in cyan in accordance with embodiments of the present disclosure. FIG. 3C shows a representative image of the BL2 loop conformational flexibility with the structure of the GRL0617-bound PLpro (electrostatic surface representation) and associated BL2 loop (cyan cartoon) superimposed with Ub-bound (orange; pdb: 6XAA) and apo structures (wheat; pdbs 6WZU, 7D47, 7JCD) where Gln269 is shown for reference in accordance with embodiments of the present disclosure. FIG. 3D shows a representative table of the structural detail of Sites I-V of PLpro targeted for drug design in accordance with embodiments of the present disclosure. FIG. 3E provides a summary of structure activity relationship of selected compounds in accordance with embodiments of the present disclosure. FIG. 3F shows a representative image of PLpro Glu167 (shown in magenta) interacting with Arg72 of ubiquitin (orange) in Site I, where GRL0617 is aligned with Site 1 and shown in cyan in accordance with embodiments of the present disclosure. FIG. 3G shows predicted angle between the amide plane and the aryl plane of GRL analogs. Calculations use quantum mechanics (B3LYP/6-31G*) with a polarizable continuum model (PCM) as the continuum solvation method for water. The tortional angle in aniline part of the molecule was locked using experimental angles determined in PDB, 7LBR. FIG. 3H shows a model of ZN3-56 (light blue) bound to PLpro. The electrostatic interactions between ZN-3-56 and Arg166 and Glu167 are highlighted with dashed lines.

FIG. 4A shows structures of the exemplary PLpro inhibitors GRL0167, ZN-2-184, ZN-3-80, XR8-24, XR8-23, and XR8-89 and representative graphs of the dose responses of those inhibitors in accordance with embodiments of the present disclosure. FIGS. 4B-4C show representative graphs of the association and dissociation rates, respectively, as determined by SPR of the PLpro inhibitors of FIG. 4A in accordance with embodiments of the present disclosure. FIG. 4D shows a representative graph comparing the KD measured by SPR and the IC₅₀ of enzyme inhibition assay of the PLpro inhibitors of FIG. 4A in accordance with embodiments of the present disclosure. FIGS. 4E-4F show representative graphs of the inhibition of deubiquitinating (FIG. 4E) and de-ISGgylating activities (FIG. 4F) of the PLpro inhibitors of FIG. 4A at three concentrations (e.g., 30 μM, 3 μM, and 0.3 μM) in accordance with embodiments of the present disclosure. FIG. 4G shows SPR binding sensorgrams of GRL0617 and analogs. The response sensorgrams were double referenced with a reference channel and zero concentration (2% DMSO) responses, and reference subtracted sensorgrams were fitted with a 1:1 Langmuir kinetic model using Biacore Insight evaluation software, producing two rate constants (ka and kd). The equilibrium dissociation constants (KD) were determined from the two rate constants (KD=kd/ka).

FIGS. 5A-5B show representatives image of an 2Fₒ-F_{c} electron density map revealing the structural details of PLpro inhibitors XR8-24 (FIG. 5A) and XR8-89 (FIG. 5B) complexed with SARS CoV-2 PLpro where the maps are shown by blue mesh and are contoured at 1 sigma around the PLpro inhibitors and hydrogen bonds are indicated by dashed lines with distances (in A) indicated in italics in accordance with embodiments of the present disclosure. FIG. 5C shows a representative image of the superposition of SARS-COV-2 PLpro-bound GRL0617 (cyan; pbd 7JRN) with XR8-24 (yellow) and XR8-89 (orange) in accordance with embodiments of the present disclosure. FIG. 5D shows a representative image of the interaction of XR8-24 with the BL2 groove in accordance with embodiments of the present disclosure. FIG. 5E shows a representative image comparing a PLpro ligand and PLpro inhibitor binding surfaces on PLpro where the surface of the body of ubiquitin is shown in orange and its 5 C-terminal residues are shown as orange sticks (pdb 6XAA), GRL is shown in cyan (pdb 7JRN), a covalent peptide-based inhibitor (pdb 6WUU) is shown in magenta, XR8-24 is shown in yellow, and the binding surface unique to XR8-24 and close analogs is highlighted by a yellow circle in accordance with embodiments of the present disclosure. FIG. 5F shows superposition of five PLpro:XR8 inhibitor crystal structures. The chemical structures of inhibitors and their associated pdb IDs are shown at right, with colored bars corresponding to the coloring used at left.

FIGS. 6A-6D show representative graphs and images of PLpro inhibitors GRL0617, XR8-23, XR8-24, and XR8-89 against SARS-CoV-2 infected Vero E6 and A549 cells showing an EC50 of 2 μM in accordance with embodiments of the present disclosure. FIGS. 6A-6B show improved PLpro inhibitors demonstrating potent antiviral efficacy. FIG. 6A shows plaque reduction of SARS-CoV-2 infected Vero E6 cells at MOI=0.0001 treated with GRL0617, XR8-23, XR8-24, and XR8-89 from 20 μM to 0.156 μM in the presence of 1.5 μM CP-100356. Cy-totoxicity in Vero E6 cells was measured by CellTiter-Glo assay shown in FIG. 6B. To measure reduction in virus yield, A549-hACE2 cells were infected with MOI=0.01 of SARS-CoV-2 cultured in Vero E6 cells with and without various concentrations GRL0617, XR8-23, XR8-24 after 48 hours, supernatants were harvested, RNA isolated and quantified by RT-qPCR. The data show mean±S.D. FIG. 6C shows dose dependent plaque reduction of XR8-23 and XR8-24. FIG. 6D shows cell viability of GRL0617, XR8-23 and XR8-24 in A549-hACE2 cells.

### DETAILED DESCRIPTION

Provided herein is a library of SARS-CoV-2 PLpro inhibitors, designed and synthesized utilizing a structure-based drug design approach. The studies described herein led to the identification of exemplary compounds with low nanomolar potency against SARS-CoV-2 PLpro as well as co-crystal structures of a number of the disclosed inhibitors with SARS-CoV-2 PLpro (e.g., PDB: 7LBR, 7LBS and 7LLF). These crystal structures revealed a previously unidentified “BL2 groove” formed by closing of the BL2 loop (blocking loop 2, AA266-271) and the palm domain. Overall, structure-based optimization led to PLpro inhibitors with increased metabolic stability, low nanomolar potency against PLpro enzyme activity, a relatively slow dissociation rate, and low micromolar potency against infectious SARS-CoV-2. The compounds described herein represent the most potent inhibitors of PLpro reported to date with activity translating to live virus assays in mammalian and human host cells.

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Definitions

Terms used herein may be preceded and/or followed by a single dash, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and —OC(O)C₁-C₆ alkyl indicate the same functionality; similarly, arylalkyl and -alkylaryl indicate the same functionality.

The term “alkyl” refers to a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “alkoxy” refers an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkynyl” refers to a straight or branched chain hydrocarbon group containing from 2 to 10 carbon atoms and containing at least one carbon-carbon triple bond. Representative examples of alkynyl include, but are not limited, to ethynyl, 1-propynyl, 2-propynyl, 3-butynyl, 2-pentynyl, and 1-butynyl. In one embodiment the alkynyl is ethynyl.

The terms “cyano” and “nitrile” refer to a —CN group.

The term “cycloalkyl” refers to a monocyclic or a bicyclic cycloalkyl ring system. Monocyclic ring systems are cyclic hydrocarbon groups containing from 3 to 8 carbon atoms, where such groups can be saturated or unsaturated, but not aromatic.

In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclicheterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thio. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thio. In certain embodiments of the disclosure, the cycloalkyl is cyclopentyl, cyclohexyl, or cycloheptyl.

The term “haloalkyl” refers to the present of at least one halogen appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl. In certain embodiments, each “haloalkyl” is a fluoroalkyl, for example, apolyfluoroalkyl such as a substantially perfluorinated alkyl.

The term “pharmaceutically acceptable salts” refers to salts or zwitterionic forms of the present compounds. Salts of the present compounds can be prepared during the final isolation and purification of the compounds or separately by reacting the compound with an acid having a suitable cation. The pharmaceutically acceptable salts of the present compounds can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, tartaric, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides.

The term “subject” refers to a mammalian subject, preferably a human. A “subject in need thereof” refers to a subject who has been infected with a coronavirus, has been diagnosed of a disease caused by a coronavirus, or is at an increased risk of infection or developing a severe illness caused by a coronavirus. The phrases “subject” and “patient” are used interchangeably herein.

The term “treatment” in relation a given disease, disorder or viral infection, includes, but is not limited to, inhibiting the disease, disorder or viral infection, for example, arresting the development of the disease, disorder, or viral infection; relieving the disease, disorder, or viral infection for example, causing regression of the disease, disorder, or viral infection; or relieving a condition caused by or resulting from the disease, disorder, or viral infection for example, relieving or treating symptoms of the disease, disorder, or viral infection. The term “prevention” in relation to a given disease, disorder, or viral infection means: preventing the onset of disease, disorder, or viral infection development if none had occurred, preventing the disease, disorder, or viral infection from occurring in a subject that may be predisposed to the disorder, disease, or viral infection but has not yet been diagnosed as having the disorder, disease, or viral infection and/or preventing further disease/disorder/infection development if already present.

The term “prevention” in relation to a given disease, disorder, or viral infection means: preventing the onset of disease development if none had occurred, preventing the disease, disorder, or viral infection from occurring in a subject that may be predisposed to the disorder, disease, or viral infection but has not yet been diagnosed as having the disorder, disease, or viral infection and/or preventing further disease/disorder/viral infection development if already present.

The term “therapeutically effective amount” refers to an amount that produces a desired effect in a subject for treating and/or preventing a condition, e.g., a therapeutic effect. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited to, the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely, by monitoring a subject's response to administration of the composition and adjusting the dosage accordingly. For additional guidance, see, for example, Remington: The Science and Practice of Pharmacy, 22nd ed., Pharmaceutical Press, London, 2012, and Goodman & Gilman's The Pharmacological Basis of Therapeutics, 12th ed., McGraw-Hill, New York, NY, 2011, the entire disclosures of which are incorporated by reference herein.

The term “prodrug” refers to a pharmacologically inactive substance that is converted in the body (e.g., via enzymatic or non-enzymatic action, including pH-dependent bioactivation) into a pharmacologically active drug. In certain embodiments of such prodrugs, the prodrug may contain the drug promoiety linked to an auxophore in a prodrug designed to take advantage of cellular transporters (e.g. wherein the auxophore is a saccharide or disaccharide, or an amino acid or dipeptide). Such a prodrug may exhibit enhanced active transport across cellular membranes in the body; alternatively such a prodrug may inhibit efflux of drug and prodrug via interaction with cellular efflux transporters in the body.

The term “hybrid” refers to a chimeric drug that contains a drug linked to a second pharmacophore, wherein the conjugation of the drug to the second pharmacophore in the chimeric hybrid may be stable or may be labile to bioactivation as described for prodrugs. Thus, a hybrid drug may also be a prodrug if the linker conjugating the drug to the second pharmacophore is converted in the body by enzymatic or non-enzymatic means into the pharmacologically active drug. The second pharmacophore in a hybrid drug may have beneficial biological activity through interaction with a second viral or host target in the body. A hybrid drug that is not a prodrug possesses two pharmacophores that do not require enzymatic or non-enzymatic action to be converted into pharmacologically active drug in the body.

The term “proteoloysis-targeting chimera (PROTAC)” refers to a molecule generally having three components, an E3 ubiquitin ligase binding group (E3LB), a linker L2, and a protein binding group (PB). The E3LB-L2 conjugate in PROTACs constitutes a degron that targets the protein bound by the PB for ubiquitin-dependent proteolysis. Degrons include conjugates that cause ubiquitin-dependent and -independent proteolysis. Ubiquitin-independent degrons include short intrinsic amino acid sequences, such as the D-element, the PEST sequence, unstructured initiation sites, or short sequences rich in acceptor lysines, which regulate target protein stability by promoting ubiquitin-independent proteolysis.

### Compounds

In certain embodiments, compounds of the present disclosure include, without limitation, compounds of Formula I:

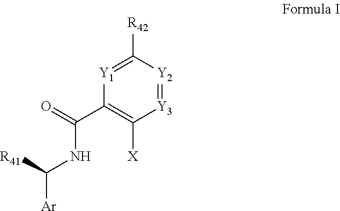

or a pharmaceutically acceptable salt thereof, wherein
X is -Me, -Et, —OMe, or —CH═CH₂;
Y₁-Y₃ are independently selected from —N and —CH;
Ar is selected from aryl, heteroaryl, heterocyclyl, cycloalkyl, and cycloalkenyl, wherein each Ar group can be substituted with 1, 2, 3, 4, or 5 R^{d} groups;
R₄₁ and R₄₂ are independently selected from —C₁-C₆ alkyl, —(C₁-C₆ alkylenyl)NR^{a}R^{b}, —OR^{a}, —(C₁-C₆alkylenyl)NC(O)R^{a}, —(C₁-C₆ alkylenyl) C(O)NR^{a}, N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b} are independently selected at each occurrence from —H, —C₁-C₆ alkenyl, —C₁-C₆ alkynyl, —C₁-C₆ haloalkyl, —R^{c}, and —C₁-C₆ alkyl, wherein the —C₁-C₆ alkyl can be substituted with a substituent selected from —OR^{e}, —NR^{e}R^{f}, —C(O)OR^{e}, —C(O)NR^{e}R^{f}, —S(O)₂R^{e}, —S(O)₂NR^{e}R^{f}, and
—R^{c}; R^{c} and R^{c′} are independently selected at each occurrence from aryl, heteroaryl, heterocyclyl, cycloalkyl, and cycloalkenyl, wherein each R^{c} group can be substituted with 1, 2, 3, 4, or 5 R^{d} groups; and R^{d} is independently selected at each occurrence from —C₁-C₆ alkyl, —C₂-C₆ alkenyl, —C₂-C₆ alkynyl, halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f},
wherein R^{e} and R^{f}, are independently selected at each occurrence from —H, —C₁-C₆ alkyl, —C₁-C₆ cycloalkyl, aryl, heteroaryl and —C₁-C₆ haloalkyl.

In certain embodiments, compounds of the present disclosure include, without limitation, compounds of Formula II:

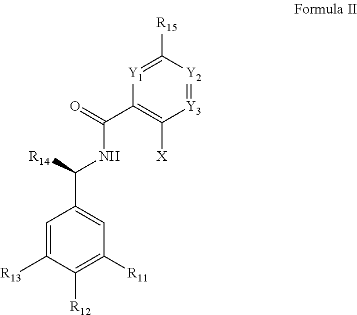

or a pharmaceutically acceptable salt thereof, wherein
X is -Me, -Et, —OMe, or —CH═CH₂;
Y₁-Y₃ are independently selected from —N or —CH;
R₁ is selected from aryl, a sulfur-containing heteroaryl, an oxygen-containing heteroaryl nitrogen-containing heteroaryl, each of which can be substituted with 1, 2, or 3 substituents independently selected from —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R²)S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C⁶ alkylenyl)R^{c}R^{c′};
R₁₂, and R₁₃ are independently selected from —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C⁶ alkylenyl)R^{c}R^{c′};
R₁₄ is selected from —C₁-C₅ alkenyl, —C₁-C₆ alkynyl, —C₁-C₆ haloalkyl, —R^{c}, and —C₁-C₆ alkyl, where the —C₁-C₆ alkyl can be substituted with a substituent selected from —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R₁₅ is selected from —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C⁶ alkylenyl)R^{c}R^{c′};
R^{a} and R^{b} are independently selected at each occurrence from —H, —C₁-C₆ alkenyl, —C₁-C₆ alkynyl, —C₁-C₆ haloalkyl, R^{c}, and —C₁-C₆ alkyl, where the —C₁-C₆ alkyl can be substituted with a substituent selected from —OR^{e}, —NR^{e}R^{f}, —C(O)OR^{e}, —C(O)NR^{e}R^{f}, —S(O)₂R^{e}, and —S(O)₂NR^{e}R^{f}, and —R^{c};
R^{c} and R^{c′} are independently selected at each occurrence from aryl, heteroaryl, heterocyclyl, cycloalkyl, and cycloalkenyl, wherein each R^{c} group can be substituted with 1, 2, 3, 4, or 5 R^{d} groups; and
R^{d} is independently selected at each occurrence from —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, wherein R^{e} and R^{f} are independently selected at each occurrence from —H, —C₁-C₆ alkyl, —C₁-C₆ cycloalkyl, aryl, heteroaryl and —C₁-C₆ haloalkyl.

In certain embodiments, compounds of the present disclosure include, without limitation, compounds of Formula III:

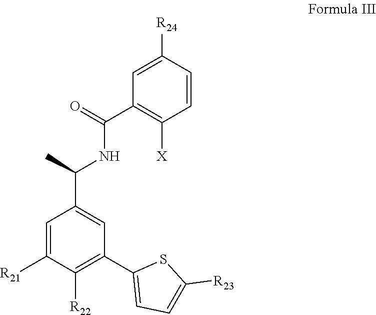

or a pharmaceutically acceptable salt thereof, wherein
X is -Me, -Et, —OMe, or —CH═CH₂;
R₂₁, R₂₂, R₂₃ and R₂₄ are independently selected from —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b} are independently selected at each occurrence from —H, —C₁-C₆ alkenyl, —C₁-C₆ alkynyl, —C₁-C₆ haloalkyl, R^{c}, and —C₁-C₆ alkyl, wherein the —C₁-C₆ alkyl can be substituted with —OR^{e}, —NR^{e}R^{f}, —C(O)OR^{e}, —C(O)NR^{e}R^{f}, —S(O)₂R^{e}, —S(O)₂NR^{e}R^{f}, or R^{c}; and
R^{c} and R^{c′} are independently selected at each occurrence from aryl, heteroaryl, heterocyclyl, cycloalkyl, or cycloalkenyl, wherein each R^{c} group can be substituted with 1, 2, 3, 4, or 5 R^{d} groups; and R^{d} is independently selected at each occurrence from —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, wherein R^{e} and R^{f} are independently selected at each occurrence from —H, —C₁-C₆ alkyl, —C₁-C₆ cycloalkyl, aryl, heteroaryl and —C₁-C₆ haloalkyl.

In certain embodiments, compounds of the present disclosure include, without limitation, compounds of Formula IV:

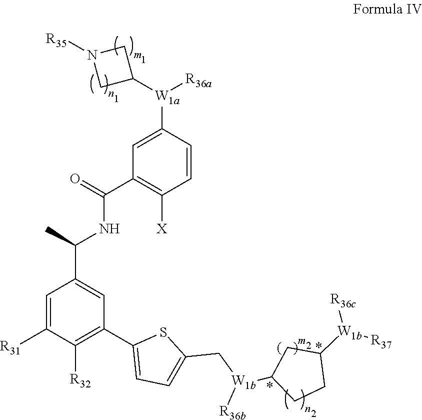

or a pharmaceutically acceptable salt thereof, wherein
X is -Me, -Et, —OMe, —CH═CH₂;
W₁ is —N, —C or —C; wherein if W₁═O, R₃₆ does not exist,
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₃, R₃₄, R₃₅, R₃₆, and R₃₇ are independently selected from the group of H, -═O, —═S, —OH, —SH, —(C₁-C₆ alkylenyl)NR^{a}R^{b}, —(C₁-C₆ alkylenyl)NC(O)R^{a}, —(C₁-C₆ alkylenyl), C(O)R², —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
R₃₇ could also be selected from PROTACs, hybrid compounds, and/or Prodrugs described before; and
m₁, m₂, n₁ and n₂=1-3.

In certain embodiments, compounds of the present disclosure include, without limitation, compounds of Formula V:

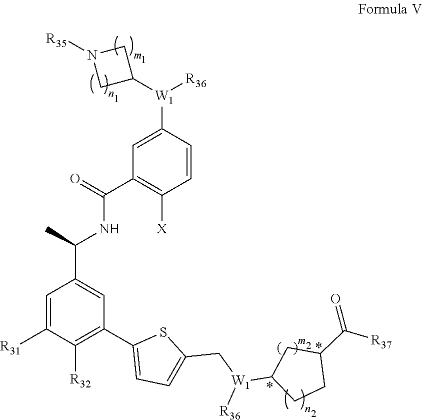

or a pharmaceutically acceptable salt thereof, wherein
X is -Me, -Et, —OMe, or —CH═CH₂;
W₁ is N or O; wherein if W₁═O, R₃₆ does not exist;
R₃₁ and R₃₂ is independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₃, R₃₄, R₃₅, R₃₆, and R₃₇ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;

R^{d}, at each occurrence, is independently selected from the group of: a C₁-C₆alkyl, a C₂-C₆ alkenyl, a C₂-C₆ alkynyl, a halogen, a C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, a —(C₁-C₆ alkylenyl)-C(O)NR^{e}R^{f}, a —(C₁-C₆ alkylenyl)-NR^{e}R^{f}, and a —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
R₃₇ could also be selected from PROTACs, hybrid compounds, and/or Prodrugs described before; and
m₁, m₂, n₁ and n₂=1-3.

In certain embodiments, compounds of the present disclosure include, without limitation, compounds of Formula VI:

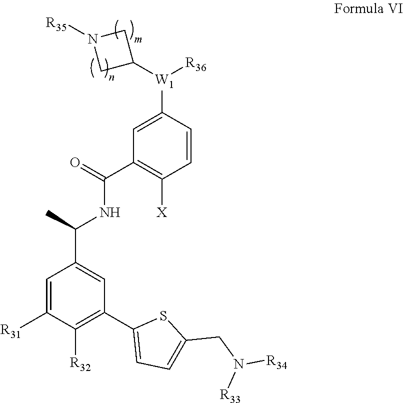

or a pharmaceutically acceptable salt thereof, wherein
X is -Me, -Et, —OMe, or —CH═CH₂;
W₁ is —N or —O; wherein if W₁═O, R₃₆ does not exist;
R₃₁ and R₃₂ are independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₃, R₃₄, R₃₅, and R₃₆ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
R₃₃ could also be selected from PROTACs, hybrid compounds, and/or Prodrugs described before; and
m and n=1-3.

In certain embodiments, compounds of the present disclosure include, without limitation, compounds of Formula VII:

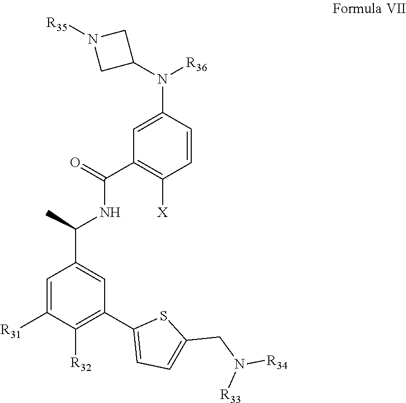

or a pharmaceutically acceptable salt thereof, wherein
X is -Me, -Et, —OMe, or —CH═CH₂;
R₃₁ and R₃₂ are independently selected from —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, N(R^{a})S(O)₂R^{b}, —S(O)₂NR^{a}R^{b}, —C(O)NR^{a}R^{b}, —N(R^{a})C(O)R^{b}, —NR^{a}R^{b}—(C₁-C₆ alkylenyl)R^{c}, —(C₁-C₃ cycloalkylenyl)R^{c}, and —(C₁-C₆ alkylenyl)R^{c}R^{c′};

R₃₃, R₃₄, R₃₅, and R₃₆ are independently selected from —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′}; R^{a} and R^{b} are independently selected at each occurrence from —H, —C₁-C₆ alkenyl, —C₁-C₆ alkynyl, —C₁-C₆ haloalkyl, —R^{c}, and —C₁-C₆ alkyl, wherein the —C₁-C₆ alkyl can be substituted with a substituent selected from —OR^{e}, —NR^{e}R^{f}, —C(O)OR^{e}, —C(O)NR^{e}R^{f}, —S(O)₂R^{e}, —S(O)₂NR^{e}R^{f}, and R^{c}, and
R^{c} and R^{c′}, at each occurrence, are each independently selected from aryl, heteroaryl, heterocyclyl, cycloalkyl, or a cycloalkenyl, wherein each R^{c} group can be substituted with 1, 2, 3, 4, or 5R^{d} groups; and R^{d} is independently selected at each occurrence from C₁—C alkyl, C₂—C alkenyl, C₂-C₆ alkynyl, halogen, C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, wherein R^{e} and R^{f} are independently selected at each occurrence from —H, —C₁-C₆ alkyl, —C₁-C₆ cycloalkyl, aryl, heteroaryl and —C₁-C₆ haloalkyl, and
R₃₃ could also be selected from PROTACs, hybrid compounds, and/or Prodrugs described before.

In certain embodiments, compounds of the present disclosure include, without limitation, compounds of Formula VIII:

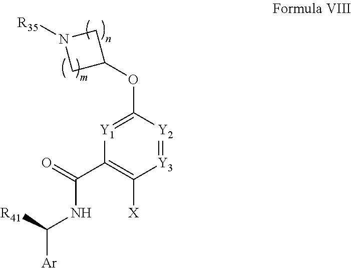

or a pharmaceutically acceptable salt thereof, wherein
X is -Me, -Et, —OMe, or —CH═CH₂;
Y₁-Y₃ are —N or —CH;
Ar is an aryl, a heteroaryl, a heterocycle, a cycloalkyl, or a cycloalkenyl, and where each Ar group can be substituted with 1, 2, 3, 4, or 5 R^{d} groups,
R₃₅ and R₄₂ are independently selected from the group of —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{G}, and —(C₁-C₆ alkylenyl)R^{c}R^{c′},
R^{a} and R^{b}, at each occurrence, are each independently selected from the group of: —H, —C₁-C₆ alkenyl, a C₁-C₆ alkynyl, a C₁-C₆ haloalkyl, R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and R^{c};
R^{c} and R^{c′}, at each occurrence, are each independently selected from: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆alkylenyl)-OR^{e},—(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m and n=1-3.

In certain embodiments, compounds of the present disclosure include, without limitation, compounds of Formula IX:

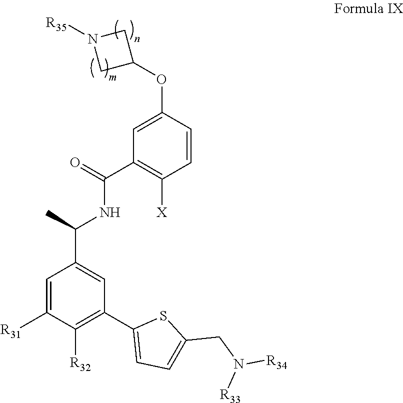

or a pharmaceutically acceptable salt thereof, wherein
X is -Me, -Et, —OMe, or —CH═CH₂;
R₃₁ and R₃₂ are independently selected from the group of —H, halogen, —(C₁-C₆ alkylenyl)NR^{a}R^{b}, —OR², —(C₁-C₆ alkylenyl)NC(O)R², —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₃, R₃₄, R₃₅, and R₃₆ are independently selected from the group of H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′},
R² and R^{b}, at each occurrence, are each independently selected from the group of: —H, —C₁-C₆ alkenyl, —C₁-C₆ alkynyl, —C₁-C₆ haloalkyl, —R^{c}, or a —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₅ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
R₃₃ could also be selected from PROTACs, hybrid compounds, and/or Prodrugs described before; and
m and n=1-3.

In certain embodiments, compounds of the present disclosure include, without limitation, compounds of Formula X:

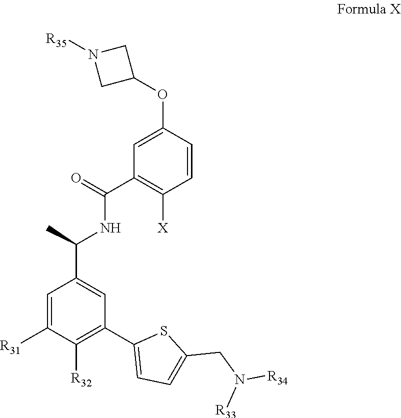

or a pharmaceutically acceptable salt thereof, wherein
X is -Me, -Et, —OMe, or —CH═CH₂;
R₃₁ and R₃₂ are independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₃, R₃₄, R₃₅, and R₃₆ are independently selected from the group of —H, —(C₁-C₆ alkylenyl)NR^{a}R^{b}, —(C₁-C₆ alkylenyl)NC(O)R^{a}, —(C₁-C₆ alkylenyl), —C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c},
R^{c} and R^{c′} at each occurrence, are each independently selected the group of: an aryl, a heteroaryl, a heterocycle, a cycloalkyl, or a cycloalkenyl, and where each R^{c} group can be substituted with 1, 2, 3, 4, or 5 R^{d} groups, where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e},—(C₁-C₆ alkylenyl)-C(O)NR^{e}R^{f},—(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
R₃₃ could also be selected from PROTACs, hybrid compounds, and/or Prodrugs described before.

In some embodiments, the compounds of Formula I-VII are selected from one or more compounds of Table 1.

TABLE 1
Exemplary Compounds of Formulas I-VII and Chemical Characterization

| | | | LC-MS |
|---|---|---|---|
| | | | characterization |
| Examples | Structure | Chemical name | (M + H⁺) |
| 1 | 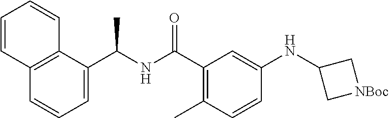 [SMILES: Cc1ccc(NC2CN(C(=O)OC(C)(C)C)C2)cc1C(=O)N[C@H](C)c1cccc2ccccc12] | Tert-butyl (R)-3-((4- methyl-3-((1-(naphthalen- 1- yl)ethyl)carbamoyl)phenyl) amino)azetidine-1- carboxylate | 460.2590 |
| 2 | 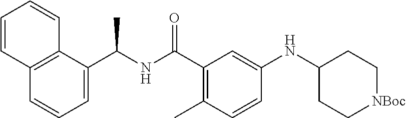 [SMILES: Cc1ccc(NC2CCN(C(=O)OC(C)(C)C)CC2)cc1C(=O)N[C@H](C)c1cccc2ccccc12] | Tert-butyl (R)-4-((4- methyl-3-((1-(naphthalen- 1- yl)ethyl)carbamoyl)phenyl) amino)piperidine-1- carboxylate | 488.2957 |
| 3 | 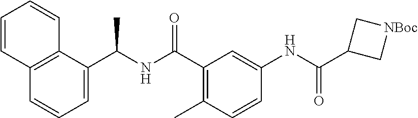 [SMILES: Cc1ccc(NC(=O)C2CN(C(=O)OC(C)(C)C)C2)cc1C(=O)N[C@H](C)c1cccc2ccccc12] | Tert-butyl (R)-3-((4- methyl-3-((1-(naphthalen- 1- yl)ethyl)carbamoyl)phenyl) carbamoyl)azetidine-1- carboxylate | 488.2534 |
| 4 | 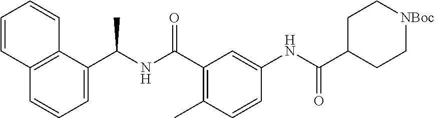 [SMILES: Cc1ccc(NC(=O)C2CCN(C(=O)OC(C)(C)C)CC2)cc1C(=O)N[C@H](C)c1cccc2ccccc12] | Tert-butyl (R)-4-((4- methyl-3-((1-(naphthalen- 1- yl)ethyl)carbamoyl)phenyl) carbamoyl)piperidine-1- carboxylate | 516.2892 |
| 5 | 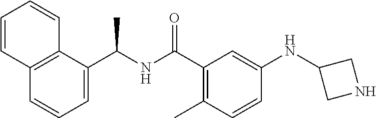 [SMILES: Cc1ccc(NC2CNC2)cc1C(=O)N[C@H](C)c1cccc2ccccc12] | (R)-5-(azetidin-3-ylamino)- 2-methyl-N-(1- (naphthalen-1- yl)ethyl)benzamide | 360.2076 |
| 6 | 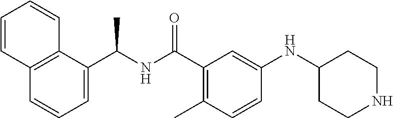 [SMILES: Cc1ccc(NC2CCNCC2)cc1C(=O)N[C@H](C)c1cccc2ccccc12] | (R)-2-methyl-N-(1- (naphthalen-1-yl)ethyl)-5- (piperidin-4- ylamino)benzamide | 388.2389 |
| 7 | 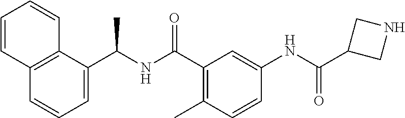 [SMILES: Cc1ccc(NC(=O)C2CNC2)cc1C(=O)N[C@H](C)c1cccc2ccccc12] | (R)-N-(4-methyl-3-((1- (naphthalen-1- yl)ethyl)carbamoyl)phenyl) azetidine-3-carboxamide | 388.2028 |
| 8 | 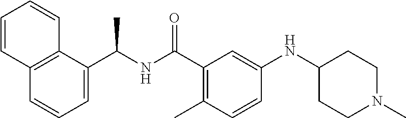 [SMILES: Cc1ccc(NC2CCN(C)CC2)cc1C(=O)N[C@H](C)c1cccc2ccccc12] | (R)-2-methyl-5-((1- methylpiperidin-4- yl)amino)-N-(1- (naphthalen-1- yl)ethyl)benzamide | 416.2545 |
| 9 | 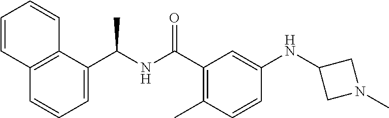 [SMILES: Cc1ccc(NC2CN(C)C2)cc1C(=O)N[C@H](C)c1cccc2ccccc12] | (R)-2-methyl-5-((1- methylazetidin-3- yl)amino)-N-(1- (naphthalen-1- yl)ethyl)benzamide | 388.2230 |
| 10 | 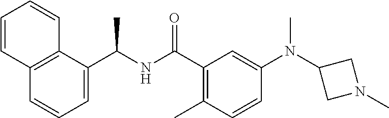 [SMILES: Cc1ccc(N(C)C2CN(C)C2)cc1C(=O)N[C@H](C)c1cccc2ccccc12] | (R)-2-methyl-5-(methyl(1- methylazetidin-3- yl)amino)-N-(1- (naphthalen-1- yl)ethyl)benzamide | 388.2389 |
| 11 | 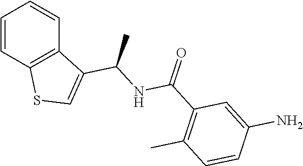 [SMILES: Cc1ccc(N)cc1C(=O)N[C@H](C)c1csc2ccccc12] | (R)-5-amino-N-(1- (benzo[b]thiophen-3- yl)ethyl)-2- methylbenzamide | 324.1341 |
| 12 | 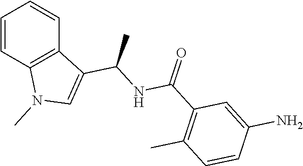 [SMILES: Cc1ccc(N)cc1C(=O)N[C@H](C)c1cn(C)c2ccccc12] | (R)-5-amino-2-methyl-N- (1-(1-methyl-1H-indol-3- yl)ethyl)benzamide | 308.1753 |
| 13 | 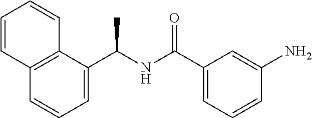 [SMILES: C[C@@H](NC(=O)c1cccc(N)c1)c1cccc2ccccc12] | (R)-3-(((1-(naphthalen-1- yl)ethyl)amino)methyl)aniline | 277.1709 |
| 14 | 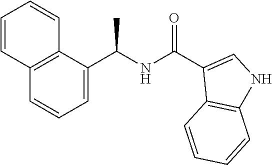 [SMILES: C[C@@H](NC(=O)c1c[nH]c2ccccc12)c1cccc2ccccc12] | (R)-N-(1-(naphthalen-1- yl)ethyl)-1H-indole-3- carboxamide | 315.1498 |
| 15 | 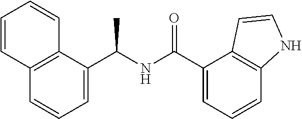 [SMILES: C[C@@H](NC(=O)c1cccc2[nH]ccc12)c1cccc2ccccc12] | (R)-N-(1-(naphthalen-1- yl)ethyl)-1H-indole-4- carboxamide | 315.1491 |
| 16 | 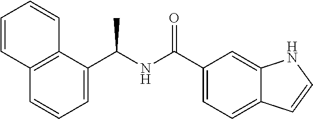 [SMILES: C[C@@H](NC(=O)c1ccc2cc[nH]c2c1)c1cccc2ccccc12] | (R)-N-(1-(naphthalen-1- yl)ethyl)-1H-indole-6- carboxamide | 315.1498 |
| 17 | 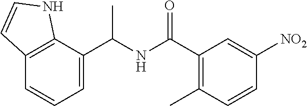 [SMILES: Cc1ccc([N+](=O)[O-])cc1C(=O)NC(C)c1cccc2cc[nH]c12] | N-(1-(1H-indol-7-yl)ethyl)- 2-methyl-5- nitrobenzamide | 311.1215 |
| 18 | 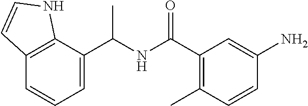 [SMILES: Cc1ccc(N)cc1C(=O)NC(C)c1cccc2cc[nH]c12] | N-(1-(1H-indol-7-yl)ethyl)- 5-amino-2- methylbenzamide | 294.1606 |
| 19 | 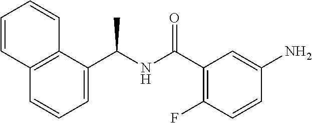 [SMILES: C[C@@H](NC(=O)c1cc(N)ccc1F)c1cccc2ccccc12] | (R)-5-amino-2-fluoro-N-(1- (naphthalen-1- yl)ethyl)benzamide | 309.1404 |
| 20 | 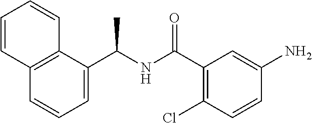 [SMILES: C[C@@H](NC(=O)c1cc(N)ccc1Cl)c1cccc2ccccc12] | (R)-5-amino-2-chloro-N- (1-(naphthalen-1- yl)ethyl)benzamide | 325.1101 |
| 21 | 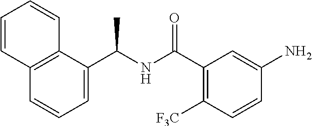 [SMILES: C[C@@H](NC(=O)c1cc(N)ccc1C(F)(F)F)c1cccc2ccccc12] | (R)-5-amino-N-(1- (naphthalen-1-yl)ethyl)-2- (trifluoromethyl)benzamide | 359.1371 |
| 22 | 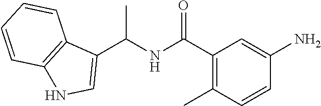 [SMILES: Cc1ccc(N)cc1C(=O)NC(C)c1c[nH]c2ccccc12] | N-(1-(1H-indol-3-yl)ethyl)- 5-amino-2- methylbenzamide | 294.1649 |
| 23 | 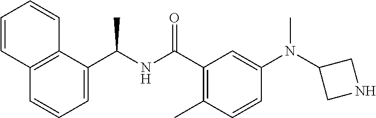 [SMILES: Cc1ccc(N(C)C2CNC2)cc1C(=O)N[C@H](C)c1cccc2ccccc12] | (R)-5-(azetidin-3- yl(methyl)amino)-2- methyl-N-(1-(naphthalen- 1-yl)ethyl)benzamide | 374.2231 |
| 24 | 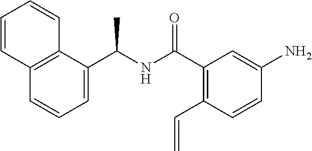 [SMILES: C=Cc1ccc(N)cc1C(=O)N[C@H](C)c1cccc2ccccc12] | (R)-5-amino-N-(1- (naphthalen-1-yl)ethyl)-2- vinylbenzamide | 374.2231 |
| 25 | 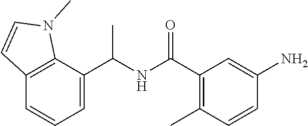 [SMILES: Cc1ccc(N)cc1C(=O)NC(C)c1cccc2ccn(C)c12] | 5-amino-2-methyl-N-(1-(1- methyl-1H-indol-7- yl)ethyl)benzamide | 308.1709 |
| 26 | 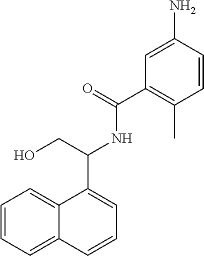 [SMILES: Cc1ccc(N)cc1C(=O)NC(CO)c1cccc2ccccc12] | 5-amino-N-(2-hydroxy-1- (naphthalen-1-yl)ethyl)-2- methylbenzamide | 308.1589 |
| 27 | 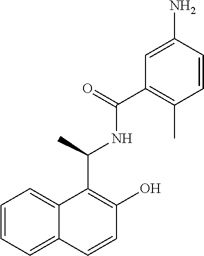 [SMILES: Cc1ccc(N)cc1C(=O)N[C@H](C)c1c(O)ccc2ccccc12] | (R)-5-amino-N-(1-(2- hydroxynaphthalen-1- yl)ethyl)-2- methylbenzamide | 321.1599 |
| 28 | 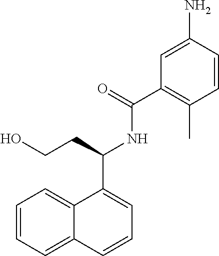 [SMILES: Cc1ccc(N)cc1C(=O)N[C@H](CCO)c1cccc2ccccc12] | (R)-5-amino-N-(3- hydroxy-1-(naphthalen-1- yl)propyl)-2- methylbenzamide | 335.1760 |
| 29 | 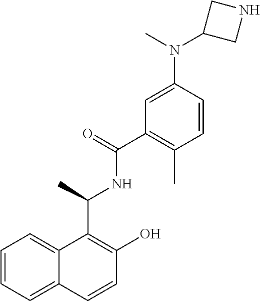 [SMILES: Cc1ccc(N(C)C2CNC2)cc1C(=O)N[C@H](C)c1c(O)ccc2ccccc12] | (R)-5-(azetidin-3- yl(methyl)amino)-N-(1-(2- hydroxynaphthalen-1- yl)ethyl)-2- methylbenzamide | 390.2185 |
| 30 | 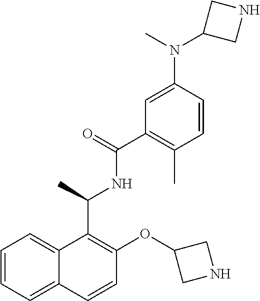 [SMILES: Cc1ccc(N(C)C2CNC2)cc1C(=O)N[C@H](C)c1c(OC2CNC2)ccc2ccccc12] | (R)-5-(azetidin-3- yl(methyl)amino)-N-(1-(2- (azetidin-3- yloxy)naphthalen-1- yl)ethyl)-2- methylbenzamide | 445.2587 |
| 31 | 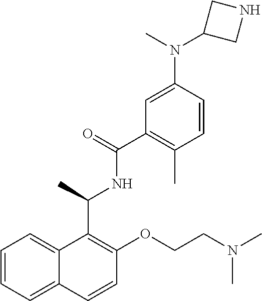 [SMILES: Cc1ccc(N(C)C2CNC2)cc1C(=O)N[C@H](C)c1c(OCCN(C)C)ccc2ccccc12] | (R)-5-(azetidin-3- yl(methyl)amino)-N-(1-(2- (2- (dimethylamino)ethoxy) naphthalen-1-yl)ethyl)-2- methylbenzamide | 461.2918 |
| 32 | 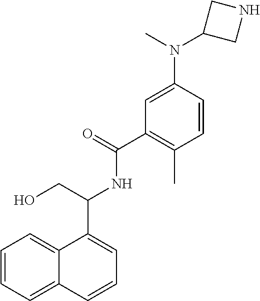 [SMILES: Cc1ccc(N(C)C2CNC2)cc1C(=O)NC(CO)c1cccc2ccccc12] | 5-(azetidin-3- yl(methyl)amino)-N-(2- hydroxy-1-(naphthalen-1- yl)ethyl)-2- methylbenzamide | 461.2918 |
| 33 | 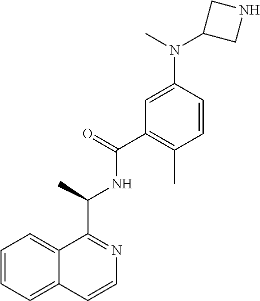 [SMILES: Cc1ccc(N(C)C2CNC2)cc1C(=O)N[C@H](C)c1nccc2ccccc12] | (R)-5-(azetidin-3- yl(methyl)amino)-N-(1- (isoquinolin-1-yl)ethyl)-2- methylbenzamide | 375.2183 |
| 34 | 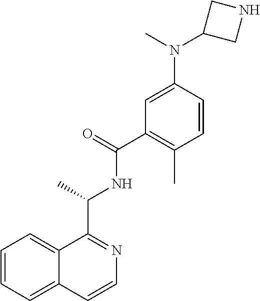 [SMILES: Cc1ccc(N(C)C2CNC2)cc1C(=O)N[C@@H](C)c1nccc2ccccc12] | (S)-5-(azetidin-3- yl(methyl)amino)-N-(1- (isoquinolin-1-yl)ethyl)-2- methylbenzamide | 375.2185 |
| 35 | 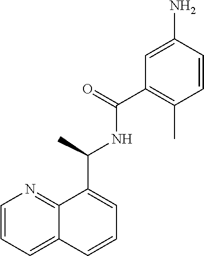 [SMILES: Cc1ccc(N)cc1C(=O)N[C@H](C)c1cccc2cccnc12] | (R)-5-amino-2-methyl-N- (1-(quinolin-8- yl)ethyl)benzamide | 306.1600 |
| 36 | 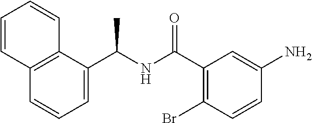 [SMILES: C[C@@H](NC(=O)c1cc(N)ccc1Br)c1cccc2ccccc12] | (R)-5-amino-2-bromo-N- (1-(naphthalen-1- yl)ethyl)benzamide | 369.0599 |
| 37 | 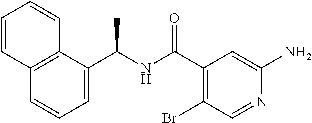 [SMILES: C[C@@H](NC(=O)c1cc(N)ncc1Br)c1cccc2ccccc12] | (R)-2-amino-5-bromo-N- (1-(naphthalen-1- yl)ethyl)isonicotinamide | 370.0556 |
| 38 | 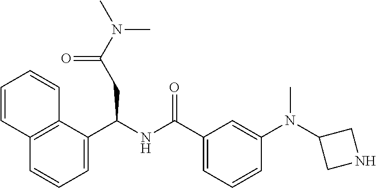 [SMILES: CN(C)C(=O)C[C@@H](NC(=O)c1cccc(N(C)C2CNC2)c1)c1cccc2ccccc12] | (R)-3-(azetidin-3- yl(methyl)amino)-N-(3- (dimethylamino)-1- (naphthalen-1-yl)-3- oxopropyl)benzamide | 432.2437 |
| 39 | 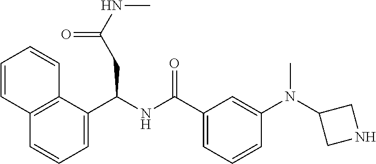 [SMILES: CNC(=O)C[C@@H](NC(=O)c1cccc(N(C)C2CNC2)c1)c1cccc2ccccc12] | (R)-3-(azetidin-3- yl(methyl)amino)-N-(3- (methylamino)-1- (naphthalen-1-yl)-3- oxopropyl)benzamide | 417.2290 |
| 40 | 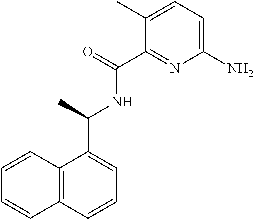 [SMILES: Cc1ccc(N)nc1C(=O)N[C@H](C)c1cccc2ccccc12] | (R)-6-amino-3-methyl-N- (1-(naphthalen-1- yl)ethyl)picolinamide | 306.1602 |
| 41 | 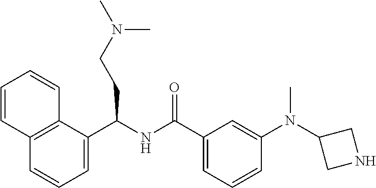 [SMILES: CN(C)CC[C@@H](NC(=O)c1cccc(N(C)C2CNC2)c1)c1cccc2ccccc12] | (R)-3-(azetidin-3- yl(methyl)amino)-N-(3- (dimethylamino)-1- (naphthalen-1- yl)propyl)benzamide | 417.2650 |
| 42 | 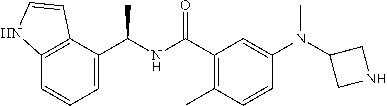 [SMILES: Cc1ccc(N(C)C2CNC2)cc1C(=O)N[C@H](C)c1cccc2[nH]ccc12] | (R)-N-(1-(1H-indol-4- yl)ethyl)-5-(azetidin-3- yl(methyl)amino)-2- methylbenzamide | 363.2183 |
| 43 | 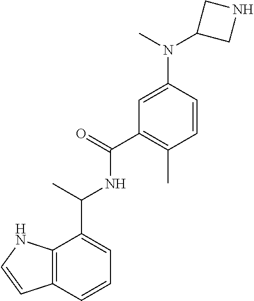 [SMILES: Cc1ccc(N(C)C2CNC2)cc1C(=O)NC(C)c1cccc2cc[nH]c12] | N-(1-(1H-indol-7-yl)ethyl)- 5-(azetidin-3- yl(methyl)amino)-2- methylbenzamide | 363.2179 |
| 44 | 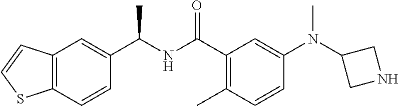 [SMILES: Cc1ccc(N(C)C2CNC2)cc1C(=O)N[C@H](C)c1ccc2sccc2c1] | (R)-5-(azetidin-3- yl(methyl)amino)-N-(1- (benzo[b]thiophen-5- yl)ethyl)-2- methylbenzamide | 380.1798 |
| 45 | 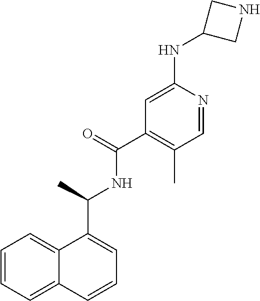 [SMILES: Cc1cnc(NC2CNC2)cc1C(=O)N[C@H](C)c1cccc2ccccc12] | (R)-2-(azetidin-3-ylamino)- 5-methyl-N-(1- (naphthalen-1- yl)ethyl)isonicotinamide | 361.2024 |
| 46 | 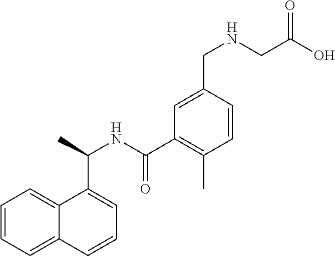 [SMILES: Cc1ccc(CNCC(=O)O)cc1C(=O)N[C@H](C)c1cccc2ccccc12] | (R)-(4-methyl-3-((1- (naphthalen-1- yl)ethyl)carbamoyl)benzyl) glycine | 377.1857 |
| 47 | 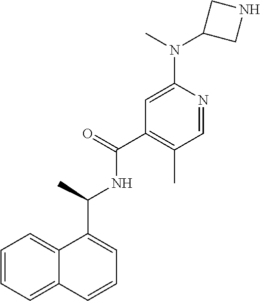 [SMILES: Cc1cnc(N(C)C2CNC2)cc1C(=O)N[C@H](C)c1cccc2ccccc12] | (R)-2-(azetidin-3- yl(methyl)amino)-5- methyl-N-(1-(naphthalen- 1-yl)ethyl)isonicotinamide | 375.2185 |
| 48 | 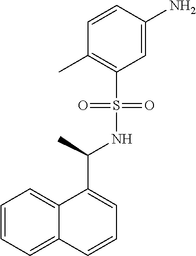 [SMILES: Cc1ccc(N)cc1S(=O)(=O)N[C@H](C)c1cccc2ccccc12] | (R)-5-amino-2-methyl-N- (1-(naphthalen-1- yl)ethyl)benzenesulfonamide | 341.1314 |
| 49 | 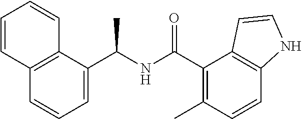 [SMILES: Cc1ccc2[nH]ccc2c1C(=O)N[C@H](C)c1cccc2ccccc12] | (R)-5-methyl-N-(1- (naphthalen-1-yl)ethyl)- 1H-indole-4-carboxamide | 329.1649 |
| 50 | 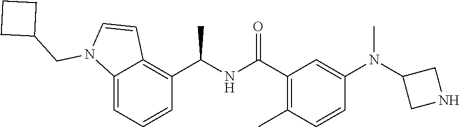 [SMILES: Cc1ccc(N(C)C2CNC2)cc1C(=O)N[C@H](C)c1cccc2c1ccn2CC1CCC1] | (R)-5-(azetidin-3- yl(methyl)amino)-N-(1-(1- (cyclobutylmethyl)-1H- indol-4-yl)ethyl)-2- methylbenzamide | 431.2811 |
| 51 | 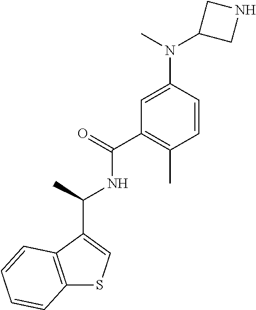 [SMILES: Cc1ccc(N(C)C2CNC2)cc1C(=O)N[C@H](C)c1csc2ccccc12] | (R)-5-(azetidin-3- yl(methyl)amino)-N-(1- (benzo[b]thiophen-3- yl)ethyl)-2- methylbenzamide | 380.1791 |
| 52 | 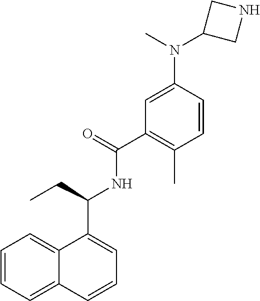 [SMILES: CC[C@@H](NC(=O)c1cc(N(C)C2CNC2)ccc1C)c1cccc2ccccc12] | (R)-5-(azetidin-3- yl(methyl)amino)-2- methyl-N-(1-(naphthalen- 1-yl)propyl)benzamide | 388.2389 |
| 53 | 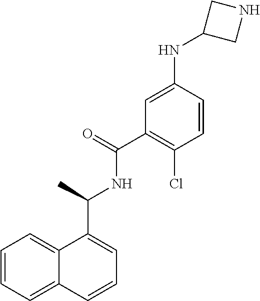 [SMILES: C[C@@H](NC(=O)c1cc(NC2CNC2)ccc1Cl)c1cccc2ccccc12] | (R)-5-(azetidin-3-ylamino)- 2-chloro-N-(1- (naphthalen-1- yl)ethyl)benzamide | 388.1532 |
| 54 | 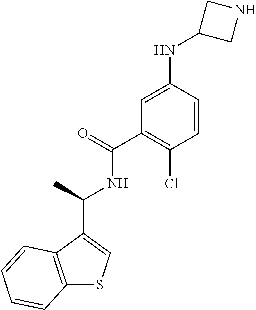 [SMILES: C[C@@H](NC(=O)c1cc(NC2CNC2)ccc1Cl)c1csc2ccccc12] | (R)-5-(azetidin-3-ylamino)- N-(1-(benzo[b]thiophen-3- yl)ethyl)-2- chlorobenzamide | 386.1094 |
| 55 | 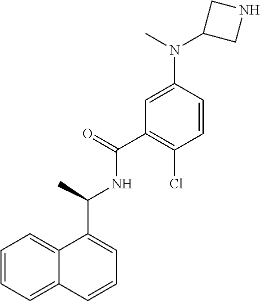 [SMILES: C[C@@H](NC(=O)c1cc(N(C)C2CNC2)ccc1Cl)c1cccc2ccccc12] | (R)-5-(azetidin-3- yl(methyl)amino)-2-chloro- N-(1-(naphthalen-1- yl)ethyl)benzamide | 394.1677 |
| 56 | 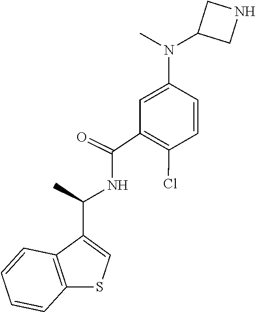 [SMILES: C[C@@H](NC(=O)c1cc(N(C)C2CNC2)ccc1Cl)c1csc2ccccc12] | (R)-5-(azetidin-3- yl(methyl)amino)-N-(1- (benzo[b]thiophen-3- yl)ethyl)-2- chlorobenzamide | 400.1252 |
| 57 | 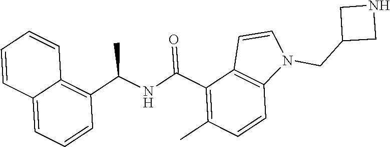 [SMILES: Cc1ccc2c(ccn2CC2CNC2)c1C(=O)N[C@H](C)c1cccc2ccccc12] | (R)-1-(azetidin-3- ylmethyl)-5-methyl-N-(1- (naphthalen-1-yl)ethyl)- 1H-indole-4-carboxamide | 398.2230 |
| 58 | 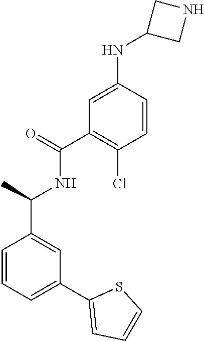 [SMILES: C[C@@H](NC(=O)c1cc(NC2CNC2)ccc1Cl)c1cccc(-c2cccs2)c1] | (R)-5-(azetidin-3-ylamino)- 2-chloro-N-(1-(3- (thiophen-2- yl)phenyl)ethyl)benzamide | 412.1250 |
| 59 | 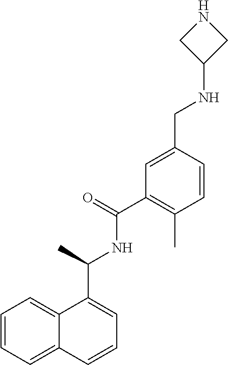 [SMILES: Cc1ccc(CNC2CNC2)cc1C(=O)N[C@H](C)c1cccc2ccccc12] | (R)-5-((azetidin-3- ylamino)methyl)-2-methyl- N-(1-(naphthalen-1- yl)ethyl)benzamide | 374.2231 |
| 60 | 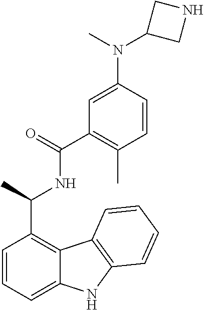 [SMILES: Cc1ccc(N(C)C2CNC2)cc1C(=O)N[C@H](C)c1cccc2[nH]c3ccccc3c12] | (R)-N-(1-(9H-carbazol-4- yl)ethyl)-5-(azetidin-3- yl(methyl)amino)-2- methylbenzamide | 413.2333 |
| 61 | 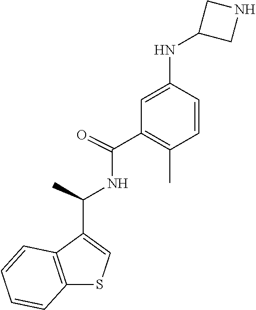 [SMILES: Cc1ccc(NC2CNC2)cc1C(=O)N[C@H](C)c1csc2ccccc12] | (R)-5-(azetidin-3-ylamino)- N-(1-(benzo[b]thiophen-3- yl)ethyl)-2- methylbenzamide | 366.1640 |
| 62 | 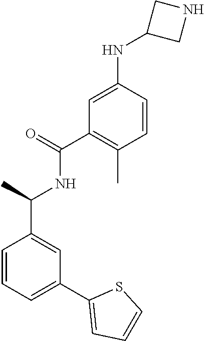 [SMILES: Cc1ccc(NC2CNC2)cc1C(=O)N[C@H](C)c1cccc(-c2cccs2)c1] | (R)-5-(azetidin-3-ylamino)- 2-methyl-N-(1-(3- (thiophen-2- yl)phenyl)ethyl)benzamide | 392.1790 |
| 63 | 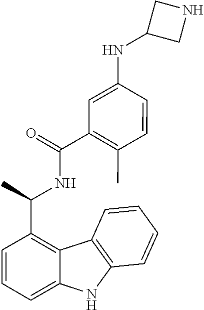 [SMILES: Cc1ccc(NC2CNC2)cc1C(=O)N[C@H](C)c1cccc2[nH]c3ccccc3c12] | (R)-N-(1-(9H-carbazol-4- yl)ethyl)-5-(azetidin-3- ylamino)-2- methylbenzamide | 399.27 |
| 64 | 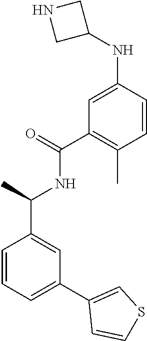 [SMILES: Cc1ccc(NC2CNC2)cc1C(=O)N[C@H](C)c1cccc(-c2ccsc2)c1] | (R)-5-(azetidin-3-ylamino)- 2-methyl-N-(1-(3- (thiophen-3- yl)phenyl)ethyl)benzamide | 392.11 |
| 65 | 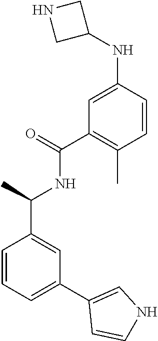 [SMILES: Cc1ccc(NC2CNC2)cc1C(=O)N[C@H](C)c1cccc(-c2cc[nH]c2)c1] | (R)-N-(1-(3-(1H-pyrrol-3- yl)phenyl)ethyl)-5- (azetidin-3-ylamino)-2- methylbenzamide | 375.22 |
| 66 | 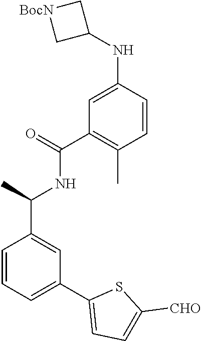 [SMILES: Cc1ccc(NC2CN(C(=O)OC(C)(C)C)C2)cc1C(=O)N[C@H](C)c1cccc(-c2ccc(C=O)s2)c1] | Tert-butyl (R)-3-((3-((1-(3- (5-formylthiophen-2- yl)phenyl)ethyl)carbamoyl)-4- methylphenyl)amino) azetidine-1-carboxylate | 520.19 |
| 67 | 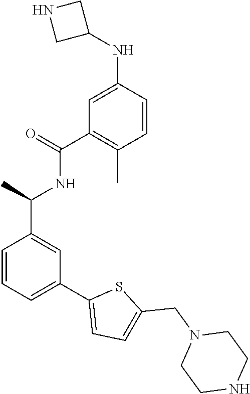 [SMILES: Cc1ccc(NC2CNC2)cc1C(=O)N[C@H](C)c1cccc(-c2ccc(CN3CCNCC3)s2)c1] | (R)-5-(azetidin-3-ylamino)- 2-methyl-N-(1-(3-(5- (piperazin-1- ylmethyl)thiophen-2- yl)phenyl)ethyl)benzamide | 490.22 |
| 68 | 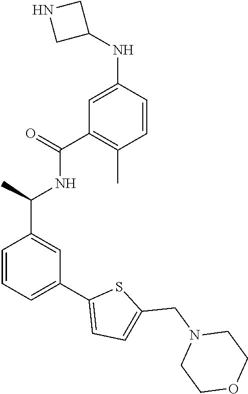 [SMILES: Cc1ccc(NC2CNC2)cc1C(=O)N[C@H](C)c1cccc(-c2ccc(CN3CCOCC3)s2)c1] | (R)-5-(azetidin-3-ylamino)- 2-methyl-N-(1-(3-(5- (morpholinomethyl) thiophen-2- yl)phenyl)ethyl)benzamide | 491.26 |
| 69 | 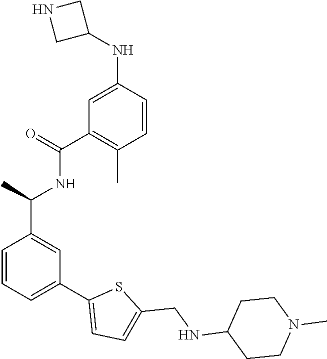 [SMILES: Cc1ccc(NC2CNC2)cc1C(=O)N[C@H](C)c1cccc(-c2ccc(CNC3CCN(C)CC3)s2)c1] | (R)-5-(azetidin-3-ylamino)- 2-methyl-N-(1-(3-(5-(((1- methylpiperidin-4- yl)amino)methyl)thiophen- 2- yl)phenyl)ethyl)benzamide | 518.31 |
| 70 | 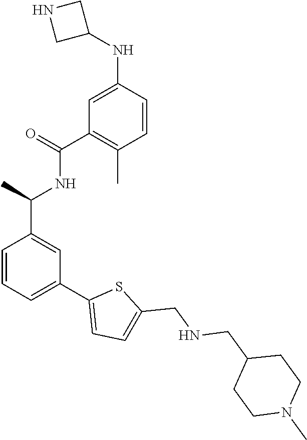 [SMILES: Cc1ccc(NC2CNC2)cc1C(=O)N[C@H](C)c1cccc(-c2ccc(CNCC3CCN(C)CC3)s2)c1] | (R)-5-(azetidin-3-ylamino)- 2-methyl-N-(1-(3-(5-((((1- methylpiperidin-4- yl)methyl)amino)methyl) thiophen-2- yl)phenyl)ethyl)benzamide | 532.30 |
| 71 | 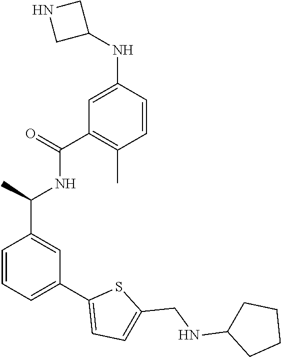 [SMILES: Cc1ccc(NC2CNC2)cc1C(=O)N[C@H](C)c1cccc(-c2ccc(CNC3CCCC3)s2)c1] | (R)-5-(azetidin-3-ylamino)- N-(1-(3-(5- ((cyclopentylamino)methyl) thiophen-2- yl)phenyl)ethyl)-2- methylbenzamide | 489.26 |
| 72 | 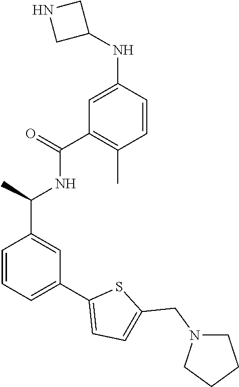 [SMILES: Cc1ccc(NC2CNC2)cc1C(=O)N[C@H](C)c1cccc(-c2ccc(CN3CCCC3)s2)c1] | (R)-5-(azetidin-3-ylamino)- 2-methyl-N-(1-(3-(5- (pyrrolidin-1- ylmethyl)thiophen-2- yl)phenyl)ethyl)benzamide | 475.22 |
| 73 | 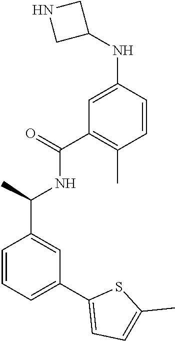 [SMILES: Cc1ccc(-c2cccc([C@@H](C)NC(=O)c3cc(NC4CNC4)ccc3C)c2)s1] | (R)-5-(azetidin-3-ylamino)- 2-methyl-N-(1-(3-(5- methylthiophen-2- yl)phenyl)ethyl)benzamide | 406.22 |
| 74 | 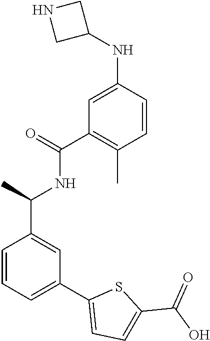 [SMILES: Cc1ccc(NC2CNC2)cc1C(=O)N[C@H](C)c1cccc(-c2ccc(C(=O)O)s2)c1] | (R)-5-(3-(1-(5-(azetidin-3- ylamino)-2- methylbenzamido)ethyl) phenyl)thiophene-2- carboxylic acid | 436.11 |
| 75 | 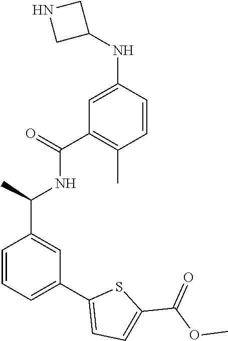 [SMILES: COC(=O)c1ccc(-c2cccc([C@@H](C)NC(=O)c3cc(NC4CNC4)ccc3C)c2)s1] | Methyl (R)-5-(3-(1-(5- (azetidin-3-ylamino)-2- methylbenzamido)ethyl) phenyl)thiophene-2- carboxylate | 450.12 |
| 76 | 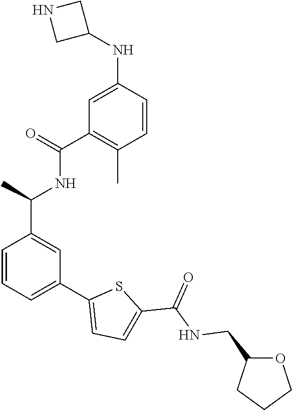 | 5-(3-((R)-1-(5-(azetidin-3- ylamino)-2- methylbenzamido)ethyl) phenyl)-N-(((R)- tetrahydrofuran-2- yl)methyl)thiophene-2- carboxamide | 519.27 |
| 77 | 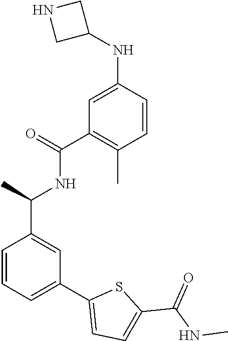 [SMILES: CNC(=O)c1ccc(-c2cccc([C@@H](C)NC(=O)c3cc(NC4CNC4)ccc3C)c2)s1] | (R)-5-(3-(1-(5-(azetidin-3- ylamino)-2- methylbenzamido)ethyl) phenyl)-N-methylthiophene- 2-carboxamide | 449.19 |
| 78 | 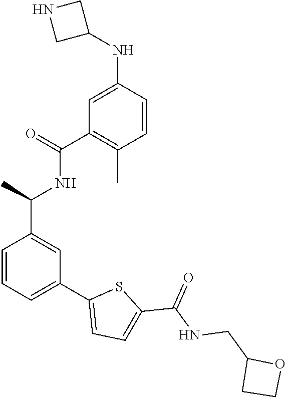 [SMILES: Cc1ccc(NC2CNC2)cc1C(=O)N[C@H](C)c1cccc(-c2ccc(C(=O)NCC3CCO3)s2)c1] | 5-(3-((R)-1-(5-(azetidin-3- ylamino)-2- methylbenzamido)ethyl) phenyl)-N-(oxetan-2- ylmethyl)thiophene-2- carboxamide | 505.24 |
| 79 | 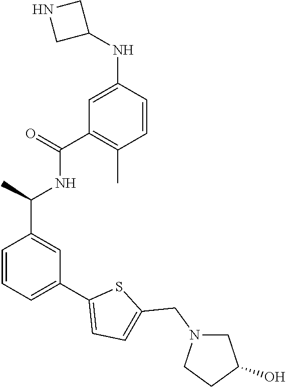 [SMILES: Cc1ccc(NC2CNC2)cc1C(=O)N[C@H](C)c1cccc(-c2ccc(CN3CC[C@@H](O)C3)s2)c1] | 5-(azetidin-3-ylamino)-N- ((R)-1-(3-(5-(((R)-3- hydroxypyrrolidin-1- yl)methyl)thiophen-2- yl)phenyl)ethyl)-2- methylbenzamide | 491.22 |
| 80 | 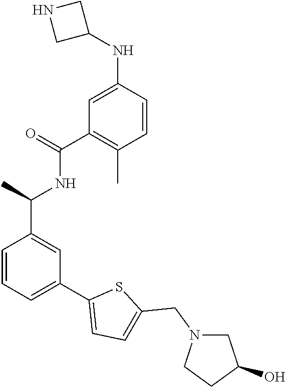 [SMILES: Cc1ccc(NC2CNC2)cc1C(=O)N[C@H](C)c1cccc(-c2ccc(CN3CC[C@H](O)C3)s2)c1] | 5-(azetidin-3-ylamino)-N- ((R)-1-(3-(5-(((S)-3- hydroxypyrrolidin-1- yl)methyl)thiophen-2- yl)phenyl)ethyl)-2- methylbenzamide | 491.23 |
| 81 | 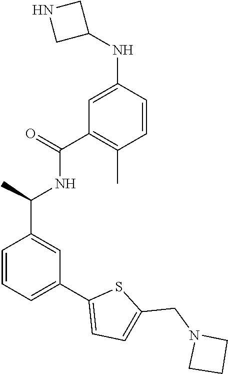 [SMILES: Cc1ccc(NC2CNC2)cc1C(=O)N[C@H](C)c1cccc(-c2ccc(CN3CCC3)s2)c1] | (R)-N-(1-(3-(5-(azetidin-1- ylmethyl)thiophen-2- yl)phenyl)ethyl)-5- (azetidin-3-ylamino)-2- methylbenzamide | 491.27 |
| 82 | 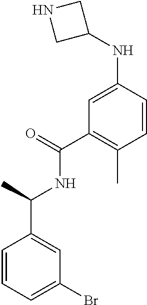 [SMILES: Cc1ccc(NC2CNC2)cc1C(=O)N[C@H](C)c1cccc(Br)c1] | (R)-5-(azetidin-3-ylamino)- N-(1-(3- bromophenyl)ethyl)-2- methylbenzamide | 388.13 |
| 83 | 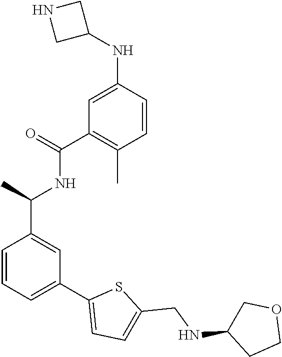 [SMILES: Cc1ccc(NC2CNC2)cc1C(=O)N[C@H](C)c1cccc(-c2ccc(CN[C@@H]3CCOC3)s2)c1] | 5-(azetidin-3-ylamino)-2- methyl-N-((R)-1-(3-(5- ((((R)-tetrahydrofuran-3- yl)amino)methyl)thiophen- 2- yl)phenyl)ethyl)benzamide | 491.26 |
| 84 | 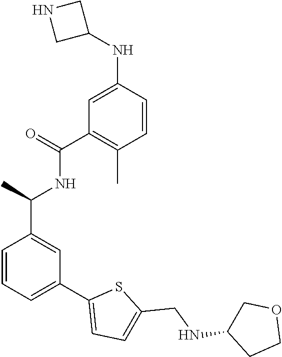 [SMILES: Cc1ccc(NC2CNC2)cc1C(=O)N[C@H](C)c1cccc(-c2ccc(CN[C@H]3CCOC3)s2)c1] | 5-(azetidin-3-ylamino)-2- methyl-N-((R)-1-(3-(5- ((((S)-tetrahydrofuran-3- yl)amino)methyl)thiophen- 2- yl)phenyl)ethyl)benzamide | 491.26 |
| 85 | 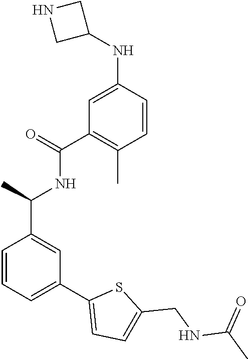 [SMILES: CC(=O)NCc1ccc(-c2cccc([C@@H](C)NC(=O)c3cc(NC4CNC4)ccc3C)c2)s1] | (R)-N-(1-(3-(5- (acetamidomethyl)thiophen- 2-yl)phenyl)ethyl)-5- (azetidin-3-ylamino)-2- methylbenzamide | 463.27 |
| 86 | 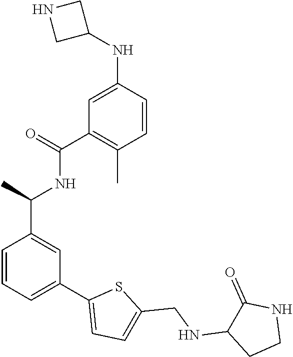 [SMILES: Cc1ccc(NC2CNC2)cc1C(=O)N[C@H](C)c1cccc(-c2ccc(CNC3CCNC3=O)s2)c1] | 5-(azetidin-3-ylamino)-2- methyl-N-((1R)-1-(3-(5- (((2-oxopyrrolidin-3- yl)amino)methyl)thiophen- 2- yl)phenyl)ethyl)benzamide | 504.21 |
| 87 | 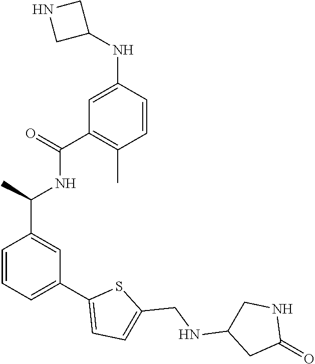 [SMILES: Cc1ccc(NC2CNC2)cc1C(=O)N[C@H](C)c1cccc(-c2ccc(CNC3CNC(=O)C3)s2)c1] | 5-(azetidin-3-ylamino)-2- methyl-N-((1R)-1-(3-(5- (((5-oxopyrrolidin-3- yl)amino)methyl)thiophen-2- yl)phenyl)ethyl)benzamide | 504.25 |
| 88 | 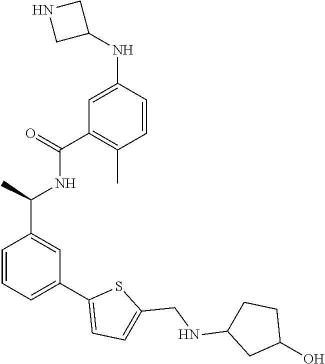 [SMILES: Cc1ccc(NC2CNC2)cc1C(=O)N[C@H](C)c1cccc(-c2ccc(CNC3CCC(O)C3)s2)c1] | 5-(azetidin-3-ylamino)-N- ((1R)-1-(3-(5-(((3- hydroxycyclopentyl)amino) methyl)thiophen-2- yl)phenyl)ethyl)-2- methylbenzamide | 505.22 |
| 89 | 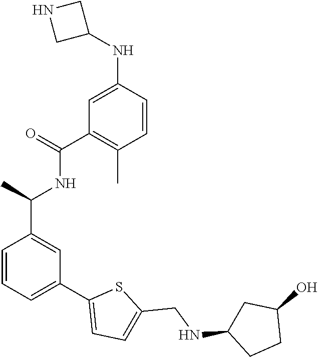 [SMILES: Cc1ccc(NC2CNC2)cc1C(=O)N[C@H](C)c1cccc(-c2ccc(CN[C@@H]3CC[C@H](O)C3)s2)c1] | 5-(azetidin-3-ylamino)-N- ((R)-1-(3-(5-((((1R,3S)-3- hydroxycyclopentyl)amino) methyl)thiophen-2- yl)phenyl)ethyl)-2- methylbenzamide | 505.27 |
| 90 | 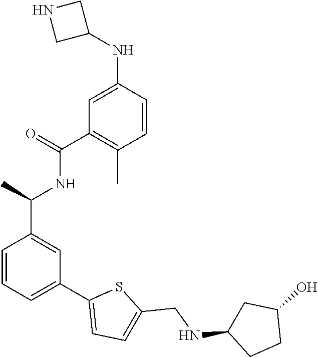 [SMILES: Cc1ccc(NC2CNC2)cc1C(=O)N[C@H](C)c1cccc(-c2ccc(CN[C@@H]3CC[C@@H](O)C3)s2)c1] | 5-(azetidin-3-ylamino)-N- ((R)-1-(3-(5-((((1R,3R)-3- hydroxycyclopentyl)amino )methyl)thiophen-2- yl)phenyl)ethyl)-2- methylbenzamide | 505.25 |
| 91 | 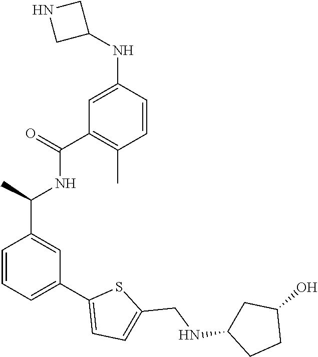 [SMILES: Cc1ccc(NC2CNC2)cc1C(=O)N[C@H](C)c1cccc(-c2ccc(CN[C@H]3CC[C@@H](O)C3)s2)c1] | 5-(azetidin-3-ylamino)-N- ((R)-1-(3-(5-((((1S,3R)-3- hydroxycyclopentyl)amino) methyl)thiophen-2- yl)phenyl)ethyl)-2- methylbenzamide | 505.27 |
| 92 | 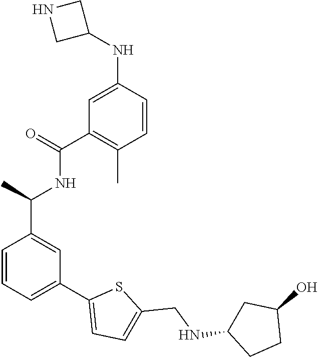 [SMILES: Cc1ccc(NC2CNC2)cc1C(=O)N[C@H](C)c1cccc(-c2ccc(CN[C@H]3CC[C@H](O)C3)s2)c1] | 5-(azetidin-3-ylamino)-N- ((R)-1-(3-(5-((((1S,3S)-3- hydroxycyclopentyl)amino) methyl)thiophen-2- yl)phenyl)ethyl)-2- methylbenzamide | 505.27 |
| 93 | 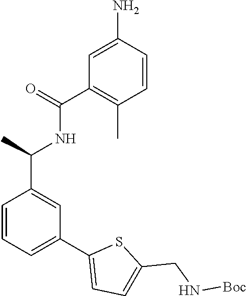 [SMILES: Cc1ccc(N)cc1C(=O)N[C@H](C)c1cccc(-c2ccc(CNC(=O)OC(C)(C)C)s2)c1] | (R)-3-((1-(3-(5-(((tert- butoxycarbonyl)amino)me thyl)thiophen-2- yl)phenyl)ethyl)carbamoyl)- 4-methylbenzenaminium | 466.23 |
| 94 | 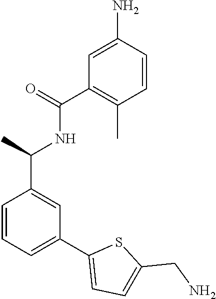 [SMILES: Cc1ccc(N)cc1C(=O)N[C@H](C)c1cccc(-c2ccc(CN)s2)c1] | (R)-5-amino-N-(1-(3-(5- (aminomethyl)thiophen-2- yl)phenyl)ethyl)-2- methylbenzamide | 366.17 |
| 95 | 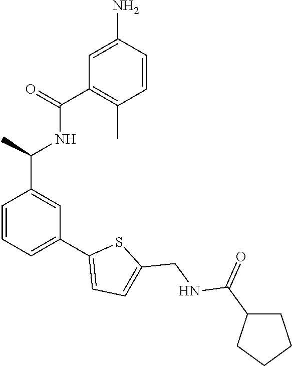 [SMILES: Cc1ccc(N)cc1C(=O)N[C@H](C)c1cccc(-c2ccc(CNC(=O)C3CCCC3)s2)c1] | (R)-5-amino-N-(1-(3-(5- (cyclopentane- carboxamidomethyl) thiophen- 2-yl)phenyl)ethyl)-2- methylbenzamide | 462.27 |
| 96 | 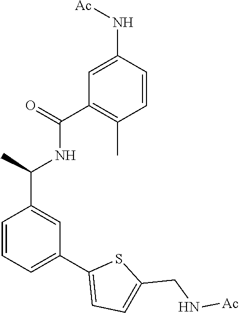 [SMILES: CC(=O)NCc1ccc(-c2cccc([C@@H](C)NC(=O)c3cc(NC(C)=O)ccc3C)c2)s1] | (R)-5-acetamido-N-(1-(3-(5- (acetamidomethyl)thiophen- 2-yl)phenyl)ethyl)-2- methylbenzamide | 450.19 |
| 97 | 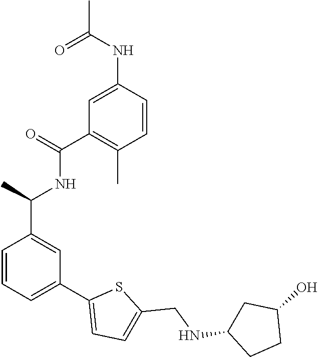 [SMILES: CC(=O)Nc1ccc(C)c(C(=O)N[C@H](C)c2cccc(-c3ccc(CN[C@H]4CC[C@@H](O)C4)s3)c2)c1] | 5-acetamido-N-((R)-1-(3- (5-((((1S,3R)-3- hydroxycyclopentyl)amino) methyl)thiophen-2- yl)phenyl)ethyl)-2- methylbenzamide | 492.24 |

In other embodiments, the compounds of Formulas I-VI and VIII-X are selected from one or more compounds of Table 2.

TABLE 2
Exemplary Compounds of Formulas I- VI and VIII-X and Chemical Characterization

| | | | LC-MS |
|---|---|---|---|
| | | | characterization |
| Examples | Structure | Chemical name | on (M + H⁺) |
| 98 | 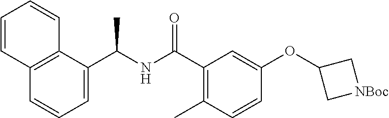 [SMILES: Cc1ccc(OC2CN(C(=O)OC(C)(C)C)C2)cc1C(=O)N[C@H](C)c1cccc2ccccc12] | tert-butyl (R)-3-(4- methyl-3-((1- (naphthalen-1- yl)ethyl)carbamoyl) phenoxy)azetidine-1- carboxylate | 461.22 |
| 99 | 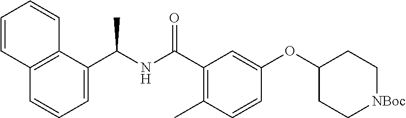 [SMILES: Cc1ccc(OC2CCN(C(=O)OC(C)(C)C)CC2)cc1C(=O)N[C@H](C)c1cccc2ccccc12] | tert-butyl (R)-4-(4- methyl-3-((1- (naphthalen-1- yl)ethyl)carbamoyl) phenoxy)piperidine-1- carboxylate | 489.22 |
| 100 | 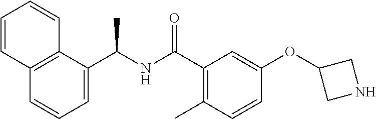 [SMILES: Cc1ccc(OC2CNC2)cc1C(=O)N[C@H](C)c1cccc2ccccc12] | (R)-5-(azetidin-3-yloxy)- 2-methyl-N-(1- (naphthalen-1- yl)ethyl)benzamide | 361.11 |
| 101 | 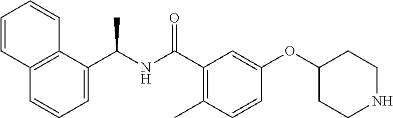 [SMILES: Cc1ccc(OC2CCNCC2)cc1C(=O)N[C@H](C)c1cccc2ccccc12] | (R)-2-methyl-N-(1- (naphthalen-1-yl)ethyl)- 5-(piperidin-4- yloxy)benzamide | 389.13 |
| 102 | 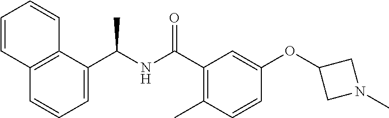 [SMILES: Cc1ccc(OC2CN(C)C2)cc1C(=O)N[C@H](C)c1cccc2ccccc12] | (R)-2-methyl-5-((1- methylazetidin-3-yl)oxy)- N-(1-(naphthalen-1- yl)ethyl)benzamide | 375.27 |
| 103 | 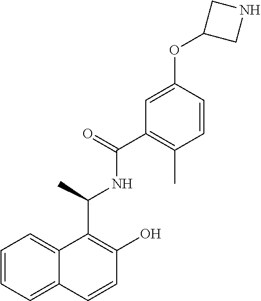 [SMILES: Cc1ccc(OC2CNC2)cc1C(=O)N[C@H](C)c1c(O)ccc2ccccc12] | (R)-5-(azetidin-3-yloxy)- N-(1-(2- hydroxynaphthalen-1- yl)ethyl)-2- methylbenzamide | 377.16 |
| 104 | 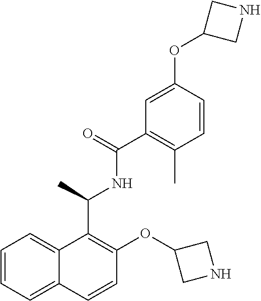 [SMILES: Cc1ccc(OC2CNC2)cc1C(=O)N[C@H](C)c1c(OC2CNC2)ccc2ccccc12] | (R)-5-(azetidin-3-yloxy)- N-(1-(2-(azetidin-3- yloxy) naphthalen-1- yl)ethyl)-2- methylbenzamide | 432.30 |
| 105 | 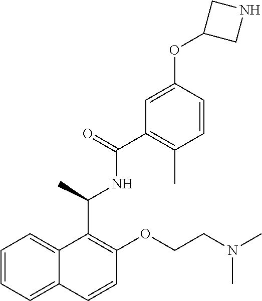 [SMILES: Cc1ccc(OC2CNC2)cc1C(=O)N[C@H](C)c1c(OCCN(C)C)ccc2ccccc12] | (R)-5-(azetidin-3-yloxy)- N-(1-(2-(2- (dimethylamino)ethoxy)n aphthalen-1-yl)ethyl)-2- methylbenzamide | 448.37 |
| 106 | 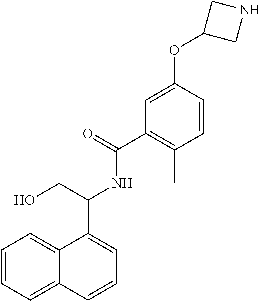 [SMILES: Cc1ccc(OC2CNC2)cc1C(=O)NC(CO)c1cccc2ccccc12] | 5-(azetidin-3-yloxy)-N- (2-hydroxy-1- (naphthalen-1-yl)ethyl)- 2-methylbenzamide | 377.16 |
| 107 | 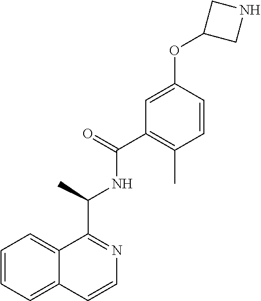 [SMILES: Cc1ccc(OC2CNC2)cc1C(=O)N[C@H](C)c1nccc2ccccc12] | (R)-5-(azetidin-3-yloxy)- N-(1-(isoquinolin-1- yl)ethyl)-2- methylbenzamide | 362.17 |
| 108 | 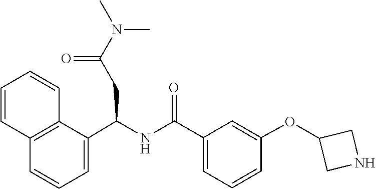 [SMILES: CN(C)C(=O)C[C@@H](NC(=O)c1cccc(OC2CNC2)c1)c1cccc2ccccc12] | (R)-3-(azetidin-3-yloxy)- N-(3-(dimethylamino)-1- (naphthalen-1-yl)-3- oxopropyl)benzamide | 418.31 |
| 109 | 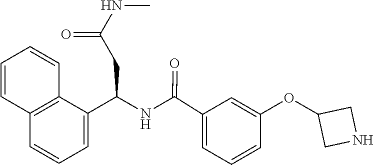 [SMILES: CNC(=O)C[C@@H](NC(=O)c1cccc(OC2CNC2)c1)c1cccc2ccccc12] | (R)-3-(azetidin-3-yloxy)- N-(3-(methylamino)-1- (naphthalen-1-yl)-3- oxopropyl)benzamide | 404.19 |
| 110 | 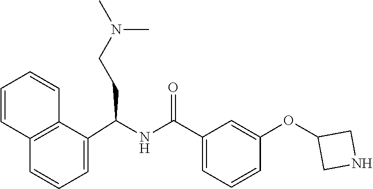 [SMILES: CN(C)CC[C@@H](NC(=O)c1cccc(OC2CNC2)c1)c1cccc2ccccc12] | (R)-3-(azetidin-3-yloxy)- N-(3-(dimethylamino)-1- (naphthalen-1- yl)propyl)benzamide | 404.13 |
| 111 | 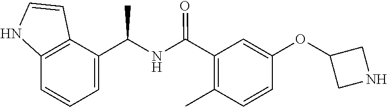 [SMILES: Cc1ccc(OC2CNC2)cc1C(=O)N[C@H](C)c1cccc2[nH]ccc12] | (R)-N-(1-(1H-indol-4- yl)ethyl)-5-(azetidin-3- yloxy)-2- methylbenzamide | 350.14 |
| 112 | 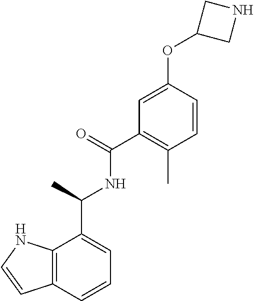 [SMILES: Cc1ccc(OC2CNC2)cc1C(=O)N[C@H](C)c1cccc2cc[nH]c12] | (R)-N-(1-(1H-indol-7- yl)ethyl)-5-(azetidin-3- yloxy)-2- methylbenzamide | 350.21 |
| 113 | 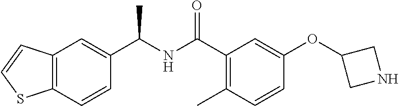 [SMILES: Cc1ccc(OC2CNC2)cc1C(=O)N[C@H](C)c1ccc2sccc2c1] | (R)-5-(azetidin-3-yloxy)- N-(1-(benzo[b]thiophen- 5-yl)ethyl)-2- methylbenzamide | 367.15 |
| 114 | 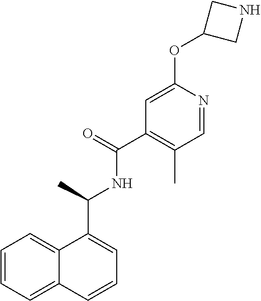 [SMILES: Cc1cnc(OC2CNC2)cc1C(=O)N[C@H](C)c1cccc2ccccc12] | (R)-5-(azetidin-3-yloxy)- N-(1-(benzo[b]thiophen- 5-yl)ethyl)-2- methylbenzamide | 362.18 |
| 115 | 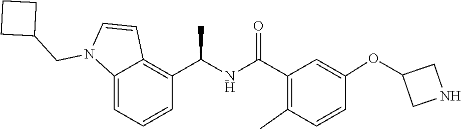 [SMILES: Cc1ccc(OC2CNC2)cc1C(=O)N[C@H](C)c1cccc2c1ccn2CC1CCC1] | (R)-5-(azetidin-3-yloxy)- N-(1-(1- (cyclobutylmethyl)-1H- indol-4-yl)ethyl)-2- methylbenzamide | 418.24 |
| 116 | 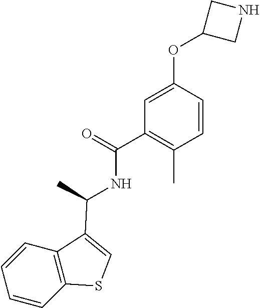 [SMILES: Cc1ccc(OC2CNC2)cc1C(=O)N[C@H](C)c1csc2ccccc12] | (R)-5-(azetidin-3-yloxy)- N-(1-(benzo[b]thiophen- 3-yl)ethyl)-2- methylbenzamide | 367.15 |
| 117 | 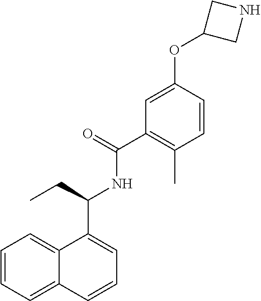 [SMILES: CC[C@@H](NC(=O)c1cc(OC2CNC2)ccc1C)c1cccc2ccccc12] | (R)-5-(azetidin-3-yloxy)- 2-methyl-N-(1- (naphthalen-1- yl)propyl)benzamide | 375.01 |
| 118 | 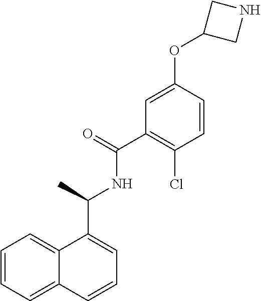 [SMILES: C[C@@H](NC(=O)c1cc(OC2CNC2)ccc1Cl)c1cccc2ccccc12] | (R)-5-(azetidin-3-yloxy)- 2-chloro-N-(1- (naphthalen-1- yl)ethyl)benzamide | 381.15 |
| 119 | 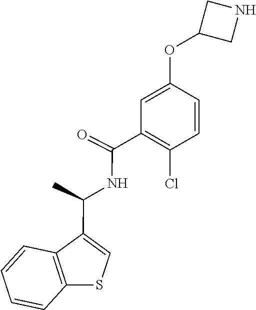 [SMILES: C[C@@H](NC(=O)c1cc(OC2CNC2)ccc1Cl)c1csc2ccccc12] | (R)-5-(azetidin-3-yloxy)- N-(1-(benzo[b]thiophen- 3-yl)ethyl)-2- chlorobenzamide | 387.14 |
| 120 | 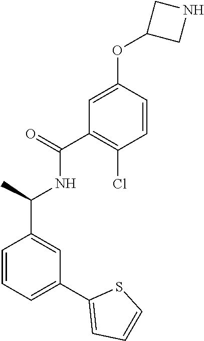 [SMILES: C[C@@H](NC(=O)c1cc(OC2CNC2)ccc1Cl)c1cccc(-c2cccs2)c1] | (R)-5-(azetidin-3-yloxy)- 2-chloro-N-(1-(3- (thiophen-2- yl)phenyl)ethyl)benzamide | 413.13 |
| 121 | 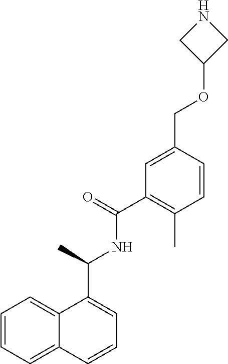 [SMILES: Cc1ccc(COC2CNC2)cc1C(=O)N[C@H](C)c1cccc2ccccc12] | (R)-5-((azetidin-3- yloxy)methyl)-2-methyl- N-(1-(naphthalen-1- yl)ethyl)benzamide | 375.21 |
| 122 | 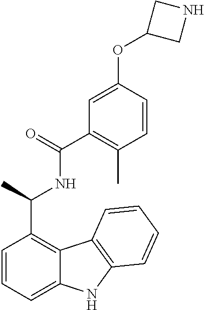 [SMILES: Cc1ccc(OC2CNC2)cc1C(=O)N[C@H](C)c1cccc2[nH]c3ccccc3c12] | (R)-N-(1-(9H-carbazol-4- yl)ethyl)-5-(azetidin-3- yloxy)-2- methylbenzamide | 400.26 |
| 123 | 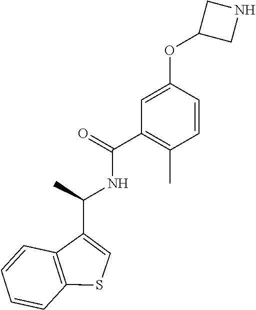 [SMILES: Cc1ccc(OC2CNC2)cc1C(=O)N[C@H](C)c1csc2ccccc12] | (R)-5-(azetidin-3-yloxy)- N-(1-(benzo[b]thiophen- 3-yl)ethyl)-2- methylbenzamide | 367.11 |
| 124 | 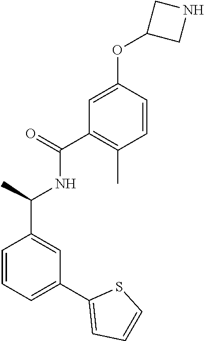 [SMILES: Cc1ccc(OC2CNC2)cc1C(=O)N[C@H](C)c1cccc(-c2cccs2)c1] | (R)-5-(azetidin-3-yloxy)- 2-methyl-N-(1-(3- (thiophen-2- yl)phenyl)ethyl)benzamide | 393.16 |
| 125 | 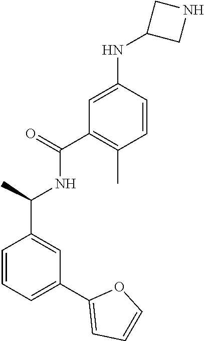 [SMILES: Cc1ccc(NC2CNC2)cc1C(=O)N[C@H](C)c1cccc(-c2ccco2)c1] | (R)-5-(azetidin-3- ylamino)-N-(1-(3-(furan- 2-yl)phenyl)ethyl)-2- methylbenzamide | 376.17 |
| 126 | 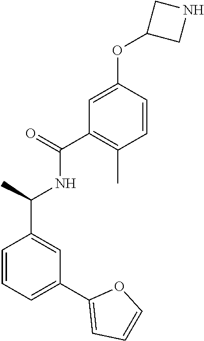 [SMILES: Cc1ccc(OC2CNC2)cc1C(=O)N[C@H](C)c1cccc(-c2ccco2)c1] | (R)-5-(azetidin-3-yloxy)- N-(1-(3-(furan-2- yl)phenyl)ethyl)-2- methylbenzamide | 377.19 |
| 127 | 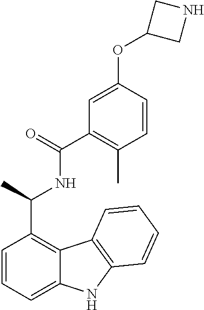 [SMILES: Cc1ccc(OC2CNC2)cc1C(=O)N[C@H](C)c1cccc2[nH]c3ccccc3c12] | (R)-N-(1-(9H-carbazol-4- yl)ethyl)-5-(azetidin-3- yloxy)-2- methylbenzamide | 400.15 |
| 128 | 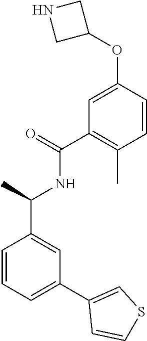 [SMILES: Cc1ccc(OC2CNC2)cc1C(=O)N[C@H](C)c1cccc(-c2ccsc2)c1] | (R)-N-(1-(3-(1H-pyrrol-3- yl)phenyl)ethyl)-5- (azetidin-3-yloxy)-2- methylbenzamide | 393.12 |
| 129 | 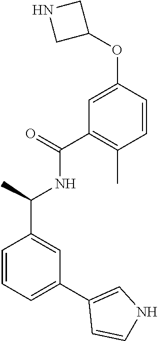 [SMILES: Cc1ccc(OC2CNC2)cc1C(=O)N[C@H](C)c1cccc(-c2cc[nH]c2)c1] | (R)-N-(1-(3-(1H-pyrrol-3- yl)phenyl)ethyl)-5- (azetidin-3-ylamino)-2- methylbenzamide | 376.18 |
| 130 | 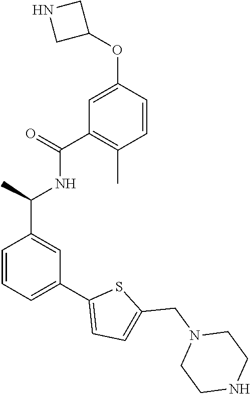 [SMILES: Cc1ccc(OC2CNC2)cc1C(=O)N[C@H](C)c1cccc(-c2ccc(CN3CCNCC3)s2)c1] | (R)-5-(azetidin-3-yloxy)- 2-methyl-N-(1-(3-(5- (piperazin-1- ylmethyl)thiophen-2- yl)phenyl)ethyl)benzamide | 491.21 |
| 131 | 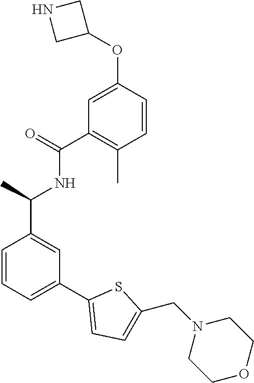 [SMILES: Cc1ccc(OC2CNC2)cc1C(=O)N[C@H](C)c1cccc(-c2ccc(CN3CCOCC3)s2)c1] | (R)-5-(azetidin-3-yloxy)- 2-methyl-N-(1-(3-(5- (morpholinomethyl)thiophen-2- yl)phenyl)ethyl)benzamide | 492.27 |
| 132 | 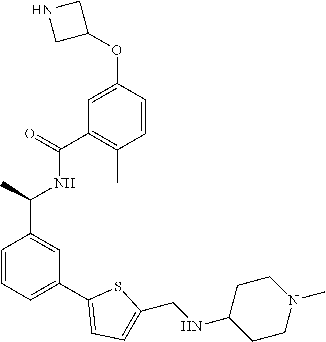 [SMILES: Cc1ccc(OC2CNC2)cc1C(=O)N[C@H](C)c1cccc(-c2ccc(CNC3CCN(C)CC3)s2)c1] | (R)-5-(azetidin-3-yloxy)- 2-methyl-N-(1-(3-(5-(((1- methylpiperidin-4- yl)amino)methyl)thiophen-2- yl)phenyl)ethyl)benzamide | 519.33 |
| 133 | 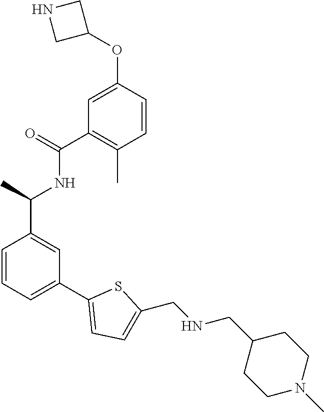 [SMILES: Cc1ccc(OC2CNC2)cc1C(=O)N[C@H](C)c1cccc(-c2ccc(CNCC3CCN(C)CC3)s2)c1] | (R)-5-(azetidin-3-yloxy)- 2-methyl-N-(1-(3-(5- ((((1-methylpiperidin-4- yl)methyl)amino)methyl)t hiophen-2- yl)phenyl)ethyl)benzamide | 533.34 |
| 134 | 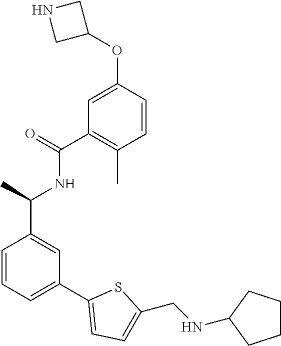 [SMILES: Cc1ccc(OC2CNC2)cc1C(=O)N[C@H](C)c1cccc(-c2ccc(CNC3CCCC3)s2)c1] | (R)-5-(azetidin-3-yloxy)- N-(1-(3-(5- ((cyclopentylamino)meth yl)thiophen-2- yl)phenyl)ethyl)-2- methylbenzamide | 490.23 |
| 135 | 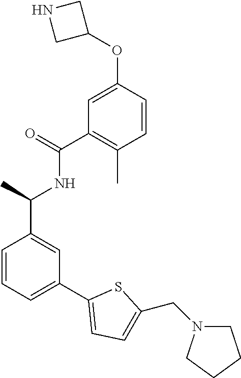 [SMILES: Cc1ccc(OC2CNC2)cc1C(=O)N[C@H](C)c1cccc(-c2ccc(CN3CCCC3)s2)c1] | (R)-5-(azetidin-3-yloxy)- 2-methyl-N-(1-(3-(5- (pyrrolidin-1- ylmethyl)thiophen-2- yl)phenyl)ethyl)benzamide | 476.21 |
| 136 | 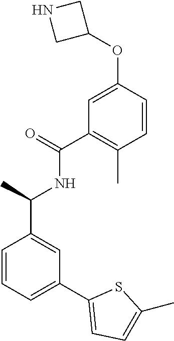 [SMILES: Cc1ccc(-c2cccc([C@@H](C)NC(=O)c3cc(OC4CNC4)ccc3C)c2)s1] | (R)-5-(azetidin-3-yloxy)- 2-methyl-N-(1-(3-(5- methylthiophen-2- yl)phenyl)ethyl)benzamide | 407.16 |
| 137 | 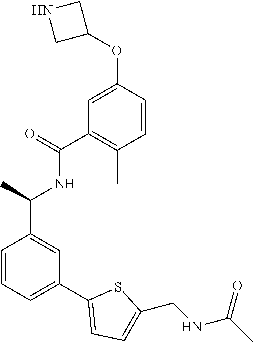 [SMILES: CC(=O)NCc1ccc(-c2cccc([C@@H](C)NC(=O)c3cc(OC4CNC4)ccc3C)c2)s1] | (R)-N-(1-(3-(5- (acetamidomethyl)thiophen- 2-yl)phenyl)ethyl)-5- (azetidin-3-yloxy)-2- methylbenzamide | 464.21 |
| 138 | 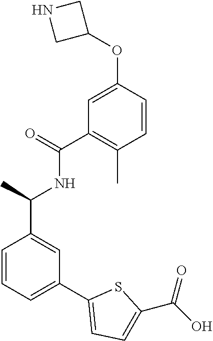 [SMILES: Cc1ccc(OC2CNC2)cc1C(=O)N[C@H](C)c1cccc(-c2ccc(C(=O)O)s2)c1] | (R)-5-(3-(1-(5-(azetidin- 3-yloxy)-2- methylbenzamido)ethyl) phenyl)thiophene-2- carboxylic acid | 437.15 |
| 139 | 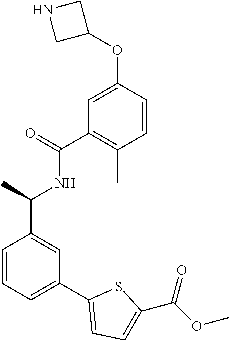 [SMILES: COC(=O)c1ccc(-c2cccc([C@@H](C)NC(=O)c3cc(OC4CNC4)ccc3C)c2)s1] | methyl (R)-5-(3-(1-(5- (azetidin-3-yloxy)-2- methylbenzamido)ethyl) phenyl)thiophene-2- carboxylate | 451.12 |
| 140 | 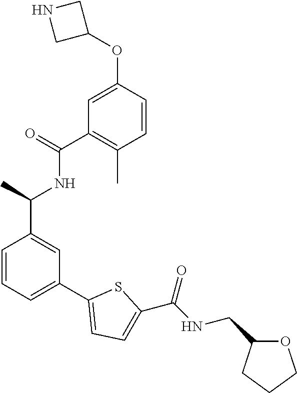 | 5-(3-((R)-1-(5-(azetidin- 3-yloxy)-2- methylbenzamido)ethyl) phenyl)-N-(((R)- tetrahydrofuran-2- yl)methyl)thiophene-2- carboxamide | 520.23 |
| 141 | 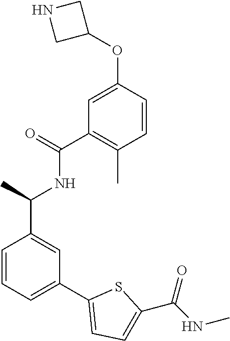 [SMILES: CNC(=O)c1ccc(-c2cccc([C@@H](C)NC(=O)c3cc(OC4CNC4)ccc3C)c2)s1] | (R)-5-(3-(1-(5-(azetidin- 3-yloxy)-2- methylbenzamido)ethyl) phenyl)-N- methylthiophene-2- carboxamide | 450.13 |
| 142 | 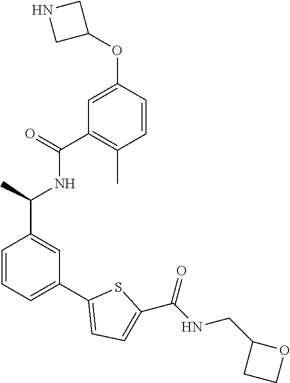 [SMILES: Cc1ccc(OC2CNC2)cc1C(=O)N[C@H](C)c1cccc(-c2ccc(C(=O)NCC3CCO3)s2)c1] | 5-(3-((R)-1-(5-(azetidin- 3-yloxy)-2- methylbenzamido)ethyl) phenyl)-N-(oxetan-2- ylmethyl)thiophene-2- carboxamide | 506.24 |
| 143 | 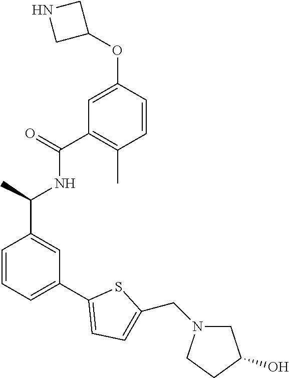 [SMILES: Cc1ccc(OC2CNC2)cc1C(=O)N[C@H](C)c1cccc(-c2ccc(CN3CC[C@@H](O)C3)s2)c1] | 5-(azetidin-3-yloxy)-N- ((R)-1-(3-(5-(((R)-3- hydroxypyrrolidin-1- yl)methyl)thiophen-2- yl)phenyl)ethyl)-2- methylbenzamide | 492.27 |
| 144 | 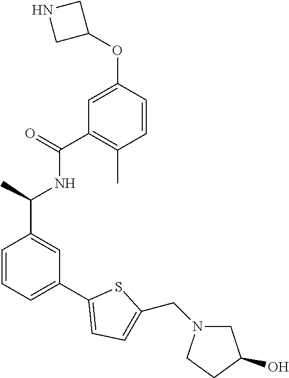 [SMILES: Cc1ccc(OC2CNC2)cc1C(=O)N[C@H](C)c1cccc(-c2ccc(CN3CC[C@H](O)C3)s2)c1] | 5-(azetidin-3-yloxy)-N- ((R)-1-(3-(5-(((S)-3- hydroxypyrrolidin-1- yl)methyl)thiophen-2- yl)phenyl)ethyl)-2- methylbenzamide | 492.24 |
| 145 | 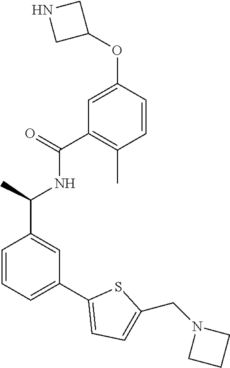 [SMILES: Cc1ccc(OC2CNC2)cc1C(=O)N[C@H](C)c1cccc(-c2ccc(CN3CCC3)s2)c1] | (R)-N-(1-(3-(5-(azetidin- 1-ylmethyl)thiophen-2- yl)phenyl)ethyl)-5- (azetidin-3-yloxy)-2- methylbenzamide | 462.23 |
| 146 | 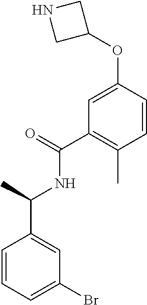 [SMILES: Cc1ccc(OC2CNC2)cc1C(=O)N[C@H](C)c1cccc(Br)c1] | (R)-5-(azetidin-3-yloxy)- N-(1-(3- bromophenyl)ethyl)-2- methylbenzamide | 389.06 |
| 147 | 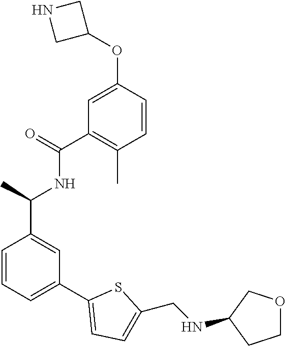 [SMILES: Cc1ccc(OC2CNC2)cc1C(=O)N[C@H](C)c1cccc(-c2ccc(CN[C@@H]3CCOC3)s2)c1] | 5-(azetidin-3-yloxy)-2- methyl-N-((R)-1-(3-(5- ((((R)-tetrahydrofuran-3- yl)amino)methyl)thiophen-2- yl)phenyl)ethyl)benzamide | 492.23 |
| 148 | 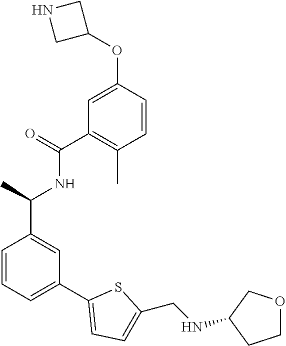 [SMILES: Cc1ccc(OC2CNC2)cc1C(=O)N[C@H](C)c1cccc(-c2ccc(CN[C@H]3CCOC3)s2)c1] | 5-(azetidin-3-ylamino)-2- methyl-N-((R)-1-(3-(5- ((((S)-tetrahydrofuran-3- yl)amino)methyl)thiophen-2- yl)phenyl)ethyl)benzamide | 492.25 |
| 149 | 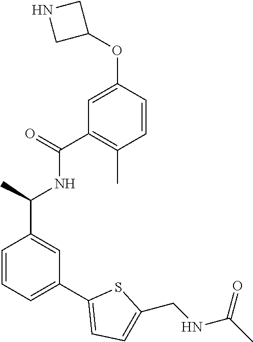 [SMILES: CC(=O)NCc1ccc(-c2cccc([C@@H](C)NC(=O)c3cc(OC4CNC4)ccc3C)c2)s1] | (R)-N-(1-(3-(5- (acetamidomethyl)thiophen- 2-yl)phenyl)ethyl)-5- (azetidin-3-yloxy)-2- methylbenzamide | 464.19 |
| 150 | 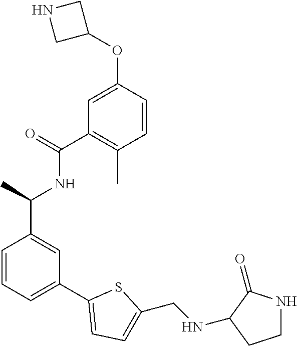 [SMILES: Cc1ccc(OC2CNC2)cc1C(=O)N[C@H](C)c1cccc(-c2ccc(CNC3CCNC3=O)s2)c1] | 5-(azetidin-3-yloxy)-2- methyl-N-((1R)-1-(3-(5- (((2-oxopyrrolidin-3- yl)amino)methyl)thiophen-2- yl)phenyl)ethyl)benzamide | 505.27 |
| 151 | 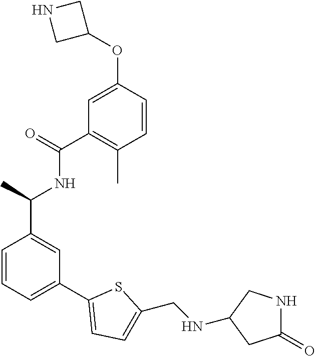 [SMILES: Cc1ccc(OC2CNC2)cc1C(=O)N[C@H](C)c1cccc(-c2ccc(CNC3CNC(=O)C3)s2)c1] | 5-(azetidin-3-yloxy)-2- methyl-N-((1R)-1-(3-(5- (((5-oxopyrrolidin-3- yl)amino)methyl)thiophen-2- yl)phenyl)ethyl)benzamide | 505.17 |
| 152 | 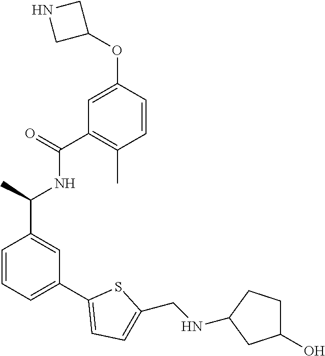 [SMILES: Cc1ccc(OC2CNC2)cc1C(=O)N[C@H](C)c1cccc(-c2ccc(CNC3CCC(O)C3)s2)c1] | 5-(azetidin-3-ylamino)- N-((1R)-1-(3-(5-(((3- hydroxycyclopentyl)amino) methyl)thiophen-2- yl)phenyl)ethyl)-2- methylbenzamide | 506.27 |
| 153 | 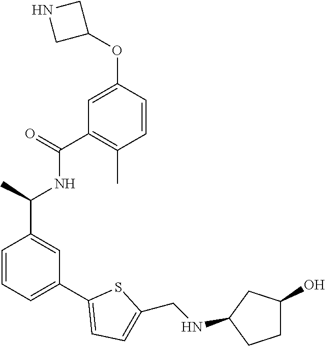 [SMILES: Cc1ccc(OC2CNC2)cc1C(=O)N[C@H](C)c1cccc(-c2ccc(CN[C@@H]3CC[C@H](O)C3)s2)c1] | 5-(azetidin-3-yloxy)-N- ((R)-1-(3-(5-((((1R,3S)-3- hydroxycyclopentyl)amino) methyl)thiophen-2- yl)phenyl)ethyl)-2- methylbenzamide | 506.25 |
| 154 | 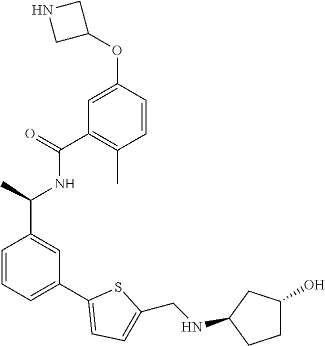 [SMILES: Cc1ccc(OC2CNC2)cc1C(=O)N[C@H](C)c1cccc(-c2ccc(CN[C@@H]3CC[C@@H](O)C3)s2)c1] | 5-(azetidin-3-yloxy)-N- ((R)-1-(3-(5-((((1R,3R)-3- hydroxycyclopentyl)amino) methyl)thiophen-2- yl)phenyl)ethyl)-2- methylbenzamide | 506.27 |
| 155 | 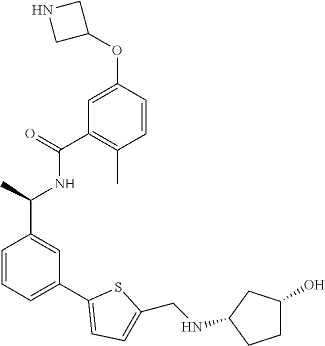 [SMILES: Cc1ccc(OC2CNC2)cc1C(=O)N[C@H](C)c1cccc(-c2ccc(CN[C@H]3CC[C@@H](O)C3)s2)c1] | 5-(azetidin-3-yloxy)-N- ((R)-1-(3-(5-((((1S,3R)-3- hydroxycyclopentyl)amino) methyl)thiophen-2- yl)phenyl)ethyl)-2- methylbenzamide | 506.23 |
| 156 | 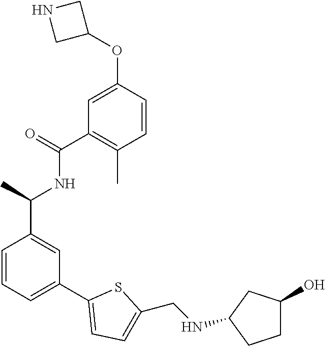 [SMILES: Cc1ccc(OC2CNC2)cc1C(=O)N[C@H](C)c1cccc(-c2ccc(CN[C@H]3CC[C@H](O)C3)s2)c1] | 5-(azetidin-3-yloxy)-N- ((R)-1-(3-(5-((((1S,3S)-3- hydroxycyclopentyl)amino) methyl)thiophen-2- yl)phenyl)ethyl)-2- methylbenzamide | 506.27 |
| 157 | 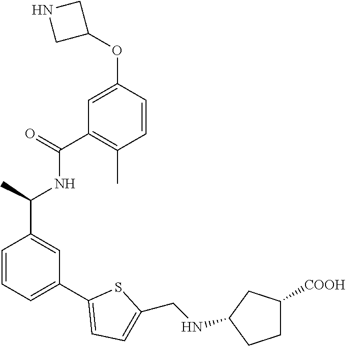 [SMILES: Cc1ccc(OC2CNC2)cc1C(=O)N[C@H](C)c1cccc(-c2ccc(CN[C@H]3CC[C@@H](C(=O)O)C3)s2)c1] | (1R,3S)-3-(((5-(3-((R)-1- (5-(azetidin-3-ylamino)-2- methylbenzamido)ethyl) phenyl)thiophen-2- yl)methyl)amino)cyclopentane- 1-carboxylic acid | 533.26 |
| 158 | 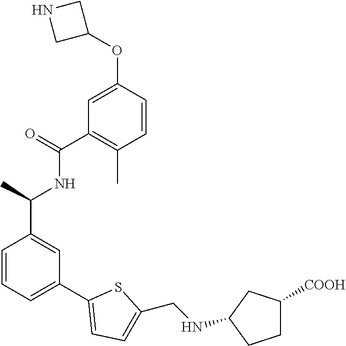 [SMILES: Cc1ccc(OC2CNC2)cc1C(=O)N[C@H](C)c1cccc(-c2ccc(CN[C@H]3CC[C@@H](C(=O)O)C3)s2)c1] | (1R,3S)-3-(((5-(3-((R)-1- (5-(azetidin-3-yloxy)-2- methylbenzamido)ethyl) phenyl)thiophen-2- yl)methyl)amino)cyclope ntane-1-carboxylic acid | 534.26 |
| 159 | 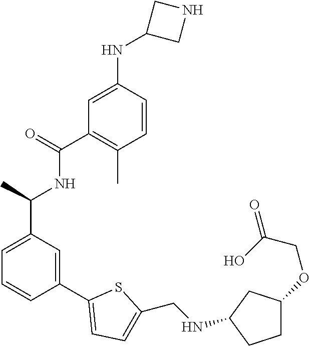 [SMILES: Cc1ccc(NC2CNC2)cc1C(=O)N[C@H](C)c1cccc(-c2ccc(CN[C@H]3CC[C@@H](OCC(=O)O)C3)s2)c1] | 2-(((1R,3S)-3-(((5-(3- ((R)-1-(5-(azetidin-3- ylamino)-2- methylbenzamido)ethyl) phenyl)thiophen-2- yl)methyl)amino)cyclope ntyl)oxy)acetic acid | 563.23 |
| 160 | 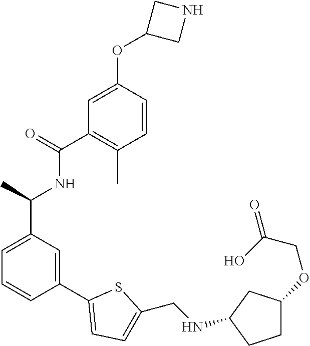 [SMILES: Cc1ccc(OC2CNC2)cc1C(=O)N[C@H](C)c1cccc(-c2ccc(CN[C@H]3CC[C@@H](OCC(=O)O)C3)s2)c1] | 2-(((1R,3S)-3-(((5-(3- ((R)-1-(5-(azetidin-3- yloxy)-2- methylbenzamido)ethyl) phenyl)thiophen-2- yl)methyl)amino)cyclopentyl) oxy)acetic acid | 564.23 |
| 161 | 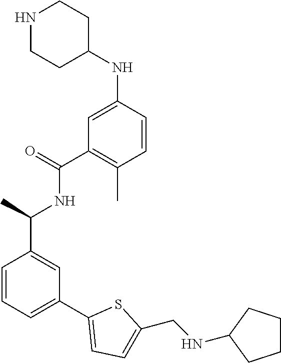 [SMILES: Cc1ccc(NC2CCNCC2)cc1C(=O)N[C@H](C)c1cccc(-c2ccc(CNC3CCCC3)s2)c1] | (R)-N-(1-(3-(5- ((cyclopentylamino) methyl)thiophen-2- yl)phenyl)ethyl)-2- methyl-5-(piperidin-4- ylamino)benzamide | 517.30 |
| 162 | 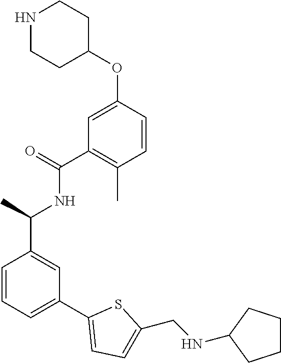 [SMILES: Cc1ccc(OC2CCNCC2)cc1C(=O)N[C@H](C)c1cccc(-c2ccc(CNC3CCCC3)s2)c1] | (R)-N-(1-(3-(5- ((cyclopentylamino) methyl)thiophen-2- yl)phenyl)ethyl)-2- methyl-5-(piperidin-4- yloxy)benzamide | 518.24 |
| 163 | 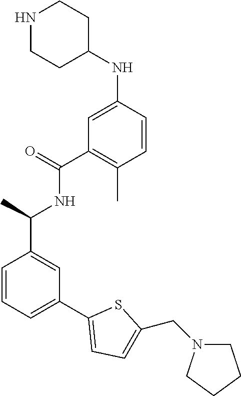 [SMILES: Cc1ccc(NC2CCNCC2)cc1C(=O)N[C@H](C)c1cccc(-c2ccc(CN3CCCC3)s2)c1] | (R)-2-methyl-5- (piperidin-4-ylamino)-N- (1-(3-(5-(pyrrolidin-1- ylmethyl)thiophen-2- yl)phenyl)ethyl)benzamide | 503.27 |
| 164 | 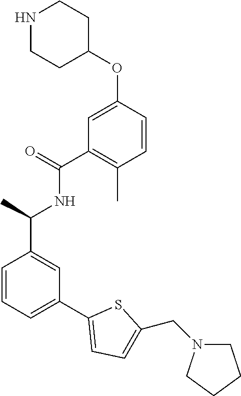 [SMILES: Cc1ccc(OC2CCNCC2)cc1C(=O)N[C@H](C)c1cccc(-c2ccc(CN3CCCC3)s2)c1] | (R)-2-methyl-5- (piperidin-4-yloxy)-N-(1- (3-(5-(pyrrolidin-1- ylmethyl)thiophen-2- yl)phenyl)ethyl)benzamide | 504.23 |
| 165 | 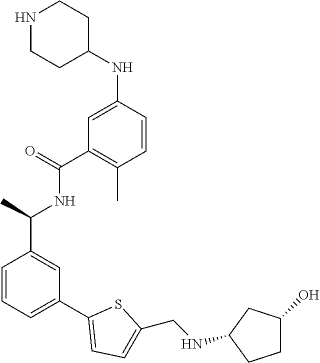 [SMILES: Cc1ccc(NC2CCNCC2)cc1C(=O)N[C@H](C)c1cccc(-c2ccc(CN[C@H]3CC[C@@H](O)C3)s2)c1] | N-((R)-1-(3-(5- ((((1S,3R)-3- hydroxycyclopentyl)amino) methyl)thiophen-2- yl)phenyl)ethyl)-2- methyl-5-(piperidin-4- ylamino)benzamide | 533.29 |
| 166 | 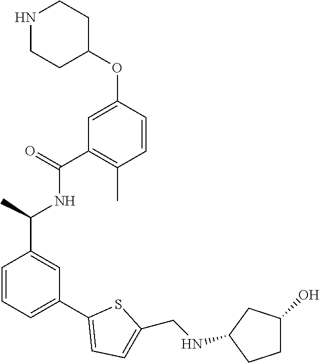 [SMILES: Cc1ccc(OC2CCNCC2)cc1C(=O)N[C@H](C)c1cccc(-c2ccc(CN[C@H]3CC[C@@H](O)C3)s2)c1] | N-((R)-1-(3-(5- ((((1S,3R)-3- hydroxycyclopentyl)amino) methyl)thiophen-2- yl)phenyl)ethyl)-2- methyl-5-(piperidin-4- yloxy)benzamide | 534.26 |
| 167 | 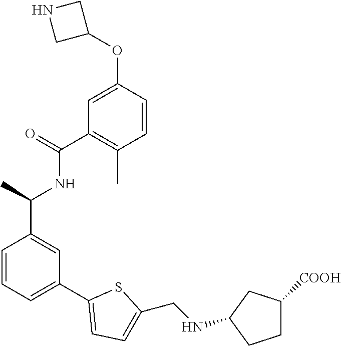 [SMILES: Cc1ccc(OC2CNC2)cc1C(=O)N[C@H](C)c1cccc(-c2ccc(CN[C@H]3CC[C@@H](C(=O)O)C3)s2)c1] | (1R,3S)-3-(((5-(3-((R)-1- (5-(azetidin-3-yloxy)-2- methylbenzamido)ethyl) phenyl)thiophen-2- yl)methyl)amino)cyclopentane- 1-carboxylic acid | 534.26 |
| 168 | 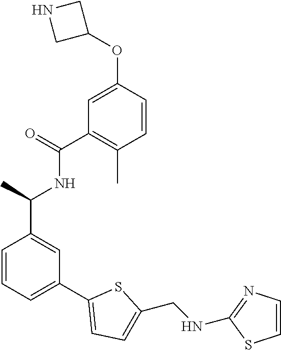 [SMILES: Cc1ccc(OC2CNC2)cc1C(=O)N[C@H](C)c1cccc(-c2ccc(CNc3nccs3)s2)c1] | (R)-5-(azetidin-3-yloxy)- 2-methyl-N-(1-(3-(5- ((thiazol-2- ylamino)methyl)thiophen-2- yl)phenyl)ethyl)benzamide | 504.6 |
| 169 | 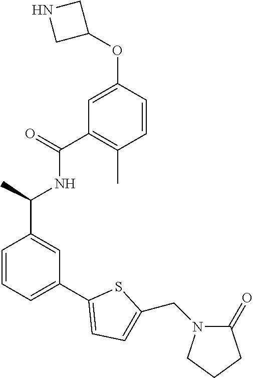 [SMILES: Cc1ccc(OC2CNC2)cc1C(=O)N[C@H](C)c1cccc(-c2ccc(CN3CCCC3=O)s2)c1] | (R)-5-(azetidin-3-yloxy)- 2-methyl-N-(1-(3-(5-((2- oxopyrrolidin-1- yl)methyl)thiophen-2- yl)phenyl)ethyl)benzamide | 489.6 |
| 170 | 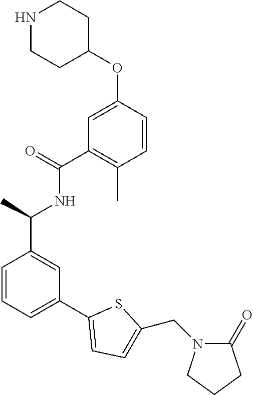 [SMILES: Cc1ccc(OC2CCNCC2)cc1C(=O)N[C@H](C)c1cccc(-c2ccc(CN3CCCC3=O)s2)c1] | (R)-2-methyl-N-(1-(3-(5- ((2-oxopyrrolidin-1- yl)methyl)thiophen-2- yl)phenyl)ethyl)-5- (piperidin-4- yloxy)benzamide | 517.9 |
| 171 | 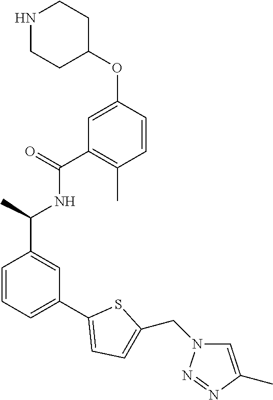 [SMILES: Cc1cn(Cc2ccc(-c3cccc([C@@H](C)NC(=O)c4cc(OC5CCNCC5)ccc4C)c3)s2)nn1] | (R)-2-methyl-N-(1-(3-(5- ((4-methyl-1H-1,2,3- triazol-1- yl)methyl)thiophen-2- yl)phenyl)ethyl)-5- (piperidin-4- yloxy)benzamide | 515.7 |
| 172 | 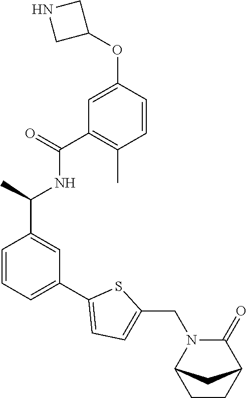 [SMILES: Cc1ccc(OC2CNC2)cc1C(=O)N[C@H](C)c1cccc(-c2ccc(CN3C(=O)[C@@H]4CC[C@H]3C4)s2)c1] | 5-(azetidin-3-yloxy)-2- methyl-N-((R)-1-(3-(5- (((1S,4R)-3-oxo-2- azabicyclo[2.2.1]heptan- 2-yl)methyl)thiophen-2- yl)phenyl)ethyl)benzamide | 516.3 |
| 173 | 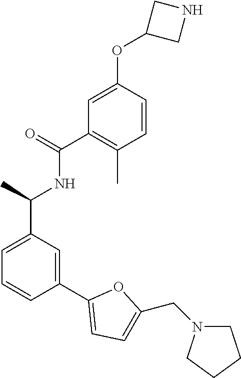 [SMILES: Cc1ccc(OC2CNC2)cc1C(=O)N[C@H](C)c1cccc(-c2ccc(CN3CCCC3)o2)c1] | (R)-5-(azetidin-3-yloxy)- 2-methyl-N-(1-(3-(5- (pyrrolidin-1- ylmethyl)furan-2- yl)phenyl)ethyl)benzamide | 460.3 |
| 174 | 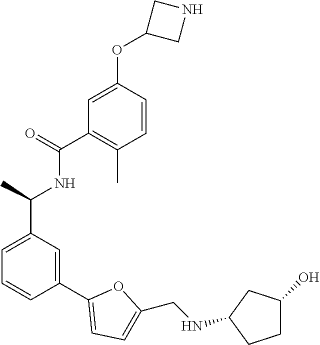 [SMILES: Cc1ccc(OC2CNC2)cc1C(=O)N[C@H](C)c1cccc(-c2ccc(CN[C@H]3CC[C@@H](O)C3)o2)c1] | 5-(azetidin-3-yloxy)-N- ((R)-1-(3-(5-((((1S,3R)-3- hydroxycyclopentyl)amino) methyl)furan-2- yl)phenyl)ethyl)-2- methylbenzamide | 490.2 |
| 175 | 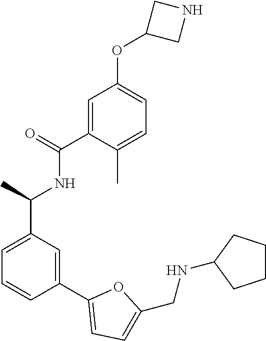 [SMILES: Cc1ccc(OC2CNC2)cc1C(=O)N[C@H](C)c1cccc(-c2ccc(CNC3CCCC3)o2)c1] | (R)-5-(azetidin-3-yloxy)- N-(1-(3-(5- ((cyclopentylamino) methyl)furan-2- yl)phenyl)ethyl)-2- methylbenzamide | 474.4 |
| 176 | 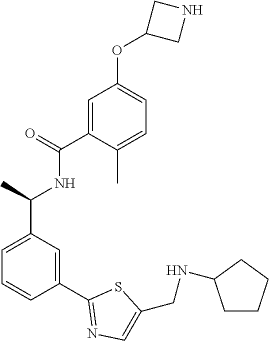 [SMILES: Cc1ccc(OC2CNC2)cc1C(=O)N[C@H](C)c1cccc(-c2ncc(CNC3CCCC3)s2)c1] | (R)-5-(azetidin-3-yloxy)- N-(1-(3-(5- ((cyclopentylamino)methyl) thiazol-2- yl)phenyl)ethyl)-2- methylbenzamide | 491.2 |
| 177 | 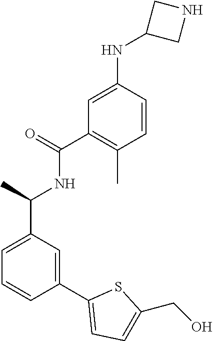 [SMILES: Cc1ccc(NC2CNC2)cc1C(=O)N[C@H](C)c1cccc(-c2ccc(CO)s2)c1] | (R)-5-(azetidin-3- ylamino)-N-(1-(3-(5- (hydroxymethyl)thiophen- 2-yl)phenyl)ethyl)-2- methylbenzamide | 422.2 |
| 178 | 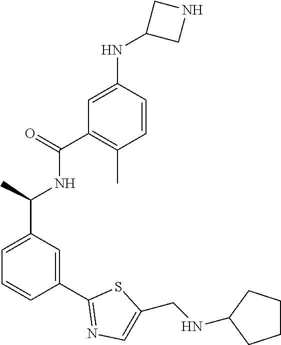 [SMILES: Cc1ccc(NC2CNC2)cc1C(=O)N[C@H](C)c1cccc(-c2ncc(CNC3CCCC3)s2)c1] | (R)-5-(azetidin-3- ylamino)-N-(1-(3-(5- ((cyclopentylamino) methyl)thiazol-2- yl)phenyl)ethyl)-2- methylbenzamide | 490.4 |
| 179 | 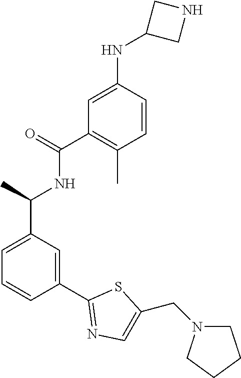 [SMILES: Cc1ccc(NC2CNC2)cc1C(=O)N[C@H](C)c1cccc(-c2ncc(CN3CCCC3)s2)c1] | (R)-5-(azetidin-3- ylamino)-2-methyl-N-(1- (3-(5-(pyrrolidin-1- ylmethyl)thiazol-2- yl)phenyl)ethyl)benzamide | 476.3 |
| 180 | 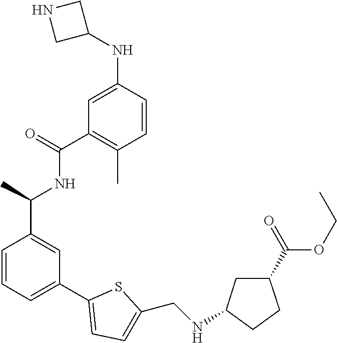 [SMILES: CCOC(=O)[C@@H]1CC[C@H](NCc2ccc(-c3cccc([C@@H](C)NC(=O)c4cc(NC5CNC5)ccc4C)c3)s2)C1] | ethyl (1R,3S)-3-(((5-(3- ((R)-1-(5-(azetidin-3- ylamino)-2- methylbenzamido)ethyl) phenyl)thiophen-2- yl)methyl)amino)cyclopentane- 1-carboxylate | 561.3 |
| 181 | 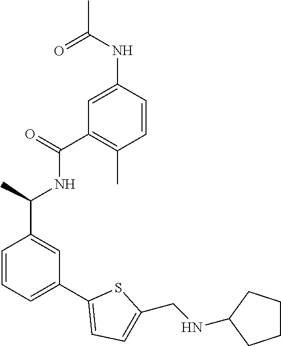 [SMILES: CC(=O)Nc1ccc(C)c(C(=O)N[C@H](C)c2cccc(-c3ccc(CNC4CCCC4)s3)c2)c1] | (R)-5-acetamido-N-(1- (3-(5- ((cyclopentylamino) methyl)thiophen-2- yl)phenyl)ethyl)-2- methylbenzamide | 476.4 |
| 182 | 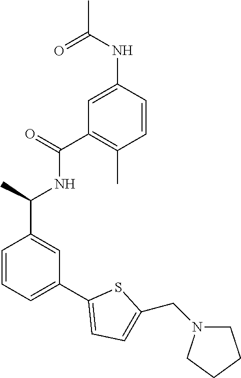 [SMILES: CC(=O)Nc1ccc(C)c(C(=O)N[C@H](C)c2cccc(-c3ccc(CN4CCCC4)s3)c2)c1] | (R)-5-acetamido-2- methyl-N-(1-(3-(5- (pyrrolidin-1- ylmethyl)thiophen-2- yl)phenyl)ethyl)benzamide | 462.3 |
| 183 | 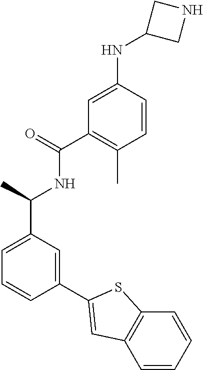 [SMILES: Cc1ccc(NC2CNC2)cc1C(=O)N[C@H](C)c1cccc(-c2cc3ccccc3s2)c1] | (R)-5-(azetidin-3- ylamino)-N-(1-(3- (benzo[b]thiophen-2- yl)phenyl)ethyl)-2- methylbenzamide | 442.3 |
| 184 | 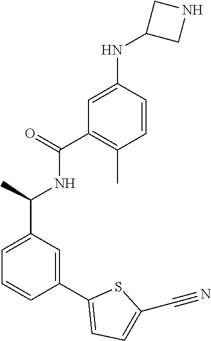 [SMILES: Cc1ccc(NC2CNC2)cc1C(=O)N[C@H](C)c1cccc(-c2ccc(C#N)s2)c1] | (R)-5-(azetidin-3- ylamino)-N-(1-(3-(5- cyanothiophen-2- yl)phenyl)ethyl)-2- methylbenzamide | 417.2 |
| 185 | 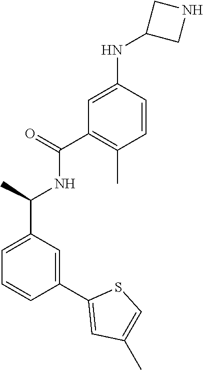 [SMILES: Cc1csc(-c2cccc([C@@H](C)NC(=O)c3cc(NC4CNC4)ccc3C)c2)c1] | (R)-5-(azetidin-3- ylamino)-2-methyl-N-(1- (3-(4-methylthiophen-2- yl)phenyl)ethyl)benzamide | 406.5 |
| 186 | 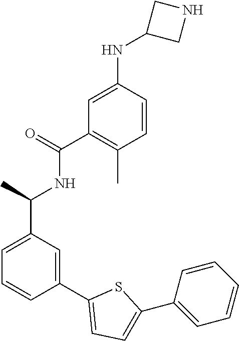 [SMILES: Cc1ccc(NC2CNC2)cc1C(=O)N[C@H](C)c1cccc(-c2ccc(-c3ccccc3)s2)c1] | (R)-5-(azetidin-3- ylamino)-2-methyl-N-(1- (3-(5-phenylthiophen-2- yl)phenyl)ethyl)benzamide | 468.3 |
| 187 | 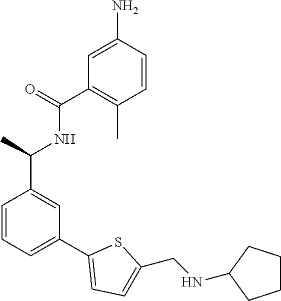 [SMILES: Cc1ccc(N)cc1C(=O)N[C@H](C)c1cccc(-c2ccc(CNC3CCCC3)s2)c1] | (R)-5-amino-N-(1-(3-(5- ((cyclopentylamino)methyl) thiophen-2- yl)phenyl)ethyl)-2- methylbenzamide | 434.2 |
| 188 | 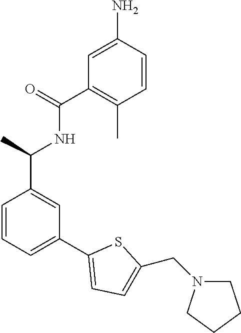 [SMILES: Cc1ccc(N)cc1C(=O)N[C@H](C)c1cccc(-c2ccc(CN3CCCC3)s2)c1] | (R)-5-amino-2-methyl-N- (1-(3-(5-(pyrrolidin-1- ylmethyl)thiophen-2- yl)phenyl)ethyl)benzamide | 420.1 |
| 189 | 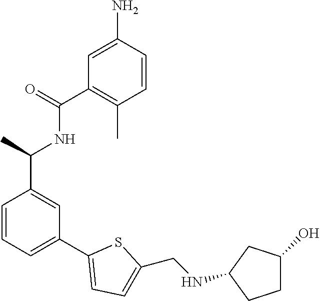 [SMILES: Cc1ccc(N)cc1C(=O)N[C@H](C)c1cccc(-c2ccc(CN[C@H]3CC[C@@H](O)C3)s2)c1] | 5-amino-N-((R)-1-(3-(5- ((((1S,3R)-3- hydroxycyclopentyl)amino) methyl)thiophen-2- yl)phenyl)ethyl)-2- methylbenzamide | 450.1 |
| 190 | 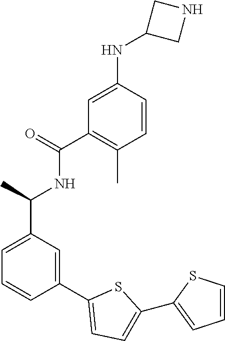 [SMILES: Cc1ccc(NC2CNC2)cc1C(=O)N[C@H](C)c1cccc(-c2ccc(-c3cccs3)s2)c1] | (R)-N-(1-(3-([2,2′-bithiophen]-5- yl)phenyl)ethyl)-5- (azetidin-3-ylamino)-2- methylbenzamide | 474.2 |
| 191 | 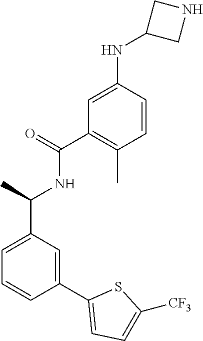 [SMILES: Cc1ccc(NC2CNC2)cc1C(=O)N[C@H](C)c1cccc(-c2ccc(C(F)(F)F)s2)c1] | (R)-5-(azetidin-3- ylamino)-2-methyl-N-(1-(3-(5- (trifluoromethyl)thiophen-2- yl)phenyl)ethyl)benzamide | 460.3 |
| 192 | 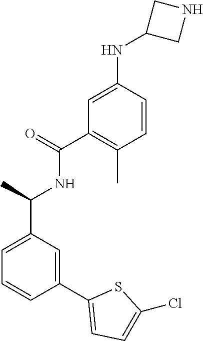 [SMILES: Cc1ccc(NC2CNC2)cc1C(=O)N[C@H](C)c1cccc(-c2ccc(Cl)s2)c1] | (R)-5-(azetidin-3- ylamino)-N-(1-(3-(5- chlorothiophen-2- yl)phenyl)ethyl)-2- methylbenzamide | 426.2 |
| 193 | 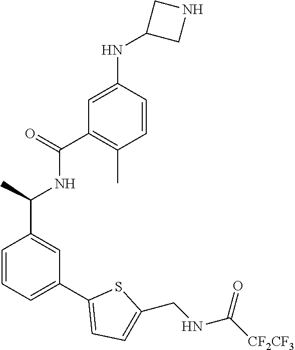 | (R)-5-(azetidin-3- ylamino)-2-methyl-N-(1- (3-(5-((2,2,3,3,3- pentafluoropropanamido) methyl)thiophen-2- yl)phenyl)ethyl)benzamide | 567.3 |
| 194 | 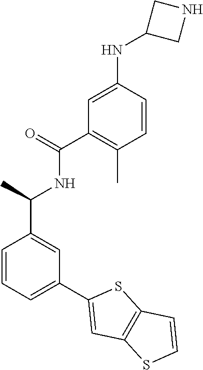 [SMILES: Cc1ccc(NC2CNC2)cc1C(=O)N[C@H](C)c1cccc(-c2cc3sccc3s2)c1] | (R)-5-(azetidin-3- ylamino)-2-methyl-N-(1- (3-(thieno[3,2-b]thiophen-2- yl)phenyl)ethyl)benzamide | 448.1 |
| 195 | 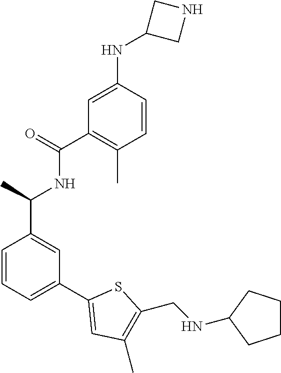 [SMILES: Cc1ccc(NC2CNC2)cc1C(=O)N[C@H](C)c1cccc(-c2cc(C)c(CNC3CCCC3)s2)c1] | (R)-5-(azetidin-3- ylamino)-N-(1-(3-(5- ((cyclopentylamino)methyl)- 4-methylthiophen-2- yl)phenyl)ethyl)-2- methylbenzamide | 503.3 |
| 196 | 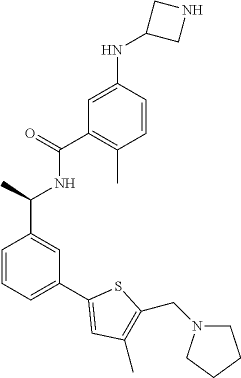 [SMILES: Cc1ccc(NC2CNC2)cc1C(=O)N[C@H](C)c1cccc(-c2cc(C)c(CN3CCCC3)s2)c1] | (R)-5-(azetidin-3- ylamino)-2-methyl-N-(1- (3-(4-methyl-5- (pyrrolidin-1- ylmethyl)thiophen-2- yl)phenyl)ethyl)benzamide | 489.1 |
| 197 | 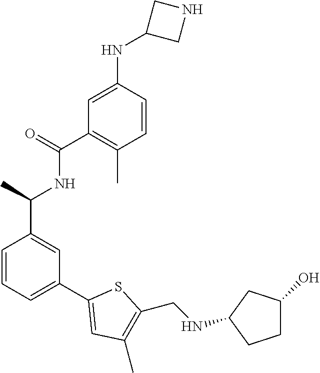 [SMILES: Cc1ccc(NC2CNC2)cc1C(=O)N[C@H](C)c1cccc(-c2cc(C)c(CN[C@H]3CC[C@@H](O)C3)s2)c1] | 5-(azetidin-3-ylamino)- N-((R)-1-(3-(5- ((((1S,3R)-3- hydroxycyclopentyl)amino) methyl)-4- methylthiophen-2- yl)phenyl)ethyl)-2- methylbenzamide | 519.2 |
| 198 | 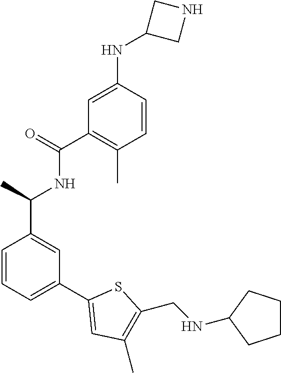 [SMILES: Cc1ccc(NC2CNC2)cc1C(=O)N[C@H](C)c1cccc(-c2cc(C)c(CNC3CCCC3)s2)c1] | (R)-5-(azetidin-3- ylamino)-N-(1-(3-(5- ((cyclopentylamino)methyl)- 4-methylthiophen-2- yl)phenyl)ethyl)-2- methylbenzamide | 503.4 |
| 199 | 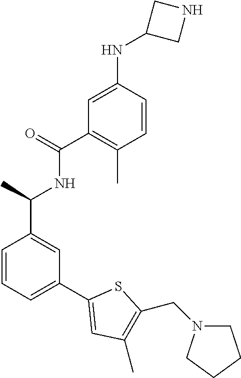 [SMILES: Cc1ccc(NC2CNC2)cc1C(=O)N[C@H](C)c1cccc(-c2cc(C)c(CN3CCCC3)s2)c1] | (R)-5-(azetidin-3- ylamino)-2-methyl-N-(1- (3-(4-methyl-5- (pyrrolidin-1- ylmethyl)thiophen-2- yl)phenyl)ethyl)benzamide | 489.3 |
| 200 | 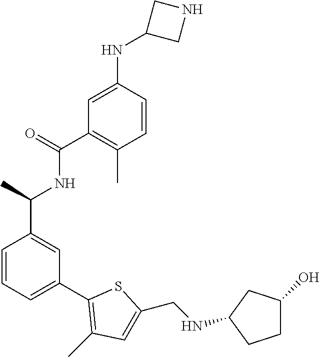 [SMILES: Cc1ccc(NC2CNC2)cc1C(=O)N[C@H](C)c1cccc(-c2sc(CN[C@H]3CC[C@@H](O)C3)cc2C)c1] | 5-(azetidin-3-ylamino)- N-((R)-1-(3-(5- ((((1S,3R)-3- hydroxycyclopentyl)amino) methyl)-3- methylthiophen-2- yl)phenyl)ethyl)-2- methylbenzamide | 519.2 |
| 201 | 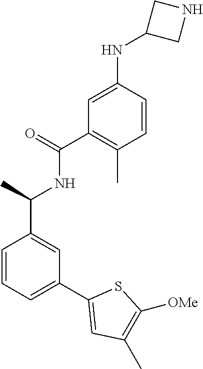 [SMILES: COc1sc(-c2cccc([C@@H](C)NC(=O)c3cc(NC4CNC4)ccc3C)c2)cc1C] | (R)-5-(azetidin-3- ylamino)-N-(1-(3-(5- methoxy-4- methylthiophen-2- yl)phenyl)ethyl)-2- methylbenzamide | 436.4 |
| 202 | 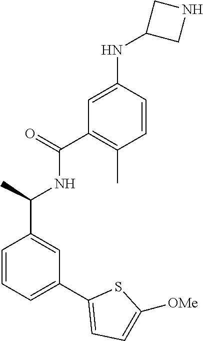 [SMILES: COc1ccc(-c2cccc([C@@H](C)NC(=O)c3cc(NC4CNC4)ccc3C)c2)s1] | (R)-5-(azetidin-3- ylamino)-N-(1-(3-(5- methoxythiophen-2- yl)phenyl)ethyl)-2- methylbenzamide | 422.3 |
| 203 | 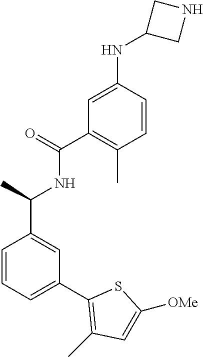 [SMILES: COc1cc(C)c(-c2cccc([C@@H](C)NC(=O)c3cc(NC4CNC4)ccc3C)c2)s1] | (R)-5-(azetidin-3- ylamino)-N-(1-(3-(5- methoxy-3- methylthiophen-2- yl)phenyl)ethyl)-2- methylbenzamide | 436.5 |
| 204 | 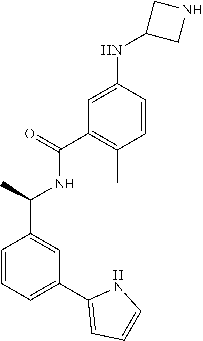 [SMILES: Cc1ccc(NC2CNC2)cc1C(=O)N[C@H](C)c1cccc(-c2ccc[nH]2)c1] | (R)-N-(1-(3-(1H-pyrrol-2- yl)phenyl)ethyl)-5- (azetidin-3-ylamino)-2- methylbenzamide | 375.2 |
| 205 | 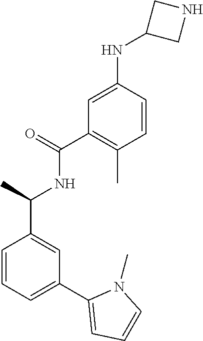 [SMILES: Cc1ccc(NC2CNC2)cc1C(=O)N[C@H](C)c1cccc(-c2cccn2C)c1] | (R)-5-(azetidin-3- ylamino)-2-methyl-N-(1- (3-(1-methyl-1H-pyrrol-2- yl)phenyl)ethyl)benzamide | 389.2 |

In certain embodiments, compounds of the present disclosure include, without limitations, compounds of Formula XI derivatized with a prodrug:

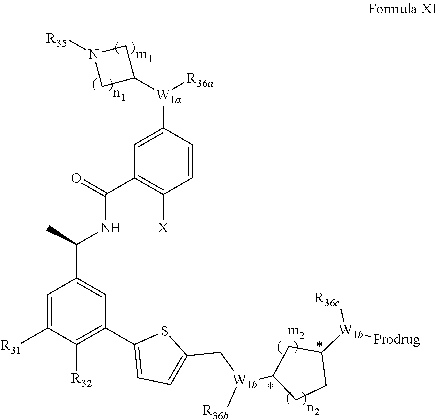

or a pharmaceutically acceptable salt thereof, wherein
X is -Me, -Et, —OMe, —CH═CH₂;
W₁ₐ and W_{1b} is —N, —O or —C; wherein if W₁ₐ═O, R₃₆ₐ does not exist and/or if W_{1b}═O, R_{36b} does not exist;
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};

R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};

R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};

—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;

R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

Where the prodrug is independently selected from hydroxyl, carboxyl, amine, phosphate, phosphonate, amidine, guanine and/or carbohydrate.

In some embodiments, the compounds of Formula XI derivatized with a prodrug are selected from one or more compounds of Table 3.

TABLE 3
Prodrug Derivatized Compounds and Chemical Characterization

| | | | LC-MS |
|---|---|---|---|
| | | | characterization |
| Examples | Structure | Chemical name | (M + H⁺) |
| 206 | 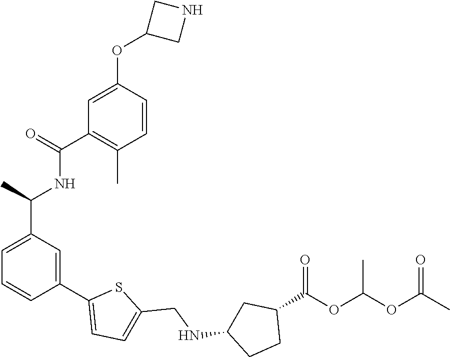 [SMILES: CC(=O)OC(C)OC(=O)[C@@H]1CC[C@H](NCc2ccc(-c3cccc([C@@H](C)NC(=O)c4cc(OC5CNC5)ccc4C)c3)s2)C1] | 1-acetoxyethyl (1R,3S)-3-(((5-(3-((R)- 1-(5-(azetidin-3- yloxy)-2- methylbenzamido) ethyl)phenyl)thiophen- 2-yl)methyl)amino)cyclo pentane-1-carboxylate | 619.8 |
| 207 | 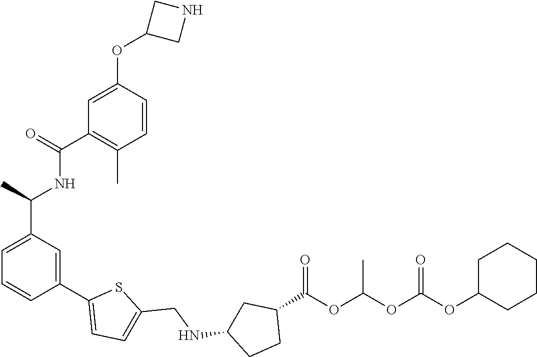 [SMILES: Cc1ccc(OC2CNC2)cc1C(=O)N[C@H](C)c1cccc(-c2ccc(CN[C@H]3CC[C@@H](C(=O)OC(C)OC(=O)OC4CCCCC4)C3)s2)c1] | 1- (((cyclohexyloxy) carbonyl)oxy)ethyl (1R,3S)-3-(((5-(3-((R)- 1-(5-(azetidin-3- yloxy)-2- methylbenzamido) ethyl)phenyl)thiophen- 2-yl)methyl)amino)cyclo pentane-1-carboxylate | 703.9 |
| 208 | 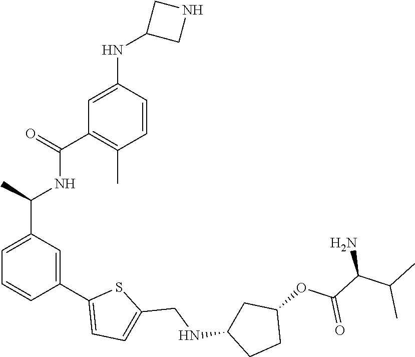 [SMILES: Cc1ccc(NC2CNC2)cc1C(=O)N[C@H](C)c1cccc(-c2ccc(CN[C@H]3CC[C@@H](OC(=O)[C@@H](N)C(C)C)C3)s2)c1] | (1R,3S)-3-(((5-(3-((R)- 1-(5-(azetidin-3- ylamino)-2- methylbenzamido) ethyl)phenyl)thiophen- 2-yl)methyl)amino)cyclo pentyl L-valinate | 604.37 |
| 209 | 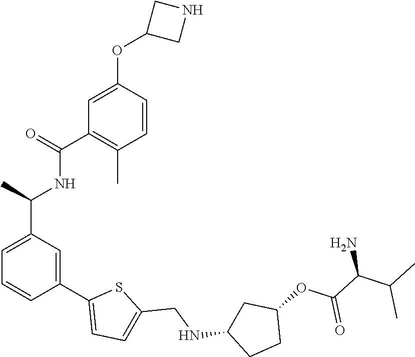 [SMILES: Cc1ccc(OC2CNC2)cc1C(=O)N[C@H](C)c1cccc(-c2ccc(CN[C@H]3CC[C@@H](OC(=O)[C@@H](N)C(C)C)C3)s2)c1] | (1R,3S)-3-(((5-(3-((R)- 1-(5-(azetidin-3- yloxy)-2- methylbenzamido) ethyl)phenyl)thiophen- 2-yl)methyl)amino)cyclo pentyl L-valinate | 605.34 |
| 210 | 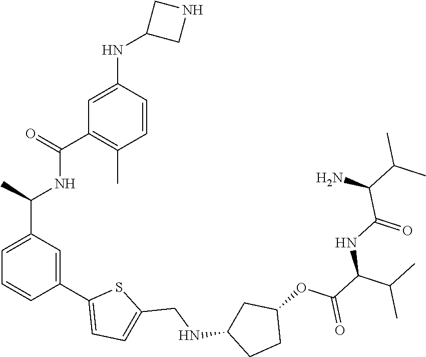 [SMILES: Cc1ccc(NC2CNC2)cc1C(=O)N[C@H](C)c1cccc(-c2ccc(CN[C@H]3CC[C@@H](OC(=O)[C@@H](NC(=O)[C@@H](N)C(C)C)C(C)C)C3)s2)c1] | (1R,3S)-3-(((5-(3-((R)- 1-(5-(azetidin-3- ylamino)-2- methylbenzamido) ethyl)phenyl)thiophen- 2-yl)methyl)amino)cyclo pentyl L-valyl-L- valinate | 703.38 |
| 211 | 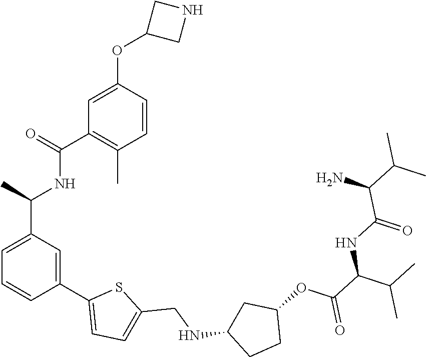 [SMILES: Cc1ccc(OC2CNC2)cc1C(=O)N[C@H](C)c1cccc(-c2ccc(CN[C@H]3CC[C@@H](OC(=O)[C@@H](NC(=O)[C@@H](N)C(C)C)C(C)C)C3)s2)c1] | (1R,3S)-3-(((5-(3-((R)- 1-(5-(azetidin-3- yloxy)-2- methylbenzamido) ethyl)phenyl)thiophen-2- yl)methyl)amino)cyclo pentyl L-valyl-L- valinate | 704.37 |
| 212 | 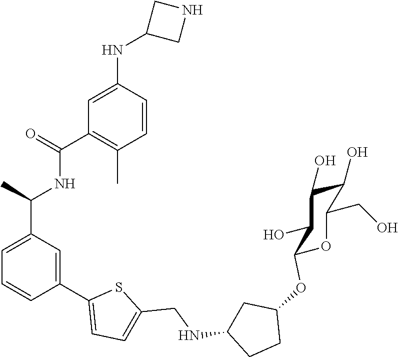 | 5-(azetidin-3- ylamino)-2-methyl-N- ((R)-1-(3-(5- ((((1S,3R)-3- (((2R,3R,4S,5S,6R)- 3,4,5-trihydroxy-6- (hydroxymethyl) tetrahydro-2H-pyran- 2-yl)oxy)cyclopentyl) amino)methyl) thiophen- 2-yl)phenyl)ethyl) benzamide | 667.32 |
| 213 | 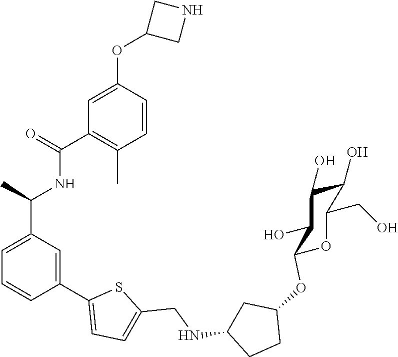 | 5-(azetidin-3-yloxy)-2- methyl-N-((R)-1-(3-(5- ((((1S,3R)-3- (((2R,3R,4S,5S,6R)- 3,4,5-trihydroxy-6- (hydroxymethyl) tetrahydro-2H-pyran- 2-yl)oxy)cyclopentyl) amino)methyl) thiophen- 2-yl)phenyl)ethyl) benzamide | 668.33 |
| 214 | 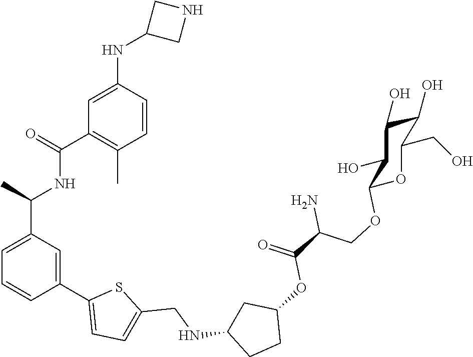 | (1R,3S)-3-(((5-(3-((R)- 1-(5-(azetidin-3- ylamino)-2- methylbenzamido) ethyl)phenyl)thiophen-2- yl)methyl)amino)cyclo pentyl O- ((2R,3R,4S,5S,6R)- 3,4,5-trihydroxy-6- (hydroxymethyl) tetrahydro-2H-pyran- 2-yl)-L- serinate | 754.37 |
| 215 | 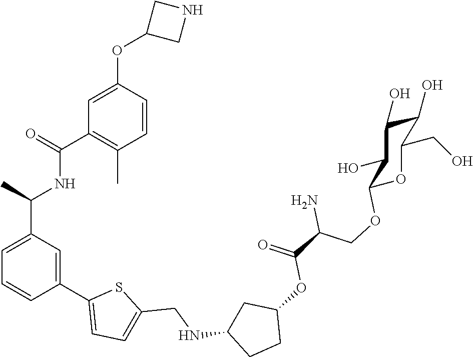 | (1R,3S)-3-(((5-(3-((R)- 1-(5-(azetidin-3- yloxy)-2- methylbenzamido) ethyl)phenyl)thiophen-2- yl)methyl)amino)cyclo pentyl O- ((2R,3R,4S,5S,6R)- 3,4,5-trihydroxy-6- (hydroxymethyl) tetrahydro-2H-pyran- 2-yl)-L-serinate | 755.34 |

In certain embodiments, compounds of the present disclosure include, without limitations, compounds of Formula XII derivatized to form a hybrid compound:

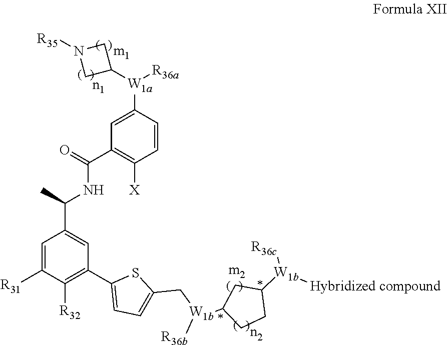

or a pharmaceutically acceptable salt thereof, wherein
X is -Me, -Et, —OMe, —CH═CH₂;
W₁ₐ and W_{1b} is —N, —O or —C; wherein if W₁ₐ═O, R₃₆ₐ does not exist and/or if W_{1b}═O, R_{36b} does not exist
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

Where the hybridized compound is selected from kinase inhibitors, NAMPT inhibitors, coronavirus inhibitors and/or virus inhibitors.

In certain embodiments, compounds of the present disclosure include, without limitation, compounds of Formula XIII derivatized to form a hybrid compound:

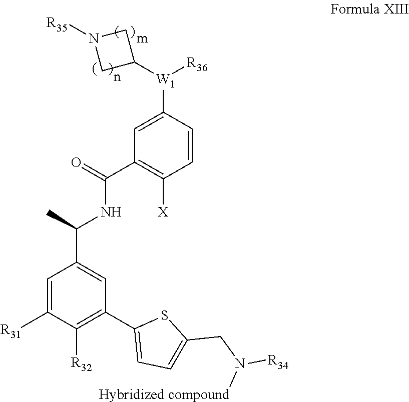

or a pharmaceutically acceptable salt thereof, wherein
X is -Me, -Et, —OMe, or —CH═CH₂;
W₁ is —N or -0; wherein if W₁═O, R₃₆ does not exist;
R₃₁ and R₃₂ are independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₄, R₃₅, and R₃₆ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
m and n=1-3.

Where the hybridized compound is selected from kinase inhibitors, NAMPT inhibitors, coronavirus inhibitors and/or virus inhibitors.

In some embodiments, the compounds of Formulas XII and XIII derivatized to form a hybrid are selected from one or more compounds of Table 4.

TABLE 4
Hybrid Compounds and Chemical Characterization

| | | | LC-MS |
|---|---|---|---|
| Ex- | | | character- |
| am- | | | ization |
| ples | Structure | Chemical name | (M + H⁺) |
| 216 | 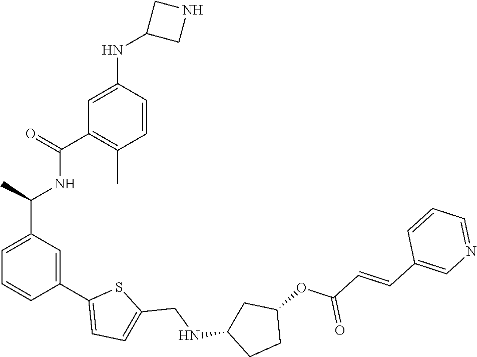 [SMILES: Cc1ccc(NC2CNC2)cc1C(=O)N[C@H](C)c1cccc(-c2ccc(CN[C@H]3CC[C@@H](OC(=O)/C=C/c4cccnc4)C3)s2)c1] | (1R,3S)-3-(((5-(3-((R)-1-(5- (azetidin-3-ylamino)-2- methylbenzamido)ethyl) phenyl)thiophen-2- yl)methyl)amino)cyclopentyl (E)-3-(pyridin-3-yl)acrylate | 636.27 |
| 217 | 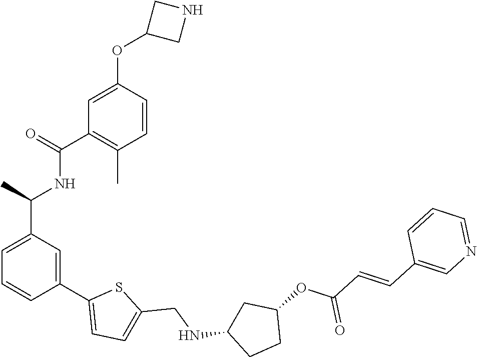 [SMILES: Cc1ccc(OC2CNC2)cc1C(=O)N[C@H](C)c1cccc(-c2ccc(CN[C@H]3CC[C@@H](OC(=O)/C=C/c4cccnc4)C3)s2)c1] | (1R,3S)-3-(((5-(3-((R)-1-(5- (azetidin-3-yloxy)-2- methylbenzamido)ethyl) phenyl)thiophen-2- yl)methyl)amino)cyclopentyl (E)-3-(pyridin-3-yl)acrylate | 637.28 |
| 218 | 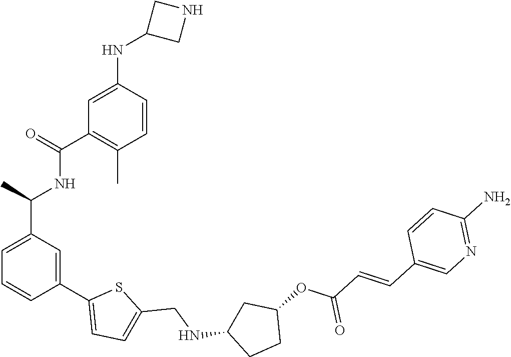 [SMILES: Cc1ccc(NC2CNC2)cc1C(=O)N[C@H](C)c1cccc(-c2ccc(CN[C@H]3CC[C@@H](OC(=O)/C=C/c4ccc(N)nc4)C3)s2)c1] | (1R,3S)-3-(((5-(3-((R)-1-(5- (azetidin-3-ylamino)-2- methylbenzamido)ethyl) phenyl)thiophen-2- yl)methyl)amino)cyclopentyl (E)-3-(6-aminopyridin-3- yl)acrylate | 651.33 |
| 219 | 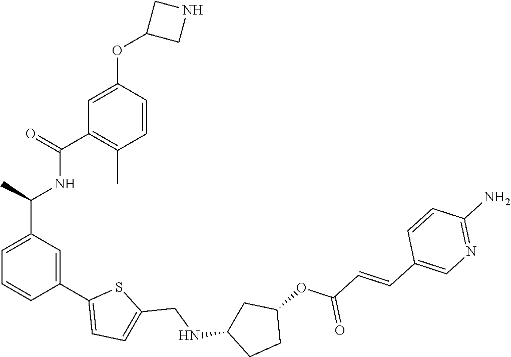 [SMILES: Cc1ccc(OC2CNC2)cc1C(=O)N[C@H](C)c1cccc(-c2ccc(CN[C@H]3CC[C@@H](OC(=O)/C=C/c4ccc(N)nc4)C3)s2)c1] | (1R,3S)-3-(((5-(3-((R)-1-(5- (azetidin-3-yloxy)-2- methylbenzamido)ethyl) phenyl)thiophen-2- yl)methyl)amino)cyclopentyl (E)-3-(6-aminopyridin-3- yl)acrylate | 652.30 |
| 220 | 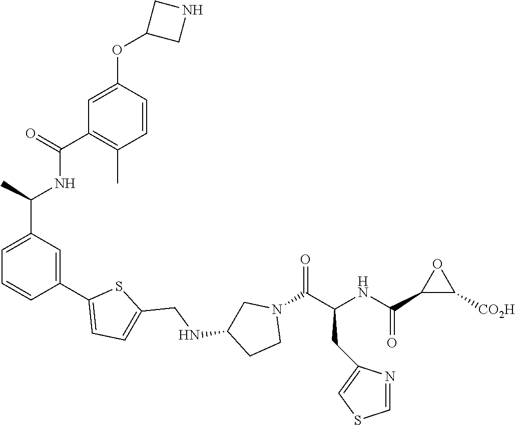 | (2S,3S)-3-(((S)-1-((S)-3- (((5-(3-((R)-1-(5-(azetidin-3- yloxy)-2- methylbenzamido)ethyl) phenyl)thiophen-2- yl)methyl)amino)pyrrolidin- 1-yl)-1-oxo-3-(thiazol-4- yl)propan-4- yl)carbamoyl)oxirane-2- carboxylic acid | 758.9 |
| 221 | 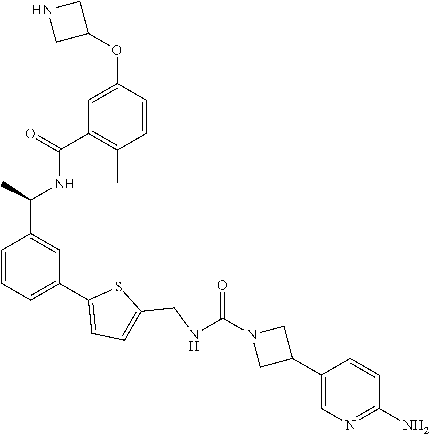 [SMILES: Cc1ccc(OC2CNC2)cc1C(=O)N[C@H](C)c1cccc(-c2ccc(CNC(=O)N3CC(c4ccc(N)nc4)C3)s2)c1] | (R)-3-(6-aminopyridin-3-yl)- N-((5-(3-(1-(5-(azetidin-3- yloxy)-2- methylbenzamido)ethyl) phenyl)thiophen-2- yl)methyl)azetidine-1- carboxamide | 596.7 |
| 222 | 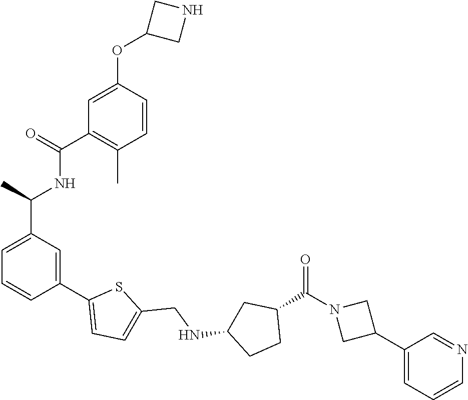 [SMILES: Cc1ccc(OC2CNC2)cc1C(=O)N[C@H](C)c1cccc(-c2ccc(CN[C@H]3CC[C@@H](C(=O)N4CC(c5cccnc5)C4)C3)s2)c1] | 5-(azetidin-3-yloxy)-2- methyl-N-((R)-1-(3-(5- ((((1S,3R)-3-(3-(pyridin-3- yl)azetidine-1- carbonyl)cyclopentyl)amino) methyl)thiophen-2- yl)phenyl)ethyl)benzamide | 649.8 |
| 223 | 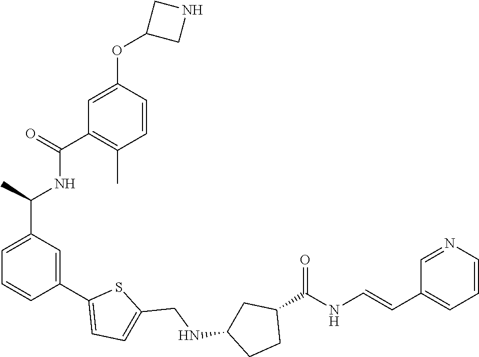 [SMILES: Cc1ccc(OC2CNC2)cc1C(=O)N[C@H](C)c1cccc(-c2ccc(CN[C@H]3CC[C@@H](C(=O)N/C=C/c4cccnc4)C3)s2)c1] | 5-(azetidin-3-yloxy)-2- methyl-N-((R)-1-(3-(5- ((((1S,3R)-3-(((E)-2- (pyridin-3- yl)vinyl)carbamoyl) cyclopentyl)amino)methyl) thiophen-2-yl)phenyl) ethyl)benzamide | 635.8 |
| 224 | 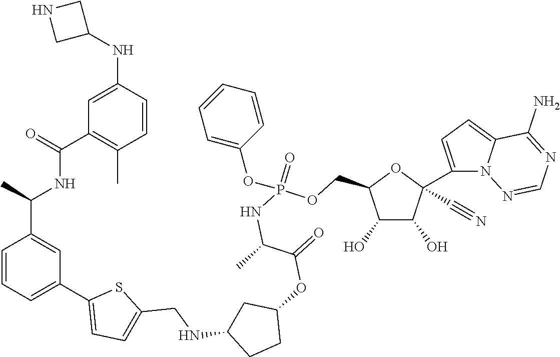 [SMILES: Cc1ccc(NC2CNC2)cc1C(=O)N[C@H](C)c1cccc(-c2ccc(CN[C@H]3CC[C@@H](OC(=O)[C@H](C)NP(=O)(OC[C@H]4O[C@@](C#N)(c5ccc6c(N)ncnn56)[C@H](O)[C@@H]4O)Oc4ccccc4)C3)s2)c1] | (1R,3S)-3-(((5-(3-((R)-1-(5- (azetidin-3-ylamino)-2- methylbenzamido)ethyl) phenyl)thiophen-2- yl)methyl)amino)cyclopentyl ((((2R,3S,4R,5R)-5-(4- aminopyrrolo[2,1- f][1,2,4]triazin-7-yl)-5- cyano-3,4- dihydroxytetrahydrofuran-2- yl)methoxy)(phenoxy) phosphoryl)-L-alaninate | 1005.4 |
| 225 | 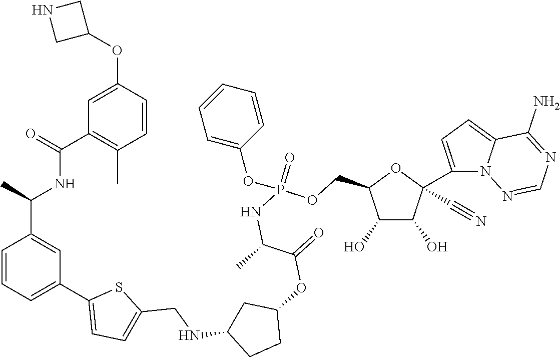 [SMILES: Cc1ccc(OC2CNC2)cc1C(=O)N[C@H](C)c1cccc(-c2ccc(CN[C@H]3CC[C@@H](OC(=O)[C@H](C)NP(=O)(OC[C@H]4O[C@@](C#N)(c5ccc6c(N)ncnn56)[C@H](O)[C@@H]4O)Oc4ccccc4)C3)s2)c1] | (1R,3S)-3-(((5-(3-((R)-1-(5- (azetidin-3-yloxy)-2- methylbenzamido)ethyl) phenyl)thiophen-2- yl)methyl)amino)cyclopentyl ((((2R,3S,4R,5R)-5-(4- aminopyrrolo[2,1- f][1,2,4]triazin-7-yl)-5- cyano-3,4- dihydroxytetrahydrofuran-2- yl)methoxy)(phenoxy) phosphoryl)-L-alaninate | 1006.4 |
| 226 | 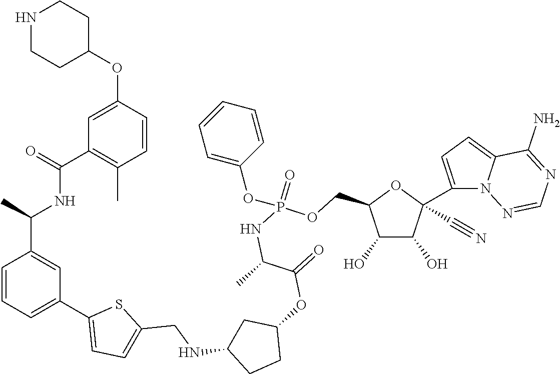 [SMILES: Cc1ccc(OC2CCNCC2)cc1C(=O)N[C@H](C)c1cccc(-c2ccc(CN[C@H]3CC[C@@H](OC(=O)[C@H](C)NP(=O)(OC[C@H]4O[C@@](C#N)(c5ccc6c(N)ncnn56)[C@H](O)[C@@H]4O)Oc4ccccc4)C3)s2)c1] | (1R,3S)-3-(((5-(3-((R)-1-(2- methyl-5-(piperidin-4- yloxy)benzamido)ethyl) phenyl)thiophen-2- yl)methyl)amino)cyclopentyl ((((2R,3S,4R,5R)-5-(4- aminopyrrolo[2,1-f][1,2,4] triazin-7-yl)-5- cyano-3,4- dihydrotetrahydrofuran-2- yl)methoxy)(phenoxy) phosphoryl)-L-alaninate | 1034.5 |
| 227 | 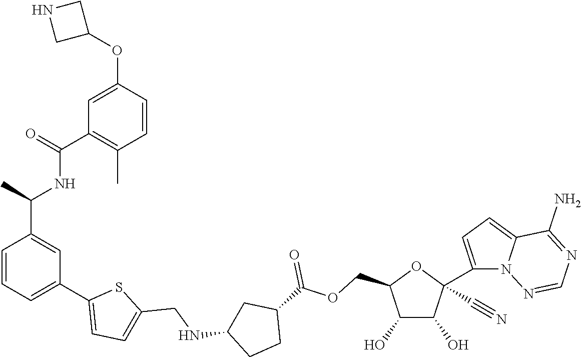 [SMILES: Cc1ccc(OC2CNC2)cc1C(=O)N[C@H](C)c1cccc(-c2ccc(CN[C@H]3CC[C@@H](C(=O)OC[C@H]4O[C@@](C#N)(c5ccc6c(N)ncnn56)[C@H](O)[C@@H]4O)C3)s2)c1] | ((2R,3S,4R,5R)-5-(4- aminopyrrolo[2,1- f][1,2,4]triazin-7-yl)-5- cyano-3,4- dihydroxytetrahydrofuran-2- yl)methyl (1R,3S)-3-(((5-(3- ((R)-1-(5-(azetidin-3-yloxy)- 2- methylbenzamido)ethyl) phenyl)thiophen-2- yl)methyl)amino) cyclopentane-1-carboxylate | 807.3 |
| 228 | 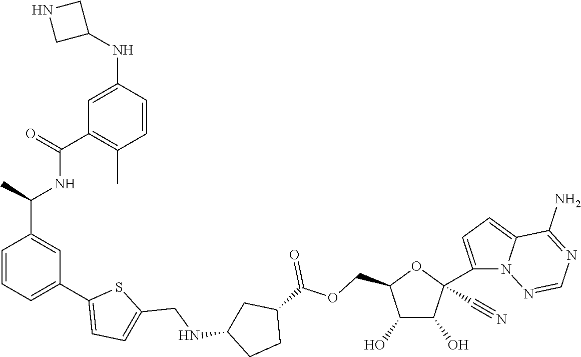 [SMILES: Cc1ccc(NC2CNC2)cc1C(=O)N[C@H](C)c1cccc(-c2ccc(CN[C@H]3CC[C@@H](C(=O)OC[C@H]4O[C@@](C#N)(c5ccc6c(N)ncnn56)[C@H](O)[C@@H]4O)C3)s2)c1] | ((2R,3S,4R,5R)-5-(4- aminopyrrolo[2,1- f][1,2,4]triazin-7-yl)-5- cyano-3,4- dihydroxytetrahydrofuran-2- yl)methyl (1R,3S)-3-(((5-(3- ((R)-1-(5-(azetidin-3- ylamino)-2- methylbenzamido)ethyl) phenyl)thiophen-2- yl)methyl)amino) cyclopentane-1-carboxylate | 806.3 |
| 229 | 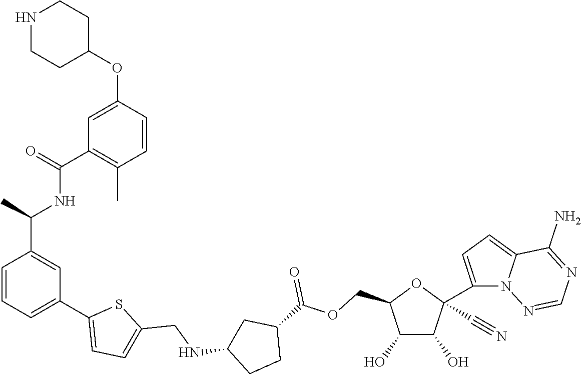 [SMILES: Cc1ccc(OC2CCNCC2)cc1C(=O)N[C@H](C)c1cccc(-c2ccc(CN[C@H]3CC[C@@H](C(=O)OC[C@H]4O[C@@](C#N)(c5ccc6c(N)ncnn56)[C@H](O)[C@@H]4O)C3)s2)c1] | ((2R,3S,4R,5R)-5-(4- aminopyrrolo[2,1- f][1,2,4]triazin-7-yl)-5- cyano-3,4- dihydroxytetrahydrofuran-2- yl)methyl (1R,3S)-3-(((5-(3- ((R)-1-(2-methyl-5- (piperidin-4- yloxy)benzamido)ethyl) phenyl)thiophen-2- yl)methyl)amino)cyclo- pentane-1-carboxylate | 835.4 |
| 230 | 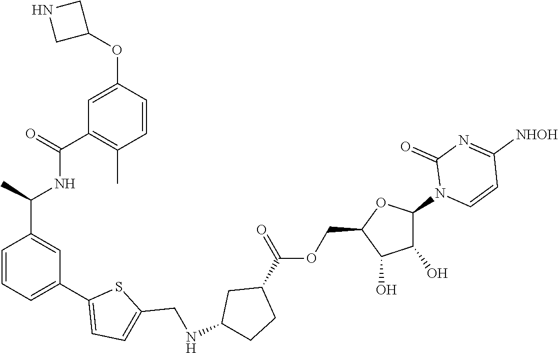 | ((2R,3S,4R,5R)-3,4- dihydroxy-5-(4- (hydroxyamino)-2- oxopyrimidin-1(2H)- yl)tetrahydrofuran-2- yl)methyl (1R,3S)-3-(((5-(3- ((R)-1-(5-(azetidin-3-yloxy)- 2- methylbenzamido)ethyl) phenyl)thiophen-2- yl)methyl)amino) cyclopentane-1-carboxylate | 775.3 |
| 231 | 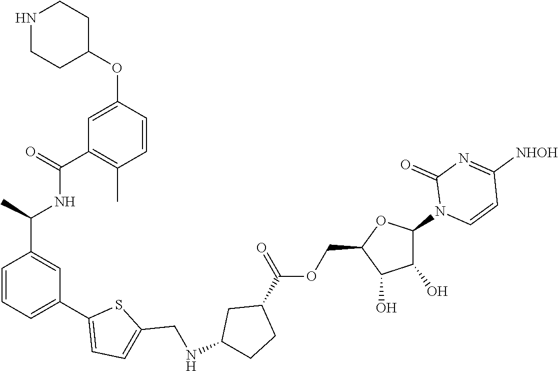 | ((2R,3S,4R,5R)-3,4- dihydroxy-5-(4- (hydroxyamino)-2- oxopyrimidin-1(2H)- yl)tetrahydrofuran-2- yl)methyl (1R,3S)-3-(((5-(3- ((R)-1-(2-methyl-5- (piperidin-4- yloxy)benzamido)ethyl) phenyl)thiophen-2- yl)methyl)amino) cyclopentane-1- carboxylate | 803.4 |
| 232 | 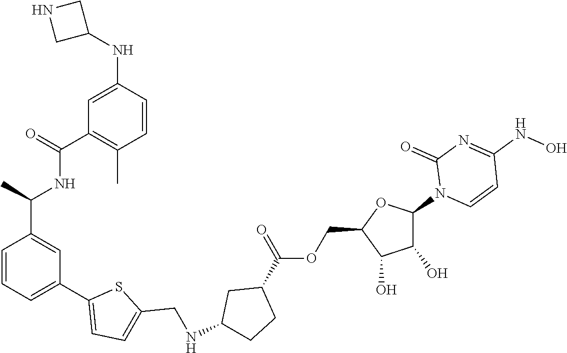 [SMILES: Cc1ccc(NC2CNC2)cc1C(=O)N[C@H](C)c1cccc(-c2ccc(CN[C@H]3CC[C@@H](C(=O)OC[C@H]4O[C@@H](n5ccc(NO)nc5=O)[C@H](O)[C@@H]4O)C3)s2)c1] | ((2R,3S,4R,5R)-3,4- dihydroxy-5-(4- (hydroxyamino)-2- oxopyrimidin-1(2H)- yl)tetrahydrofuran-2- yl)methyl (1R,3S)-3-(((5-(3- ((R)-1-(5-(azetidin-3- ylamino)-2- methylbenzamido)ethyl) phenyl)thiophen-2- yl)methyl)amino) cyclopentane-1-carboxylate | 774.4 |

In certain embodiments, compounds of the present disclosure include, without limitations, compounds of Formula XIV derivatized with a PROTAC:

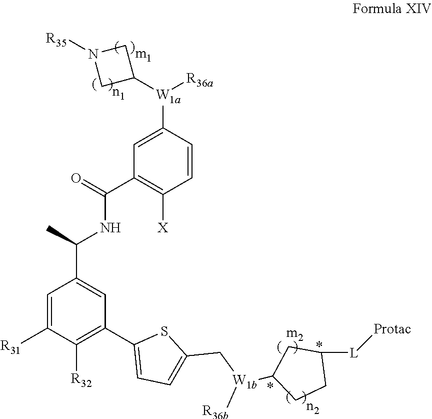

or a pharmaceutically acceptable salt thereof, wherein
X is -Me, -Et, —OMe, —CH═CH₂;
W₁ₐ and W_{1b} is —N, —O or —C; wherein if W₁═O, R₃₆ does not exist,
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl, and
m₁, m₂, n₁ and n₂=1-3.

Where the L is an optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical. In any of the embodiments of the compounds described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

Where the Protac is independently selected from hetero bifunctional molecules that connect a POI ligand to an E3 ubiquitin ligase (E3) (VHL, CRBN, IAPs, and MDM2) recruiting ligand such as thalidomide, pomalidomide, lenalidomide, VHL and so on.

In certain embodiments, compounds of the present disclosure include, without limitations, compounds of Formulas XII(a), XIII(a), or XIV(a) derivatized with a prodrug:

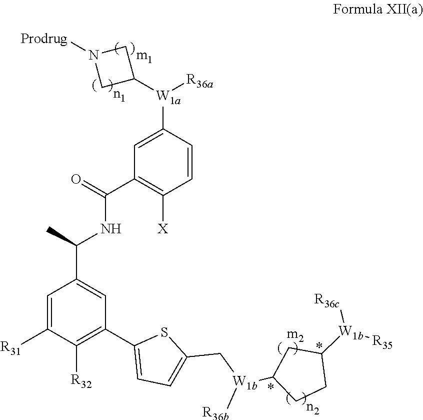

or a pharmaceutically acceptable salt thereof, wherein
X is -Me, -Et, —OMe, halogen or —CH═CH₂;
W₁ₐ and W_{1b} is —N, —O or —C; wherein if W₁═O, R₃₆ does not exist,
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₃, R₃₄, R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(Q)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

Where the prodrug is independently selected from hydroxyl, carboxyl, amine, phosphate, phosphonate, amidine, guanine and/or carbohydrate groups.

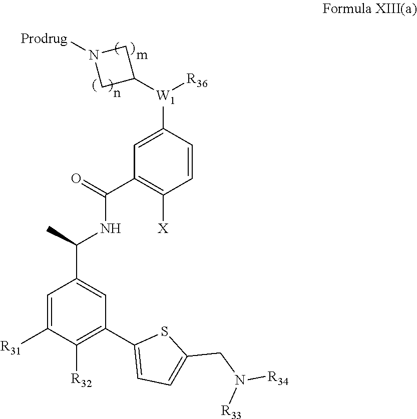

or a pharmaceutically acceptable salt thereof, wherein
X is -Me, -Et, —OMe, halogen or —CH═CH₂;
W₁ is —N or -0; wherein if W₁═O, R₃₆ does not exist;
R₃₁ and R₃₂ are independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
where R₃₃, R₃₄, and R₃₆ can be independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
R₃₃ could also be selected from PROTACs, hybrid compounds, and/or Prodrugs described before; and
m and n=1-3.

Where the prodrug is independently selected from hydroxyl, carboxyl, amine, phosphate, phosphonate, amidine, guanine and/or carbohydrate groups.

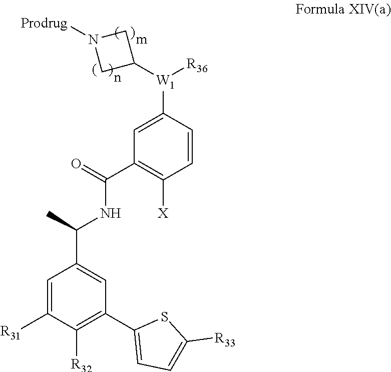

or a pharmaceutically acceptable salt thereof, wherein
X is -Me, -Et, —OMe, halogen or —CH═CH₂;
W₁ is —N or —C; wherein if W₁═O, R₃₆ does not exist;
R₃₁, R₃₂ and R₃₃ are independently selected from the group of —H, halogen, —(C₁-C₆alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
where R₃₃, R₃₄, and R₃₆ can be independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
R₃₃ could also be selected from PROTACs, hybrid compounds, and/or Prodrugs described before; and
m and n=1-3.
Where the prodrug is independently selected from hydroxyl, carboxyl, amine, phosphate, phosphonate, amidine, guanine and/or carbohydrate groups.

In some embodiments, the compounds of Formulas XII(a), XIII(a), and XIV(a) derivatized with a prodrug are selected from one or more compounds of Table 5.

TABLE 5
Exemplary Prodrug Derivatized Compounds of Formulas XII(a), XIII(a) and XIV(a) and Chemical Characterization

| | | | LC-MS |
|---|---|---|---|
| | | | characterization |
| Examples | Structure | Chemical name | (M + H⁺) |
| 233 | 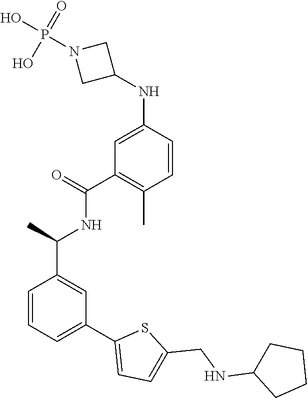 [SMILES: Cc1ccc(NC2CN(P(=O)(O)O)C2)cc1C(=O)N[C@H](C)c1cccc(-c2ccc(CNC3CCCC3)s2)c1] | (R)-(3-((3-((1-(3-(5- ((cyclopentylamino) methyl)thiophen-2- yl)phenyl)ethyl)carbamoyl)- 4-methylphenyl)amino) azetidin-1- yl)phosphonic acid | 569.3 |
| 234 | 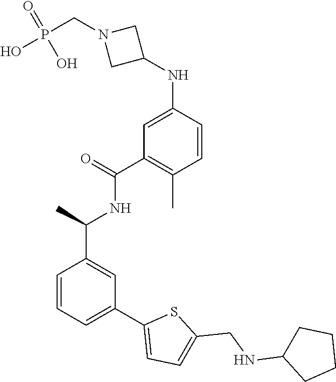 [SMILES: Cc1ccc(NC2CN(CP(=O)(O)O)C2)cc1C(=O)N[C@H](C)c1cccc(-c2ccc(CNC3CCCC3)s2)c1] | (R)-((3-((3-((1-(3-(5- ((cyclopentylamino) methyl)thiophen-2- yl)phenyl)ethyl)carbamoyl)- 4- methylphenyl)amino) azetidin-1- yl)methyl)phosphonic acid | 583.3 |
| 235 | 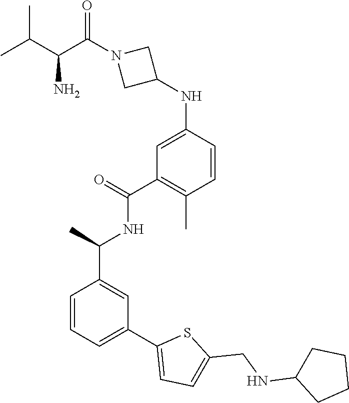 [SMILES: Cc1ccc(NC2CN(C(=O)[C@@H](N)C(C)C)C2)cc1C(=O)N[C@H](C)c1cccc(-c2ccc(CNC3CCCC3)s2)c1] | 5-((1-(L-valyl)azetidin- 3-yl)amino)-N-((R)-1- (3-(5- ((cyclopentylamino) methyl)thiophen-2- yl)phenyl)ethyl)-2- methylbenzamide | 588.4 |
| 236 | 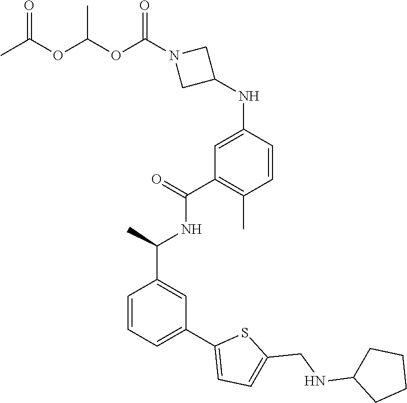 [SMILES: CC(=O)OC(C)OC(=O)N1CC(Nc2ccc(C)c(C(=O)N[C@H](C)c3cccc(-c4ccc(CNC5CCCC5)s4)c3)c2)C1] | 1-acetoxyethyl 3-((3- (((R)-1-(3-(5- ((cyclopentylamino) methyl)thiophen-2- yl)phenyl)ethyl) carbamoyl)-4- methyl)phenyl)amino) azetidine-1-carboxylate | 619.4 |
| 237 | 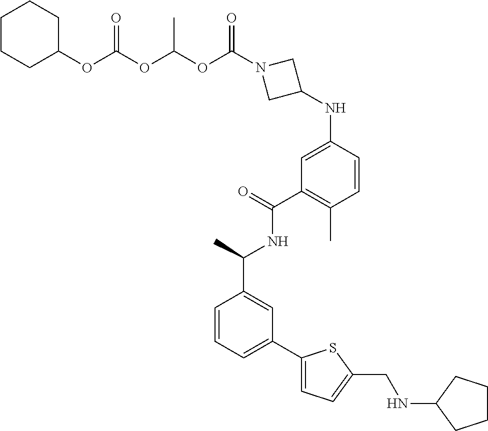 [SMILES: Cc1ccc(NC2CN(C(=O)OC(C)OC(=O)OC3CCCCC3)C2)cc1C(=O)N[C@H](C)c1cccc(-c2ccc(CNC3CCCC3)s2)c1] | 1- (((cyclohexyloxy)carbonyl) oxy)ethyl 3-((3- (((R)-1-(3-(5- ((cyclopentylamino) methyl)thiophen-2- yl)phenyl)ethyl)carbamoyl)- 4- methylphenyl)amino) azetidine-1-carboxylate | 703.5 |
| 238 | 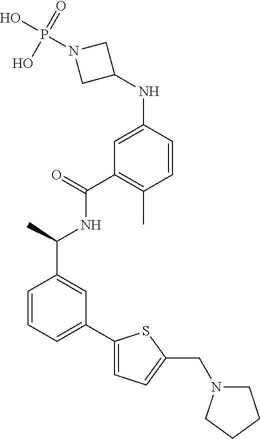 [SMILES: Cc1ccc(NC2CN(P(=O)(O)O)C2)cc1C(=O)N[C@H](C)c1cccc(-c2ccc(CN3CCCC3)s2)c1] | (R)-(3-((4-methyl-3- ((1-(3-(5-(pyrrolidin-1- ylmethyl)thiophen-2- yl)phenyl)ethyl) carbamoyl)phenyl)amino) azetidin-1-yl)phosphonic acid | 555.3 |
| 239 | 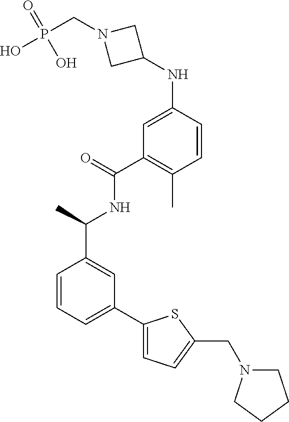 [SMILES: Cc1ccc(NC2CN(CP(=O)(O)O)C2)cc1C(=O)N[C@H](C)c1cccc(-c2ccc(CN3CCCC3)s2)c1] | (R)-((3-((4-methyl-3- ((1-(3-(5-(pyrrolidin-1- ylmethyl)thiophen-2- yl)phenyl)ethyl)carbamoyl) phenyl)amino)azetidin-1- yl)methyl)phosphonic acid | 569.4 |
| 240 | 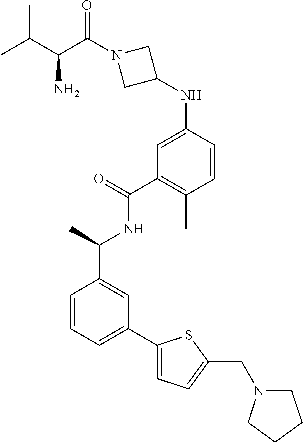 [SMILES: Cc1ccc(NC2CN(C(=O)[C@@H](N)C(C)C)C2)cc1C(=O)N[C@H](C)c1cccc(-c2ccc(CN3CCCC3)s2)c1] | 5-((1-(L-valyl)azetidin- 3-yl)amino)-2-methyl- N-((R)-1-(3-(5- (pyrrolidin-1- ylmethyl)thiophen-2- yl)phenyl)ethyl) benzamide | 574.4 |
| 241 | 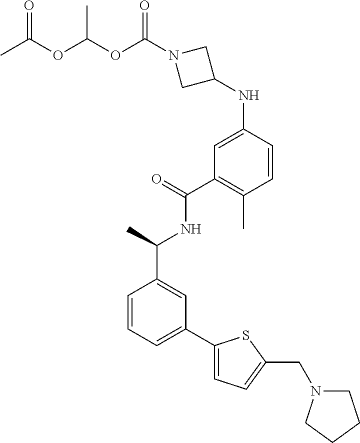 [SMILES: CC(=O)OC(C)OC(=O)N1CC(Nc2ccc(C)c(C(=O)N[C@H](C)c3cccc(-c4ccc(CN5CCCC5)s4)c3)c2)C1] | 1-acetoxyethyl 3-((4- methyl-3-(((R)-1-(3-(5- (pyrrolidin-1- ylmethyl)thiophen-2- yl)phenyl)ethyl) carbamoyl)phenyl)amino) azetidine-1-carboxylate | 605.4 |
| 242 | 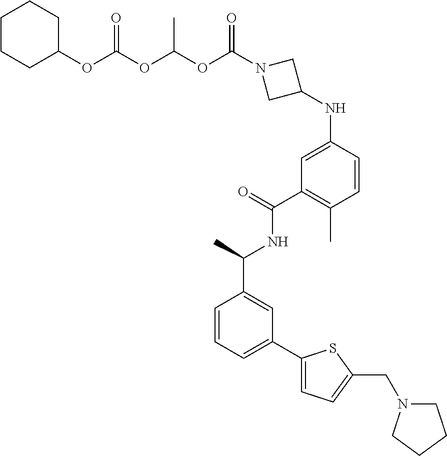 [SMILES: Cc1ccc(NC2CN(C(=O)OC(C)OC(=O)OC3CCCCC3)C2)cc1C(=O)N[C@H](C)c1cccc(-c2ccc(CN3CCCC3)s2)c1] | 1- (((cyclohexyloxy) carbonyl)oxy)ethyl) 3-((4- methyl-3-(((R)-1-(3-(5- (pyrrolidin-1- ylmethyl)thiophen-2- yl)phenyl)ethyl) carbamoyl)phenyl) amino)azetidine-1- carboxylate | 689.4 |
| 243 | 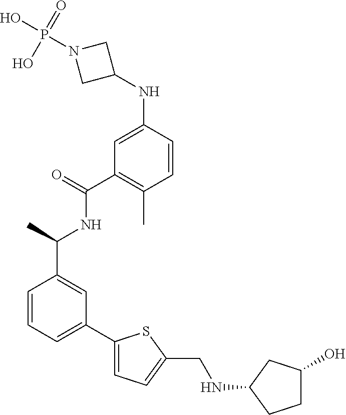 [SMILES: Cc1ccc(NC2CN(P(=O)(O)O)C2)cc1C(=O)N[C@H](C)c1cccc(-c2ccc(CN[C@H]3CC[C@@H](O)C3)s2)c1] | (3-((3-(((R)-1-(3-(5- ((((1S,3R)-3- hydroxycyclopentyl) amino)methyl)thiophen- 2-yl)phenyl)ethyl) carbamoyl)-4- methylphenyl)amino) azetidin-1- yl)phosphonic acid | 585.6 |
| 244 | 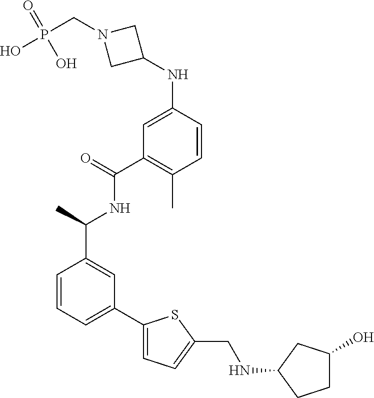 [SMILES: Cc1ccc(NC2CN(CP(=O)(O)O)C2)cc1C(=O)N[C@H](C)c1cccc(-c2ccc(CN[C@H]3CC[C@@H](O)C3)s2)c1] | ((3-((3-(((R)-1-(3-(5- ((((1S,3R)-3- hydroxycyclopentyl) amino)methyl)thiophen- 2-yl)phenyl)ethyl) carbamoyl)-4- methylphenyl)amino) azetidin-1- yl)methyl)phosphonic acid | 599.3 |
| 245 | 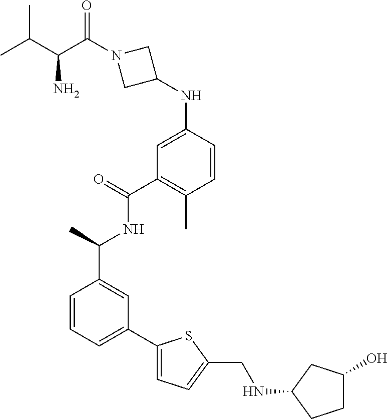 [SMILES: Cc1ccc(NC2CN(C(=O)[C@@H](N)C(C)C)C2)cc1C(=O)N[C@H](C)c1cccc(-c2ccc(CN[C@H]3CC[C@@H](O)C3)s2)c1] | 5-((1-(L-valyl)azetidin- 3-yl)amino)-N-((R)-1- (3-(5-((((1S,3R)-3- hydroxycyclopentyl) amino)methyl)thiophen- 2-yl)phenyl)ethyl)-2- methylbenzamide | 604.4 |
| 246 | 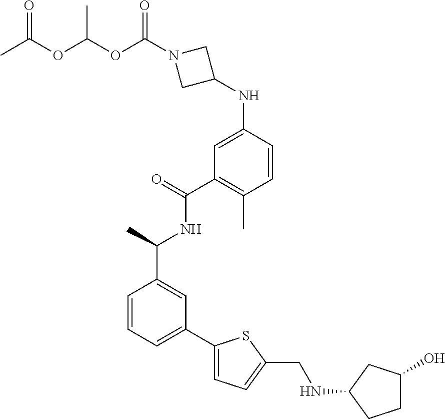 [SMILES: CC(=O)OC(C)OC(=O)N1CC(Nc2ccc(C)c(C(=O)N[C@H](C)c3cccc(-c4ccc(CN[C@H]5CC[C@@H](O)C5)s4)c3)c2)C1] | 1-acetoxyethyl 3-((3- (((R)-1-(3-(5- ((((1S,3R)-3- hydroxycyclopentyl) amino)methyl)thiophen-2- yl)phenyl)ethyl) carbamoyl)-4- methylphenyl)amino) azetidine-1-carboxylate | 635.2 |
| 247 | 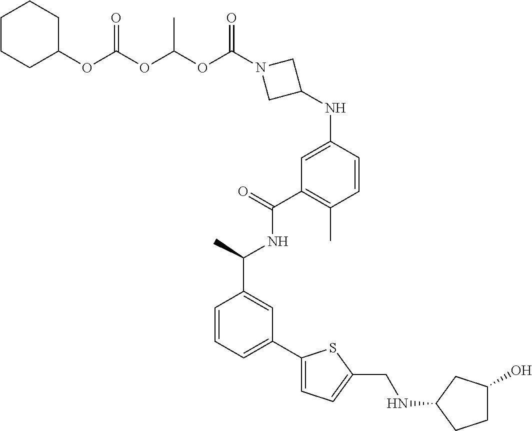 [SMILES: Cc1ccc(NC2CN(C(=O)OC(C)OC(=O)OC3CCCCC3)C2)cc1C(=O)N[C@H](C)c1cccc(-c2ccc(CN[C@H]3CC[C@@H](O)C3)s2)c1] | 1- (((cyclohexyloxy)carbonyl) oxy)ethyl 3-((3- (((R)-1-(3-(5- ((((1S,3R)-3- hydroxycyclopentyl) amino)methyl)thiophen-2- yl)phenyl)ethyl) carbamoyl)-4- methylphenyl)amino) azetidine-1-carboxylate | 719.4 |
| 248 | 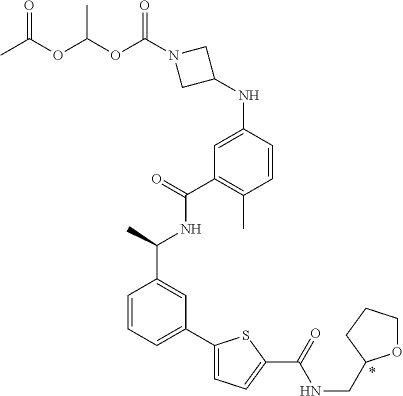 [SMILES: CC(=O)OC(C)OC(=O)N1CC(Nc2ccc(C)c(C(=O)N[C@H](C)c3cccc(-c4ccc(C(=O)NCC5CCCO5)s4)c3)c2)C1] | 1-acetoxyethyl 3-((3- (((R)-1-(3-(5- (((dihydro-2l3-furan- 2(3H)- yl)methyl)carbamoyl) thiophen-2- yl)phenyl)ethyl) carbamoyl)-4- methylphenyl)amino) azetidine-1-carboxylate | 649.5 |
| 249 | 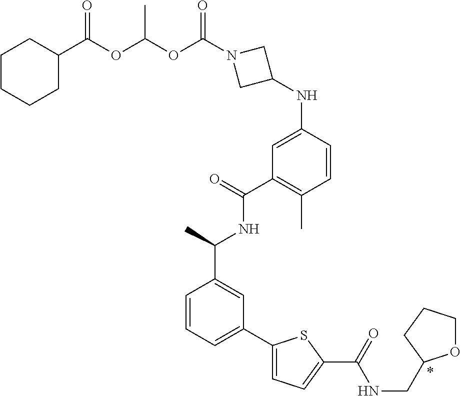 [SMILES: Cc1ccc(NC2CN(C(=O)OC(C)OC(=O)C3CCCCC3)C2)cc1C(=O)N[C@H](C)c1cccc(-c2ccc(C(=O)NCC3CCCO3)s2)c1] | 1- ((cyclohexanecarbonyl) oxy)ethyl 3-((3-(((R)- 1-(3-(5-(((dihydro-2l3- furan-2(3H)- yl)methyl)carbamoyl) thiophen-2- yl)phenyl)ethyl) carbamoyl)-4- methylphenyl)amino) azetidine-1-carboxylate | 717.5 |
| 250 | 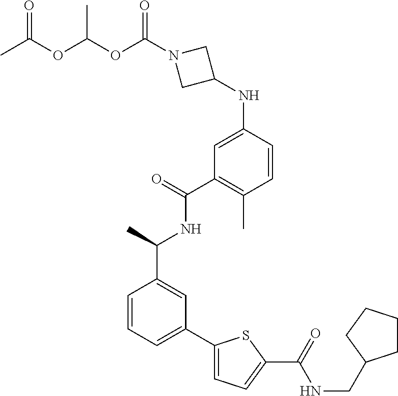 [SMILES: CC(=O)OC(C)OC(=O)N1CC(Nc2ccc(C)c(C(=O)N[C@H](C)c3cccc(-c4ccc(C(=O)NCC5CCCC5)s4)c3)c2)C1] | 1-acetoxyethyl 3-((3- (((R)-1-(3-(5- ((cyclopentylmethyl) carbamoyl)thiophen-2- yl)phenyl)ethyl) carbamoyl)-4- methylphenyl)amino) azetidine-1-carboxylate | 647.5 |
| 251 | 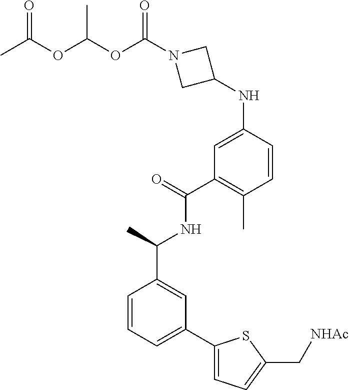 [SMILES: CC(=O)NCc1ccc(-c2cccc([C@@H](C)NC(=O)c3cc(NC4CN(C(=O)OC(C)OC(C)=O)C4)ccc3C)c2)s1] | 1-acetoxyethyl 3-((3- (((R)-1-(3-(5- (acetamidomethyl) thiophen-2- yl)phenyl)ethyl) carbamoyl)-4- methylphenyl)amino) azetidine-1-carboxylate | 593.6 |
| 252 | 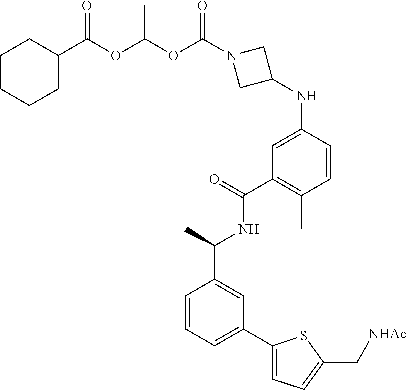 [SMILES: CC(=O)NCc1ccc(-c2cccc([C@@H](C)NC(=O)c3cc(NC4CN(C(=O)OC(C)OC(=O)C5CCCCC5)C4)ccc3C)c2)s1] | 1- ((cyclohexanecarbonyl) oxy)ethyl 3-((3-(((R)- 1-(3-(5- (acetamidomethyl) thiophen-2- yl)phenyl)ethyl) carbamoyl)-4- methylphenyl)amino) azetidine-1-carboxylate | 661.2 |
| 253 | 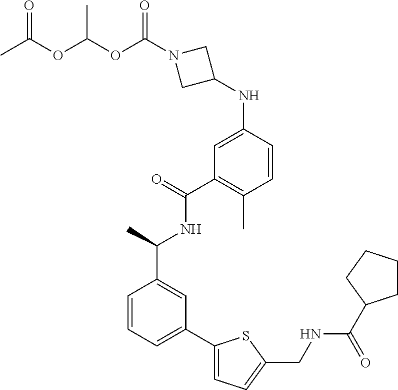 [SMILES: CC(=O)OC(C)OC(=O)N1CC(Nc2ccc(C)c(C(=O)N[C@H](C)c3cccc(-c4ccc(CNC(=O)C5CCCC5)s4)c3)c2)C1] | 1-acetoxyethyl 3-((3- (((R)-1-(3-(5- (cyclopentanecarboxamido- methyl)thiophen-2- yl)phenyl)ethyl) carbamoyl)-4- methylphenyl)amino) azetidine-1-carboxylate | 647.3 |

In some embodiments, the compounds of Formula XIV derivatized with a Protac are selected from one or more compounds of Table 6.

TABLE 6
PROTAC Compounds and Chemical Characterization

| Ex- | |
|---|---|
| am- | |
| ples | Structure |
| 254 | 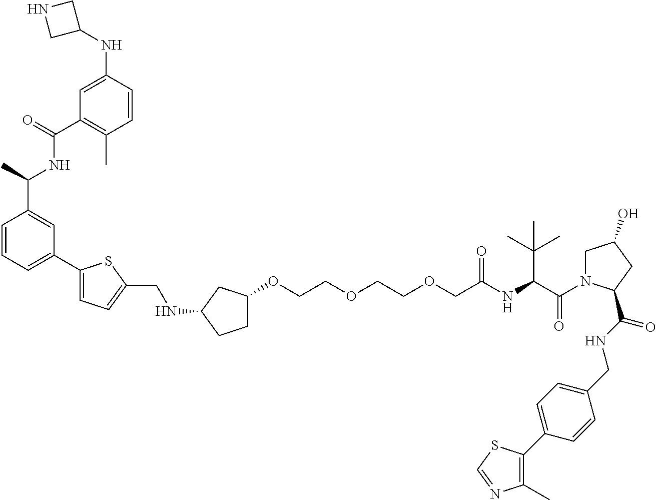 [SMILES: Cc1ccc(NC2CNC2)cc1C(=O)N[C@H](C)c1cccc(-c2ccc(CN[C@H]3CC[C@@H](OCCOCCOCC(=O)N[C@H](C(=O)N4C[C@H](O)C[C@H]4C(=O)NCc4ccc(-c5scnc5C)cc4)C(C)(C)C)C3)s2)c1] |
| 255 | 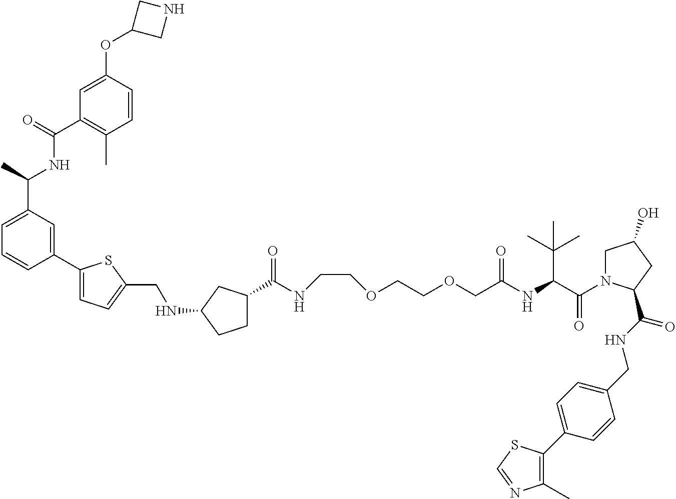 [SMILES: Cc1ccc(OC2CNC2)cc1C(=O)N[C@H](C)c1cccc(-c2ccc(CN[C@H]3CC[C@@H](C(=O)NCCOCCOCC(=O)N[C@H](C(=O)N4C[C@H](O)C[C@H]4C(=O)NCc4ccc(-c5scnc5C)cc4)C(C)(C)C)C3)s2)c1] |
| 256 | 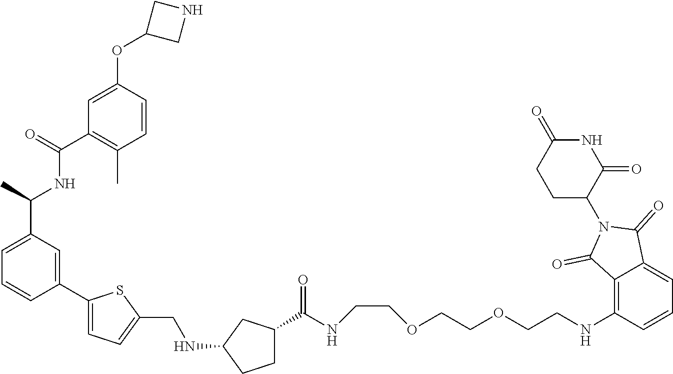 [SMILES: Cc1ccc(OC2CNC2)cc1C(=O)N[C@H](C)c1cccc(-c2ccc(CN[C@H]3CC[C@@H](C(=O)NCCOCCOCCNc4cccc5c4C(=O)N(C4CCC(=O)NC4=O)C5=O)C3)s2)c1] |
| 257 | 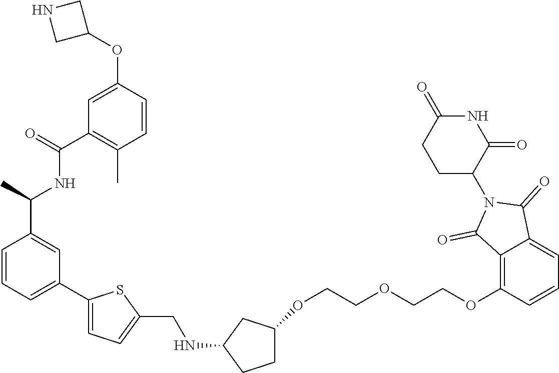 [SMILES: Cc1ccc(OC2CNC2)cc1C(=O)N[C@H](C)c1cccc(-c2ccc(CN[C@H]3CC[C@@H](OCCOCCOc4cccc5c4C(=O)N(C4CCC(=O)NC4=O)C5=O)C3)s2)c1] |
| 258 | 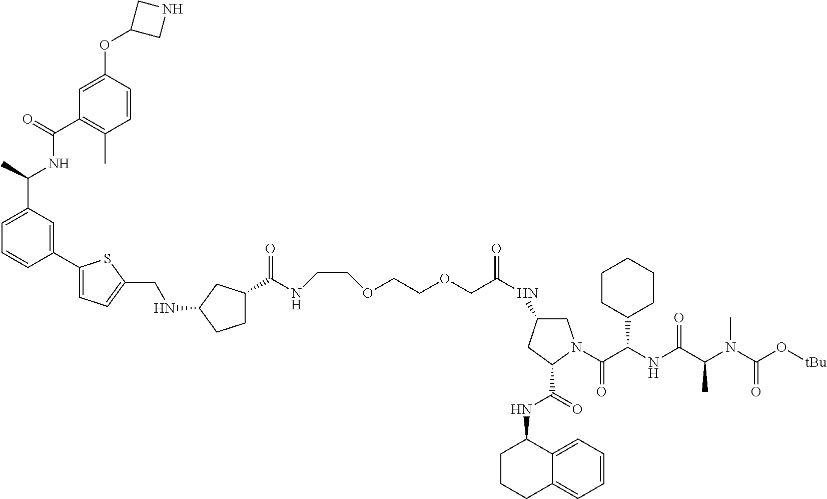 [SMILES: Cc1ccc(OC2CNC2)cc1C(=O)N[C@H](C)c1cccc(-c2ccc(CN[C@H]3CC[C@@H](C(=O)NCCOCCOCC(=O)N[C@H]4C[C@@H](C(=O)N[C@@H]5CCCc6ccccc65)N(C(=O)[C@@H](NC(=O)[C@H](C)N(C)C(=O)OC(C)(C)C)C5CCCCC5)C4)C3)s2)c1] |
| 259 | 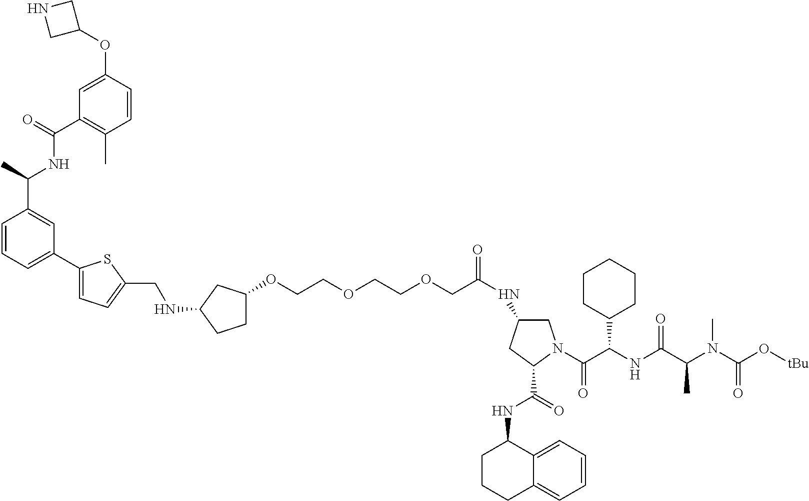 [SMILES: Cc1ccc(OC2CNC2)cc1C(=O)N[C@H](C)c1cccc(-c2ccc(CN[C@H]3CC[C@@H](OCCOCCOCC(=O)N[C@H]4C[C@@H](C(=O)N[C@@H]5CCCc6ccccc65)N(C(=O)[C@@H](NC(=O)[C@H](C)N(C)C(=O)OC(C)(C)C)C5CCCCC5)C4)C3)s2)c1] |

| | | LC-MS |
|---|---|---|
| | | characterization |
| Example | Chemical name | (M + H)⁺ |
| 254 | (2S,4R)-1-((S)-2-(2-(2-(2-(((1R,3S)-3-(((5-(3-((R)-1-(5-(azetidin-3-yloxy)-2-methylbenzamido)ethyl)phenyl) | 1064.4 |
| | thiophen-2-yl)methyl)amino)cyclopentyl)oxy)ethoxy)ethoxy)acetamido)-3,3-dimethylbutanoyl)-4-hydroxy-N- | |
| | (4-(4-methylthiazol-5-yl)benzyl)pyrrolidine-2-carboxamide | |
| 255 | (2S,4R)-1-((S)-1-((1R,3S)-3-(((5-(3-((R)-1-(5-(azetidin-3-yloxy)-2-methylbenzamido)ethyl)phenyl)thiophen-2- | 1091.4 |
| | yl)methyl)amino)cyclopentyl)-12-(tert-butyl)-1,10-dioxo-5,8-dioxa-2,11-diazatridecan-13-oyl)-4-hydroxy-N-(4-(4- | |
| | methylthiazol-5-yl)benzyl)pyrrolidine-2-carboxamide | |
| 256 | 5-(azetidin-3-yloxy)-N-((1R)-1-(3-(5-((((1S,3R)-3-((2-(2-(2-((2-(2,6-dioxopiperidin-3-yl)-1,3-dioxoisoindolin-4-yl) | 920.1 |
| | amino)ethoxy)ethoxy)ethyl)carbamoyl)cyclpentyl)amino)methyl)thiophen-2-yl)phenyl)ethyl)-2-methylbenzamide | |
| 257 | 5-(azetidin-3-yloxy)-N-((1R)-1-(3-(5-((((1S,3R)-3-(2-(2-((2-(2,6-dioxopiperidin-3-yl)-1,3-dioxoisoindolin-4- | 850.0 |
| | yl)oxy)ethoxy)ethoxy)cyclopentyl)amino)methyl)thiophen-2-yl)phenyl)ethyl)-2-methylbenzamide | |
| 258 | tert-butyl ((S)-1-(((S)-2-((2S,4S)-4-(2-(2-(2-((1R,3S)-3-(((5-(3-((R)-1-(5-(azetidin-3-yloxy)-2- | 1244.6 |
| | methylbenzamido)ethyl)phenyl)thiophen-2-yl)methyl)amino)cyclopentane-1-carboxamido)ethoxy) | |
| | ethoxy)acetamido)-2-(((R)-1,2,3,4-tetrahydronaphthalen-1-yl)carbamoyl)pyrrolidin-1-yl)-1-cyclohexyl-2- | |
| | oxoethyl)amino)-1-oxopropan-2-yl)(methyl)carbamate | |
| 259 | tert-butyl ((S)-1-(((S)-2-((2S,4S)-4-(2-(2-(2-(((1R,3S)-3-(((5-(3-((R)-1-(5-(azetidin-3-yloxy)-2- | 1217.6 |
| | methylbenzamido)ethyl)phenyl)thiophen-2-yl)methyl)amino)cyclopentyl)oxy)ethoxy)ethoxy)acetamido)-2-(((R)- | |
| | 1,2,3,4-tetrahydronaphthalen-1-yl)carbamoyl)pyrrolidin-1-yl)-1-cyclohexyl-2-oxoethyl)amino)-1-oxopropan-2- | |
| | yl)(methyl)carbamate | |

### Compositions Comprising Compounds Described Herein

In some aspects, the present disclosure provides pharmaceutical compositions comprising one or more compounds described herein and/or derivatives thereof. The pharmaceutical compositions of the present disclosure can have various formulations for different routes of administration, including, but not limited to, oral formulations, injectable formulations, and liquid formulations.

In some embodiments, the pharmaceutical compositions comprising one or more compounds described herein and/or derivatives thereof have injectable formulations or are formulated for injections through various administration routes, including, but not limited to, intranasal administration, subcutaneous administration, intravenous administration, intraperitoneal administration, intramuscular administration, intradermal administration, and intrathecal administration. In some embodiments, the pharmaceutical compositions comprising one or more compounds described herein and/or derivatives thereof is in a liquid formulation, for example, in the form of an emulsion, for intravenous administration.

In some embodiments, the pharmaceutical compositions comprising one or more compounds described herein and/or derivatives thereof are formulated for oral administration or are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

In some embodiments, the pharmaceutical compositions comprising one or more compounds described herein and/or derivatives thereof are in the form of solid, semi-solid, or liquid dosage forms. Non-limiting examples of suitable solid, semi-solid, or liquid dosage forms include tablets (e.g. suspension tablets, bite suspension tablets, rapid dispersion tablets, chewable tablets, melt tablets, effervescent tablets, bilayer tablets, etc.), caplets, capsules (e.g. a soft or a hard gelatin capsule filled with solid and/or liquids), powder (e.g. a packaged powder, a dispensable powder or an effervescent powder), lozenges, sachets, cachets, troches, pellets, granules, microgranules, encapsulated microgranules, powder aerosol formulations, solutions, suspension, elixirs, syrups, liquid aerosol formulations, or any other solid, semi-solid, or liquid dosage form reasonably adapted for oral administration. In some embodiments, the orally deliverable pharmaceutical compositions comprising one or more compounds described herein and/or derivatives thereof can be ingested directly, or they can be mixed with food or beverage prior to ingestion.

In some embodiments, the orally deliverable pharmaceutical compositions are a tablet, capsule, softgel, or an aqueous or nonaqueous solution, suspension, or syrup. In some embodiments, the orally deliverable pharmaceutical compositions comprise one or more carriers, e.g., lactose and/or corn starch. In some embodiments, the orally deliverable compositions comprise one or more lubricating agents such as magnesium stearate. In some embodiments, the orally deliverable pharmaceutical composition comprises an oral, non-toxic, pharmaceutically acceptable, inert carrier. Non-limiting examples of inert carriers include lactose, starch, sucrose, glucose, methyl cellulose, magnesium stearate, dicalcium phosphate, calcium sulfate, mannitol, and sorbitol.

In some embodiments, the compositions optionally comprise one or more non-toxic solid carriers. Non-limiting examples of non-toxic solid carries include pharmaceutical grades of mannitol, lactose, starch, magnesium stearate, sodium saccharin, talc, cellulose, glucose, sucrose, and magnesium carbonate.

In some embodiments, the pharmaceutical compositions comprising one or more compounds described herein and/or derivatives thereof encapsulated in a capsule shell. In some embodiments, the capsule is a hard gelatin capsule. In some embodiments, the capsule is a soft gelatin capsule.

In some embodiments, the pharmaceutical compositions comprising one or more compounds described herein and/or derivatives thereof are liquid pharmaceutically administrable compositions. In some embodiments, the liquid pharmaceutically administrable compositions by dissolving, dispersing, and the like, one or more compounds described herein and/or derivatives thereof and optionally, pharmaceutical adjuvants in an excipient, such as, for example, water, saline, aqueous dextrose, glycerol, ethanol, and the like, to thereby form a solution or suspension. In some embodiments, the pharmaceutical compositions comprise minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like.

In some embodiments, the pharmaceutical compositions comprising one or more compounds described herein and/or derivatives thereof optionally comprise one or more pharmaceutically acceptable excipients. The term “pharmaceutically acceptable excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition, and that does not produce unacceptable toxicity or interaction with other components in the composition.

In some embodiments, the pharmaceutical compositions comprising one or more compounds and/or derivatives thereof can be formulated to have modified rates of release. Suitable modified-release formulations include those that exhibit a delayed -or extended-release. An “extended-release” formulation can extend the period over which the pharmaceutically active compound is released or targeted to the desired site. A “delayed-release” formulation can be designed to delay the release of the pharmaceutically active compound for a specified period. Mechanisms can be dependent or independent of local pH in the stomach and/or intestine and can also rely on local enzymatic activity to achieve the desired effect. Examples of modified-release formulations are known in the art and are described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; The Handbook of Pharmaceutical Controlled Release Technology, D. L. Wise (ed.), Marcel Decker, Inc., New York (2000); and also in Treatise on Controlled Drug Delivery: Fundamentals, Optimization, and Applications, A. Kydonieus (ed.), Marcel Decker, Inc., New York, (1992), the relevant contents of each of which are hereby incorporated by reference for this purpose.

In some embodiments, the compositions optionally comprise one or more pharmaceutically acceptable diluents as excipients. Non-limiting examples of suitable diluents include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of amorphous cellulose (e.g., Rexcel™) and powdered cellulose; calcium carbonate; glycine; bentonite; polyvinylpyrrolidone; and the like. Such diluents, if present, constitute in total about 5% to about 99%, about 10% to about 85%, or about 20% to about 80%, of the total weight of the composition.

In some embodiments, the compositions optionally comprise one or more pharmaceutically acceptable disintegrants as excipients. Non-limiting examples of suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, xanthan, locust bean, karaya, pectin and tragacanth gums. Such disintegrants, if present, typically comprise in total about 0.2% to about 30%, about 0.2% to about 10%, or about 0.2% to about 5%, of the total weight of the composition.

In some embodiments, the compositions optionally comprise one or more antioxidants. Non-limiting examples of antioxidants include sodium ascorbate, vitamin E (tocopherol), ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. One or more antioxidants, if present, are typically present in a composition in an amount of about 0.001% to about 5%, about 0.005% to about 2.5%, or about 0.01% to about 1%, by weight.

In some embodiments, the compositions optionally comprise one or more pharmaceutically acceptable binding agents or adhesives as excipients. Such binding agents and adhesives can impart sufficient cohesion to a powder being tableted to allow for normal processing operations such as sizing, lubrication, compression and packaging, but still allow the tablet to disintegrate and the composition to be absorbed upon ingestion. Non-limiting examples of suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K 29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, about 0.75% to about 15%, or about 1% to about 10%, of the total weight of the composition.

In some embodiments, the compositions optionally comprise one or more pharmaceutically acceptable wetting agents as excipients. Non-limiting examples of surfactants that can be used as wetting agents in compositions include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, about 0.4% to about 10%, or about 0.5% to about 5%, of the total weight of the composition.

In some embodiments, the compositions optionally comprise one or more pharmaceutically acceptable lubricants (including anti-adherents and/or glidants) as excipients. Non-limiting examples of suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; sodium chloride; and magnesium lauryl sulfate. Such lubricants, if present, constitute in total about 0.1% to about 10%, about 0.2% to about 8%, or about 0.25% to about 5%, of the total weight of the composition.

In some embodiments, the compositions optionally comprise one or more permeation enhancer excipients. Non-limiting examples of permeation enhancer excipients include polymers such as: polycations (chitosan and its quaternary ammonium derivatives, poly-L-arginine, aminated gelatin); polyanions (N-carboxymethyl chitosan, poly-acrylic acid); and thiolated polymers (carboxymethyl cellulose-cysteine, polycarbophil-cysteine, chitosan-thiobutylamidine, chitosan-thioglycolic acid, chitosan-glutathione conjugates).

In some embodiments, the compositions optionally comprise one or more binders. Non-limiting examples of binders include starch, gelatin, natural sugars such as glucose or beta-lactose, corn sweeteners, natural and synthetic gums such as acacia, tragacanth, or sodium alginate, carboxymethylcellulose, polyethylene glycol, and waxes.

In some embodiments, the compositions optionally comprise one or more flavoring agents, sweetening agents, and/or colorants. Non-limiting examples of flavoring agents include acacia syrup, alitame, anise, apple, aspartame, banana, Bavarian cream, berry, blackcurrant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc. Flavoring agents, sweetening agents, and/or colorants can be present in the compositions in any suitable amount, for example about 0.01% to about 10%, about 0.1% to about 8%, or about 1% to about 5%, by weight.

In some embodiments, the composition is formulated for parenteral administration. Non-limiting examples of parenteral administration include intraarticular, intravenous, intramuscular, intradermal, intraperitoneal, and subcutaneous administration. In some embodiments, the compositions are an aqueous and non-aqueous, isotonic sterile injection solution optionally comprising antioxidants, buffers, bacteriostats, and solutes that render the formulation isotonic with the blood of the intended recipient. In another embodiment, the compositions are aqueous and non-aqueous sterile suspensions that can optionally include suspending agents, solubilizers, thickening agents, stabilizers, and preservatives.

In some embodiments, parenteral administration comprises administration the composition to a subject via a needle or a catheter, sterile syringe, or other mechanical device such as a continuous infusion system. In some embodiments, parenteral administration includes introducing the composition to the subject via a syringe, injector, or pump.

In some embodiments, the composition is a sterile injectable suspension comprising a suitable carrier, dispersing, or wetting agents, and suspending agents. In some embodiments, the sterile injectable suspension comprises a nontoxic parenterally acceptable diluent or solvent. Non-limiting examples of suitable diluent or solvents include water, Ringer's solution, and isotonic sodium chloride solution. In some embodiments, the sterile injectable suspension comprises fixed oils, fatty esters, or polyols.

In some embodiments, the composition is a sterile aqueous or non-aqueous solution, suspension, or emulsion. Non-limiting examples of non-aqueous solvents include propylene glycol, polyethylene glycol, vegetable oils (e.g., olive oil and corn oil), gelatin, and injectable organic esters such as ethyl oleate. In some embodiments, the composition comprises adjuvants such as preserving, wetting, emulsifying, and dispersing agents. In some embodiments, the composition is sterilized, for example, by filtering the composition through a bacterium retaining filter, by incorporating sterilizing agents into the compositions, by irradiating the compositions, or by heating the compositions. In some embodiments, the composition is formulated from sterile water, or some other sterile injectable medium, immediately before use.

In some embodiments, the composition is formulated for rectal administration. A composition for rectal administration can be prepared by mixing the one or more compounds of the present disclosure and/or derivatives thereof with a suitable nonirritating excipient which is solid at room temperature but liquid at the rectal temperature. As such, the formulation will melt in the rectum to release the drug. Non-limiting examples of nonirritating excipients include cam butter, beeswax, and polyethylene glycols.

In some embodiments, the composition is formulated for aerosol administration. In some embodiments, the aerosol administration includes intranasal administration. In some embodiments, the composition comprises one or more of a preservative, absorption promoter, or propellant. Non-limiting examples of propellants include chlorofluorocarbon (CFC) for example dichlorodifluoromethane, trichlorofluoromethane, dichlorotetrafluoroethane, carbon dioxide or other suitable gas. In some embodiments, one or more compounds of the present disclosure and/or derivatives thereof is a dry powder that can form a gel in a nasal cavity. In some embodiments, the dry powder includes one or more compounds of the present disclosure and/or derivatives thereof mixed with a suitable powder base such as lactose, starch, starch derivatives such as hydroxypropylmethyl cellulose and polyvinylpyrrolidine (PVP).

In some embodiments, the composition comprises a therapeutically effective amount of one or more compounds of the present disclosure and/or derivatives thereof, where the therapeutically effective amount includes about 0.01 mg/kg to about 250 mg/kg body weight. For example, about 0.1 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 210 mg/kg, about 220 mg/kg, about 230 mg/kg, about 240 mg/kg, or about 250 mg/kg.

### Additional Therapeutic Agents

In some aspects, the pharmaceutical compositions comprising one or more compounds described herein and/or derivatives thereof according to various embodiments of the present disclosure comprise one or more additional therapeutic agents or are used for co-administration regimens with one or more additional therapeutic agents.

In some embodiments, the one or more additional therapeutic agents may be formulated into the same pharmaceutical composition comprising one or more compounds described herein and/or derivatives thereof, for example, as a single dosage unit or as multiple dosage units for coordinated, combination, or concomitant administration, or into separate pharmaceutical compositions for combinational therapy.

In some embodiments, the one or more additional therapeutic agents may be formulated as separate pharmaceutical compositions, for example, as a single dosage unit or as multiple dosage units, for co-administration with the pharmaceutical composition comprising one or more compounds described herein and/or derivatives thereof.

Non-limiting examples of classes of additional therapeutic agents suitable for use in accordance with the present invention include: antiviral agents; anti-inflammatory agents; antimalaria agents; and biological agents.

In some embodiments, the one or more additional therapeutic agents comprise an antiviral agent. Non-limiting examples of an antiviral agent include remdesivir (e.g., Veklury®), favipiravir (e.g., Avigan®), lopinavir/ritonavir (e.g., Kaletra®, Aluvia®), nitazoxanide (e.g., Alinia®), danoprevir (e.g., Ganovo®), ASC-09, umifenovir (e.g., Arbidol®), nafamostat, brequinar, AT-527, ABX464, merimepodib, molnupiravir, opaganib (e.g., Yeliva®), ivermectin (e.g., Soolantra®, Stromectol®, Sklice®), and hydroxychloroquine.

In some embodiments, the one or more additional therapeutic agents comprise an antimalaria agent. Non-limiting examples of an antimalaria agent include hydroxychloroquine and chloroquine.

In some embodiments, the one or more additional therapeutic agents comprise a biologic agent. In some embodiments, the biological agent is an antibody, for example, an antibody recognizing at least a portion of the SARS-CoV-2 coronaviruse, such as an epitope on a spike protein.

In some embodiments, the biological agent is a vaccine, for example, a vaccine against the SARS-CoV-2 coronaviruse. In some embodiments, the vaccine is BNT162b2 (Pfizer/BioNTech), mRNA-1273 (Moderna), AZD1222/ChAdOxl (AstraZeneca/Oxford Univ), Ad5-vectored COVID-19 vaccine (CanSino Biologies), CoronaVac (Sinovac), and/or NVX-CoV2373 (Novavax).

In some embodiments, the one of more additional therapeutic agents may be formulated in various formulations, for example, injectable formulations, lyophilized formulations, liquid formulations, or oral formulations. The formulation can be selected based upon the suitable administration route.

Treatment and/or Prevention of SARS-COV-2 Infections

In some aspects, the pharmaceutical compositions comprising one or more compounds described herein and/or derivatives thereof according to various embodiments of the present disclosure may be used in the treatment and/or prevention of infections and/or diseases caused by coronaviruses, including COVID-19 caused by SARS-CoV-2. In some embodiments, the present disclosure also provides methods for treatment and/or prevention of a disease or symptoms associated thereof caused by a coronaviral infection in a subject.

In some embodiments, the present disclosure provides methods for treatment, prevention, or amelioration of one or more symptoms and/or diseases associated with a coronavirus. In some embodiments, methods for the treatment and/or prevention of COVID-19 associated with infection of the SARS-CoV-2 virus are provided. As used herein, the terms “SARS-CoV-2”, “coronavirus”, “corona”, “2019 novel coronavirus,” “2019-nCoV”, and “COVID-19” are used interchangeably throughout the present disclosure.

In some embodiments, the present disclosure provides methods for the treatment and/or prevention of infections and/or diseases caused by coronaviruses in a subject in need thereof comprising administering to the subject a pharmaceutical composition comprising a therapeutically effective amount of one or more compounds described herein and/or derivatives thereof according to various embodiments of the present disclosure.

In some embodiments, the methods further comprise administering the subject a therapeutic effect amount of one or more additional therapeutic agents according to various embodiments of the present disclosure. The pharmaceutical composition comprising one or more compounds described herein and/or derivatives thereof can be used in combination with one or more additional therapeutic agents to obtain improved or synergistic therapeutic effects. In some embodiments, the one or more additional therapeutic agents comprise an antiviral agent, an antimalaria agent, and/or a biologic agent. In some embodiments, the antiviral agent is remdesivir (e.g., Veklury®), favipiravir (e.g., Avigan®), lopinavir/ritonavir (e.g., Kaletra®, Aluvia®), nitazoxanide (e.g., Alinia®), danoprevir (e.g., Ganovo®), ASC-09, umifenovir (e.g., Arbidol®), nafamostat, brequinar, AT-527, ABX464, merimepodib, molnupiravir, opaganib (e.g., Yeliva®), ivermectin (e.g., Soolantra®, Stromectol®, Sklice®), and/or hydroxychloroquine. In some embodiments, the antimalaria agent is hydroxychloroquine or chloroquine. In some embodiments, the biologic agent is an antibody, for example, an antibody recognizing the SARS-CoV-2 coronaviruse. In some embodiments, the biological agent is a vaccine, for example, a vaccine for the SARS-CoV-2 coronavirus. In some embodiments, the vaccine is BNT162b2 (Pfizer/BioNTech), mRNA-1273 (Moderna), AZD1222/ChAdOxl (AstraZeneca/Oxford Univ), Ad5-vectored COVID-19 vaccine (CanSino Biologies), CoronaVac (Sinovac), and/or NVX-CoV2373 (Novavax).

In some embodiments, the subject is administered the one or more additional therapeutic agents before administration of the pharmaceutical composition comprising one or more compounds described herein and/or derivatives thereof. In some embodiments, the subject is co-administered the one or more additional therapeutic agents and the pharmaceutical composition comprising one or more compounds described herein and/or derivatives thereof. In some embodiments, the subject is administered the one or more additional therapeutic agents before after administration of the pharmaceutical composition comprising one or more compounds described herein and/or derivatives thereof.

It is within the purview of one of ordinary skill in the art to select a suitable administration route, such as oral administration, subcutaneous administration, intravenous administration, intramuscular administration, intradermal administration, intrathecal administration, or intraperitoneal administration, for the one or more additional therapeutic agents.

As one of ordinary skill in the art would understand, the pharmaceutical composition comprising one or more compounds described herein and/or derivatives thereof and the one or more additional therapeutic agents can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

In some embodiments, methods for treatment and/or prevention of a disease or symptoms associated thereof caused by SARS-CoV-2 in a subject are provided, wherein the subject is elderly (e.g., 65 years or greater), an infant, or an immunocompromised subject. In some embodiments, the subject has one or more underlying medical conditions resulting an increased risk of severe illness from COVID-19. Non-limiting examples of underlying medical conditions that render a subject at increased risk of severe illness from COVID-19 include cancer, cardiovascular disease, chronic kidney disease, chronic obstructive pulmonary disease (COPD), immunocompromised state, obesity (i.e., body mass index (BMI) of 30 or higher), serious heart conditions (e.g., heart failure, coronary artery disease, cardiomyopathy), sickle cell disease, Type 2 diabetes mellitus, asthma, cerebrovascular disease, cystic fibrosis, hypertension or high blood pressure, neurologic conditions (e.g., dementia), liver disease, pregnancy, pulmonary fibrosis, smoking, thalassemia, and Type 1 diabetes mellitus.

### Kits

Provided herein in certain embodiments are kits for carrying out the methods disclosed herein. In certain embodiments, the kits comprise one or more compounds of the present disclosure and/or derivatives thereof, or one or more pharmaceutical formulations comprising such compounds and/or derivatives thereof. In certain embodiments, the kits further comprise one or more additional therapeutic agents (e.g., anti-viral agent) or pharmaceutical formulations thereof. In those embodiments wherein the kits comprise two or more compounds of the present disclosure and/or derivatives thereof and an additional therapeutic agent, the two or more compounds may be present in the kit in a single composition or in separate compositions. In certain embodiments, the kits comprise instructions in a tangible medium.

The foregoing and the following working examples are merely intended to illustrate various embodiments of the present invention. The specific modifications discussed above are not to be construed as limitations on the scope of the invention.

It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### EXAMPLES

### Example 1: Exemplary Chemical Syntheses of Compounds of the Present Disclosure and Biological Assays of the Same

The following example outlines an exemplary methodology for synthesizing and the subsequent characterization of compounds of the present disclosure.

### Methods and Materials

General Chemical Experimental Information: Unless otherwise specified, reactions were performed under an inert atmosphere of argon and monitored by thin-layer chromatography (TLC) and/or LCMS. All reagents were purchased from commercial suppliers and used as provided. Synthetic intermediates were purified using CombiFlash chromatography system on 230-400 mesh silica gel. ¹H and ¹³C NMR spectra were obtained using Bruker DPX-400 or AVANCE-400 spectrometer at 400 and 100 MHz, respectively. NMR chemical shifts were described in δ (ppm) using residual solvent peaks as standard (Chloroform-d, 7.26 ppm (¹H), 77.16 ppm (¹³C); Methanol-d₄, 3.31 ppm (¹H), 49.00 ppm (¹³C); DMSO-d₆, 2.50 ppm (¹H), 39.52 ppm (¹³C); Acetone-d6, 2.05 ppm (¹H), 29.84 ppm (¹³C)). Data were reported in a format as follows: chemical shift, multiplicity (s=singlet, d=doublet, dd=doublet of doublet, t=triplet, q=quartet, br=broad, m=multiplet, abq=ab quartet), number of protons, and coupling constants. High resolution mass spectral data were measured in-house using a Shimadzu IT-TOFLC/MS for all final compounds. All compounds submitted for biological testing were confirmed to be >95% pure by analytical HPLC. Synthetic methods, spectral data, and HRMS for novel compounds are described in detail below.

General Procedure for Reductive Amination: To a solution of amine compound and ketone (or aldehyde) compound in MeOH, HOAc was added. After stirring at the indicated temperature for 2 h and cooldown, NaBH₃CN was added carefully. The reaction was continued at room temperature overnight and then concentrated under vacuum. Dissolve the mixture in EA and wash with water and brine. After that, the organic layer was dried over Na₂SO₄, filtered, and concentrated. The residue was purified by silica gel column chromatography or Prep-HPLC to provide the amination compound.

General Procedure for Amine Coupling: Amine compound, acid compound, HATU, TEA (or DIPEA) and DMAP were dissolved in dry DMF or DCM and stirred at room temperature overnight. The mixture was diluted with Ethyl Acetate and was then washed with saturated aq. NaHCO₃, water, and brine, respectively. The organic layer was dried over Na₂SO₄, filtered, and concentrated. The residue was purified by silica gel column chromatography or Prep-HPLC to provide the desired amide.

General Procedure for N-Boc Deprotection: To a solution of Boc protected compound in DCM was added HCl (4M in dioxane) at 0° C., and then warmed up to room temperature. After stirring for another 2 h, the reaction was dried under vacuum.

The residue was purified by Prep-HPLC to provide the deprotected compound.

General Procedure for Aryl Nitro Reduction: To a solution of Aryl Nitro compound in ethanol/saturated aq. NH₄Cl (4:1), Fe powder was added. The resulting solution was stirred for 2 h at 80° C. After cooling down, the mixture was extracted with ethyl acetate 3 times. The organic layer was washed with brine, dried, and concentrated under vacuum. The residue was purified by silica gel column chromatography or Prep-HPLC to obtain the desired product.

General Procedure for(R)-1-arylethan-1-amine: Ellman amine synthesis was used that utilize(R)-(+)-2-Methyl-2-propanesulfinamide as the amine source under strong Lewis acid, Ti(OEt)₄, followed by reduction with NaBH₄.

### Reaction Schemes

Synthesis of compounds of the present disclosure followed the general synthetic scheme shown in Scheme 1. The following schemes in this example are presented to provide what is believed to be the most useful and readily understood description of procedures and conceptual aspects of this invention. Each exemplified compound and intermediate was named using ChemDraw Ver. 20.0.0.41

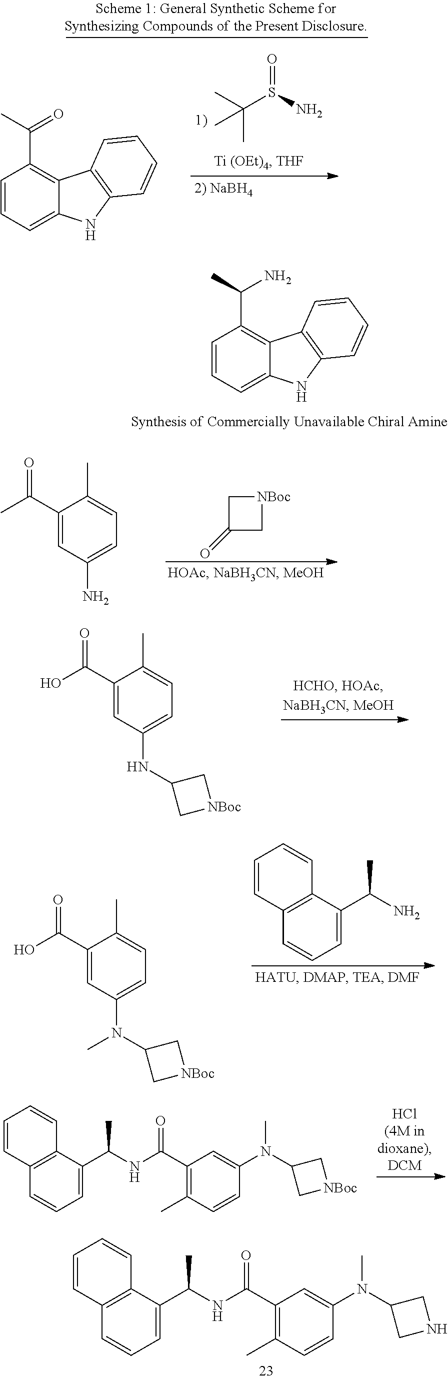

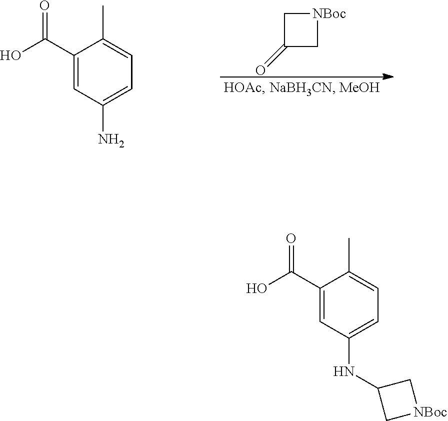 [SMILES: Cc1ccc(N)cc1C(=O)O ; CC(C)(C)OC(=O)N1CC(=O)C1 ; Cc1ccc(NC2CN(C(=O)OC(C)(C)C)C2)cc1C(=O)O]

5-((1-(tert-butoxycarbonyl)azetidin-3-yl)amino)-2-methylbenzoic acid. 5-amino-2-methylbenzoic acid (3.0 g, 19.8 mmol) and tert-butyl 3-oxoazetidine-1-carboxylate (10.1 g, 59.5 mmol) was subjected to general reductive amination procedure with MeOH (20 mL), HOAc (5 mL) at 50° C., and then NaBH₃CN (6.2 g, 99.0 mmol) was added. The purification by silica gel column chromatography (Hexenes/EtOAc, 1:1) provided the amination compound (5.1 g, yield 83%) as a white solid: ¹H NMR (400 MHz, Chloroform-d) δ 7.16 (d, J=2.6 Hz, 1H), 7.07 (d, J=8.3 Hz, 1H), 6.62 (dd, J=8.3, 2.6 Hz, 1H), 4.33-4.27 (m, 2H), 4.24-4.18 (m, 1H), 3.76-3.70 (m, 2H), 2.49 (s, 3H), 1.43 (d, J=2.1 Hz, 9H); LRMS (ESI) calcd for C₁₆H₂₃N₂O₄ [M+H]⁺307.17, found 307.14.

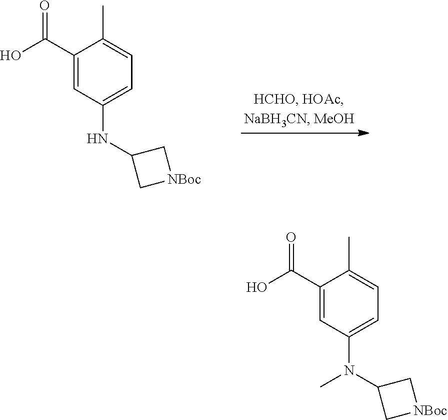 [SMILES: Cc1ccc(NC2CN(C(=O)OC(C)(C)C)C2)cc1C(=O)O ; Cc1ccc(N(C)C2CN(C(=O)OC(C)(C)C)C2)cc1C(=O)O]

5-((1-(tert-butoxycarbonyl)azetidin-3-yl)(methyl)amino)-2-methylbenzoic acid. 5-((1-(tert-butoxycarbonyl)azetidin-3-yl)amino)-2-methylbenzoic acid (2.3 g, 7.5 mmol) and formaldehyde (37 wt. % in H₂O, 2 mL) was subjected to general reductive amination procedure with MeOH (10 mL), HOAc (2 mL) at room temperature, and then NaBH₃CN (2.4 g, 37.5 mmol) was added. The purification by silica gel column chromatography (Hexenes/EtOAc, 2:1) provided the amination compound (2.1 g, yield 87%) as a colorless oil: ¹H NMR (400 MHz, Methanol-d₄) δ 7.31 (d, J=2.8 Hz, 1H), 7.13 (d, J=8.4 Hz, 1H), 6.88 (dd, J=8.3, 2.8 Hz, 1H), 4.22 (tt, J=7.0, 5.0 Hz, 1H), 4.14 (t, J=8.0 Hz, 2H), 3.84 (dd, J=8.9, 5.0 Hz, 2H), 2.83 (s, 3H), 2.46 (s, 3H), 1.43 (s, 9H); LRMS (ESI) calcd for C₁₇H₂₅N₂O₄ [M+H]⁺321.18, found 321.20.

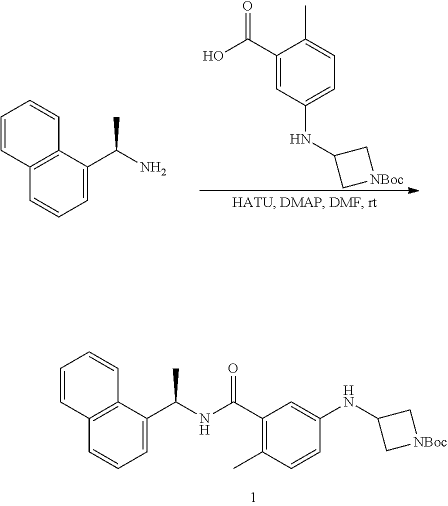 [SMILES: C[C@@H](N)c1cccc2ccccc12 ; Cc1ccc(NC2CN(C(=O)OC(C)(C)C)C2)cc1C(=O)O ; Cc1ccc(NC2CN(C(=O)OC(C)(C)C)C2)cc1C(=O)N[C@H](C)c1cccc2ccccc12]

Tert-butyl-(R)-3-((4-methyl-3-((1-(naphthalen-1-yl)ethyl)carbamoyl)phenyl)amino)azetidine-1-carboxylate. (R)-1-(naphthalen-1-yl)ethan-1-amine (300 mg, 1.75 mmol), 5-((1-(tert-butoxycarbonyl)azetidin-3-yl)amino)-2-methylbenzoic acid (482 mg, 1.58 mmol), HATU (684 mg, 1.80 mmol) and DMAP (428 mg, 3.5 mmol) was subjected to general amine coupling procedure with DMF (4 mL). The purification by Prep-HPLC afforded the compound (667 mg, yield 92%) as a white solid: [α]₅₄₆²⁵=−82.6 (c 6.7, MeCOH); ¹H NMR (400 MHz, Methanol-d₄) δ 8.69 (d, J=8.2 Hz, 1H), 8.27-8.19 (m, 1H), 7.89-7.85 (m, 1H), 7.80-7.76 (m, 1H), 7.63-7.59 (m, 1H), 7.58-7.52 (m, 1H), 7.51-7.43 (m, 2H), 6.97-6.94 (m, 1H), 6.51-6.46 (m, 2H), 6.06-5.98 (m, 1H), 4.17-4.01 (m, 3H), 3.68-3.61 (m, 2H), 2.21 (s, 3H), 1.67 (d, J=6.9 Hz, 3H), 1.43 (s, 9H). ¹³C NMR (100 MHz, Methanol-d₄) δ 172.40, 158.02, 146.11, 140.32, 138.66, 138.61, 135.38, 132.42, 132.33, 129.88, 128.93, 127.22, 126.68, 126.39, 125.13, 124.35, 123.77, 115.46, 112.84, 80.96, 46.35, 46.25, 44.09, 28.66, 21.37, 18.66; HRMS (ESI) calcd for C₂₈H₃₄N₃O₃ [M+H]⁺460.2595, found 460.2590.

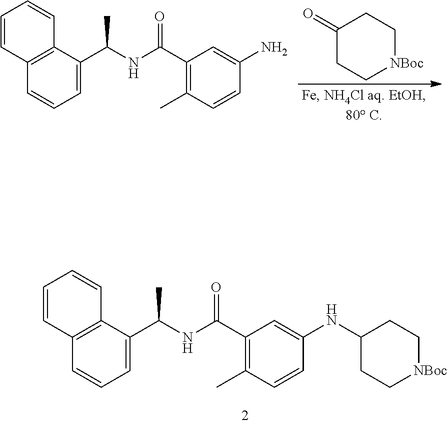 [SMILES: Cc1ccc(N)cc1C(=O)N[C@H](C)c1cccc2ccccc12 ; CC(C)(C)OC(=O)N1CCC(=O)CC1 ; Cc1ccc(NC2CCN(C(=O)OC(C)(C)C)CC2)cc1C(=O)N[C@H](C)c1cccc2ccccc12]

Tert-butyl-(R)-4-((4-methyl-3-((1-(naphthalen-1-yl)ethyl)carbamoyl)phenyl)amino)piperidine-1-carboxylate. (R)-5-amino-2-methyl-N-(1-(naphthalen-1-yl)ethyl)benzamide (30 mg, 0.10 mmol) and tert-butyl 4-oxopiperidine-1-carboxylate (100 mg, 0.50 mmol) was subjected to general reductive amination procedure with MeOH (1 mL), HOAc (0.2 mL) at 50° C., and then NaBH₃CN (32 mg, 0.50 mmol) was added. The purification by Prep-HPLC afforded the desired compound (42 mg, yield 86%) as a white solid: [α]₅₄₆²⁵=−75.0 (c 0.3, MeOH); ¹H NMR (400 MHz, Methanol-d₄) δ 8.72 (d, J=8.2 Hz, 1H), 8.28-8.21 (m, 1H), 7.90 (dd, J=8.1, 1.4 Hz, 1H), 7.81 (d, J=8.2 Hz, 1H), 7.63 (d, J=7.1 Hz, 1H), 7.61-7.43 (m, 3H), 6.98 (d, J=8.3 Hz, 1H), 6.66 (dd, J=8.2, 2.5 Hz, 1H), 6.61 (d, J=2.5 Hz, 1H), 6.02 (td, J=6.8, 4.9 Hz, 1H), 4.02-3.93 (m, 2H), 3.41-3.33 (m, 1H), 2.95-2.83 (m, 2H), 2.21 (s, 3H), 1.96-1.88 (m, 2H), 1.70 (d, J=6.9 Hz, 3H), 1.46 (s, 9H), 1.33-1.21 (m, 2H); ¹³C NMR (100 MHz, Methanol-d₄) δ 172.56, 156.48, 140.33, 138.70, 138.65, 135.48, 132.41, 129.91, 128.98, 127.24, 126.71, 126.41, 124.38, 123.80, 116.56, 113.79, 81.06, 51.60, 46.40, 46.31, 32.88, 28.68, 21.30, 18.57. HRMS (ESI) calcd for C₃₀H₃₈N₃O₃ [M+H]⁺ 488.2908, found 488.2957.

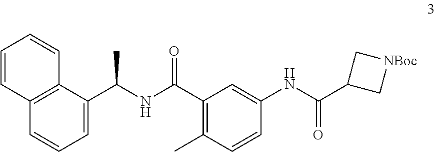 [SMILES: Cc1ccc(NC(=O)C2CN(C(=O)OC(C)(C)C)C2)cc1C(=O)N[C@H](C)c1cccc2ccccc12]

Tert-butyl-(R)-3-((4-methyl-3-((1-(naphthalen-1-yl)ethyl)carbamoyl)phenyl)carbamoyl)azetidine-1-carboxylate. (R)-5-amino-2-methyl-N-(1-(naphthalen-1-yl)ethyl)benzamide (30 mg, 0.10 mmol), 1-(tert-butoxycarbonyl)azetidine-3-carboxylic acid (30 mg, 0.15 mmol), HATU (57 mg, 0.15 mmol) and DMAP (37 mg, 0.30 mmol) was subjected to general amine coupling procedure with DMF (1 mL). The purification by Prep-HPLC afforded the product (44 mg, yield 90%) as a white solid: [α]₅₄₆²⁵=−75.0 (c 0.2, MeOH); ¹H NMR (400 MHz, Methanol-d₄) δ 8.26 (d, J=8.5 Hz, 1H), 7.93-7.88 (m, 1H), 7.83-7.78 (m, 1H), 7.66-7.63 (m, 1H), 7.60-7.46 (m, 5H), 7.18 (d, J=8.3 Hz, 1H), 6.05 (q, J=6.9 Hz, 1H), 4.08 (d, J=7.5 Hz, 4H), 3.46 (p, J=7.3 Hz, 1H), 2.30 (s, 3H), 1.71 (d, J=6.9 Hz, 3H), 1.45 (s, 9H); ¹³C NMR (100 MHz, Methanol-d₄) δ 172.71, 171.53, 158.07, 140.30, 138.38, 137.31, 135.48, 132.61, 132.37, 132.11, 129.90, 128.98, 127.30, 126.73, 126.43, 124.29, 123.71, 122.49, 119.95, 81.27, 46.37, 34.85, 28.62, 21.40, 19.06; HRMS (ESI) calcd for C₃₀H₃₈N₃O₃ [M+H]⁺ 488.2544, found 488.2534.

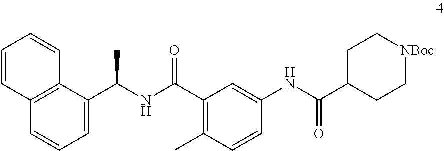 [SMILES: Cc1ccc(NC(=O)C2CCN(C(=O)OC(C)(C)C)CC2)cc1C(=O)N[C@H](C)c1cccc2ccccc12]

Tert-butyl-(R)-4-((4-methyl-3-((1-(naphthalen-1-yl)ethyl)carbamoyl)phenyl)carbamoyl)piperidine-1-carboxylate. (R)-5-amino-2-methyl-N-(1-(naphthalen-1-yl)ethyl)benzamide (30 mg, 0.10 mmol), 1-(tert-butoxycarbonyl)piperidine-4-carboxylic acid (35 mg, 0.15 mmol), HATU (57 mg, 0.15 mmol) and DMAP (37 mg, 0.30 mmol) was subjected to general amine coupling procedure with DMF (1 mL). The purification by Prep-HPLC afforded the product (48 mg, yield 93%) as a white solid: [α]₅₄₆²⁵=−77.0 (c 0.1, MeCOH); ¹H NMR (400 MHz, Methanol-d₄) δ 8.25 (d, J=8.5 Hz, 1H), 7.92-7.88 (m, 1H), 7.83-7.79 (m, 1H), 7.65-7.62 (m, 1H), 7.60-7.44 (m, 5H), 7.17 (d, J=8.3 Hz, 1H), 6.05 (q, J=6.9 Hz, 1H), 4.17-4.09 (m, 2H), 2.89-2.76 (m, 2H), 2.56-2.47 (m, 1H), 2.30 (s, 3H), 1.84-1.77 (m, 2H), 1.70 (d, J=6.9 Hz, 3H), 1.68-1.57 (m, 2H), 1.47 (s, 9H); ¹³C NMR (100 MHz, Methanol-d₄) δ 175.92, 171.58, 156.42, 140.30, 139.22, 138.31, 137.47, 135.47, 132.42, 132.06, 129.90, 128.98, 127.29, 126.73, 126.43, 124.29, 123.70, 122.56, 120.03, 81.17, 46.36, 44.72, 29.64, 28.68, 21.42, 19.06; HRMS (ESI) calcd for C₃₁H₃₈N₃O₄ [M+H]⁺516.2857, found 516.2892.

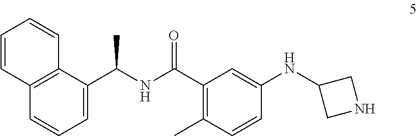 [SMILES: Cc1ccc(NC2CNC2)cc1C(=O)N[C@H](C)c1cccc2ccccc12]

(R)-5-(azetidin-3-ylamino)-2-methyl-N-(1-(naphthalen-1-yl)ethyl)benzamide. General procedure for N-Boc deprotection was used with tert-butyl (R)-3-((4-methyl-3-((1-(naphthalen-1-yl)ethyl)carbamoyl)phenyl)amino)azetidine-1-carboxylate (20 mg, 0.04 mmol) in DCM (1 mL) and HCl (4M in dioxane, 100 μL). The purification by Prep-HPLC afforded the product (12 mg, yield 84%) as a light brown solid: [α]₅₄₆²⁵=−87.4 (c 0.8, MeCOH); 1H NMR (400 MHz, Methanol-d₄) δ 8.53 (s, 1H), 8.28-8.21 (m, 1H), 7.93-7.88 (m, 1H), 7.84-7.78 (m, 1H), 7.64-7.61 (m, 1H), 7.60-7.45 (m, 3H), 7.03-6.99 (m, 1H), 6.57-6.52 (m, 1H), 6.51-6.48 (m, 1H), 6.07-6.00 (m, 1H), 4.43 (p, J=7.0 Hz, 1H), 4.33-4.25 (m, 2H), 3.93-3.85 (m, 2H), 2.21 (s, 3H), 1.70 (d, J=6.9 Hz, 3H); ¹³C NMR (100 MHz, Methanol-d₄) δ 172.26, 145.38, 140.32, 138.85, 135.47, 132.61, 132.36, 129.93, 128.98, 127.24, 126.73, 126.42, 125.82, 124.32, 123.80, 115.53, 112.83, 54.85, 46.86, 46.32, 21.35, 18.59; HRMS (ESI) calcd for C₂₃H₂₆N₃O [M+H]⁺ 360.2070, found 360.2076.

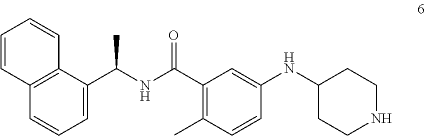 [SMILES: Cc1ccc(NC2CCNCC2)cc1C(=O)N[C@H](C)c1cccc2ccccc12]

(R)-2-methyl-N-(1-(naphthalen-1-yl)ethyl)-5-(piperidin-4-ylamino)benzamide. General procedure for N-Boc deprotection was used with tert-butyl (R)-4-((4-methyl-3-((1-(naphthalen-1-yl)ethyl)carbamoyl)phenyl)amino)piperidine-1-carboxylate (20 mg, 0.04 mmol) in DCM (1 mL) and HCl (4M in dioxane, 100 μL). The purification by Prep-HPLC afforded the product (14 mg, yield 90%) as a light brown solid: [α]₅₄₆²⁵=−75.4 (c 1.2, MeCOH); ¹H NMR (400 MHz, Methanol-d₄) δ 8.48 (s, 1H), 8.27-8.22 (m, 1H), 7.93-7.87 (m, 1H), 7.84-7.77 (m, 1H), 7.63 (d, J=7.2 Hz, 1H), 7.59-7.45 (m, 3H), 7.01-6.97 (m, 1H), 6.67-6.63 (m, 1H), 6.61-6.59 (m, 1H), 6.03 (q, J=6.9 Hz, 1H), 3.51 (tt, J=9.3, 3.5 Hz, 1H), 3.41-3.35 (m, 2H), 3.09-2.99 (m, 2H), 2.20 (s, 3H), 2.17-2.10 (m, 2H), 1.69 (d, J=7.0 Hz, 3H), 1.66-1.54 (m, 2H); ¹³C NMR (100 MHz, Methanol-d₄) δ 172.58, 146.22, 140.37, 138.72, 135.46, 132.46, 132.36, 129.92, 128.96, 127.24, 126.71, 126.44, 124.62, 124.36, 123.80, 115.92, 113.18, 48.32, 46.32, 43.92, 29.92, 21.37, 18.57; HRMS (ESI) calcd for C₂₅H₃₀N₃O [M+H]⁺388.2383, found 388.2389.

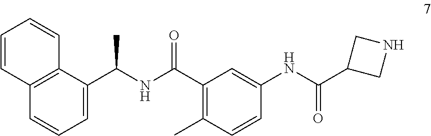 [SMILES: Cc1ccc(NC(=O)C2CNC2)cc1C(=O)N[C@H](C)c1cccc2ccccc12]

(R)—N-(4-methyl-3-((1-(naphthalen-1-yl)ethyl)carbamoyl)phenyl)azetidine-3-carboxamide. General procedure for N-Boc deprotection was used with tert-butyl (R)-3-((4-methyl-3-((1-(naphthalen-1-yl)ethyl)carbamoyl)phenyl)carbamoyl)azetidine-1-carboxylate (20 mg, 0.04 mmol) in DCM (1 mL) and HCl (4M in dioxane, 100 μL). The purification by Prep-HPLC afforded the product (14 mg, yield 88%) as a white solid: [α]₅₄₆²⁵=−94.9 (c 0.5, MeOH); ¹H NMR (400 MHz, Methanol-d₄) δ 8.34 (s, 1H), 8.26 (d, J=8.5 Hz, 1H), 7.90 (dd, J=8.2, 1.5 Hz, 1H), 7.81 (d, J=8.1 Hz, 1H), 7.67-7.42 (m, 6H), 7.19 (td, J=7.6, 7.0, 4.8 Hz, 1H), 6.11-6.01 (m, 1H), 4.25 (t, J=6.6 Hz, 2H), 3.99-3.73 (m, 2H), 3.43 (dd, J=14.0, 9.1 Hz, 1H), 2.31 (s, 3H), 1.71 (d, J=6.9 Hz, 3H); ¹³C NMR (100 MHz, Methanol-d₄) δ 171.49, 169.74, 140.26, 138.14, 137.02, 135.46, 132.81, 132.35, 132.10, 129.91, 129.00, 127.31, 126.74, 126.42, 124.27, 123.71, 122.51, 119.95, 47.60, 46.36, 39.88, 21.43, 19.09; HRMS (ESI) calcd for C₂₄H₂₆N₃O₂ [M+H]⁺388.2020, found 388.2028.

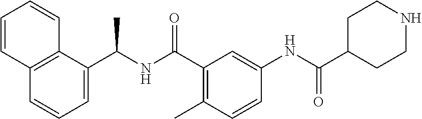 [SMILES: Cc1ccc(NC(=O)C2CCNCC2)cc1C(=O)N[C@H](C)c1cccc2ccccc12]

(R)—N-(4-methyl-3-((1-(naphthalen-1-yl)ethyl)carbamoyl)phenyl)piperidine-4-carboxamide. General procedure for N-Boc deprotection was used with tert-butyl (R)-4-((4-methyl-3-((1-(naphthalen-1-yl)ethyl)carbamoyl)phenyl)carbamoyl)piperidine-1-carboxylate (20 mg, 0.04 mmol) in DCM (1 mL) and HCl (4M in dioxane, 100 μL). The purification by Prep-HPLC afforded the product (14 mg, yield 84%) as a white solid: [α]₅₄₆²⁵=−96.1 (c 0.6, MeCOH); ¹H NMR (400 MHz, Methanol-d₄) δ 8.52 (s, 1H), 8.28-8.22 (m, 1H), 7.93-7.88 (m, 1H), 7.83-7.78 (m, 1H), 7.66-7.62 (m, 1H), 7.60-7.45 (m, 5H), 7.20-7.16 (m, 1H), 6.05 (q, J=6.9 Hz, 1H), 3.45 (dt, J=13.0, 3.8 Hz, 2H), 3.04 (td, J=12.5, 3.4 Hz, 2H), 2.67 (tt, J=10.8, 4.0 Hz, 1H), 2.31 (s, 3H), 2.09-1.89 (m, 4H), 1.70 (d, J=6.9 Hz, 3H); ¹³C NMR (100 MHz, MeOD) δ 174.20, 171.52, 140.28, 138.41, 137.28, 135.47, 132.55, 132.36, 132.11, 129.91, 128.99, 127.30, 126.74, 126.42, 124.28, 123.71, 122.52, 119.99, 46.35, 44.24, 41.59, 26.65, 21.41, 19.07; HRMS (ESI) calcd for C₂₆H₃₀N₃O₂ [M+H]⁺ 416.2333, found 416.2340.

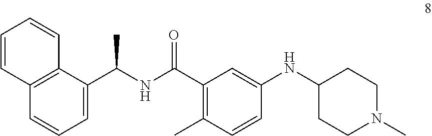 [SMILES: Cc1ccc(NC2CCN(C)CC2)cc1C(=O)N[C@H](C)c1cccc2ccccc12]

(R)-2-methyl-5-((1-methylpiperidin-4-yl)amino)-N-(1-(naphthalen-1-yl)ethyl)benzamide. (R)-5-amino-2-methyl-N-(1-(naphthalen-1-yl)ethyl)benzamide (30 mg, 0.10 mmol) and 1-methylpiperidin-4-one (60 mg, 0.50 mmol) was subjected to general reductive amination procedure with MeOH (1 mL), HOAc (0.2 mL) at 50° C., and then NaBH₃CN (32 mg, 0.50 mmol) was added. The purification by Prep-HPLC afforded the product (34 mg, yield 85%) as a white solid: [α]₅₄₆²⁵=−76.9 (c 3.0, MeOH); ¹H NMR (400 MHz, Methanol-d₄) δ 8.54-8.48 (m, 11H), 8.24 (d, J=8.4 Hz, 11H), 7.93-7.87 (m, 1H), 7.83-7.76 (m, 1H), 7.65-7.62 (m, 1H), 7.59-7.45 (m, 3H), 7.00-6.97 (m, 1H), 6.66-6.59 (m, 2H), 6.02 (q, J=6.9 Hz, 1H), 3.51-3.44 (m, 1H), 3.39-3.33 (m, 2H), 3.07-2.95 (m, 2H), 2.75 (s, 3H), 2.21 (s, 3H), 2.15-2.06 (m, 2H), 1.71-1.58 (m, 5H); ¹³C NMR (100 MHz, Methanol-d₄) δ 169.70, 146.22, 140.48, 138.70, 135.40, 132.49, 132.28, 129.93, 128.92, 127.25, 126.73, 126.49, 124.60, 124.31, 123.75, 115.87, 113.21, 53.95, 47.46, 46.36, 43.58, 30.21, 21.48, 18.60; HRMS (ESI) calcd for C₂₆H₃₂N₃O [M+H]⁺ 402.2540, found 416.2545.

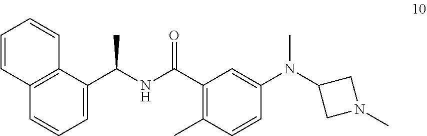 [SMILES: Cc1ccc(N(C)C2CN(C)C2)cc1C(=O)N[C@H](C)c1cccc2ccccc12]

(R)-2-methyl-5-(methyl(1-methylazetidin-3-yl)amino)-N-(1-(naphthalen-1-yl)ethyl)benzamide. (R)-2-methyl-5-((1-methylazetidin-3-yl)amino)-N-(1-(naphthalen-1-yl)ethyl)benzamide (16 mg, 0.04 mmol) and formaldehyde (37 wt. % in H₂O, 200 mL) was subjected to general reductive amination procedure with MeOH (1 mL), HOAc (50 mL) at room temperature, and then NaBH₃CN (14 mg, 0.21 mmol) was added. The purification by Prep-HPLC afforded the product (110 mg, yield 95%) as a white solid: [α]₅₄₆²⁵=−80.9 (c 0.8, MeOH); ¹H NMR (400 MHz, Methanol-d₄) δ 8.31 (s, 1H), 8.25 (d, J=8.5 Hz, 1H), 7.93-7.89 (m, 1H), 7.82 (d, J=8.2 Hz, 1H), 7.64-7.46 (m, 4H), 7.14-7.10 (m, 1H), 6.80 (dd, J=8.3, 2.7 Hz, 1H), 6.72-6.70 (m, 1H), 6.04 (q, J=6.9 Hz, 1H), 4.37-4.24 (m, 3H), 4.09-4.00 (m, 2H), 2.91 (s, 3H), 2.80 (s, 3H), 2.25 (s, 3H), 1.71 (d, J=6.9 Hz, 3H); ¹³C NMR (100 MHz, Methanol-d₄) δ 171.90, 148.35, 140.26, 138.88, 135.48, 132.64, 132.36, 129.97, 129.04, 128.96, 127.25, 126.75, 126.45, 124.33, 123.86, 120.07, 117.43, 61.16, 50.84, 46.44, 42.60, 37.95, 21.31, 18.70; HRMS (ESI) calcd for C₂₅H₃₀N₃O [M+H]⁺388.2383, found 388.2389.

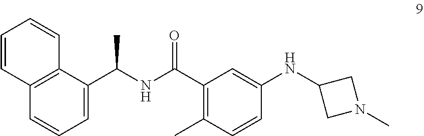 [SMILES: Cc1ccc(NC2CN(C)C2)cc1C(=O)N[C@H](C)c1cccc2ccccc12]

(R)-2-methyl-5-((1-methylazetidin-3-yl)amino)-N-(1-(naphthalen-1-yl)ethyl)benzamide. (R)-5-amino-2-methyl-N-(1-(naphthalen-1-yl)ethyl)benzamide (100 mg, 0.31 mmol) and 1-methylazetidin-3-one (40 mg, 0.49 mmol) was subjected to general reductive amination procedure with MeOH (4 mL), HOAc (1 mL) at 50° C., and then NaBH₃CN (98 mg, 1.55 mmol) was added. The purification by Prep-HPLC afforded the product (110 mg, yield 95%) as a white solid: [α]₅₄₆²⁵=−82.1 (c 1.3, MeCOH); ¹H NMR (400 MHz, Methanol-d₄) δ 8.46 (s, 1H), 8.24 (d, J=8.4 Hz, 1H), 7.93-7.87 (m, 1H), 7.81 (d, J=8.2 Hz, 1H), 7.65-7.45 (m, 4H), 7.01 (d, J=8.2 Hz, 1H), 6.58-6.48 (m, 2H), 6.03 (q, J=6.9 Hz, 1H), 4.44-4.32 (m, 3H), 3.94-3.84 (m, 2H), 2.90 (s, 3H), 2.21 (s, 3H), 1.69 (d, J=6.9 Hz, 3H); ¹³C NMR (100 MHz, Methanol-d₄) δ 172.22, 145.34, 140.35, 138.84, 135.45, 132.62, 132.34, 129.93, 128.97, 127.25, 126.73, 126.44, 125.94, 124.32, 123.79, 115.57, 112.94, 63.95, 46.34, 44.05, 42.70, 21.38, 18.61; HRMS (ESI) calcd for C₂₄H₂₈N₃O [M+H]⁺374.2227, found 388.2230.

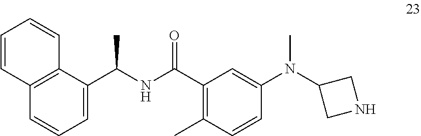 [SMILES: Cc1ccc(N(C)C2CNC2)cc1C(=O)N[C@H](C)c1cccc2ccccc12]

(R)-5-(azetidin-3-yl(methyl)amino)-2-methyl-N-(1-(naphthalen-1-yl)ethyl)benzamide. (R)-1-(naphthalen-1-yl)ethan-1-amine (100 mg, 0.58 mmol), 5-((1-(tert-butoxycarbonyl)azetidin-3-yl)(methyl)amino)-2-methylbenzoic acid (189 mg, 0.58 mmol), HATU (221 mg, 0.58 mmol) and DMAP (213 mg, 1.74 mmol) was subjected to general amine coupling procedure with DMF (4 mL). After purification by Prep-HPLC, the product was subjected to general N-Boc deprotection procedure with HCl (4M in dioxane, 1.4 mL) and DCM (5 mL). The purification by Prep-HPLC afforded the product (150 mg, yield 69% for 2 steps) as a white solid: [α]₅₄₆²⁵=−78.9 (c 1.1, MeOH); ¹H NMR (400 MHz, Methanol-d₄) δ 8.47 (s, 1H), 8.28-8.22 (m, 1H), 7.94-7.87 (m, 1H), 7.81 (d, J=8.2 Hz, 1H), 7.66-7.44 (m, 4H), 7.11 (d, J=8.3 Hz, 1H), 6.80 (dd, J=8.4, 2.7 Hz, 1H), 6.72 (d, J=2.7 Hz, 1H), 6.04 (q, J=6.9 Hz, 1H), 4.53-4.41 (m, 1H), 4.23-4.14 (m, 2H), 4.09-4.00 (m, 2H), 2.82 (s, 3H), 2.24 (s, 3H), 1.71 (d, J=6.9 Hz, 3H); ¹³C NMR (100 MHz, Methanol-d₄) δ 171.98, 148.37, 140.27, 138.83, 135.48, 132.61, 132.34, 129.96, 129.02, 128.56, 127.24, 126.74, 126.44, 124.31, 123.84, 119.66, 116.96, 53.31, 51.99, 46.45, 37.03, 21.34, 18.68. HRMS (ESI) calcd for C₂₄H₂₈N₃O [M+H]⁺374.2227, found 374.2231.

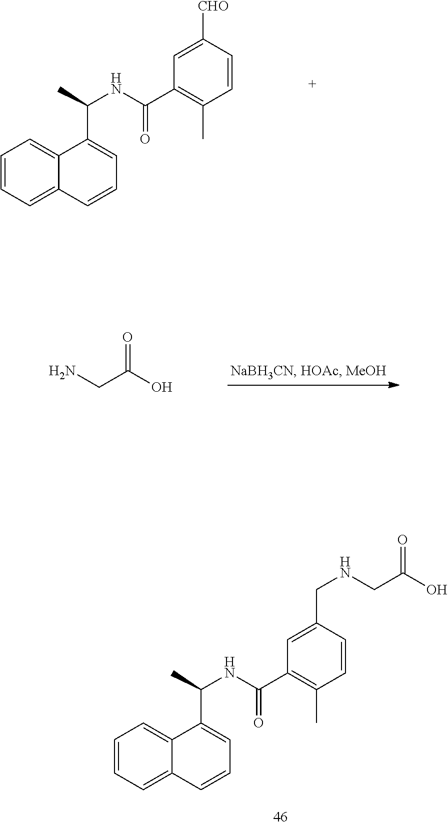 [SMILES: Cc1ccc(C=O)cc1C(=O)N[C@H](C)c1cccc2ccccc12 ; NCC(=O)O ; Cc1ccc(CNCC(=O)O)cc1C(=O)N[C@H](C)c1cccc2ccccc12]

(R)-(4-methyl-3-((1-(naphthalen-1-yl)ethyl)carbamoyl)benzyl)qlycine. (R)-5-formyl-2-methyl-N-(1-(naphthalen-1-yl)ethyl)benzamide (30 mg, 0.10 mmol) and glycine (11 mg, 0.15 mmol) was subjected to general reductive amination procedure with MeOH (1 mL), HOAc (200 μL) at 50° C., and then NaBH₃CN (19 mg, 0.30 mmol) was added. The purification by Prep-HPLC provided the amination compound (20 mg, yield 53%) as a white solid: [α]54=−64.4 (c 0.7, MeOH); ¹H NMR (400 MHz, Methanol-d₄) δ 8.27 (d, J=8.5 Hz, 1H), 7.91 (d, J=8.1 Hz, 1H), 7.82 (d, J=8.2 Hz, 1H), 7.66 (d, J=7.3 Hz, 1H), 7.60-7.43 (m, 5H), 7.34 (d, J=8.1 Hz, 1H), 6.09 (q, J=6.9 Hz, 1H), 4.24 (s, 2H), 3.89 (s, 2H), 2.38 (s, 3H), 1.73 (d, J=6.9 Hz, 3H); ¹³C NMR (100 MHz, Methanol-d₄) δ 171.08, 168.15, 140.05, 138.86, 138.59, 135.47, 132.62, 132.43, 132.33, 129.94, 129.68, 129.52, 129.08, 127.34, 126.78, 126.47, 124.23, 123.85, 68.13, 47.53, 46.41, 21.38, 19.46; HRMS (ESI) calcd for C₂₃H₂₅N₂O₃ [M+H]⁺377.1860, found 377.1857.

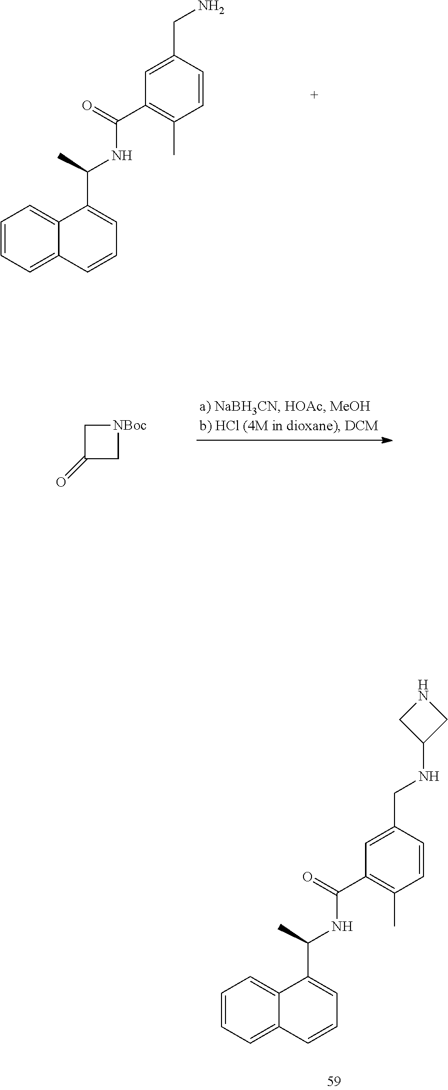 [SMILES: Cc1ccc(CN)cc1C(=O)N[C@H](C)c1cccc2ccccc12 ; CC(C)(C)OC(=O)N1CC(=O)C1 ; Cc1ccc(CNC2CNC2)cc1C(=O)N[C@H](C)c1cccc2ccccc12]

(R)-5-((azetidin-3-ylamino)methyl)-2-methyl-N-(1-(naphthalen-1-yl)ethyl)benzamide. (R)-5-(aminomethyl)-2-methyl-N-(1-(naphthalen-1-yl)ethyl)benzamide (30 mg, 0.09 mmol) and tert-butyl 3-oxoazetidine-1-carboxylate (31 mg, 0.18 mmol) was subjected to general reductive amination procedure with MeCOH (2 mL), HOAc (500 μL) at 50° C., and then NaBH₃CN (17 mg, 0.27 mmol) was added. After purification by Prep-HPLC, the product was subjected to general N-Boc deprotection procedure with HCl (4M in dioxane, 100 μL) and DCM (2 mL). The purification by Prep-HPLC afforded the (26 mg, yield 77% for 2 steps) as a white solid: ¹H NMR (400 MHz, Methanol-d₄) δ 8.31 (s, 1H), 8.27 (d, J=8.6 Hz, 1H), 7.93-7.89 (m, 1H), 7.82 (d, J=8.2 Hz, 1H), 7.64 (d, J=7.1 Hz, 1H), 7.60-7.46 (m, 3H), 7.31-7.26 (m, 2H), 7.20 (d, J=7.7 Hz, 1H), 6.07 (q, J=6.8 Hz, 1H), 4.05-3.98 (m, 2H), 3.86-3.73 (m, 3H), 3.71 (s, 2H), 2.33 (s, 3H), 1.71 (d, J=7.0 Hz, 3H); ¹³C NMR (100 MHz, Methanol-d₄) δ 171.85, 140.23, 138.40, 135.86, 135.49, 132.38, 131.95, 130.78, 129.94, 129.04, 128.01, 127.29, 126.76, 126.45, 124.31, 123.81, 54.80, 51.33, 51.02, 46.33, 21.38, 19.32; HRMS (ESI) calcd for C₂₄H₂₈N₃O [M+H]⁺ 374.2227, found 374.2231.

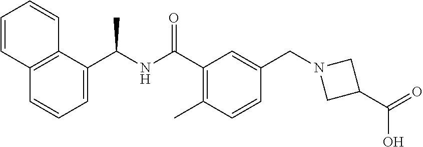 [SMILES: Cc1ccc(CN2CC(C(=O)O)C2)cc1C(=O)N[C@H](C)c1cccc2ccccc12]

(R)-1-(4-methyl-3-((1-(naphthalen-1-yl)ethyl)carbamoyl)benzyl)azetidine-3-carboxylic acid. ¹H NMR (400 MHz, Acetone-d₆) δ 8.48 (d, J=8.1 Hz, 1H), 8.28 (d, J=8.5 Hz, 1H), 7.94-7.87 (m, 1H), 7.78 (d, J=8.1 Hz, 1H), 7.71-7.63 (m, 2H), 7.57-7.42 (m, 3H), 7.32 (d, J=7.7 Hz, 1H), 7.15 (d, J=7.6 Hz, 1H), 6.05 (p, J=7.0 Hz, 1H), 4.12 (s, 2H), 4.00-3.88 (m, 4H), 3.22-3.15 (m, 1H), 2.35 (s, 3H), 1.63 (d, J=6.9 Hz, 3H); ¹³C NMR (100 MHz, Acetone-d₆) δ 176.13, 164.02, 141.08, 138.09, 137.92, 134.86, 131.98, 131.78, 131.60, 130.18, 129.62, 128.37, 126.97, 126.44, 126.35, 124.34, 123.52, 67.58, 58.97, 57.17, 45.49, 35.10, 21.78, 19.85; HRMS (ESI) calcd for C₂₅H₂₇N₂O₃ [M+H]⁺403.2016, found 403.2019.

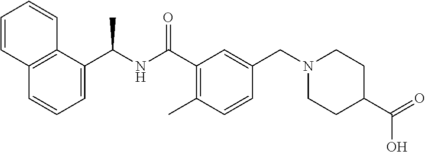 [SMILES: Cc1ccc(CN2CCC(C(=O)O)CC2)cc1C(=O)N[C@H](C)c1cccc2ccccc12]

(R)-1-(4-methyl-3-((1-(naphthalen-1-yl)ethyl)carbamoyl)benzyl)piperidine-4-carboxylic acid. ¹H NMR (400 MHz, Acetone-d₆) δ 8.33 (d, J=8.5 Hz, 1H), 8.00 (d, J=8.1 Hz, 1H), 7.93 (dd, J=8.2, 1.5 Hz, 1H), 7.83 (d, J=8.2 Hz, 1H), 7.71 (d, J=7.1 Hz, 1H), 7.63-7.57 (m, 1H), 7.55-7.44 (m, 3H), 7.28 (dd, J=7.8, 1.8 Hz, 1H), 7.16 (d, J=7.7 Hz, 1H), 6.11 (p, J=7.2 Hz, 1H), 2.90 (d, J=11.2 Hz, 2H), 2.37 (s, 3H), 2.34-2.18 (m, 3H), 2.07-2.07 (m, 2H), 1.92-1.84 (m, 2H), 1.79-1.67 (m, 5H); ¹³C NMR (100 MHz, Acetone-d₆) δ 176.16, 163.15, 140.80, 138.02, 137.99, 136.02, 134.92, 132.10, 131.34, 131.16, 129.61, 128.94, 128.48, 126.99, 126.48, 126.29, 124.40, 123.61, 62.35, 53.03, 45.43, 45.33, 40.81, 28.43, 21.57, 19.63; HRMS (ESI) calcd for C₂₇H₃₁N₂O₃ [M+H]⁺431.2329, found 431.2333.

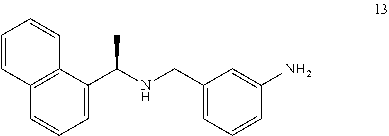 [SMILES: C[C@@H](NCc1cccc(N)c1)c1cccc2ccccc12]

(R)-3-(((1-(naphthalen-1-yl)ethyl)amino)methyl)aniline. ¹H NMR (400 MHz, Methanol-d₄) δ 8.40-8.31 (m, 2H), 8.14-8.06 (m, 1H), 8.04-7.95 (m, 2H), 7.83-7.77 (m, 1H), 7.68-7.57 (m, 3H), 6.93 (d, J=7.9 Hz, 1H), 6.74-6.63 (m, 2H), 5.44 (q, J=6.8 Hz, 1H), 3.98 (dd, J=91.8, 13.1 Hz, 2H), 1.83 (d, J=6.7 Hz, 3H); ¹³C NMR (100 MHz, Methanol-d₄) δ 147.53, 135.57, 134.31, 132.86, 132.38, 131.22, 131.11, 130.42, 128.56, 127.60, 127.33, 126.63, 125.07, 122.97, 118.09, 118.00, 53.94, 20.21, 17.95; HRMS (ESI) calcd for C₁₉H₂₁N₂ [M+H]⁺ 277.1699, found 277.1709.

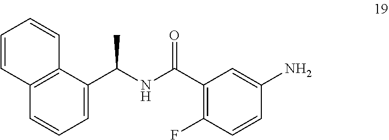 [SMILES: C[C@@H](NC(=O)c1cc(N)ccc1F)c1cccc2ccccc12]

(R)-5-amino-2-fluoro-N-(1-(naphthalen-1-yl)ethyl)benzamide. (R)-1-(naphthalen-1-yl)ethan-1-amine (221 mg, 1.29 mmol), 5-amino-2-fluorobenzoic acid (100 mg, 0.64 mmol), HATU (269 mg, 0.71 mmol), TEA (200 μL), and DMAP (8 mg, 0.06 mmol) was subjected to general amine coupling procedure with DCM (4 mL). The purification by Prep-HPLC afforded the product (167 mg, yield 85%) as a white solid: [α]₅₄₆²⁵=−72.2 (c 13.3, MeOH); ¹H NMR (400 MHz, Methanol-d₄) δ 8.19-8.15 (m, 1H), 7.82 (dd, J=8.2, 1.5 Hz, 1H), 7.73 (d, J=8.2 Hz, 1H), 7.60 (d, J=7.1 Hz, 1H), 7.53-7.38 (m, 3H), 7.00 (dd, J=6.0, 2.9 Hz, 1H), 6.89-6.83 (m, 1H), 6.76-6.71 (m, 1H), 6.03 (q, J=6.8 Hz, 1H), 1.63 (d, J=6.9 Hz, 3H); ¹¹C NMR (100 MHz, Methanol-d₄) δ 166.07, 154.04 (d, J=237.9 Hz), 145.48, 140.27, 135.21, 132.01, 129.81, 128.83, 127.20, 126.61, 126.41, 124.21 (d, J=14.8 Hz), 124.06, 123.49, 119.76 (d, J=8.0 Hz), 117.36 (d, J=24.2 Hz), 116.43, 46.71, 38.78, 21.70; HRMS (ESI) calcd for C₁₉H₁₈FN₂O [M+H]⁺309.1398, found 309.1404.

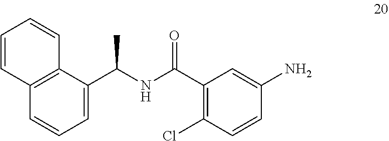 [SMILES: C[C@@H](NC(=O)c1cc(N)ccc1Cl)c1cccc2ccccc12]

(R)-5-amino-2-chloro-N-(1-(naphthalen-1-yl)ethyl)benzamide. (R)-1-(naphthalen-1-yl)ethan-1-amine (200 mg, 1.16 mmol), 5-amino-2-chlorobenzoic acid (100 mg, 0.58 mmol), HATU (243 mg, 0.64 mmol), TEA (200 μL), and DMAP (8 mg, 0.06 mmol) was subjected to general amine coupling procedure with DCM (4 mL). The purification by Prep-HPLC afforded the product (147 mg, yield 78%) as a white solid: [α]₅₆=−75.6 (c 12.8, MeOH); ¹H NMR (400 MHz, Methanol-d₄) δ 8.22 (d, J=8.5 Hz, 1H), 7.88 (dd, J=8.1, 1.5 Hz, 1H), 7.79 (d, J=8.2 Hz, 1H), 7.64 (d, J=7.2 Hz, 1H), 7.60-7.42 (m, 3H), 7.11 (dd, J=8.0, 1.0 Hz, 1H), 6.72 (d, J=8.0 Hz, 2H), 6.01 (q, J=6.9 Hz, 1H), 1.68 (d, J=6.9 Hz, 3H); ¹³C NMR (100 MHz, Methanol-d₄) δ 169.43, 147.36, 140.07, 137.96, 135.39, 132.28, 131.34, 129.83, 128.92, 127.24, 126.66, 126.39, 124.32, 123.77, 119.88, 118.69, 116.13, 46.58, 21.41; HRMS (ESI) calcd for C₁₉H₁₈ClN₂O [M+H]⁺325.1102, found 325.1101.

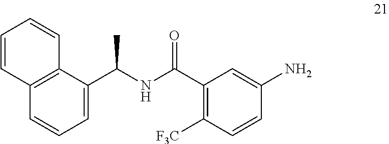 [SMILES: C[C@@H](NC(=O)c1cc(N)ccc1C(F)(F)F)c1cccc2ccccc12]

(R)-5-amino-N-(1-(naphthalen-1-yl)ethyl)-2-(trifluoromethyl)benzamide. (R)-1-(naphthalen-1-yl)ethan-1-amine (200 mg, 1.16 mmol), 5-amino-2-(trifluoromethyl)benzoic acid (119 mg, 0.58 mmol), HATU (243 mg, 0.64 mmol), TEA (200 μL), and DMAP (8 mg, 0.06 mmol) was subjected to general amine coupling procedure with DCM (4 mL). The purification by Prep-HPLC afforded the product (154 mg, yield 74%) as a white solid: [α]₅₄₆²⁵=−66.2 (c 3.4, MeOH); ¹H NMR (400 MHz, Methanol-d₄) δ 8.25-8.19 (m, 1H), 7.88 (dd, J=8.2, 1.4 Hz, 1H), 7.79 (d, J=8.2 Hz, 1H), 7.64-7.61 (m, 1H), 7.59-7.54 (m, 1H), 7.52-7.44 (m, 2H), 7.37 (d, J=8.6 Hz, 1H), 6.70 (dd, J=8.8, 2.4 Hz, 1H), 6.64-6.62 (m, 1H), 6.02 (q, J=6.9 Hz, 1H), 1.66 (d, J=6.9 Hz, 3H); ¹³C NMR (100 MHz, Methanol-d₄) δ 170.40, 152.88, 140.00, 138.39, 135.38, 132.32, 129.82, 128.92, 128.70 (q, J=4.8 Hz), 127.29, 126.67, 126.39, 126.10 (q, J=270.7 Hz), 124.23, 123.74, 114.94, 114.17, 46.39, 21.22; HRMS (ESI) calcd for C₂₀H₁₈F₃N₂O [M+H]⁺359.1366, found 359.1371.

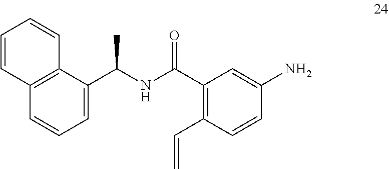 [SMILES: C=Cc1ccc(N)cc1C(=O)N[C@H](C)c1cccc2ccccc12]

(R)-5-amino-N-(1-(naphthalen-1-yl)ethyl)-2-vinylbenzamide. (R)-1-(naphthalen-1-yl)ethan-1-amine (100 mg, 0.58 mmol), 5-amino-2-vinylbenzoic acid (47 mg, 0.29 mmol), HATU (118 mg, 0.31 mmol), TEA (100 μL), and DMAP (4 mg, 0.03 mmol) was subjected to general amine coupling procedure with DCM (4 mL). The purification by Prep-HPLC afforded the product (77 mg, yield 84%) as a brown solid: [α]₅₄₆²⁵=−69.8 (c 0.2, MeOH); ¹H NMR (400 MHz, Methanol-d₄) δ 8.25 (d, J=8.5 Hz, 1H), 7.93-7.87 (m, 1H), 7.81 (d, J=8.2 Hz, 1H), 7.64-7.44 (m, 4H), 7.40 (d, J=8.4 Hz, 1H), 6.80-6.70 (m, 2H), 6.64-6.62 (m, 1H), 6.03 (q, J=6.9 Hz, 1H), 5.50 (dd, J=17.5, 1.3 Hz, 1H), 4.96 (dd, J=11.1, 1.3 Hz, 1H), 1.68 (d, J=7.0 Hz, 3H); ¹³C NMR (100 MHz, Methanol-d₄) δ 172.09, 148.86, 140.39, 138.15, 135.46, 135.07, 132.34, 129.88, 128.93, 127.39, 127.27, 126.70, 126.41, 125.93, 124.30, 123.73, 117.56, 114.00, 111.86, 46.39, 21.43; HRMS (ESI) calcd for C₂₁H₂₁N₂O [M+H]⁺317.1648, found 374.2231.

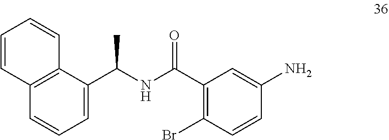 [SMILES: C[C@@H](NC(=O)c1cc(N)ccc1Br)c1cccc2ccccc12]

(R)-5-amino-2-bromo-N-(1-(naphthalen-1-yl)ethyl)benzamide. Light yellow solid (yield 82%): ¹H NMR (400 MHz, Methanol-d₄) δ 8.24 (d, J=8.5 Hz, 1H), 7.93-7.86 (m, 1H), 7.80 (d, J=8.2 Hz, 1H), 7.65 (d, J=7.2 Hz, 1H), 7.59-7.44 (m, 3H), 7.24 (d, J=8.6 Hz, 1H), 6.67 (d, J=2.8 Hz, 1H), 6.62 (dd, J=8.5, 2.8 Hz, 1H), 6.01 (q, J=6.9 Hz, 1H), 1.69 (d, J=6.9 Hz, 3H); LRMS (ESI) calcd for C₁₉H₁₈BrN₂O [M+H]⁺369.06, found 369.03.

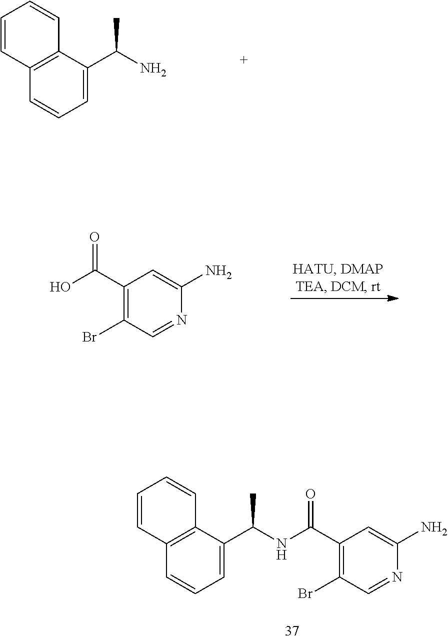 [SMILES: C[C@@H](N)c1cccc2ccccc12 ; Nc1cc(C(=O)O)c(Br)cn1 ; C[C@@H](NC(=O)c1cc(N)ncc1Br)c1cccc2ccccc12]

(R)-2-amino-5-bromo-N-(1-(naphthalen-1-yl)ethyl)isonicotinamide. (R)-1-(naphthalen-1-yl)ethan-1-amine (41 mg, 0.24 mmol), 2-amino-5-bromoisonicotinic acid (25 mg, 0.12 mmol), HATU (46 mg, 0.12 mmol), TEA (100 μL), and DMAP (3 mg, 0.02 mmol) was subjected to general amine coupling procedure with DCM (2 mL). The purification by Prep-HPLC afforded the product (31 mg, 71%) as a brown solid: [α]₅₆=−37.4 (c 0.5, MeOH); ¹H NMR (400 MHz, Methanol-d₄) δ 8.22 (d, J=8.7 Hz, 1H), 8.00 (s, 1H), 7.91-7.88 (m, 1H), 7.81 (d, J=8.3 Hz, 1H), 7.64 (d, J=7.1 Hz, 1H), 7.60-7.45 (m, 3H), 6.52 (s, 1H), 6.01 (q, J=6.9 Hz, 1H), 1.70 (d, J=7.0 Hz, 3H); ¹³C NMR (100 MHz, Methanol-d₄) δ 168.04, 160.22, 150.46, 148.32, 139.70, 135.44, 132.31, 129.88, 129.11, 127.37, 126.76, 126.39, 124.31, 123.89, 109.01, 103.71, 46.50, 21.28; HRMS (ESI) calcd for C₁₈H₁₇BrN₃O [M+H]⁺370.0550, found 370.0556.

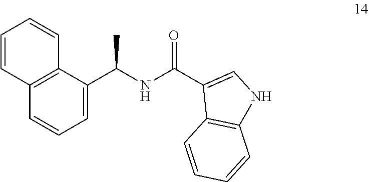 [SMILES: C[C@@H](NC(=O)c1c[nH]c2ccccc12)c1cccc2ccccc12]

(R)—N-(1-(naphthalen-1-yl)ethyl)-1H-indole-3-carboxamide. (R)-1-(naphthalen-1-yl)ethan-1-amine (233 mg, 1.36 mmol), 1H-indole-3-carboxylic acid (200 mg, 1.24 mmol), HATU (708 mg, 1.86 mmol) and DIPEA (0.6 mL, 3.72 mmol) was subjected to general amine coupling procedure with DMF (5 mL). The purification by Prep-HPLC afforded the product (334 mg, yield 86%) as a white solid: [α]₅₄₆²⁵=−14.8 (c 0.5, MeCOH); ¹H NMR (400 MHz, Chloroform-d) δ 9.06 (s, 1H), 8.25 (d, J=8.4 Hz, 1H), 7.90-7.84 (m, 2H), 7.80 (d, J=8.2 Hz, 1H), 7.69 (d, J=2.9 Hz, 1H), 7.63 (d, J=6.4 Hz, 1H), 7.55-7.44 (m, 3H), 7.39 (dd, J=6.8, 1.6 Hz, 1H), 7.20 (pd, J=7.1, 1.4 Hz, 2H), 6.23-6.16 (m, 1H), 1.81 (d, J=6.5 Hz, 2H); ¹³C NMR (100 MHz, Chloroform-d) δ 164.35, 138.82, 136.40, 134.00, 131.21, 128.72, 128.29, 127.92, 126.56, 125.83, 125.25, 124.75, 123.64, 122.78, 122.59, 121.49, 119.94, 111.93, 44.75, 21.22; HRMS (ESI) calcd for C₂₁H₁₉N₂O [M+H]⁺315.1492, found 315.1498.

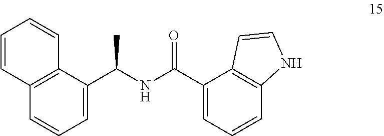 [SMILES: C[C@@H](NC(=O)c1cccc2[nH]ccc12)c1cccc2ccccc12]

(R)—N-(1-(naphthalen-1-yl)ethyl)-1H-indole-4-carboxamide. (R)-1-(naphthalen-1-yl)ethan-1-amine (94 mg, 0.55 mmol), 1H-indole-4-carboxylic acid (80 mg, 0.50 mmol), HATU (283 mg, 0.74 mmol) and DIPEA (0.26 mL, 1.5 mmol) was subjected to general amine coupling procedure with DMF (5 mL). The purification by Prep-HPLC afforded the product (117 mg, yield 75%) as a white solid: ¹H NMR (400 MHz, DMSO-d₆) δ 11.24 (s, 1H), 8.76 (d, J=7.9 Hz, 1H), 8.29 (d, J=8.4 Hz, 1H), 7.95 (dd, J=8.0, 1.6 Hz, 1H), 7.83 (d, J=8.1 Hz, 1H), 7.69 (d, J=6.7 Hz, 1H), 7.59 (ddd, J=8.5, 6.8, 1.6 Hz, 1H), 7.56-7.47 (m, 4H), 7.39 (t, J=2.8 Hz, 1H), 7.16-7.11 (m, 1H), 6.80-6.69 (m, 1H), 6.03 (p, J=7.1 Hz, 1H), 1.63 (d, J=6.9 Hz, 3H); ¹³C NMR (100 MHz, DMSO-d₆) δ 167.02, 140.83, 136.46, 133.36, 130.45, 128.61, 127.06, 126.66, 126.31, 126.06, 126.00, 125.49, 123.25, 122.55, 120.02, 118.53, 114.02, 101.74, 44.43, 39.52, 21.61; HRMS (ESI) calcd for C₂₁H₁₉N₂O [M+H]⁺315.1492, found 315.1491.

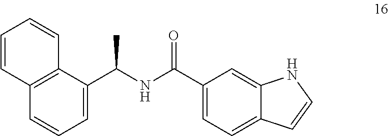 [SMILES: C[C@@H](NC(=O)c1ccc2cc[nH]c2c1)c1cccc2ccccc12]

(R)—N-(1-(naphthalen-1-yl)ethyl)-1H-indole-6-carboxamide. (R)-1-(naphthalen-1-yl)ethan-1-amine (58 mg, 0.34 mmol), 1H-indole-6-carboxylic acid (50 mg, 0.31 mmol), HATU (171 mg, 0.46 mmol) and DIPEA (0.15 mL, 0.93 mmol) was subjected to general amine coupling procedure with DMF (5 mL). The purification by Prep-HPLC afforded the product (69 mg, yield 71%) as a white solid: [α]₅₄₆²⁵=−5.0 (c 2.5, MeOH); ¹H NMR (400 MHz, DMSO-d₆) δ 11.34 (s, 1H), 8.86 (d, J=7.8 Hz, 1H), 8.25 (d, J=8.4 Hz, 1H), 8.00 (s, 1H), 7.99-7.92 (m, 1H), 6.49 (td, J=2.0, 0.9 Hz, 1H), 6.01 (p, J=7.1 Hz, 1H), 1.64 (d, J=6.9 Hz, 3H); ¹³C NMR (100 MHz, DMSO-d₆) δ 166.92, 141.31, 135.66, 133.84, 130.95, 130.25, 129.09, 128.39, 127.82, 127.56, 126.57, 125.98, 125.93, 123.69, 123.04, 119.75, 118.69, 111.88, 101.66, 45.12, 22.06; HRMS (ESI) calcd for C₂₁H₁₉N₂O [M+H]⁺ 315.1492, found 315.1498.

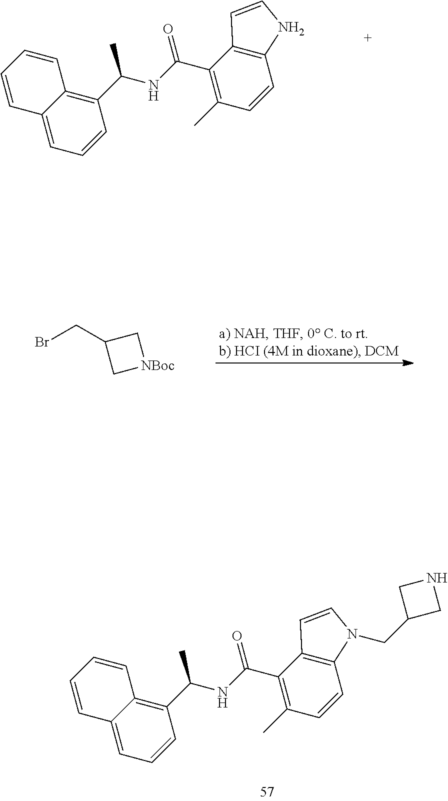 [SMILES: Cc1ccc2[nH]ccc2c1C(=O)N[C@H](C)c1cccc2ccccc12 ; CC(C)(C)OC(=O)N1CC(CBr)C1 ; Cc1ccc2c(ccn2CC2CNC2)c1C(=O)N[C@H](C)c1cccc2ccccc12]

(R)-1-(azetidin-3-ylmethyl)-5-methyl-N-(1-(naphthalen-1-yl)ethyl)-1H-indole-4-carboxamide. To a solution of (R)-5-methyl-N-(1-(naphthalen-1-yl)ethyl)-1H-indole-4-carboxamide (40 mg, 0.12 mmol) in dry THE (5 mL), sodium hydride (12 mg 0.25 mmol) was added at 0° C. After 15 minutes, the mixture was added tert-butyl 3-(bromomethyl)azetidine-1-carboxylate (30 mg, 0.16 mmol) and stirred at 25° C. for another 16 hours. Quench the reaction with methanol and remove the solvent. The residue was purified by preparative HPLC system to obtain the desired product.

The product from previous step was subjected to general N-Boc deprotection procedure with HCl (4M in dioxane, 200 μL) and DCM (4 mL). The purification by Prep-HPLC afforded the product (39 mg, yield 82% for 2 steps) as a white solid: [α]₅₄₆²⁵=−63.9 (c 1.1, MeOH); ¹H NMR (400 MHz, Methanol-d₄) δ 8.53 (s, 1H), 7.33 (d, J=8.1 Hz, 1H), 7.25 (d, J=3.2 Hz, 1H), 7.18-7.07 (m, 3H), 6.79 (dd, J=8.3, 2.7 Hz, 1H), 6.73 (d, J=2.6 Hz, 1H), 6.66 (d, J=3.7 Hz, 1H), 5.62 (q, J=7.0 Hz, 1H), 4.46 (p, J=7.2 Hz, 1H), 4.19 (dd, J=12.3, 7.4 Hz, 4H), 4.05 (dd, J=10.3, 7.4 Hz, 2H), 2.83 (s, 3H), 2.23 (s, 3H), 2.06-1.96 (m, 2H), 1.93-1.79 (m, 4H), 1.65 (d, J=7.0 Hz, 3H). ¹³C NMR (100 MHz, Methanol-d₄) δ 148.36, 139.08, 137.99, 136.30, 132.56, 129.04, 128.62, 127.88, 122.25, 119.64, 117.05, 116.55, 111.39, 109.89, 99.99, 53.41, 52.28, 52.02, 49.00, 37.58, 37.12, 27.10, 21.24, 18.98, 18.65; HRMS (ESI) calcd for C₂₆H₂₈N₃O [M+H]⁺398.2227, found 398.2230.

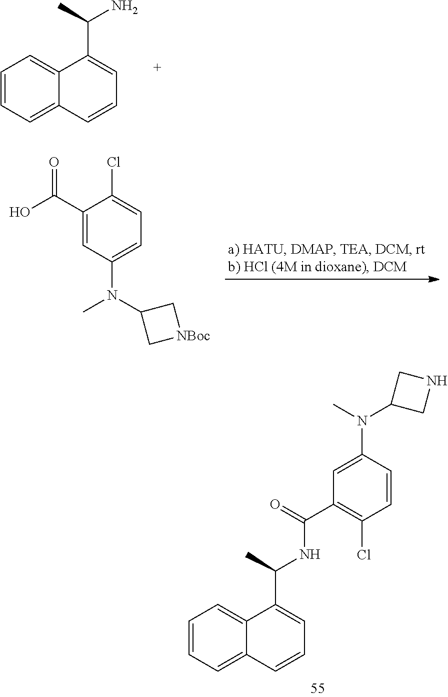

(R)-5-(azetidin-3-yl(methyl)amino)-2-chloro-N-(1-(naphthalen-1-yl)ethyl)benzamide. (R)-1-(naphthalen-1-yl)ethan-1-amine (39 mg, 0.23 mmol), 5-((1-(tert-butoxycarbonyl)azetidin-3-yl)(methyl)amino)-2-chlorobenzoic acid (51 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (49 mg, yield 83% for 2 steps) as a white solid: [α]₅₄₆²⁵=−56.6 (c 1.3, MeCOH); ¹H NMR (400 MHz, Methanol-d₄) δ 8.50 (s, 1H), 8.26-8.22 (m, 1H), 7.92-7.88 (m, 1H), 7.81 (d, J=8.2 Hz, 1H), 7.67-7.64 (m, 1H), 7.60-7.45 (m, 3H), 7.29 (d, J=8.8 Hz, 1H), 6.85 (dd, J=8.8, 3.1 Hz, 1H), 6.76 (d, J=3.0 Hz, 1H), 6.02 (q, J=6.9 Hz, 1H), 4.65-4.56 (m, 1H), 4.24-4.17 (m, 2H), 4.14-4.09 (m, 2H), 2.89 (s, 3H), 1.71 (d, J=6.9 Hz, 3H); ¹³C NMR (100 MHz, Methanol-d₄) δ 169.63, 149.30, 140.02, 138.14, 135.45, 132.28, 131.65, 129.91, 129.02, 127.24, 126.72, 126.43, 124.33, 123.93, 122.34, 119.54, 117.04, 52.73, 51.96, 46.81, 35.70, 21.39; HRMS (ESI) calcd for C₂₃H₂₅ClN₃O [M+H]⁺ 394.1681, found 394.1677.

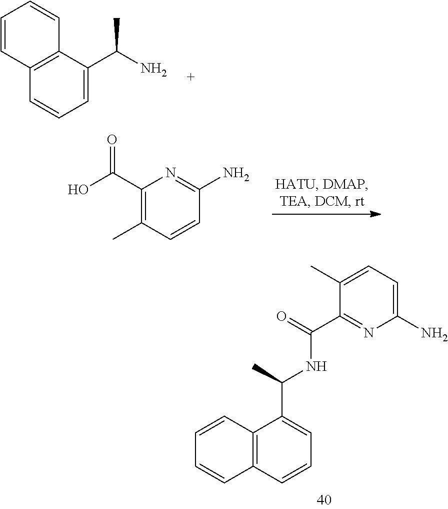

(R)-6-amino-3-methyl-N-(1-(naphthalen-1-yl)ethyl)picolinamide. (R)-1-(naphthalen-1-yl)ethan-1-amine (55 mg, 0.32 mmol), 6-amino-3-methylpicolinic acid (25 mg, 0.16 mmol), HATU (61 mg, 0.16 mmol), TEA (100 μL), and DMAP (3 mg, 0.02 mmol) was subjected to general amine coupling procedure with DCM (2 mL). The purification by Prep-HPLC afforded the product (43 mg, yield 87%) as a brown solid: [α]₅₄₆²⁵=−116.1 (c 0.6, MeOH); ¹H NMR (400 MHz, Methanol-d₄) δ 8.21-8.16 (m, 1H), 7.89-7.86 (m, 1H), 7.80-7.77 (m, 1H), 7.65-7.62 (m, 1H), 7.56-7.43 (m, 4H), 7.31 (d, J=8.4 Hz, 1H), 6.60 (d, J=8.3 Hz, 1H), 5.97 (q, J=6.9 Hz, 1H), 2.39 (s, 3H), 1.70 (d, J=6.9 Hz, 3H); ¹³C NMR (100 MHz, Methanol-d₄) δ 168.28, 162.59, 158.18, 146.77, 143.24, 140.42, 135.45, 132.25, 129.86, 128.93, 127.25, 126.68, 126.45, 124.20, 123.54, 112.72, 46.02, 21.73, 18.70; HRMS (ESI) calcd for C₁₉H₂₀N₃O [M+H]⁺306.1601, found 306.1602.

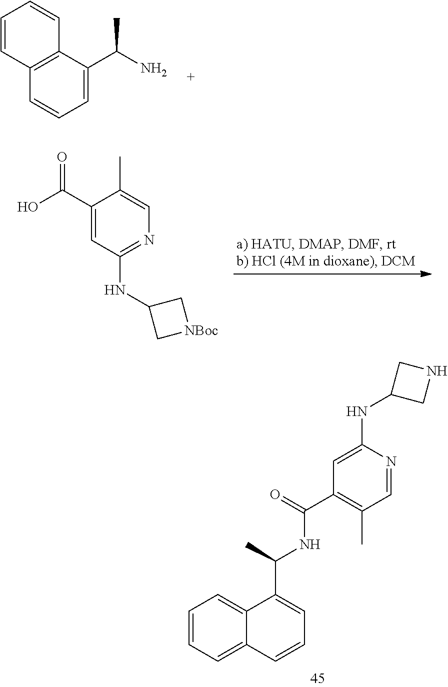

(R)-2-(azetidin-3-ylamino)-5-methyl-N-(1-(naphthalen-1-yl)ethyl)isonicotinamide. (R)-1-(naphthalen-1-yl)ethan-1-amine (35 mg, 0.20 mmol), 2-((1-(tert-butoxycarbonyl)azetidin-3-yl)amino)-5-methylisonicotinic acid (30 mg, 0.10 mmol), HATU (46 mg, 0.12 mmol) and DMAP (37 mg, 0.30 mmol) was subjected to general amine coupling procedure with DMF (1.5 mL). After purification by Prep-HPLC, the product was subjected to general N-Boc deprotection procedure with HCl (4M in dioxane, 100 μL) and DCM (2 mL). The purification by Prep-HPLC afforded the product (31 mg, yield 86% for 2 steps) as a white solid: [α]₅₄₆²⁵=−88.9 (c 1.0, MeOH); ¹H NMR (400 MHz, Methanol-d₄) δ 8.45 (s, 1H), 8.24-8.17 (m, 1H), 7.99-7.79 (m, 3H), 7.66-7.46 (m, 4H), 6.95 (d, J=5.8 Hz, 1H), 6.06-5.99 (m, 1H), 4.82-4.47 (m, 3H), 4.36-4.05 (m, 1H), 3.24-3.12 (m, 1H), 2.23-2.15 (m, 3H), 1.77-1.72 (m, 3H); ¹³C NMR (100 MHz, Methanol-d₄) δ 169.57, 166.46, 155.84, 154.27, 149.40, 139.32, 137.43, 135.49, 132.26, 130.04, 129.38, 127.47, 126.89, 126.43, 124.03, 123.93, 108.11, 55.83, 55.40, 54.39, 46.47, 21.02, 15.45; HRMS (ESI) calcd for C₂₂H₂₅N₄O [M+H]⁺ 361.2023, found 361.2024.

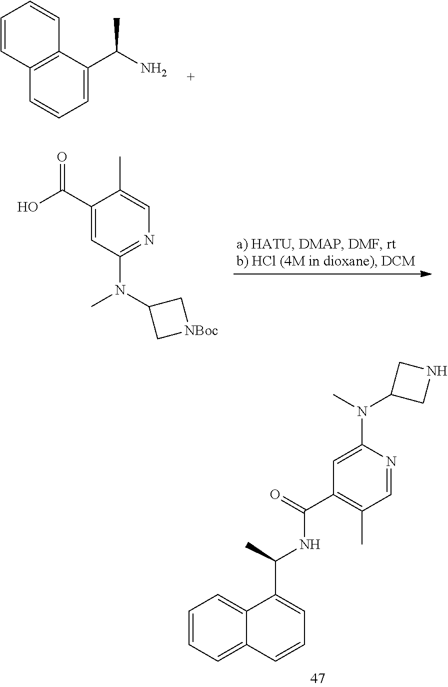

(R)-2-(azetidin-3-yl(methyl)amino)-5-methyl-N-(1-(naphthalen-1-yl)ethyl)isonicotinamide. (R)-1-(naphthalen-1-yl)ethan-1-amine (42 mg, 0.24 mmol), 2-((1-(tert-butoxycarbonyl)azetidin-3-yl)(methyl)amino)-5-methylisonicotinic acid (50 mg, 0.16 mmol), HATU (73 mg, 0.19 mmol) and DMAP (39 mg, 0.32 mmol) was subjected to general amine coupling procedure with DMF (1 mL). After purification by Prep-HPLC, the product was subjected to general N-Boc deprotection procedure with HCl (4M in dioxane, 200 μL) and DCM (4 mL). The purification by Prep-HPLC afforded the product (44 mg, yield 73% for 2 steps) as a white solid: [α]₅₄₆²⁵=−109.3 (c 0.2, MeOH); ¹H NMR (400 MHz, Methanol-d₄) δ 8.42 (s, 1H), 8.22 (d, J=8.5 Hz, 1H), 7.98 (s, 1H), 7.92 (d, J=8.1 Hz, 1H), 7.85 (d, J=8.2 Hz, 1H), 7.68-7.63 (m, 1H), 7.62-7.48 (m, 3H), 7.05 (d, J=4.7 Hz, 1H), 6.06 (q, J=6.9 Hz, 1H), 4.82-4.75 (m, 1H), 4.63-4.54 (m, 1H), 4.32 (d, J=6.1 Hz, 1H), 3.23-3.15 (m, 1H), 3.13 (s, 3H), 3.08-3.02 (m, 1H), 2.17 (d, J=4.8 Hz, 3H), 1.76 (d, J=6.9 Hz, 3H); ¹³C NMR (100 MHz, Methanol-d₄) δ 169.57, 165.77, 161.93, 144.52, 137.97, 135.38, 132.30, 130.06, 129.41, 127.48, 126.92, 126.45, 124.09, 123.99, 122.19, 106.54, 54.03, 46.52, 41.31, 31.14, 21.11, 15.38; HRMS (ESI) calcd for C₂₃H₂₇N₄O [M+H]⁺ 375.2179, found 375.2185.

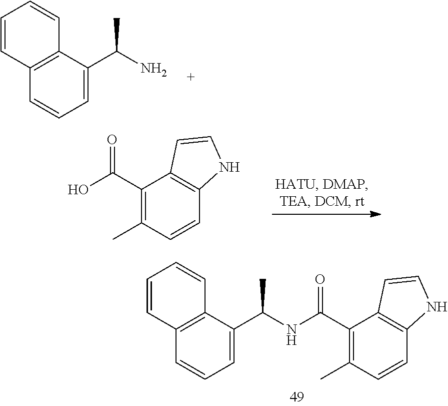

(R)-5-methyl-N-(1-(naphthalen-1-yl)ethyl)-1H-indole-4-carboxamide. (R)-1-(naphthalen-1-yl)ethan-1-amine (150 mg, 0.88 mmol), 5-methyl-1H-indole-4-carboxylic acid (128 mg, 0.73 mmol), HATU (333 mg, 0.88 mmol), and TEA (400 μL) was subjected to general amine coupling procedure with DMF (5 mL). The purification by Prep-HPLC afforded the product (209 mg, yield 87%) as a brown solid: ¹H NMR (400 MHz, Methanol-d₄) δ 7.91 (d, J=8.1 Hz, 1H), 7.82 (d, J=8.2 Hz, 1H), 7.66 (d, J=7.1 Hz, 1H), 7.60 (ddd, J=8.4, 6.9, 1.3 Hz, 1H), 7.56-7.45 (m, 2H), 7.30 (d, J=8.3 Hz, 1H), 7.16 (d, J=3.2 Hz, 1H), 6.96 (d, J=8.3 Hz, 1H), 6.28-6.24 (m, 1H), 6.20 (q, J=6.9 Hz, 1H), 2.38 (s, 3H), 1.76 (d, J=6.9 Hz, 3H). ¹³C NMR (100 MHz, Methanol-d₄) δ 140.30, 135.47, 132.46, 129.87, 128.97, 127.25, 126.73, 126.40, 125.99, 124.79, 124.60, 123.97, 113.18, 100.86, 49.00, 46.16, 21.46, 19.13; HRMS (ESI) calcd for C₂₂H₂₂N₂O [M+H]⁺329.1648, found 329.1649.

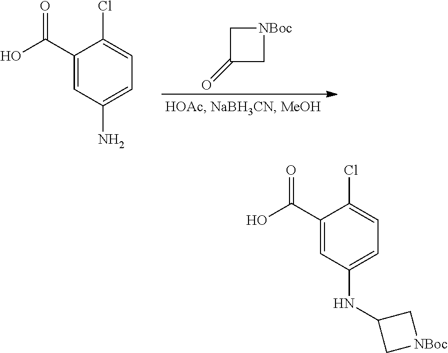 [SMILES: Nc1ccc(Cl)c(C(=O)O)c1 ; CC(C)(C)OC(=O)N1CC(=O)C1 ; CC(C)(C)OC(=O)N1CC(Nc2ccc(Cl)c(C(=O)O)c2)C1]

5-((1-(tert-butoxycarbonyl)azetidin-3-yl)amino)-2-chlorobenzoic acid. 5-amino-2-chlorobenzoic acid (3.0 g, 17.5 mmol) and tert-butyl 3-oxoazetidine-1-carboxylate (3.6 g, 21.0 mmol) was subjected to general reductive amination procedure with MeOH (20 mL), HOAc (4 mL) at 50° C., and then NaBH₃CN (3.3 g, 52.5 mmol) was added. The purification by silica gel column chromatography (Hexenes/EtOAc, 1:1) provided the amination compound (4.8 g, yield 84%) as a white solid: 1H NMR (400 MHz, Methanol-d₄) δ 7.20 (d, J=8.7 Hz, 1H), 6.96 (d, J=2.9 Hz, 1H), 6.64 (dd, J=8.7, 2.9 Hz, 1H), 4.30-4.16 (m, 3H), 3.72 (dd, J=8.7, 4.5 Hz, 2H), 1.44 (s, 9H); LRMS (ESI) calcd for C₁₅H₂₀ClN₂O₄ [M+H]⁺ 327.11, found 327.19.

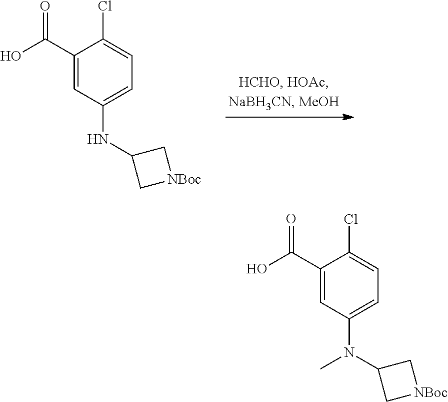 [SMILES: CC(C)(C)OC(=O)N1CC(Nc2ccc(Cl)c(C(=O)O)c2)C1 ; CN(c1ccc(Cl)c(C(=O)O)c1)C1CN(C(=O)OC(C)(C)C)C1]

5-((1-(tert-butoxycarbonyl)azetidin-3-yl)(methyl)amino)-2-chlorobenzoic acid. 5-((1-(tert-butoxycarbonyl)azetidin-3-yl)amino)-2-chlorobenzoic acid (1.8 g, 5.5 mmol) and formaldehyde (37 wt. % in H₂O, 2 mL) was subjected to general reductive amination procedure with MeOH (10 mL), HOAc (2 mL) at room temperature, and then NaBH₃CN (1.1 g, 16.5 mmol) was added. The purification by silica gel column chromatography (Hexenes/EtOAc, 2:1) provided the amination compound (1.7 g, yield 91%) as a white solid: ¹H NMR (400 MHz, Methanol-d₄) δ 7.25 (d, J=8.8 Hz, 1H), 7.15 (d, J=3.1 Hz, 1H), 6.83 (dd, J=8.9, 3.1 Hz, 1H), 4.34 (tt, J=7.5, 5.3 Hz, 1H), 4.19-4.13 (m, 2H), 3.88 (dd, J=9.1, 5.3 Hz, 2H), 2.88 (s, 3H), 1.43 (s, 9H); ¹³C NMR (100 MHz, Methanol-d₄) δ 169.12, 157.88, 149.62, 132.36, 123.42, 120.40, 118.49, 81.17, 54.89, 50.22, 35.75, 28.63; LRMS (ESI) calcd for C₁₆H₂₂ClN₂O₄ [M+H]⁺ 341.13, found 341.12.

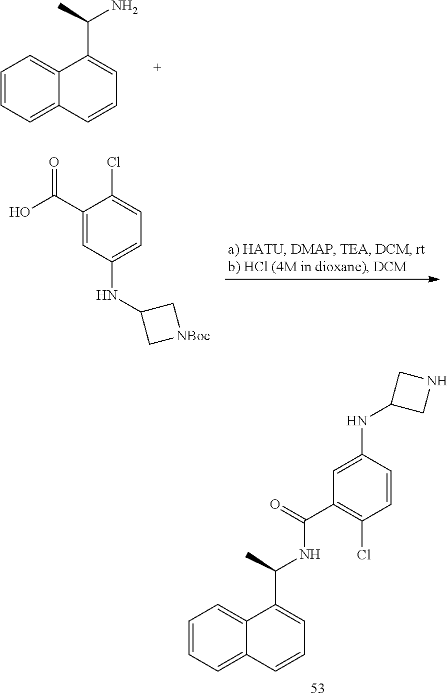

(R)-5-(azetidin-3-ylamino)-2-chloro-N-(1-(naphthalen-1-yl)ethyl)benzamide. (R)-1-(naphthalen-1-yl)ethan-1-amine (39 mg, 0.23 mmol), 5-((1-(tert-butoxycarbonyl)azetidin-3-yl)amino)-2-chlorobenzoic acid (50 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (43 mg, yield 75% for 2 steps) as a white solid: [α]₅₄₆²⁵=−54.0 (c 2.3, MeOH); ¹H NMR (400 MHz, Methanol-d₄) δ 8.51 (s, 1H), 8.25-8.20 (m, 1H), 7.92-7.87 (m, 1H), 7.80 (d, J=8.2 Hz, 1H), 7.67-7.63 (m, 1H), 7.59-7.44 (m, 3H), 7.18 (d, J=8.7 Hz, 1H), 6.62-6.53 (m, 2H), 6.02 (q, J=6.9 Hz, 1H), 4.48-4.39 (m, 1H), 4.34-4.26 (m, 2H), 3.94-3.87 (m, 2H), 1.69 (d, J=7.0 Hz, 3H); ¹³C NMR (100 MHz, Methanol-d₄) δ 169.24, 146.53, 140.08, 138.22, 135.42, 132.27, 131.68, 129.89, 128.98, 127.24, 126.70, 126.42, 124.31, 123.87, 120.16, 116.24, 113.82, 54.65, 46.71, 46.49, 21.41; HRMS (ESI) calcd for C₂₂H₂₃ClN₃O [M+H]⁺ 380.1524, found 388.1532.

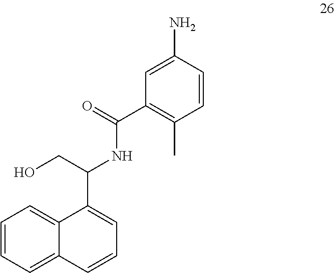 [SMILES: Cc1ccc(N)cc1C(=O)NC(CO)c1cccc2ccccc12]

5-amino-N-(2-hydroxy-1-(naphthalen-1-yl)ethyl)-2-methylbenzamide. 2-amino-2-(naphthalen-1-yl)ethan-1-ol (100 mg, 0.53 mmol), 2-methyl-5-nitrobenzoic acid (87 mg, 0.48 mmol), HATU (202 mg, 0.53 mmol), TEA (200 μL), and DMAP (6 mg, 0.05 mmol) was subjected to general amine coupling procedure with DCM (4 mL). After purification by Prep-HPLC, the product was applied to the general Aryl Nitro reduction procedure with ethanol/saturated aq. NH₄Cl (4 mL/1 mL) and Iron Powder (148 mg, 2.65 mmol). The purification by Prep-HPLC gave the product (92 mg, yield 60% for 2 steps) as a white solid: ¹H NMR (400 MHz, Methanol-d₄) δ 8.34-8.27 (m, 1H), 7.92-7.85 (m, 1H), 7.84-7.77 (m, 1H), 7.67-7.42 (m, 5H), 7.25-6.95 (m, 1H), 6.80-6.67 (m, 1H), 6.10-6.02 (m, 1H), 4.08-3.99 (m, 1H), 3.94-3.83 (m, 1H), 2.27 (s, 3H); ¹³C NMR (100 MHz, Methanol-d₄) δ 173.30, 146.27, 138.45, 136.75, 135.38, 132.31, 129.91, 129.10, 127.35, 126.72, 126.34, 124.77, 124.06, 122.34, 119.79, 118.10, 115.19, 65.35, 53.20, 18.74; HRMS (ESI) calcd for C₂₀H₂₁N₂O₂ [M+H]⁺ 321.1598, found 308.1589.

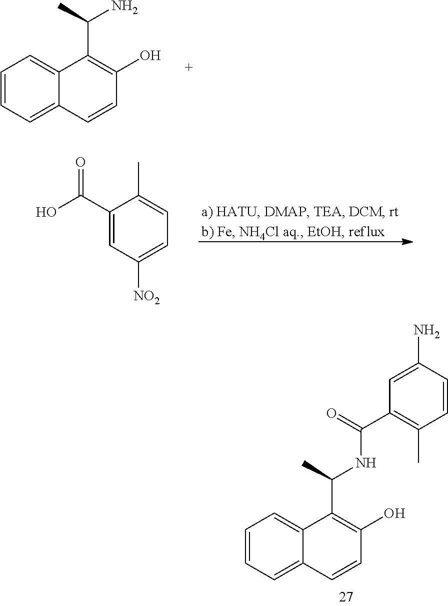

(R)-5-amino-N-(1-(2-hydroxynaphthalen-1-yl)ethyl)-2-methylbenzamide. (R)-1-(1-aminoethyl)naphthalen-2-ol (100 mg, 0.53 mmol), 2-methyl-5-nitrobenzoic acid (87 mg, 0.48 mmol), HATU (202 mg, 0.53 mmol), TEA (200 μL), and DMAP (6 mg, 0.05 mmol) was subjected to general amine coupling procedure with DCM (4 mL). After purification by Prep-HPLC, the product was applied to the general Aryl Nitro reduction procedure with ethanol/saturated aq. NH₄Cl (4 mL/1 mL) and Iron Powder (148 mg, 2.65 mmol). The purification by Prep-HPLC gave the product (87 mg, yield 57% for 2 steps) as a light brown solid: [α]5=−62.4 (c 0.3, MeCOH); ¹H NMR (400 MHz, Methanol-d₄) δ 8.15 (d, J=8.8 Hz, 1H), 7.77 (dd, J=8.2, 1.4 Hz, 1H), 7.69 (d, J=8.9 Hz, 1H), 7.50 (ddd, J=8.5, 6.8, 1.4 Hz, 1H), 7.35-7.26 (m, 1H), 7.12 (d, J=8.8 Hz, 1H), 6.96 (d, J=8.1 Hz, 1H), 6.77-6.67 (m, 2H), 6.19 (q, J=7.0 Hz, 1H), 2.21 (s, 3H), 1.65 (d, J=7.0 Hz, 3H); ¹¹C NMR (100 MHz, Methanol-d₄) δ 171.98, 153.95, 146.47, 138.44, 133.26, 132.57, 130.42, 130.13, 129.73, 127.76, 125.65, 123.92, 122.98, 120.89, 119.26, 118.30, 114.94, 44.76, 20.68, 18.67; HRMS (ESI) calcd for C₂₀H₂₁N₂O₂ [M+H]⁺321.1598, found 321.1599.

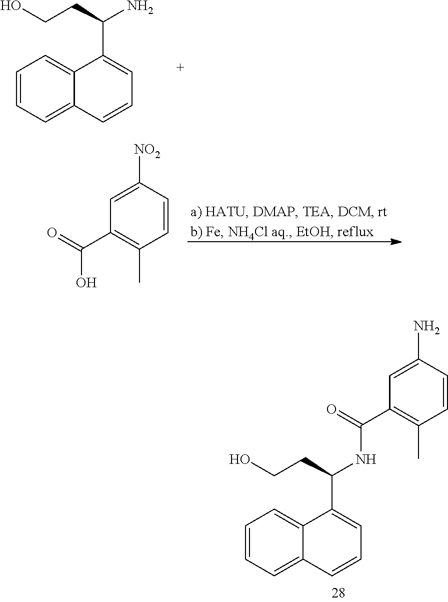

(R)-5-amino-N-(3-hydroxy-1-(naphthalen-1-yl)propyl)-2-methylbenzamide. (R)-3-amino-3-(naphthalen-1-yl)propan-1-ol (100 mg, 0.50 mmol), 2-methyl-5-nitrobenzoic acid (60 mg, 0.33 mmol), HATU (124 mg, 0.33 mmol), TEA (100 μL), and DMAP (5 mg, 0.04 mmol) was subjected to general amine coupling procedure with DCM (4 mL). After purification by Prep-HPLC, the product was applied to the general Aryl Nitro reduction procedure with ethanol/saturated aq. NH₄Cl (4 mL/1 mL) and Iron Powder (92 mg, 1.65 mmol). The purification by Prep-HPLC gave the product (62 mg, yield 56% for 2 steps) as a white solid: ¹H NMR (400 MHz, Methanol-d₄) δ 8.33 (dd, J=8.7, 4.8 Hz, 1H), 7.91-7.86 (m, 1H), 7.81 (d, J=8.2 Hz, 1H), 7.64-7.45 (m, 5H), 6.98-6.95 (m, 1H), 6.73-6.69 (m, 2H), 6.15-6.07 (m, 1H), 3.85-3.71 (m, 2H), 2.29-2.12 (m, 5H); ¹³C NMR (100 MHz, Methanol-d₄) δ 173.07, 146.00, 139.67, 138.40, 135.43, 132.38, 129.87, 128.90, 127.28, 126.75, 126.39, 125.88, 124.30, 124.10, 118.30, 115.25, 60.12, 47.73, 39.41, 18.73; LRMS (ESI) calcd for C₂₁H₂₃N₂O₂ [M+H]⁺ 335.18, found 335.18.

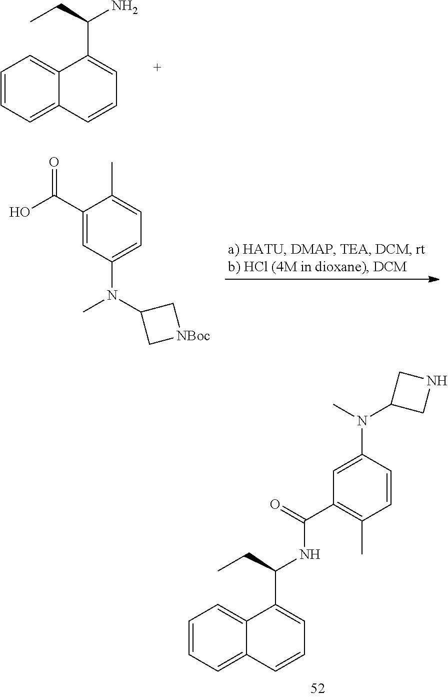

(R)-5-(azetidin-3-yl(methyl)amino)-2-methyl-N-(1-(naphthalen-1-yl)propyl)benzamide. (R)-1-(naphthalen-1-yl)propan-1-amine (17 mg, 0.09 mmol), 5-((1-(tert-butoxycarbonyl)azetidin-3-yl)(methyl)amino)-2-methylbenzoic acid (20 mg, 0.06 mmol), HATU (24 mg, 0.06 mmol) and DMAP (15 mg, 0.13 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (18 mg, yield 77% for 2 steps) as a white solid: [α]₅₄₆²⁵=−65.4 (c 0.9, MeOH); ¹H NMR (400 MHz, Methanol-d₄) δ 8.40 (s, 1H), 8.30 (d, J=8.5 Hz, 1H), 7.92-7.89 (m, 1H), 7.81 (d, J=8.2 Hz, 1H), 7.60-7.46 (m, 4H), 7.12 (d, J=8.4 Hz, 1H), 6.81 (dd, J=8.4, 2.7 Hz, 1H), 6.69 (d, J=2.7 Hz, 1H), 5.84 (dd, J=9.0, 5.7 Hz, 1H), 4.51-4.41 (m, 1H), 4.22-4.15 (m, 2H), 4.09-4.01 (m, 2H), 2.83 (s, 3H), 2.23 (s, 3H), 2.15-1.95 (m, 2H), 1.13 (t, J=7.3 Hz, 3H); ¹³C NMR (100 MHz, Methanol-d₄) δ 172.56, 148.38, 139.84, 139.05, 135.48, 132.60, 129.96, 128.89, 128.49, 127.22, 126.72, 126.41, 124.30, 124.26, 119.66, 116.94, 53.33, 52.51, 51.97, 37.10, 29.84, 18.73, 11.92; HRMS (ESI) calcd for C₂₅H₃₀N₃O [M+H]⁺ 388.2383, found 388.2389.

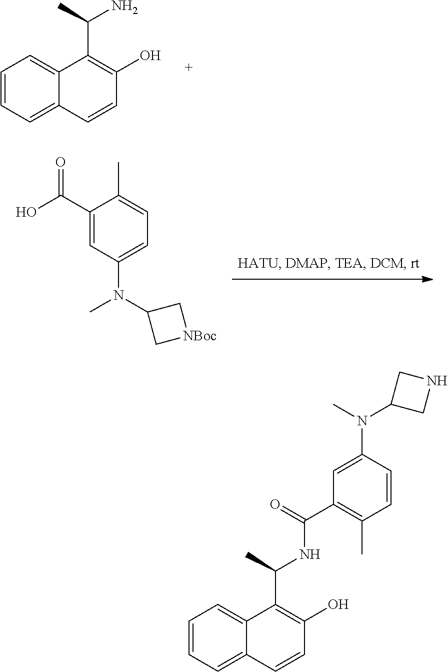 [SMILES: C[C@@H](N)c1c(O)ccc2ccccc12 ; Cc1ccc(N(C)C2CN(C(=O)OC(C)(C)C)C2)cc1C(=O)O ; Cc1ccc(N(C)C2CNC2)cc1C(=O)N[C@H](C)c1c(O)ccc2ccccc12]

Tert-butyl (R)-3-((3-((1-(2-hydroxynaphthalen-1-yl)ethyl)carbamoyl)-4-methylphenyl)(methyl)amino)azetidine-1-carboxylate. (R)-1-(1-aminoethyl)naphthalen-2-ol (178 mg, 0.95 mmol), 5-((1-(tert-butoxycarbonyl)azetidin-3-yl)(methyl)amino)-2-methylbenzoic acid (254 mg, 0.79 mmol), HATU (300 mg, 0.79 mmol), TEA (500 μL), and DMAP (10 mg, 0.08 mmol) was subjected to general amine coupling procedure with DCM (4 mL). The purification by Prep-HPLC gave the desired compound (325 mg, yield 84%) as a white solid: ¹H NMR (400 MHz, Chloroform-d) δ 9.07 (s, 1H), 8.15 (d, J=8.7 Hz, 1H), 7.81-7.72 (m, 1H), 7.61 (d, J=8.8 Hz, 1H), 7.49-7.44 (m, 1H), 7.34-7.30 (m, 1H), 7.18 (d, J=8.8 Hz, 1H), 7.01 (d, J=8.3 Hz, 1H), 6.75-6.56 (m, 2H), 6.31-6.19 (m, 1H), 4.17-4.04 (m, 3H), 3.84 (td, J=8.1, 7.6, 5.3 Hz, 2H), 2.70 (s, 3H), 2.32 (s, 3H), 1.65 (d, J=6.9 Hz, 3H), 1.45 (s, 9H); LRMS (ESI) calcd for C₂₉H₃₆N₃O₄ [M+H]⁺490.27, found 490.27.

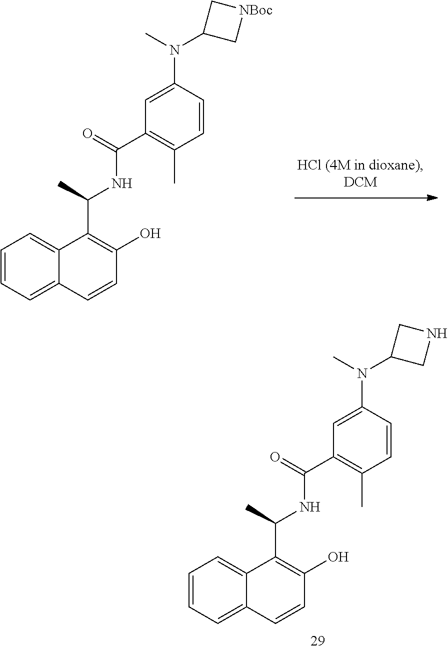 [SMILES: Cc1ccc(N(C)C2CN(C(=O)OC(C)(C)C)C2)cc1C(=O)N[C@H](C)c1c(O)ccc2ccccc12 ; Cc1ccc(N(C)C2CNC2)cc1C(=O)N[C@H](C)c1c(O)ccc2ccccc12]

(R)-5-(azetidin-3-yl(methyl)amino)-N-(1-(2-hydroxynaphthalen-1-yl)ethyl)-2-methylbenzamide. General procedure for N-Boc deprotection was used with tert-butyl (R)-3-((3-((1-(2-hydroxynaphthalen-1-yl)ethyl)carbamoyl)-4-methylphenyl)(methyl)amino)azetidine-1-carboxylate (40 mg, 0.08 mmol) in DCM (2 mL) and HCl (4M in dioxane, 200 μL). The purification by Prep-HPLC afforded the product (23 mg, yield 72%) as a light brown solid: [α]₅₄₆²⁵=−70.4 (c 0.4, MeOH); ¹H NMR (400 MHz, Methanol-d₄) δ 8.55 (s, 1H), 8.17 (d, J=8.7 Hz, 1H), 7.81-7.75 (m, 1H), 7.70 (d, J=8.8 Hz, 1H), 7.53-7.48 (m, 1H), 7.33-7.29 (m, 1H), 7.16-7.12 (m, 2H), 6.83 (dd, J=8.4, 2.7 Hz, 1H), 6.78-6.76 (m, 1H), 6.20 (q, J=6.9 Hz, 1H), 4.47 (p, J=7.2 Hz, 1H), 4.23-4.16 (m, 2H), 4.07-4.01 (m, 2H), 2.85 (s, 3H), 2.27 (s, 3H), 1.67 (d, J=7.0 Hz, 3H); ¹³C NMR (100 MHz, Methanol-d₄) δ 169.52, 153.96, 148.60, 138.71, 133.28, 132.89, 130.43, 130.19, 129.78, 128.42, 127.74, 123.92, 123.01, 120.93, 119.94, 119.25, 116.70, 53.46, 52.12, 44.89, 37.05, 20.51, 18.74; HRMS (ESI) calcd for C₂₄H₂₈N₃O₂ [M+H]⁺ 390.2176, found 390.2185.

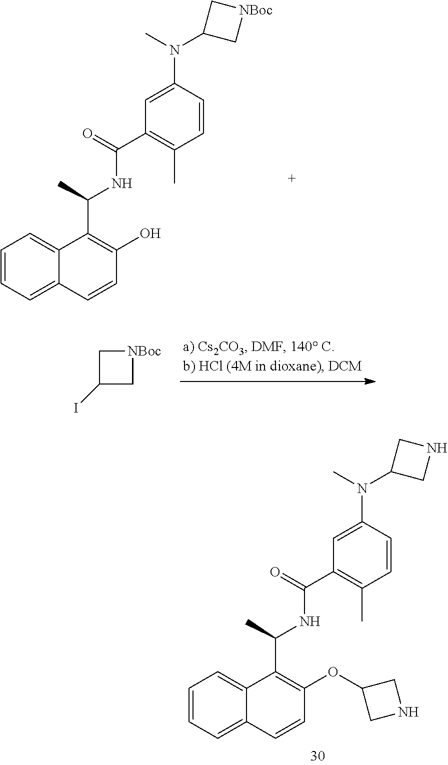 [SMILES: Cc1ccc(N(C)C2CN(C(=O)OC(C)(C)C)C2)cc1C(=O)N[C@H](C)c1c(O)ccc2ccccc12 ; CC(C)(C)OC(=O)N1CC(I)C1 ; Cc1ccc(N(C)C2CNC2)cc1C(=O)N[C@H](C)c1c(OC2CNC2)ccc2ccccc12]

(R)-5-(azetidin-3-yl(methyl)amino)-N-(1-(2-(azetidin-3-yloxy)naphthalen-1-yl)ethyl)-2-methylbenzamide. To a solution of tert-butyl (R)-3-((3-((1-(2-hydroxynaphthalen-1-yl)ethyl)carbamoyl)-4-methylphenyl)(methyl)amino)azetidine-1-carboxylate (20 mg, 0.04 mmol) and tert-butyl 3-iodoazetidine-1-carboxylate (14 mg, 0.05 mol) in DMF (1 mL), Cs₂CO₃ (26 mg, 0.08 mmol) was added. The reaction was stirred at 140° C. for 5 h. After cooling down, quench the reaction with MeOH (1 mL). The mixture was diluted with Ethyl Acetate and was then washed with saturated aq. NaHCO₃, water, and brine, respectively. The organic layer was dried over Na₂SO₄, filtered, and concentrated. After purification by Prep-HPLC, the product was applied to the general procedure for N-Boc deprotection with HCl (4M in dioxane, 50 μL) and DCM (1 mL). The purification by Prep-HPLC afforded the product (8 mg, yield 45% for 2 steps) as a light brown solid: [α]₅₄₆²⁵=−87 (c 0.6, MeCOH); ¹H NMR (400 MHz, Methanol-d₄) δ 8.53 (s, 1H), 8.41 (d, J=8.7 Hz, 1H), 7.91-7.82 (m, 2H), 7.57-7.52 (m, 1H), 7.46-7.06 (m, 3H), 6.80-6.75 (m, 1H), 6.65-6.62 (m, 1H), 6.24 (dq, J=9.8, 7.4 Hz, 1H), 5.44-5.09 (m, 1H), 4.59-4.28 (m, 3H), 4.22-4.12 (m, 2H), 4.07-3.97 (m, 3H), 3.56-3.43 (m, 1H), 2.84 (s, 3H), 2.10 (d, J=9.0 Hz, 3H), 1.77 (d, J=7.4 Hz, 3H); ¹³C NMR (100 MHz, Methanol-d₄) δ 172.23, 152.57, 148.36, 146.49, 138.90, 138.51, 132.55, 131.04, 130.42, 130.28, 127.61, 125.15, 125.05, 119.45, 116.92, 115.78, 114.98, 70.75, 54.93, 54.77, 53.39, 52.07, 44.41, 36.58, 20.06, 18.47; HRMS (ESI) calcd for C₂₇H₃₃N₄O₂ [M+H]⁺445.2598, found 445.2587.

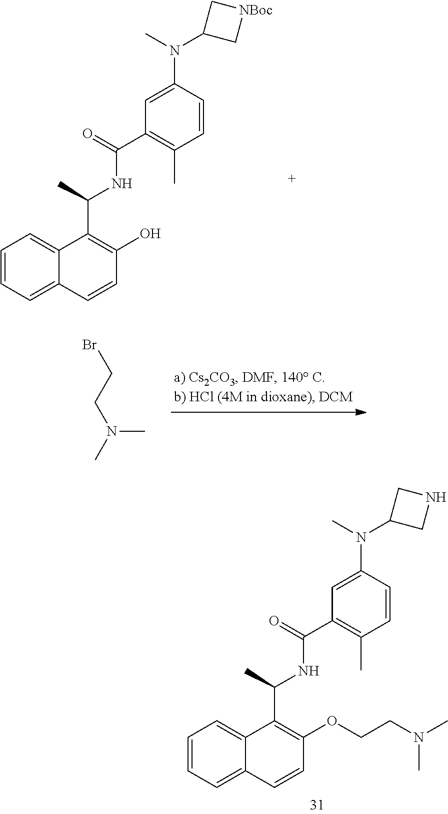

(R)-5-(azetidin-3-yl(methyl)amino)-N-(1-(2-(2-(dimethylamino)ethoxy)naphthalen-1-yl)ethyl)-2-methylbenzamide. To a solution of tert-butyl (R)-3-((3-((1-(2-hydroxynaphthalen-1-yl)ethyl)carbamoyl)-4-methylphenyl)(methyl)amino)azetidine-1-carboxylate (20 mg, 0.04 mmol) and 2-bromo-N,N-dimethylethan-1-amine (12 mg, 0.05 mol) in DMF (1 mL), Cs₂CO₃ (26 mg, 0.08 mmol) was added. The reaction was stirred at 140° C. for 5 h. After cooling down, quench the reaction with MeOH (1 mL). The mixture was diluted with Ethyl Acetate and was then washed with saturated aq. NaHCO₃, water, and brine, respectively. The organic layer was dried over Na₂SO₄, filtered, and concentrated. After purification by Prep-HPLC, the product was applied to the general procedure for N-Boc deprotection with HCl (4M in dioxane, 50 μL) and DCM (1 mL). The purification by Prep-HPLC afforded the product (9 mg, yield 49% for 2 steps) as a light brown solid: [α]₅₄₆²⁵=−57.8 (c 0.5, MeOH); ¹H NMR (400 MHz, Methanol-d₄) δ 8.53 (s, 1H), 8.31 (d, J=8.9 Hz, 1H), 7.88-7.83 (m, 2H), 7.58-7.52 (m, 1H), 7.44-7.36 (m, 2H), 7.12 (d, J=8.4 Hz, 1H), 6.80 (dd, J=8.4, 2.7 Hz, 1H), 6.65 (d, J=2.7 Hz, 1H), 6.36 (q, J=7.3 Hz, 1H), 4.51-3.99 (m, 9H), 2.83 (s, 3H), 2.26-2.03 (m, 9H), 1.72 (d, J=7.1 Hz, 3H); ¹³C NMR (100 MHz, Methanol-d₄) δ 172.40, 148.53, 132.90, 132.54, 130.75, 130.01, 128.07, 127.85, 124.74, 120.95, 119.30, 116.70, 115.42, 66.20, 59.41, 53.35, 52.08, 45.01, 44.06, 36.94, 20.61, 18.43; HRMS (ESI) calcd for C₂₈H₃₇N₄O₂ [M+H]⁺461.2911, found 461.2918.

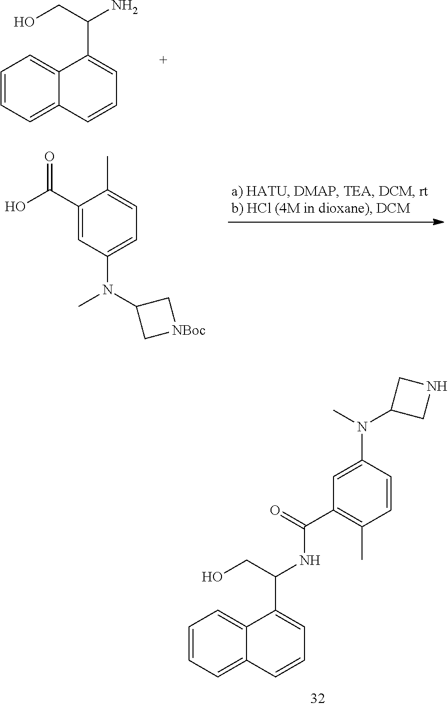

5-(azetidin-3-yl(methyl)amino)-N-(2-hydroxy-1-(naphthalen-1-yl)ethyl)-2-methylbenzamide. 2-amino-2-(naphthalen-1-yl)ethan-1-ol (100 mg, 0.53 mmol), 5-((1-(tert-butoxycarbonyl)azetidin-3-yl)(methyl)amino)-2-methylbenzoic acid (170 mg, 0.53 mmol), HATU (201 mg, 0.53 mmol), DMAP (6 mg, 0.05 mmol) and TEA (200 μL) was subjected to general amine coupling procedure with DCM (4 mL). After purification by Prep-HPLC, the product was subjected to general N-Boc deprotection procedure with HCl (4M in dioxane, 200 μL) and DCM (4 mL). The purification by Prep-HPLC afforded the product (151 mg, yield 73% for 2 steps) as a white solid: ¹H NMR (400 MHz, Methanol-d₄) δ 8.62-8.59 (m, 1H), 8.34-8.29 (m, 1H), 7.94-7.89 (m, 1H), 7.85-7.81 (m, 1H), 7.63-7.45 (m, 4H), 7.09 (d, J=8.1 Hz, 1H), 6.85-6.79 (m, 2H), 6.09-6.03 (m, 1H), 4.53-4.47 (m, 1H), 4.29-4.21 (m, 2H), 4.11-4.02 (m, 3H), 3.90 (dd, J=11.5, 8.3 Hz, 1H), 2.87 (s, 3H), 2.26 (s, 3H); ¹³C NMR (100 MHz, Methanol-d₄) δ 162.40, 148.38, 138.93, 138.53, 135.46, 132.64, 132.19, 129.99, 129.07, 128.88, 127.41, 126.82, 126.34, 124.29, 124.05, 119.96, 117.17, 65.42, 53.43, 52.22, 46.99, 37.30, 18.75; HRMS (ESI) calcd for C₂₈H₃₇N₄O₂ [M+H]⁺461.2911, found 461.2918.

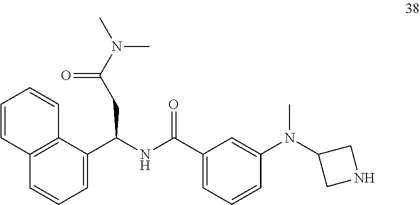 [SMILES: CN(C)C(=O)C[C@@H](NC(=O)c1cccc(N(C)C2CNC2)c1)c1cccc2ccccc12]

(R)-3-(azetidin-3-yl(methyl)amino)-N-(3-(dimethylamino)-1-(naphthalen-1-yl)-3-oxopropyl)benzamide. [α]₅₄₆²⁵=−17.9 (c 0.5, MeOH); ¹H NMR (400 MHz, Methanol-d₄) δ 8.55 (s, 1H), 8.37 (d, J=8.1 Hz, 1H), 7.92 (d, J=7.8 Hz, 1H), 7.83 (d, J=8.8 Hz, 1H), 7.63-7.45 (m, 5H), 7.12 (d, J=8.1 Hz, 1H), 6.84-6.80 (m, 1H), 6.76-6.74 (m, 1H), 6.43-6.38 (m, 1H), 4.48 (p, J=7.3 Hz, 1H), 4.20-4.15 (m, 2H), 4.06-3.99 (m, 2H), 2.98-2.81 (m, 5H), 2.71 (s, 3H), 2.23 (s, 3H); ¹³C NMR (100 MHz, Methanol-d₄) δ 173.41, 170.23, 148.51, 138.64, 138.55, 135.51, 132.67, 132.15, 129.98, 129.30, 128.52, 127.48, 126.90, 126.35, 124.25, 119.81, 116.82, 53.68, 52.24, 42.52, 37.09, 26.45, 18.69; HRMS (ESI) calcd for C₂₆H₃₁N₄O₂ [M+H]⁺431.2442, found 432.2437.

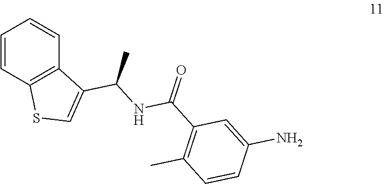 [SMILES: Cc1ccc(N)cc1C(=O)N[C@H](C)c1csc2ccccc12]

(R)-5-amino-N-(1-(benzo[b]thiophen-3-yl)ethyl)-2-methylbenzamide. Light yellow solid (yield 83%): [α]₅₄₆²⁵=−34.7 (c 1.4, MeOH); ¹H NMR (400 MHz, Chloroform-d) δ 7.97-7.92 (m, 1H), 7.88-7.83 (m, 1H), 7.45-7.30 (m, 3H), 6.96-6.91 (m, 1H), 6.60-6.55 (m, 2H), 5.80-5.69 (m, 1H), 2.29 (s, 3H), 1.75 (d, J=6.7 Hz, 3H); ¹³C NMR (100 MHz, Chloroform-d) δ 172.91, 144.23, 140.71, 137.90, 131.99, 125.47, 124.89, 124.53, 122.98, 122.46, 122.35, 116.86, 116.82, 113.47, 113.44, 43.15, 20.44, 18.87; HRMS (ESI) calcd for C₁₈H₁₈N₃O₃ [M+H]⁺324.1343, found 324.1341.

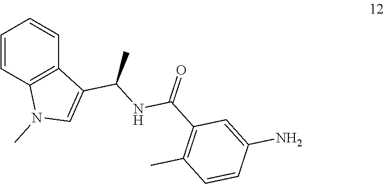 [SMILES: Cc1ccc(N)cc1C(=O)N[C@H](C)c1cn(C)c2ccccc12]

(R)-5-amino-2-methyl-N-(1-(1-methyl-1H-indol-3-yl)ethyl)benzamide. White solid (yield 79%): ¹H NMR (400 MHz, Chloroform-d) δ 7.73 (dt, J=7.9, 1.0 Hz, 1H), 7.34-7.23 (m, 2H), 7.17-7.11 (m, 1H), 7.05-7.00 (m, 1H), 6.94 (d, J=8.1 Hz, 1H), 6.64 (d, J=2.6 Hz, 1H), 6.59 (dd, J=8.1, 2.5 Hz, 1H), 5.92 (d, J=8.2 Hz, 1H), 5.64 (tt, J=7.4, 6.4 Hz, 1H), 3.77 (s, 3H), 2.32 (s, 3H), 1.73 (d, J=6.7 Hz, 3H); LRMS (ESI) calcd for C₁₉H₂₂N₃O [M+H]⁺ 308.18, found 308.14.

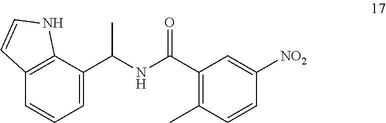 [SMILES: Cc1ccc([N+](=O)[O-])cc1C(=O)NC(C)c1cccc2cc[nH]c12]

N-(1-(1H-indol-7-yl)ethyl)-2-methyl-5-nitrobenzamide. 1-(1H-indol-7-yl)ethan-1-amine (100 mg, 0.62 mmol), 2-methyl-5-nitrobenzoic acid (101 mg, 0.56 mmol), HATU (213 mg, 0.56 mmol) and DMAP (205 mg, 1.68 mmol) was subjected to general amine coupling procedure with DMF (3 mL). The purification by Prep-HPLC afforded the product (142 mg, yield 78%) as a brown solid: ¹H NMR (400 MHz, Methanol-d₄) δ 8.16-8.12 (m, 2H), 7.51-7.48 (m, 1H), 7.44-7.40 (m, 1H), 7.28 (d, J=3.2 Hz, 1H), 7.18-7.15 (m, 1H), 7.05-7.00 (m, 1H), 6.49 (d, J=3.2 Hz, 1H), 5.71 (q, J=7.0 Hz, 1H), 2.37 (s, 3H), 1.70 (d, J=7.0 Hz, 3H); ¹³C NMR (100 MHz, Methanol-d₄) δ 169.94, 147.22, 144.92, 139.02, 135.22, 132.90, 129.95, 127.31, 125.58, 125.21, 122.99, 120.69, 120.25, 118.45, 103.00, 46.58, 20.38, 19.70; HRMS (ESI) calcd for C₁₈H₁₉N₂OS [M+H]⁺311.1213, found 311.1215.

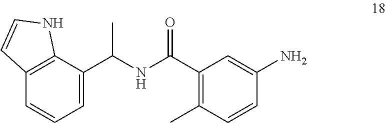 [SMILES: Cc1ccc(N)cc1C(=O)NC(C)c1cccc2cc[nH]c12]

N-(1-(1H-indol-7-yl)ethyl)-5-amino-2-methylbenzamide. To a solution of N-(1-(1H-indol-7-yl)ethyl)-2-methyl-5-nitrobenzamide (80 mg, 0.25 mmol) in ethanol/saturated aq. NH₄Cl (4/1 mL), Fe powder (67 mg, 1.2 mmol) was added. The resulting solution was stirred for 2 h at 80° C. and then concentrated under vacuum. The residue was extracted with 3×10 mL of ethyl acetate and the organic layers combined. The organic layer was washed with 5 mL of brine, dried and concentrated under vacuum to remove the solvent. The residue was purified by Prep-HPLC to obtain the desired product (62 mg, yield 85%) as a light brown solid: ¹H NMR (400 MHz, Methanol-d₄) δ 7.50-7.46 (m, 1H), 7.25 (d, J=3.1 Hz, 1H), 7.18-7.14 (m, 1H), 7.05-6.98 (m, 1H), 6.94-6.91 (m, 1H), 6.66 (d, J=7.2 Hz, 2H), 6.48 (d, J=3.2 Hz, 1H), 5.67 (q, J=7.0 Hz, 1H), 2.15 (s, 3H), 1.67 (d, J=7.0 Hz, 3H); ¹³C NMR (100 MHz, Methanol-d₄) δ 173.02, 146.38, 138.36, 135.29, 132.29, 129.83, 127.59, 125.60, 125.44, 120.54, 120.19, 118.61, 118.02, 115.03, 102.98, 102.91, 46.29, 20.26, 18.49; HRMS (ESI) calcd for C₁₈H₂₀N₃O [M+H]⁺ 294.1601, found 294.1606.

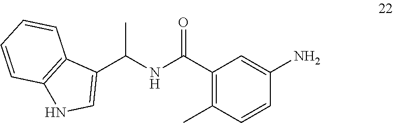 [SMILES: Cc1ccc(N)cc1C(=O)NC(C)c1c[nH]c2ccccc12]

N-(1-(1H-indol-3-yl)ethyl)-5-amino-2-methylbenzamide. 1-(1H-indol-3-yl)ethan-1-amine (50 mg, 0.31 mmol), 5-amino-2-(trifluoromethyl)benzoic acid (47 mg, 0.31 mmol), HATU (118 mg, 0.31 mmol), TEA (100 μL), and DMAP (4 mg, 0.03 mmol) was subjected to general amine coupling procedure with DCM (2 mL). The purification by Prep-HPLC afforded the product (55 mg, yield 60%) as a brown solid: ¹H NMR (400 MHz, Methanol-d₄) δ 7.72-7.67 (m, 1H), 7.36-7.33 (m, 1H), 7.23-7.21 (m, 1H), 7.17-6.98 (m, 3H), 6.95-6.89 (m, 1H), 6.67-6.62 (m, 1H), 5.66-5.55 (m, 1H), 2.22 (s, 3H), 1.68 (d, J=6.8 Hz, 3H); ¹³C NMR (100 MHz, Methanol-d₄) δ 172.38, 161.60, 146.27, 138.70, 138.35, 132.25, 125.56, 122.73, 122.62, 119.97, 119.93, 119.89, 117.95, 115.12, 112.34, 43.03, 20.85, 18.66; HRMS (ESI) calcd for C₁₈H₂₀N₃O [M+H]⁺294.1601, found 294.1649.

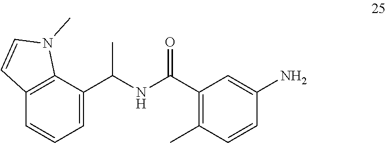 [SMILES: Cc1ccc(N)cc1C(=O)NC(C)c1cccc2ccn(C)c12]

5-amino-2-methyl-N-(1-(1-methyl-1H-indol-7-yl)ethyl)benzamide. N-(1-(1H-indol-7-yl)ethyl)-2-methyl-5-nitrobenzamide (50 mg, 0.15 mmol) and formaldehyde (37 wt. % in H₂O, 100 mL) was subjected to general reductive amination procedure with MeOH (2 mL), HOAc (50 mL) at room temperature, and then NaBH₃CN (47 mg, 0.75 mmol) was added. After purification by Prep-HPLC, the product was dissolved in ethanol/saturated aq. NH₄Cl (4 mL/1 mL), and then Fe powder (42 mg, 0.75 mmol) was added. The resulting solution was stirred for 2 h at 80° C. and then concentrated under vacuum. The residue was extracted with 3×10 mL of ethyl acetate and the organic layers were combined. The organic mixture was washed with brine, dried, and concentrated under vacuum. The residue was purified by Prep-HPLC to obtain the desired product (31 mg, yield 67%) as a brown solid: ¹H NMR (400 MHz, Methanol-d₄) δ 7.58-7.51 (m, 1H), 7.33 (d, J=7.4 Hz, 1H), 7.14-6.93 (m, 3H), 6.71-6.62 (m, 2H), 6.51-6.42 (m, 2H), 4.08 (s, 3H), 2.43 (s, 3H), 1.74 (d, J=6.8 Hz, 3H); ¹³C NMR (100 MHz, Methanol-d₄) δ 174.63, 147.31, 137.70, 132.89, 132.32, 130.95, 125.77, 122.91, 122.38, 121.54, 120.09, 119.60, 117.35, 102.21, 46.05, 32.01, 17.89, 15.94; HRMS (ESI) calcd for C₁₉H₂₂N₃O [M+H]⁺308.1757, found 308.1709.

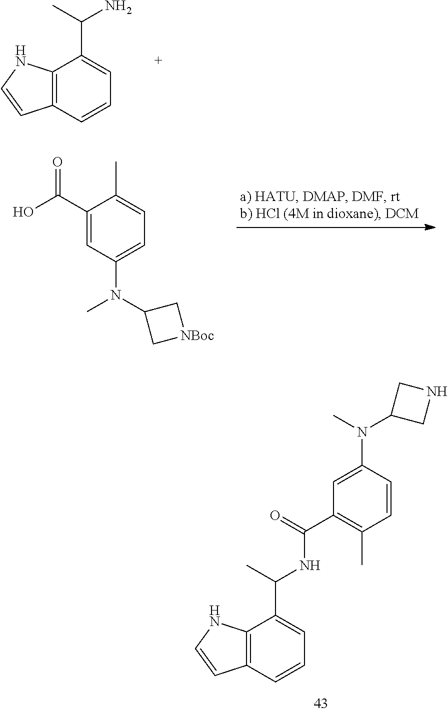

N-(1-(1H-indol-7-yl)ethyl)-5-(azetidin-3-yl(methyl)amino)-2-methylbenzamide. 1-(1H-indol-7-yl)ethan-1-amine (24 mg, 0.15 mmol), 5-((1-(tert-butoxycarbonyl)azetidin-3-yl)(methyl)amino)-2-methylbenzoic acid (40 mg, 0.13 mmol), HATU (48 mg, 0.13 mmol) and DMAP (31 mg, 0.25 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (33 mg, yield 70% for 2 steps) as a white solid: ¹H NMR (400 MHz, Methanol-d₄) δ 8.38 (s, 1H), 7.49 (dd, J=7.9, 1.1 Hz, 1H), 7.28 (d, J=3.2 Hz, 1H), 7.14 (dd, J=18.8, 7.8 Hz, 2H), 7.05-6.99 (m, 1H), 6.81 (dd, J=8.3, 2.7 Hz, 1H), 6.72 (d, J=2.7 Hz, 1H), 6.49 (d, J=3.1 Hz, 1H), 5.69 (q, J=6.9 Hz, 1H), 4.52-4.43 (m, 1H), 4.23-4.17 (m, 2H), 4.09-4.03 (m, 2H), 2.84 (s, 3H), 2.20 (s, 3H), 1.69 (d, J=7.0 Hz, 3H); ¹³C NMR (100 MHz, Methanol-d₄) δ 172.48, 138.84, 135.24, 132.63, 129.91, 128.54, 127.61, 125.49, 120.59, 120.22, 119.76, 118.60, 116.90, 102.97, 53.34, 52.04, 46.50, 37.07, 20.40, 18.53; HRMS (ESI) calcd for C₂₂H₂₇N₄O [M+H]⁺ 363.2179, found 363.2179.

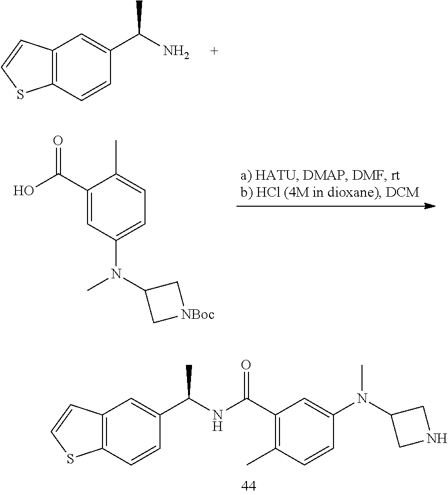

(R)-5-(azetidin-3-yl(methyl)amino)-N-(1-(benzo[b]thiophen-5-yl)ethyl)-2-methylbenzamide. (R)-1-(benzo[b]thiophen-5-yl)ethan-1-amine (21 mg, 0.12 mmol), 5-((1-(tert-butoxycarbonyl)azetidin-3-yl)(methyl)amino)-2-methylbenzoic acid (32 mg, 0.10 mmol), HATU (76 mg, 0.20 mmol) and DMAP (44 mg, 0.36 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (30 mg, yield 79% for 2 steps) as a white solid: ¹H NMR (400 MHz, Methanol-d₄) δ 8.54 (s, 1H), 7.92-7.85 (m, 2H), 7.57 (d, J=5.4 Hz, 1H), 7.45-7.39 (m, 1H), 7.36 (d, J=5.4 Hz, 1H), 7.13 (d, J=8.4 Hz, 1H), 6.81 (dd, J=8.3, 2.6 Hz, 1H), 6.76 (d, J=2.5 Hz, 1H), 5.33 (q, J=7.0 Hz, 1H), 4.51 (p, J=7.3 Hz, 1H), 4.22 (dd, J=11.3, 7.6 Hz, 2H), 4.13-4.02 (m, 2H), 2.86 (s, 3H), 2.24 (s, 3H), 1.61 (s, 3H); ¹³C NMR (100 MHz, Methanol-d₄) δ 172.16, 148.44, 141.53, 141.35, 139.91, 138.96, 132.60, 128.45, 128.07, 124.88, 124.00, 123.48, 122.08, 119.65, 116.87, 53.35, 52.01, 50.66, 49.00, 37.03, 22.50, 18.64; HRMS (ESI) calcd for C₂₂H₂₆N₃OS [M+H]⁺380.1791, found 380.1798.

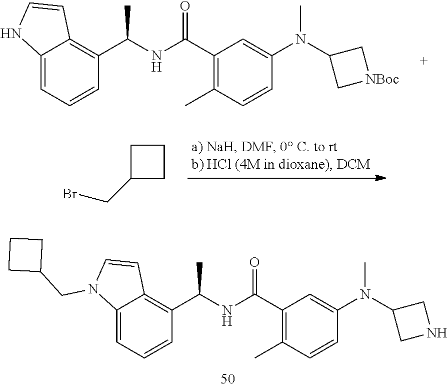

(R)-5-(azetidin-3-yl(methyl)amino)-N-(1-(1-(cyclobutylmethyl)-1H-indol-4-yl)ethyl)-2-methylbenzamide. To a solution of tert-butyl (R)-3-((3-((1-(1H-indol-4-yl)ethyl)carbamoyl)-4-methylphenyl)(methyl)amino)azetidine-1-carboxylate (25 mg, 0.05 mmol) in dry DMF (2 mL), sodium hydride (4 mg, 0.08 mmol) was added at 0° C. After 15 minutes, the mixture was added (bromomethyl)cyclobutane (9 mg, 0.06 mmol) and stirred at 25° C. for another 16 hours. Quench the reaction with methanol and remove the solvent. The residue was purified by preparative HPLC system to obtain the desired product.

The product from previous step was subjected to general N-Boc deprotection procedure with HCl (4M in dioxane, 200 μL) and DCM (4 mL). The purification by Prep-HPLC afforded the product (17 mg, yield 80% for 2 steps) as a white solid: ¹H NMR (400 MHz, Methanol-d₄) δ 8.53 (s, 1H), 7.33 (d, J=8.1 Hz, 1H), 7.25 (d, J=3.2 Hz, 1H), 7.18-7.07 (m, 3H), 6.79 (dd, J=8.3, 2.7 Hz, 1H), 6.73 (d, J=2.6 Hz, 1H), 6.66 (d, J=3.7 Hz, 1H), 5.62 (q, J=7.0 Hz, 1H), 4.46 (p, J=7.2 Hz, 1H), 4.19 (dd, J=12.3, 7.4 Hz, 4H), 4.05 (dd, J=10.3, 7.4 Hz, 2H), 2.83 (s, 3H), 2.23 (s, 3H), 2.06-1.96 (m, 2H), 1.93-1.79 (m, 4H), 1.65 (d, J=7.0 Hz, 3H). ¹³C NMR (100 MHz, Methanol-d₄) δ 148.36, 139.08, 137.99, 136.30, 132.56, 129.04, 128.62, 127.88, 122.25, 119.64, 117.05, 116.55, 111.39, 109.89, 99.99, 53.41, 52.28, 52.02, 49.00, 37.58, 37.12, 27.10, 21.24, 18.98, 18.65; HRMS (ESI) calcd for C₂₇H₃₅N₄O [M+H]⁺431.2805, found 431.2811

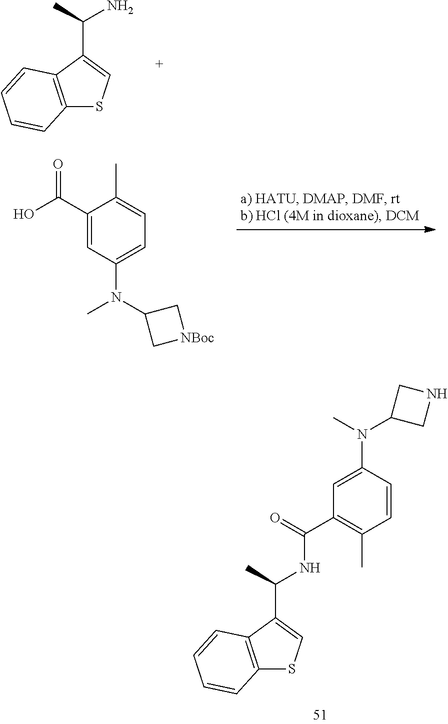

(R)-5-(azetidin-3-yl(methyl)amino)-N-(1-(benzo[b]thiophen-3-yl)ethyl)-2-methylbenzamide. (R)-1-(benzo[b]thiophen-3-yl)ethan-1-amine (30 mg, 0.14 mmol), 5-((1-(tert-butoxycarbonyl)azetidin-3-yl)(methyl)amino)-2-methylbenzoic acid (49 mg, 0.15 mmol), HATU (57 mg, 0.15 mmol) and DMAP (34 mg, 0.28 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (45 mg, yield 85% for 2 steps) as a white solid: [α]₅₄₆²⁵=−29.2 (c 0.5, MeOH); ¹H NMR (400 MHz, Methanol-d₄) δ 8.41 (s, 1H), 7.98 (dt, J=8.1, 0.9 Hz, 1H), 7.94-7.86 (m, 1H), 7.53 (d, J=1.0 Hz, 1H), 7.47-7.33 (m, 2H), 7.11 (d, J=8.4 Hz, 1H), 6.80 (dd, J=8.4, 2.7 Hz, 1H), 6.70 (d, J=2.7 Hz, 1H), 5.73-5.63 (m, 1H), 4.46 (p, J=7.2 Hz, 1H), 4.18 (dd, J=10.9, 7.7 Hz, 2H), 4.05 (dd, J=11.1, 7.0 Hz, 2H), 2.82 (s, 3H), 2.24 (s, 3H), 1.72 (d, J=6.9 Hz, 3H); ¹³C NMR (100 MHz, Methanol-d₄) δ 172.08, 148.38, 142.09, 139.24, 139.19, 138.76, 132.63, 128.53, 125.62, 125.13, 123.91, 123.51, 123.09, 119.68, 116.92, 53.30, 51.96, 44.61, 37.00, 20.55, 18.67; HRMS (ESI) calcd for C₂₂H₂₆N₃OS [M+H]⁺380.1791, found 380.1791.

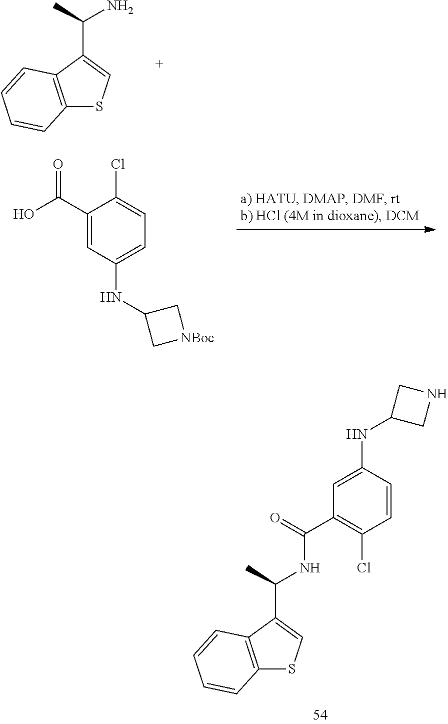

(R)-5-(azetidin-3-ylamino)-N-(1-(benzo[b]thiophen-3-yl)ethyl)-2-chlorobenzamide. (R)-1-(benzo[b]thiophen-3-yl)ethan-1-amine (41 mg, 0.23 mmol), 5-((1-(tert-butoxycarbonyl)azetidin-3-yl)amino)-2-chlorobenzoic acid (50 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (39 mg, yield 68% for 2 steps) as a white solid: [α]₅₄₆²⁵=−24.6 (c 1.7, MeCOH); ¹H NMR (400 MHz, Methanol-d₄) δ 8.49 (s, 1H), 8.00-7.95 (m, 1H), 7.90-7.85 (m, 1H), 7.54 (d, J=1.1 Hz, 1H), 7.44-7.34 (m, 2H), 7.18 (d, J=8.7 Hz, 1H), 6.60 (dd, J=8.7, 2.8 Hz, 1H), 6.54 (d, J=2.8 Hz, 1H), 5.68-5.61 (m, 1H), 4.48-4.40 (m, 1H), 4.36-4.27 (m, 2H), 3.95-3.86 (m, 2H), 1.71 (d, J=6.9 Hz, 3H); ¹³C NMR (100 MHz, Methanol-d₄) δ 169.63, 146.53, 142.03, 139.13, 139.02, 138.17, 131.68, 125.58, 125.13, 123.80, 123.48, 123.17, 120.13, 116.28, 113.73, 54.66, 46.49, 44.88, 20.54; HRMS (ESI) calcd for C₂₀H₂₁ClN₃OS [M+H]⁺ 386.1088, found 386.1094.

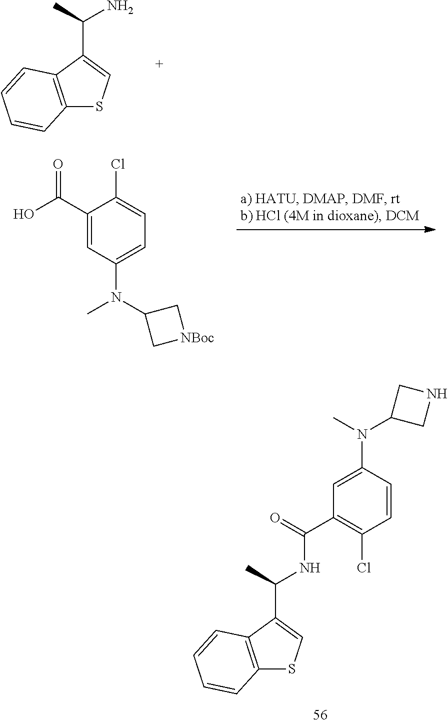

(R)-5-(azetidin-3-yl(methyl)amino)-N-(1-(benzo[b]thiophen-3-yl)ethyl)-2-chlorobenzamide. (R)-1-(benzo[b]thiophen-3-yl)ethan-1-amine (41 mg, 0.23 mmol), 5-((1-(tert-butoxycarbonyl)azetidin-3-yl)(methyl)amino)-2-chlorobenzoic acid (51 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (44 mg, yield 74% for 2 steps) as a white solid: [α]5; =−24.7 (c 1.4, MeOH); ¹H NMR (400 MHz, Methanol-d₄) δ 8.47 (s, 1H), 8.00-7.96 (m, 1H), 7.90-7.86 (m, 1H), 7.55 (d, J=1.0 Hz, 1H), 7.44-7.34 (m, 2H), 7.28 (d, J=8.8 Hz, 1H), 6.84 (dd, J=8.8, 3.0 Hz, 1H), 6.75 (d, J=3.0 Hz, 1H), 5.68-5.62 (m, 1H), 4.66-4.58 (m, 1H), 4.24-4.08 (m, 4H), 2.89 (s, 3H), 1.72 (d, J=6.9 Hz, 3H); ¹¹C NMR (100 MHz, Methanol-d₄) δ 169.30, 149.29, 142.06, 139.11, 138.98, 138.07, 131.64, 125.60, 125.11, 123.84, 123.55, 123.19, 122.23, 119.47, 116.93, 52.67, 51.92, 45.00, 35.61, 20.55; HRMS (ESI) calcd for C₂₁H₂₃ClN₃OS [M+H]⁺ 400.1245, found 400.1252.

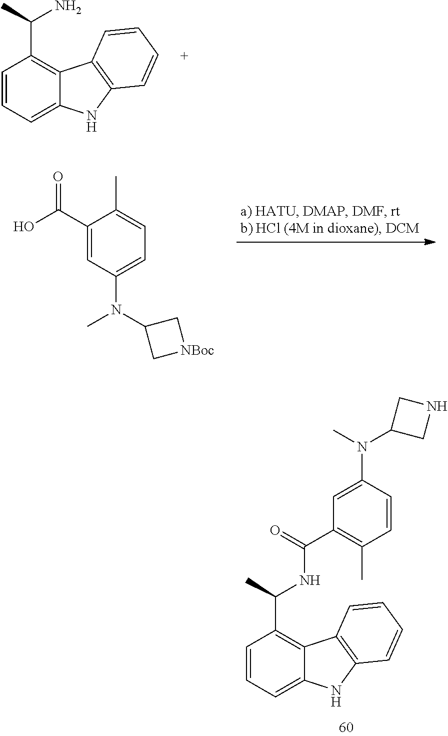 [SMILES: C[C@@H](N)c1cccc2[nH]c3ccccc3c12 ; Cc1ccc(N(C)C2CN(C(=O)OC(C)(C)C)C2)cc1C(=O)O ; Cc1ccc(N(C)C2CNC2)cc1C(=O)N[C@H](C)c1cccc2[nH]c3ccccc3c12]

(R)—N-(1-(9H-carbazol-4-yl)ethyl)-5-(azetidin-3-yl(methyl)amino)-2-methylbenzamide. (R)-1-(9H-carbazol-4-yl)ethan-1-amine (32 mg, 0.15 mmol), 5-((1-(tert-butoxycarbonyl)azetidin-3-yl)(methyl)amino)-2-methylbenzoic acid (48 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (53 mg, yield 86% for 2 steps) as a white solid: [α]₅₄₆²⁵=−42.7 (c 0.8, MeCOH); ¹H NMR (400 MHz, Methanol-d₄) δ 8.37 (s, 1H), 8.24 (d, J=8.1 Hz, 1H), 7.51 (d, J=8.2 Hz, 1H), 7.44-7.37 (m, 3H), 7.30-7.27 (m, 1H), 7.23-7.18 (m, 1H), 7.12 (d, J=8.3 Hz, 1H), 6.79 (dd, J=8.3, 2.8 Hz, 1H), 6.66 (d, J=2.8 Hz, 1H), 6.14 (q, J=6.8 Hz, 1H), 4.39-4.30 (m, 1H), 4.12-3.94 (m, 4H), 2.77 (s, 3H), 2.30 (s, 3H), 1.79 (d, J=6.8 Hz, 3H); ¹¹C NMR (100 MHz, Methanol-d₄) δ 172.53, 148.29, 141.94, 141.69, 138.86, 132.61, 128.78, 126.54, 126.29, 123.87, 123.43, 121.45, 119.94, 119.83, 117.10, 116.24, 111.85, 111.10, 53.35, 51.94, 48.25, 37.12, 20.53, 18.69; HRMS (ESI) calcd for C₂₆H₂₉N₄O [M+H]⁺413.2336, found 413.2341.

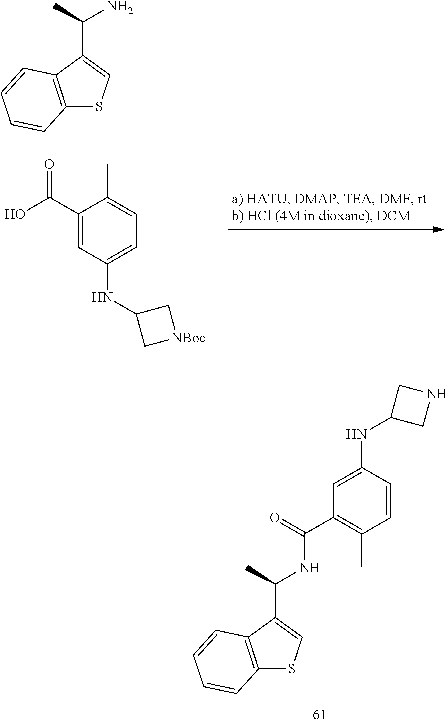

(R)-5-(azetidin-3-ylamino)-N-(1-(benzo[b]thiophen-3-yl)ethyl)-2-methylbenzamide. (R)-1-(benzo[b]thiophen-3-yl)ethan-1-amine (41 mg, 0.23 mmol), 5-((1-(tert-butoxycarbonyl)azetidin-3-yl)amino)-2-methylbenzoic acid (46 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (46 mg, yield 84% for 2 steps) as a white solid: [α]₅₄₆²⁵=−31.7 (c 2.0, MeCOH); ¹H NMR (400 MHz, Methanol-d₄) δ 8.48 (s, 1H), 7.99-7.94 (m, 1H), 7.90-7.85 (m, 1H), 7.52 (s, 1H), 7.44-7.33 (m, 2H), 7.00 (d, J=8.3 Hz, 1H), 6.54 (dd, J=8.3, 2.6 Hz, 1H), 6.48 (d, J=2.6 Hz, 1H), 5.66 (q, J=6.9 Hz, 1H), 4.42 (p, J=7.0 Hz, 1H), 4.31-4.23 (m, 2H), 3.93-3.84 (m, 2H), 2.21 (s, 3H), 1.70 (d, J=6.9 Hz, 3H); ¹³C NMR (100 MHz, Methanol-d₄) δ 172.33, 145.38, 142.04, 139.31, 139.19, 138.71, 132.62, 125.76, 125.59, 125.13, 123.86, 123.41, 123.07, 115.58, 112.80, 54.76, 46.79, 44.50, 20.58, 18.61; HRMS (ESI) calcd for C₂₁H₂₄N₃OS [M+H]⁺366.1635, found 366.1640.

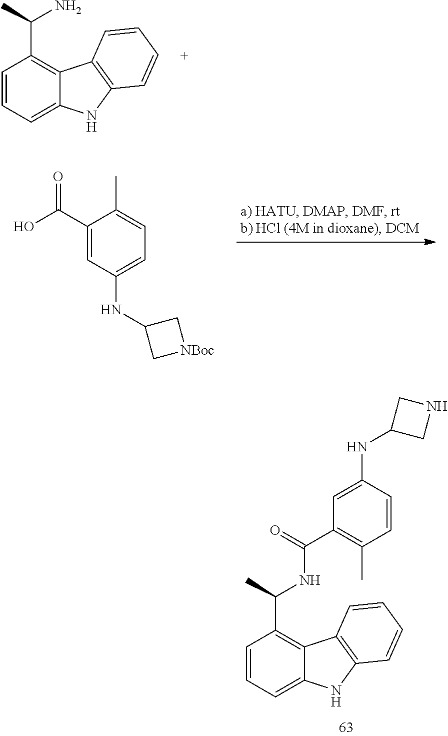 [SMILES: C[C@@H](N)c1cccc2[nH]c3ccccc3c12 ; Cc1ccc(NC2CN(C(=O)OC(C)(C)C)C2)cc1C(=O)O ; Cc1ccc(NC2CNC2)cc1C(=O)N[C@H](C)c1cccc2[nH]c3ccccc3c12]

(R)—N-(1-(9H-carbazol-4-yl)ethyl)-5-(azetidin-3-ylamino)-2-methylbenzamide. (R)-1-(9H-carbazol-4-yl)ethan-1-amine (32 mg, 0.15 mmol), 5-((1-(tert-butoxycarbonyl)azetidin-3-yl)amino)-2-methylbenzoic acid (46 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (47 mg, yield 78% for 2 steps) as a white solid: ¹H NMR (400 MHz, Methanol-d₄) δ 8.53 (s, 2H), 8.24 (d, J=8.2 Hz, 1H), 7.53-7.48 (m, 1H), 7.44-7.36 (m, 3H), 7.30-7.26 (m, 1H), 7.23-7.17 (m, 1H), 7.02 (d, J=8.2 Hz, 1H), 6.55 (dd, J=8.4, 2.8 Hz, 1H), 6.48-6.43 (m, 1H), 6.15 (q, J=6.5 Hz, 1H), 4.39-4.30 (m, 1H), 4.25-4.15 (m, 2H), 3.88-3.80 (m, 2H), 2.27 (s, 3H), 1.77 (d, J=6.8 Hz, 3H); ¹³C NMR (100 MHz, Methanol-d₄) δ 172.78, 145.33, 141.93, 141.70, 139.03, 138.91, 132.62, 126.52, 126.28, 126.07, 123.89, 123.46, 121.44, 119.96, 116.18, 115.84, 112.78, 111.81, 111.02, 54.83, 48.14, 46.97, 20.68, 18.62; HRMS (ESI) calcd for C₂₅H₂₇N₄O [M+H]⁺ 399.2179, found 399.2176.

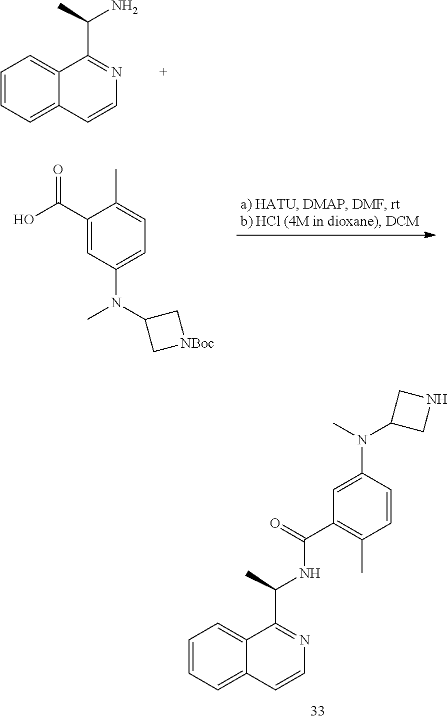 [SMILES: C[C@@H](N)c1nccc2ccccc12 ; Cc1ccc(N(C)C2CN(C(=O)OC(C)(C)C)C2)cc1C(=O)O ; Cc1ccc(N(C)C2CNC2)cc1C(=O)N[C@H](C)c1nccc2ccccc12]

(R)-5-(azetidin-3-yl(methyl)amino)-N-(1-(isoquinolin-1-yl)ethyl)-2-methylbenzamide. (R)-1-(isoquinolin-1-yl)ethan-1-amine (35 mg, 0.20 mmol), 5-((1-(tert-butoxycarbonyl)azetidin-3-yl)(methyl)amino)-2-methylbenzoic acid (96 mg, 0.30 mmol), HATU (114 mg, 0.30 mmol) and DMAP (110 mg, 0.90 mmol) was subjected to general amine coupling procedure with DMF (1.5 mL). After purification by Prep-HPLC, the product was subjected to general N-Boc deprotection procedure with HCl (4M in dioxane, 200 μL) and DCM (4 mL). The purification by Prep-HPLC afforded the product (63 mg, yield 84% for 2 steps) as a white solid: [α]₅₄₆²⁵=−49.6 (c 0.8, MeOH); ¹H NMR (400 MHz, Methanol-d₄) δ 8.54 (s, 1H), 8.47-8.40 (m, 2H), 8.01-7.94 (m, 1H), 7.82-7.71 (m, 3H), 7.14 (d, J=8.0 Hz, 1H), 6.88-6.81 (m, 2H), 6.17 (q, J=6.9 Hz, 1H), 4.56-4.46 (m, 1H), 4.26-4.19 (m, 2H), 4.11-4.04 (m, 2H), 2.87 (s, 3H), 2.28 (s, 3H), 1.69 (d, J=6.8 Hz, 3H); ¹³C NMR (100 MHz, Methanol-d₄) δ 171.96, 161.77, 148.51, 142.19, 138.47, 138.16, 132.77, 131.71, 128.97, 128.75, 126.89, 125.55, 121.88, 119.91, 117.01, 53.46, 52.13, 48.09, 37.09, 21.35, 18.78; HRMS (ESI) calcd for C₂₃H₂₇N₄O [M+H]⁺375.2179, found 375.2183.

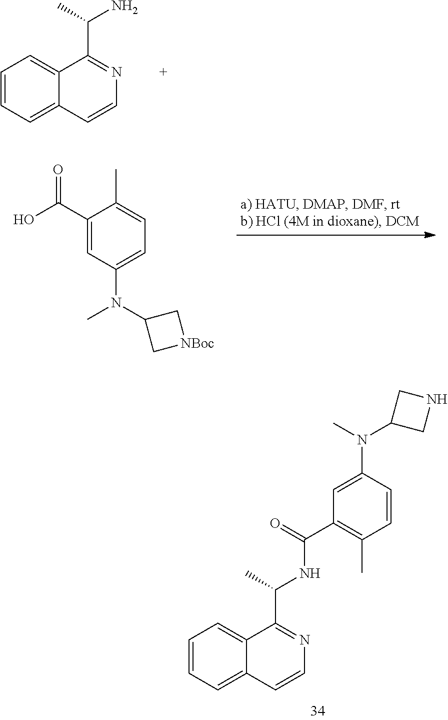 [SMILES: C[C@H](N)c1nccc2ccccc12 ; Cc1ccc(N(C)C2CN(C(=O)OC(C)(C)C)C2)cc1C(=O)O ; Cc1ccc(N(C)C2CNC2)cc1C(=O)N[C@@H](C)c1nccc2ccccc12]

(S)-5-(azetidin-3-yl(methyl)amino)-N-(1-(isoquinolin-1-yl)ethyl)-2-methylbenzamide. (S)-1-(isoquinolin-1-yl)ethan-1-amine (10 mg, 0.06 mmol), 5-((1-(tert-butoxycarbonyl)azetidin-3-yl)(methyl)amino)-2-methylbenzoic acid (29 mg, 0.09 mmol), HATU (34 mg, 0.09 mmol) and DMAP (33 mg, 0.27 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (16 mg, yield 71% for 2 steps) as a white solid: [α]₅₄₆²⁵=+40.7 (c 0.4, MeOH);¹H NMR (400 MHz, Methanol-d₄) δ 8.51 (s, 1H), 8.44 (t, J=7.5 Hz, 2H), 7.99-7.95 (m, 1H), 7.83-7.71 (m, 3H), 7.13 (dd, J=10.6, 8.1 Hz, 1H), 6.87-6.81 (m, 2H), 6.20-6.12 (m, 1H), 4.56-4.46 (m, 1H), 4.29-4.21 (m, 2H), 4.08 (dd, J=11.2, 7.1 Hz, 2H), 2.87 (s, 3H), 2.28 (s, 3H), 1.69 (d, J=6.9 Hz, 3H); ¹³C NMR (100 MHz, Methanol-d₄) 171.90, 161.77, 148.51, 142.18, 138.47, 138.16, 132.78, 131.72, 128.98, 128.76, 125.55, 121.89, 119.96, 117.06, 53.40, 52.13, 48.08, 37.13, 21.36, 18.78; HRMS (ESI) calcd for C₂₃H₂₇N₄O [M+H]⁺375.2179, found 375.2185.

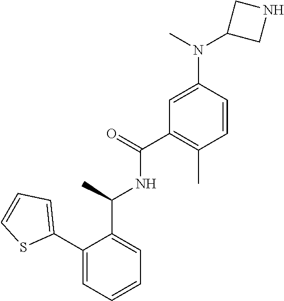 [SMILES: Cc1ccc(N(C)C2CNC2)cc1C(=O)N[C@H](C)c1ccccc1-c1cccs1]

(R)-5-(azetidin-3-yl(methyl)amino)-2-methyl-N-(1-(2-(thiophen-2-yl)phenyl)ethyl)benzamide. ¹H NMR (400 MHz, Acetone-d₆) δ 7.79-7.71 (m, 1H), 7.55 (dd, J=5.2, 1.2 Hz, 1H), 7.46-7.27 (m, 4H), 7.18 (dd, J=5.2, 3.4 Hz, 1H), 7.02 (d, J=8.4 Hz, 1H), 6.77 (d, J=2.7 Hz, 1H), 6.72 (dd, J=8.3, 2.8 Hz, 1H), 5.65-5.55 (m, 1H), 3.99 (p, J=6.7 Hz, 1H), 3.65-3.55 (m, 2H), 3.39-3.24 (m, 2H), 2.81 (s, 3H), 2.21 (s, 3H), 1.43 (d, J=7.0 Hz, 3H); ¹³C NMR (100 MHz, Acetone-d₆) δ 162.86, 148.98, 144.94, 142.60, 138.59, 133.94, 131.91, 129.41, 128.15, 128.06, 127.52, 126.81, 126.64, 126.27, 117.24, 115.29, 52.57, 52.52, 50.31, 46.80, 36.49, 23.20, 18.79; HRMS (ESI) calcd for C₂₄H₂₈N₃OS [M+H]⁺406.1948, found 406.1953

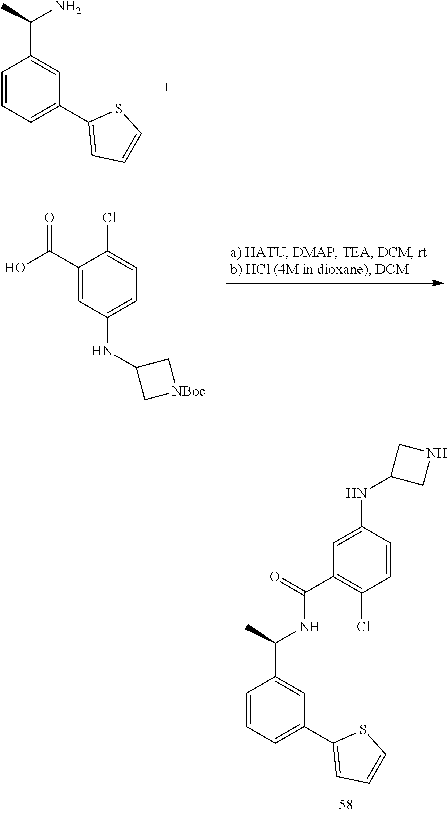 [SMILES: C[C@@H](N)c1cccc(-c2cccs2)c1 ; CC(C)(C)OC(=O)N1CC(Nc2ccc(Cl)c(C(=O)O)c2)C1 ; C[C@@H](NC(=O)c1cc(NC2CNC2)ccc1Cl)c1cccc(-c2cccs2)c1]

(R)-5-(azetidin-3-ylamino)-2-chloro-N-(1-(3-(thiophen-2-yl)phenyl)ethyl)benzamide. (R)-1-(3-(thiophen-2-yl)phenyl)ethan-1-amine (30 mg, 0.15 mmol), 5-((1-(tert-butoxycarbonyl)azetidin-3-yl)amino)-2-chlorobenzoic acid (50 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the (48 mg, yield 77% for 2 steps) as a white solid: [α]₅₄₆²⁵=+58.5 (c 1.5, MeOH); ¹H NMR (400 MHz, Methanol-d₄) δ 8.42 (s, 1H), 7.73-7.70 (m, 1H), 7.55-7.51 (m, 1H), 7.41-7.32 (m, 4H), 7.20 (d, J=8.5 Hz, 1H), 7.08 (dd, J=5.1, 3.6 Hz, 1H), 6.64-6.58 (m, 2H), 5.21 (q, J=7.0 Hz, 1H), 4.51-4.43 (m, 1H), 4.36-4.30 (m, 2H), 3.95-3.89 (m, 2H), 1.56 (d, J=7.1 Hz, 3H); ¹³C NMR (100 MHz, Methanol-d₄) 5169.47, 146.59, 145.89, 145.40, 138.26, 135.99, 131.68, 130.17, 129.10, 126.43, 125.91, 125.52, 124.65, 124.35, 120.11, 116.39, 113.71, 54.62, 50.74, 46.50, 22.41; HRMS (ESI) calcd for C₂₂H₂₃ClN₃OS [M+H]⁺412.1245, found 412.1250.

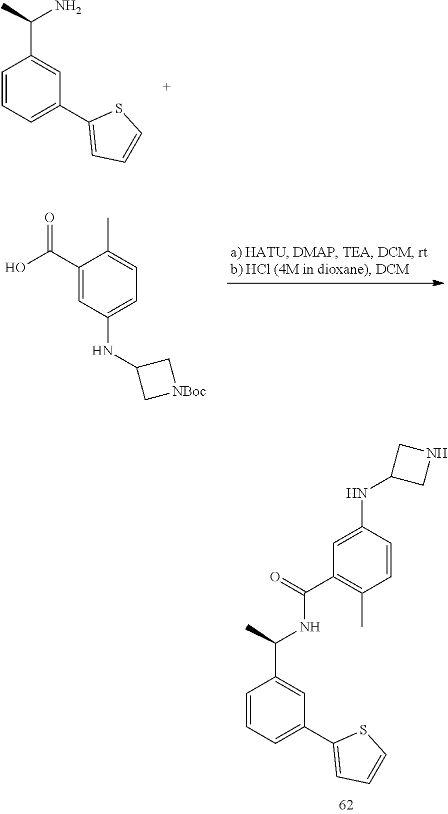 [SMILES: C[C@@H](N)c1cccc(-c2cccs2)c1 ; Cc1ccc(NC2CN(C(=O)OC(C)(C)C)C2)cc1C(=O)O ; Cc1ccc(NC2CNC2)cc1C(=O)N[C@H](C)c1cccc(-c2cccs2)c1]

(R)-5-(azetidin-3-ylamino)-2-methyl-N-(1-(3-(thiophen-2-yl)phenyl)ethyl)benzamide. (R)-1-(3-(thiophen-2-yl)phenyl)ethan-1-amine (30 mg, 0.15 mmol), 5-((1-(tert-butoxycarbonyl)azetidin-3-yl)amino)-2-methylbenzoic acid (46 mg, 0.15 mmol), HATU (70 mg, 0.18 mmol) and DMAP (56 mg, 0.46 mmol) was subjected to general amine coupling procedure with DMF (2 mL). After purification by Prep-HPLC, the product was subjected to general N-Boc deprotection procedure with HCl (4M in dioxane, 200 μL) and DCM (4 mL). The purification by Prep-HPLC afforded the product (47 mg, yield 80% for 2 steps) as a white solid: [α]²6=+25.2 (c 0.8, MeOH); ¹H NMR (400 MHz, Methanol-d₄) δ 8.51 (s, 1H), 7.70-7.68 (m, 1H), 7.56-7.51 (m, 1H), 7.41-7.31 (m, 4H), 7.09 (dd, J=5.1, 3.6 Hz, 1H), 7.03 (d, J=8.2 Hz, 1H), 6.60-6.52 (m, 2H), 5.22 (q, J=7.0 Hz, 1H), 4.48 (p, J=7.0 Hz, 1H), 4.38-4.29 (m, 2H), 3.95-3.88 (m, 2H), 2.22 (s, 3H), 1.55 (d, J=7.1 Hz, 3H); ¹¹C NMR (100 MHz, Methanol-d₄) δ 172.56, 146.28, 145.45, 145.41, 138.93, 136.04, 132.62, 130.23, 129.12, 126.38, 125.93, 125.71, 125.51, 124.62, 124.34, 115.64, 112.68, 54.85, 50.51, 46.86, 22.39, 18.62; HRMS (ESI) calcd for C₂₃H₂₆N₃OS [M+H]⁺392.1791, found 392.1790.

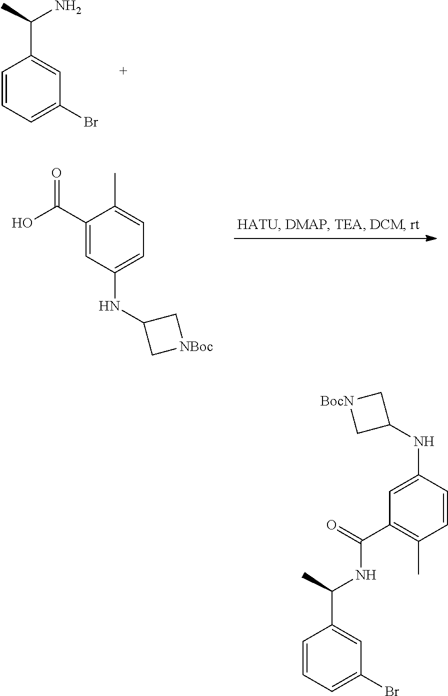

Tert-butyl-(R)-3-((3-((1-(3-bromophenyl)ethyl)carbamoyl)-4-methylphenyl)amino)azetidine-1-carboxylate. (R)-1-(3-bromophenyl)ethan-1-amine (200 mg, 1.00 mmol), 5-((1-(tert-butoxycarbonyl)azetidin-3-yl)amino)-2-methylbenzoic acid (306 mg, 1.00 mmol), HATU (380 mg, 1.00 mmol), DMAP (12 mg, 0.10 mmol), and TEA (200 μL) was subjected to general amine coupling procedure with DMF (5 mL). The purification by Prep-HPLC afforded the (435 mg, yield 89%) as a white solid: ¹H NMR (400 MHz, Chloroform-d) δ 7.51-7.50 (m, 1H), 7.40 (ddd, J=7.8, 2.0, 1.2 Hz, 1H), 7.32-7.28 (m, 1H), 7.24-7.19 (m, 1H), 7.01 (d, J=8.2 Hz, 1H), 6.52 (d, J=2.6 Hz, 1H), 6.46 (dd, J=8.2, 2.6 Hz, 1H), 6.02 (d, J=8.0 Hz, 1H), 5.25 (p, J=7.1 Hz, 1H), 4.26 (dd, J=8.7, 7.1 Hz, 2H), 4.20-4.13 (m, 1H), 3.68 (dd, J=9.0, 4.6 Hz, 2H), 2.28 (s, 3H), 1.55 (d, J=7.0 Hz, 3H), 1.43 (s, 9H); ¹³C NMR (100 MHz, Chloroform-d) δ 169.40, 156.30, 145.71, 144.24, 137.28, 132.17, 130.63, 130.45, 129.30, 125.28, 125.09, 122.93, 114.74, 111.81, 79.88, 48.75, 43.41, 38.74, 28.50, 21.99, 18.83; LRMS (ESI) calcd for C₂₄H₃₁BrN₃O₃ [M+H]⁺488.15, found 488.19.

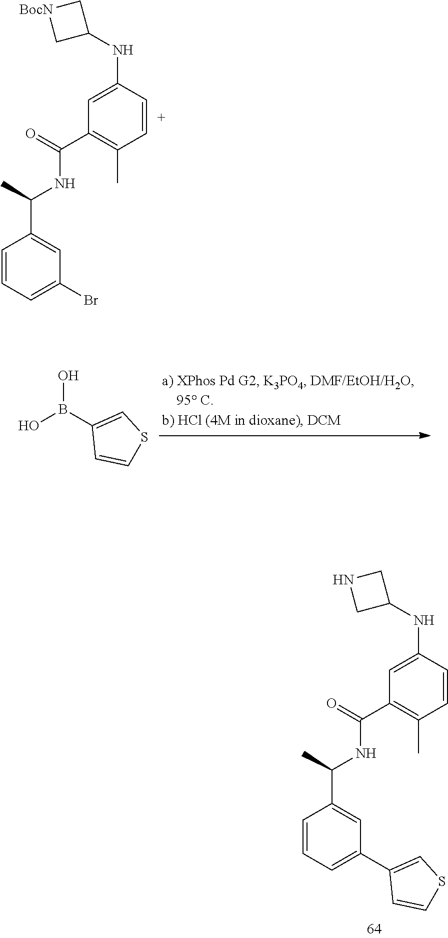 [SMILES: Cc1ccc(NC2CN(C(=O)OC(C)(C)C)C2)cc1C(=O)N[C@H](C)c1cccc(Br)c1 ; OB(O)c1ccsc1 ; Cc1ccc(NC2CNC2)cc1C(=O)N[C@H](C)c1cccc(-c2ccsc2)c1]

(R)-5-(azetidin-3-ylamino)-2-methyl-N-(1-(3-(thiophen-3-yl)phenyl)ethyl)benzamide. A flask fitted with a rubber septum was charged with tert-butyl (R)-3-((3-((1-(3-bromophenyl)ethyl)carbamoyl)-4-methylphenyl)amino)azetidine-1-carboxylate (49 mg, 0.10 mmol), thiophen-3-ylboronic acid (19 mg, 0.15 mmol), XPhos Pd G2 (8 mg, 0.01 mmol), K₃PO₄ (64 mg, 0.3 mmol), DMF/EtOH/H₂O (1 mL/1 mL/0.5 mL) and then purged with argon. The mixture was stirred at 95° C. overnight. The reaction mixture was then cooled to room temperature, diluted with ethyl acetate (20 mL), filtered through celite and concentrated in vacuo. After purification by Prep-HPLC, the product was subjected to general N-Boc deprotection procedure with HCl (4M in dioxane, 100 μL) and DCM (2 mL). The purification by Prep-HPLC afforded the product (14 mg, yield 35% for 2 steps) as a white solid: [α]₅₄₆²⁵=+12.3 (c 0.6, MeCOH); ¹H NMR (400 MHz, Methanol-d₄) δ 8.41 (s, 1H), 7.72-7.69 (m, 1H), 7.63-7.60 (m, 1H), 7.58-7.53 (m, 1H), 7.50-7.45 (m, 2H), 7.41-7.31 (m, 2H), 7.02 (d, J=8.2 Hz, 1H), 6.61-6.50 (m, 2H), 5.23 (q, J=7.0 Hz, 1H), 4.52-4.42 (m, 1H), 4.35-4.29 (m, 2H), 3.95-3.88 (m, 2H), 2.21 (s, 3H), 1.56 (d, J=7.0 Hz, 3H); ¹³C NMR (100 MHz, Methanol-d₄) δ 172.53, 146.00, 145.45, 143.51, 138.97, 137.47, 132.63, 130.10, 127.36, 127.17, 126.13, 125.92, 125.70, 125.29, 121.38, 115.58, 112.72, 54.84, 50.62, 46.84, 22.47, 18.57; HRMS (ESI) calcd for C₂₃H₂₆N₃OS [M+H]⁺ 392.1791, found 392.1800.

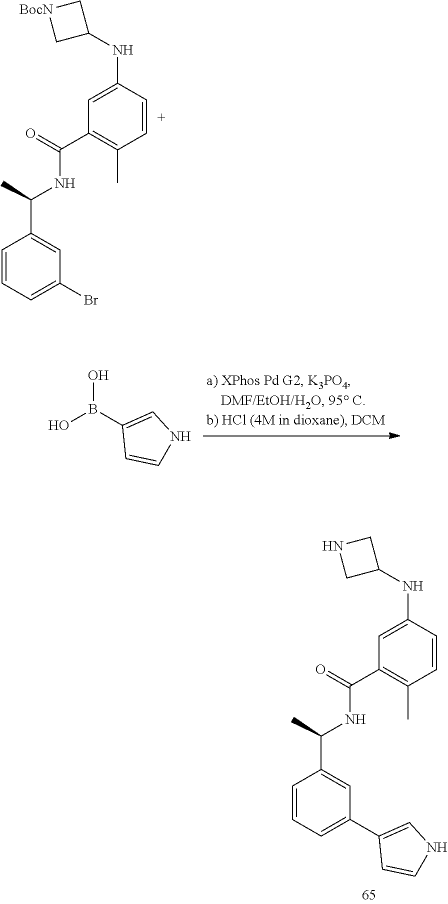 [SMILES: Cc1ccc(NC2CN(C(=O)OC(C)(C)C)C2)cc1C(=O)N[C@H](C)c1cccc(Br)c1 ; OB(O)c1cc[nH]c1 ; Cc1ccc(NC2CNC2)cc1C(=O)N[C@H](C)c1cccc(-c2cc[nH]c2)c1]

(R)—N-(1-(3-(1H-pyrrol-3-yl)phenyl)ethyl)-5-(azetidin-3-ylamino)-2-methylbenzamide. A flask fitted with a rubber septum was charged with tert-butyl (R)-3-((3-((1-(3-bromophenyl)ethyl)carbamoyl)-4-methylphenyl)amino)azetidine-1-carboxylate (49 mg, 0.10 mmol), (1H-pyrrol-3-yl)boronic acid (17 mg, 0.15 mmol), XPhos Pd G2 (8 mg, 0.01 mmol), K₃PO₄ (64 mg, 0.3 mmol), DMF/EtOH/H₂O (1 mL/1 mL/0.5 mL) and then purged with argon. The mixture was stirred at 95° C. overnight. The reaction mixture was then cooled to room temperature, diluted with ethyl acetate (20 mL), filtered through celite and concentrated in vacuo. After purification by Prep-HPLC, the product was subjected to general N-Boc deprotection procedure with HCl (4M in dioxane, 100 μL) and DCM (2 mL). The purification by Prep-HPLC afforded the product (15 mg, yield 41% for 2 steps) as a white solid: [α]56=+12.1 (c 1.7, MeOH); ¹H NMR (400 MHz, Methanol-d₄) δ 8.44 (s, 1H), 7.59-7.56 (m, 1H), 7.44-7.40 (m, 1H), 7.29-7.24 (m, 1H), 7.16-7.10 (m, 2H), 7.02 (d, J=8.3 Hz, 1H), 6.79-6.76 (m, 1H), 6.56 (dd, J=8.3, 2.6 Hz, 1H), 6.51 (d, J=2.6 Hz, 1H), 6.48-6.44 (m, 1H), 5.19 (q, J=7.0 Hz, 1H), 4.49-4.39 (m, 1H), 4.33-4.27 (m, 2H), 3.90 (ddd, J=11.4, 6.7, 2.0 Hz, 2H), 2.22 (s, 3H), 1.54 (d, J=7.1 Hz, 3H); ¹³C NMR (100 MHz, Methanol-d₄) δ 172.50, 145.42, 139.03, 138.29, 132.64, 132.61, 129.72, 125.71, 125.36, 124.64, 123.70, 119.83, 115.72, 115.69, 115.65, 112.63, 106.47, 54.83, 50.67, 46.83, 22.49, 18.58; HRMS (ESI) calcd for C₂₃H₂₇N₄O [M+H]⁺ 375.2179, found 375.2183.

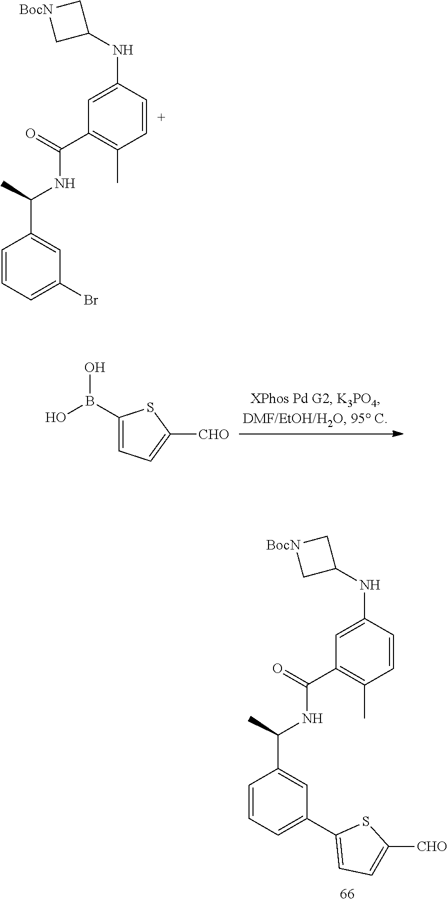 [SMILES: Cc1ccc(NC2CN(C(=O)OC(C)(C)C)C2)cc1C(=O)N[C@H](C)c1cccc(Br)c1 ; O=Cc1ccc(B(O)O)s1 ; Cc1ccc(NC2CN(C(=O)OC(C)(C)C)C2)cc1C(=O)N[C@H](C)c1cccc(-c2ccc(C=O)s2)c1]

Tert-butyl (R)-3-((3-((1-(3-(5-formylthiophen-2-yl)phenyl)ethyl)carbamoyl)-4-methylphenyl)amino)azetidine-1-carboxylate. A flask fitted with a rubber septum was charged with tert-butyl (R)-3-((3-((1-(3-bromophenyl)ethyl)carbamoyl)-4-methylphenyl)amino)azetidine-1-carboxylate (490 mg, 1.00 mmol), (5-formylthiophen-2-yl)boronic acid (170 mg, 1.50 mmol), XPhos Pd G2 (40 mg, 0.05 mmol), K₃PO₄ (531 mg, 2.5 mmol), DMF/EtOH/H₂O (5 mL/5 mL/2.5 mL) and then purged with argon. The mixture was stirred at 95° C. overnight. The reaction mixture was then cooled to room temperature, diluted with ethyl acetate (50 mL), filtered through celite and concentrated in vacuo. The purification by Prep-HPLC afforded the product (166 mg, yield 32%) as a white solid: ¹H NMR (400 MHz, Chloroform-d) δ 9.88 (s, 1H), 7.74 (d, J=3.9 Hz, 1H), 7.67 (s, 1H), 7.61-7.57 (m, 1H), 7.46-7.38 (m, 3H), 7.02 (d, J=8.2 Hz, 1H), 6.55 (d, J=2.5 Hz, 1H), 6.47 (dd, J=8.2, 2.6 Hz, 1H), 6.06 (d, J=7.9 Hz, 1H), 5.34 (p, J=7.1 Hz, 1H), 4.25 (ddd, J=9.2, 6.9, 2.6 Hz, 2H), 4.12 (q, J=7.2 Hz, 1H), 3.68 (dd, J=8.9, 4.5 Hz, 2H), 2.29 (s, 3H), 1.61 (d, J=7.0 Hz, 3H), 1.43 (s, 9H); ¹³C NMR (100 MHz, Chloroform-d) b 182.92, 169.49, 154.08, 144.65, 144.28, 142.72, 137.52, 137.36, 133.64, 132.20, 129.79, 127.32, 125.64, 125.22, 124.47, 124.37, 114.75, 111.84, 79.88, 49.07, 43.42, 28.51, 22.12, 18.89; LRMS (ESI) calcd for C₂₉H₃₄N₃O₄S [M+H]⁺ 520.23, found 520.25.

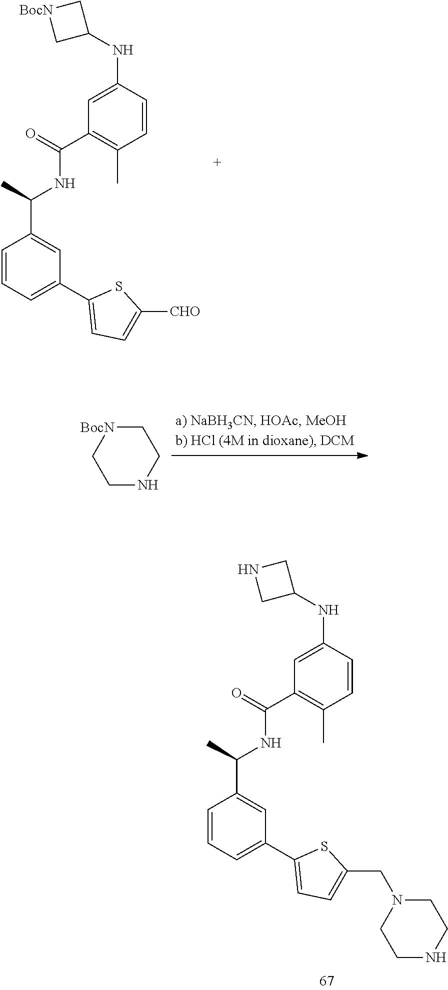 [SMILES: Cc1ccc(NC2CN(C(=O)OC(C)(C)C)C2)cc1C(=O)N[C@H](C)c1cccc(-c2ccc(C=O)s2)c1 ; CC(C)(C)OC(=O)N1CCNCC1 ; Cc1ccc(NC2CNC2)cc1C(=O)N[C@H](C)c1cccc(-c2ccc(CN3CCNCC3)s2)c1]

(R)-5-(azetidin-3-ylamino)-2-methyl-N-(1-(3-(5-(piperazin-1-ylmethyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and tert-butyl piperazine-1-carboxylate (17 mg, 0.09 mmol) was subjected to general reductive amination procedure with MeOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (20 mg, yield 68% for 2 steps) as a white solid: [α]₅₄₆²⁵=−6.9 (c 1.7, MeOH); ¹H NMR (400 MHz, Methanol-d₄) δ 8.34 (s, 1H), 7.67-7.64 (m, 1H), 7.53-7.48 (m, 1H), 7.41-7.32 (m, 2H), 7.26 (d, J=3.7 Hz, 1H), 7.03 (d, J=7.9 Hz, 1H), 6.99-6.97 (m, 1H), 6.61-6.55 (m, 2H), 5.21 (q, J=7.1 Hz, 1H), 4.56-4.45 (m, 1H), 4.36 (dd, J=11.4, 7.4 Hz, 2H), 3.95 (dd, J=11.2, 6.7 Hz, 2H), 3.83 (s, 2H), 3.26-3.21 (m, 4H), 2.80-2.73 (m, 4H), 2.21 (s, 3H), 1.55 (d, J=7.0 Hz, 3H); ¹³C NMR (100 MHz, Methanol-d₄) δ 172.57, 146.33, 145.64, 145.47, 141.01, 138.86, 135.95, 132.64, 130.28, 129.15, 126.33, 125.73, 125.27, 124.53, 123.90, 115.59, 112.84, 57.59, 54.92, 50.54, 50.31, 46.84, 44.87, 22.36, 18.64; HRMS (ESI) calcd for C₂₈H₃₆N₅OS [M+H]⁺490.2635, found 490.2632.

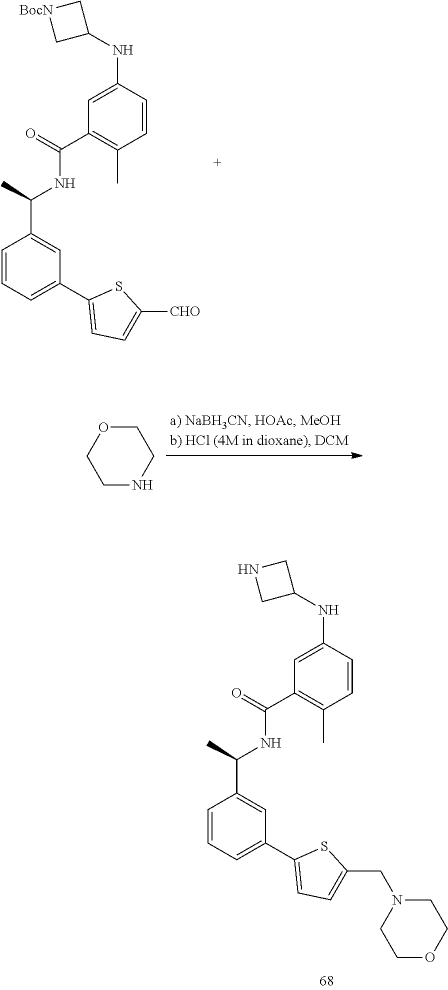 [SMILES: Cc1ccc(NC2CN(C(=O)OC(C)(C)C)C2)cc1C(=O)N[C@H](C)c1cccc(-c2ccc(C=O)s2)c1 ; C1COCCN1 ; Cc1ccc(NC2CNC2)cc1C(=O)N[C@H](C)c1cccc(-c2ccc(CN3CCOCC3)s2)c1]

(R)-5-(azetidin-3-ylamino)-2-methyl-N-(1-(3-(5-(morpholinomethyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and morpholine (8 mg, 0.09 mmol) was subjected to general reductive amination procedure with MeOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (19 mg, yield 65% for 2 steps) as a white solid: [α]₅₄₆²⁵=−9.6 (c 1.2, MeCOH); ¹H NMR (400 MHz, Methanol-d₄) δ 8.09 (s, 1H), 7.73-7.69 (m, 1H), 7.60-7.54 (m, 1H), 7.46-7.37 (m, 4H), 7.08 (d, J=8.6 Hz, 1H), 6.72-6.66 (m, 2H), 5.22 (q, J=7.0 Hz, 1H), 4.64 (s, 2H), 4.59-4.52 (m, 1H), 4.42-4.32 (m, 2H), 4.13-3.99 (m, 4H), 3.89-3.79 (m, 2H), 3.52-3.43 (m, 2H), 3.29-3.19 (m, 2H), 2.22 (s, 3H), 1.56 (d, J=7.1 Hz, 3H); ¹³C NMR (100 MHz, Methanol-d₄) δ 172.36, 149.60, 146.57, 144.17, 138.73, 135.32, 134.98, 132.81, 130.51, 129.40, 127.29, 127.09, 125.68, 124.98, 124.86, 116.51, 113.87, 64.99, 55.70, 54.64, 52.49, 50.63, 47.39, 22.40, 18.71; HRMS (ESI) calcd for C₂₈H₃₅N₄O₂S [M+H]⁺491.2475, found 491.2475.

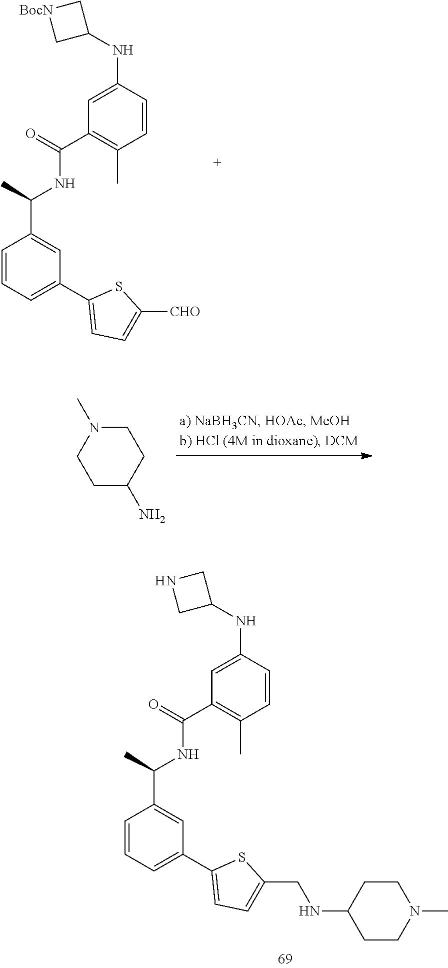

(R)-5-(azetidin-3-ylamino)-2-methyl-N-(1-(3-(5-(((1-methylpiperidin-4-yl)amino)methyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and 1-methylpiperidin-4-amine (10 mg, 0.09 mmol) was subjected to general reductive amination procedure with MeCOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (23 mg, yield 74% for 2 steps) as a white solid: [α]₅₄₆²⁵=−9.6 (c 1.7, MeOH); ¹H NMR (400 MHz, Methanol-d₄) δ 8.09 (s, 1H), 7.70 (t, J=1.9 Hz, 1H), 7.56 (dt, J=6.6, 2.1 Hz, 1H), 7.46-7.34 (m, 4H), 7.08-7.01 (m, 1H), 6.65-6.58 (m, 2H), 5.21 (q, J=7.2 Hz, 1H), 4.62-4.47 (m, 3H), 4.42-4.33 (m, 2H), 4.05-3.96 (m, 2H), 3.70-3.57 (m, 3H), 3.25-3.13 (m, 2H), 2.89 (s, 3H), 2.49 (d, J=14.2 Hz, 2H), 2.21 (s, 3H), 2.20-2.06 (m, 2H), 1.56 (d, J=7.1 Hz, 3H); ¹³C NMR (100 MHz, Methanol-d₄) δ 172.52, 148.59, 146.64, 145.18, 138.77, 135.12, 133.42, 132.69, 132.18, 130.47, 127.10, 126.05, 125.58, 124.80, 124.78, 115.75, 113.11, 54.94, 53.48, 52.86, 50.58, 46.94, 44.07, 43.70, 27.36, 22.42, 18.66; HRMS (ESI) calcd for C₃₀H₄₀N₅OS [M+H]⁺518.2948, found 518.2952.

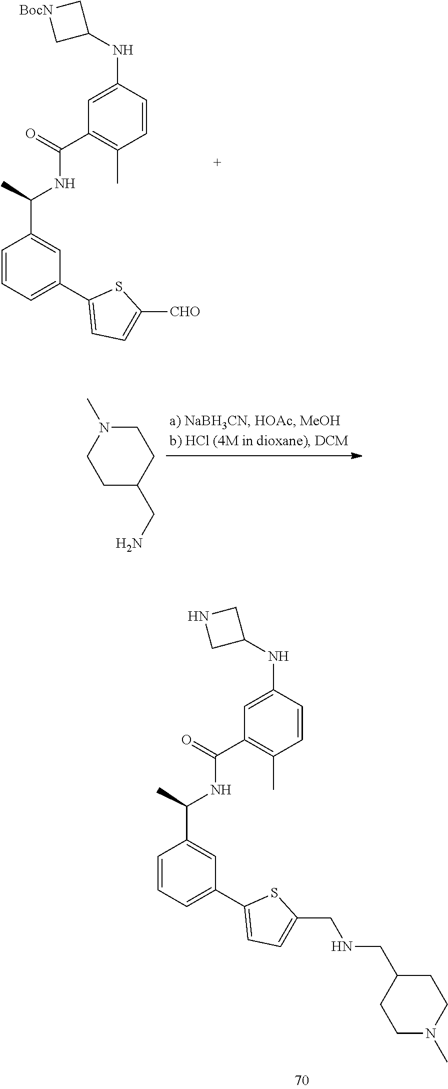

(R)-5-(azetidin-3-ylamino)-2-methyl-N-(1-(3-(5-((((1-methylpiperidin-4-yl)methyl)amino)methyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and (1-methylpiperidin-4-yl)methanamine (12 mg, 0.09 mmol) was subjected to general reductive amination procedure with MeCOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the (24 mg, yield 76% for 2 steps) as a white solid: [α]5_{f}=−11.0 (c 0.6, MeOH); ¹H NMR (400 MHz, Methanol-d₄) δ 8.16 (s, 1H), 7.72-7.67 (m, 1H), 7.55 (tt, J=5.7, 2.1 Hz, 1H), 7.44-7.32 (m, 4H), 7.04 (d, J=9.0 Hz, 1H), 6.61-6.56 (m, 2H), 5.24-5.16 (m, 1H), 4.55-4.47 (m, 3H), 4.40-4.34 (m, 2H), 4.00-3.93 (m, 2H), 3.47-3.37 (m, 2H), 3.09-2.98 (m, 4H), 2.21 (s, 3H), 2.18-2.02 (m, 3H), 1.56 (d, J=7.1 Hz, 5H); ¹³C NMR (100 MHz, Methanol-d₄) δ 172.60, 148.54, 146.64, 145.47, 138.83, 135.15, 133.43, 132.66, 132.50, 130.47, 127.02, 125.76, 125.60, 124.90, 124.73, 115.55, 112.84, 55.06, 52.47, 50.56, 47.01, 46.85, 44.47, 32.54, 27.57, 22.37, 18.63; HRMS (ESI) calcd for C₃₁H₄₂N₅OS [M+H]⁺532.3105, found 518.2952.

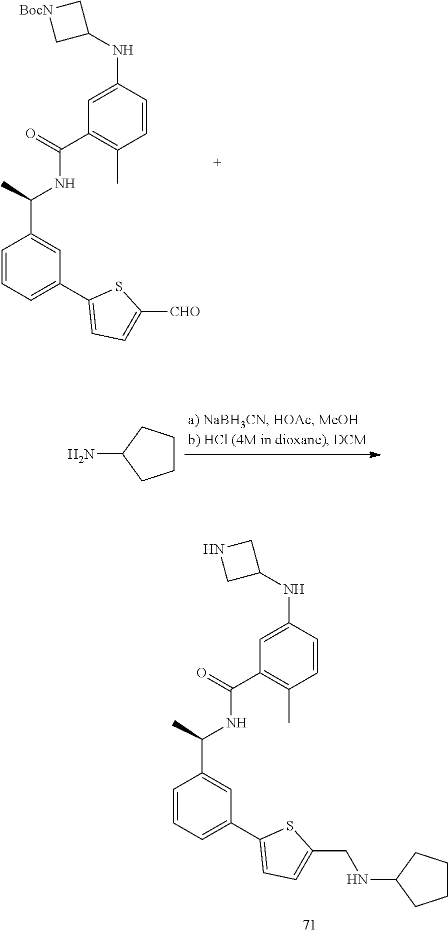 [SMILES: Cc1ccc(NC2CN(C(=O)OC(C)(C)C)C2)cc1C(=O)N[C@H](C)c1cccc(-c2ccc(C=O)s2)c1 ; NC1CCCC1 ; Cc1ccc(NC2CNC2)cc1C(=O)N[C@H](C)c1cccc(-c2ccc(CNC3CCCC3)s2)c1]

(R)-5-(azetidin-3-ylamino)-N-(1-(3-(5-((cyclopentylamino)methyl)thiophen-2-yl)phenyl)ethyl)-2-methylbenzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and cyclopentanamine (8 mg, 0.09 mmol) was subjected to general reductive amination procedure with MeCOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (21 mg, yield 71% for 2 steps) as a white solid: [α]₅₄₆²⁵=−3.1 (c 0.2, MeOH); ¹H NMR (400 MHz, Methanol-d₄) δ 8.45 (s, 1H), 7.72-7.67 (m, 1H), 7.57-7.52 (m, 1H), 7.43-7.37 (m, 3H), 7.28 (d, J=3.8 Hz, 1H), 7.03 (d, J=8.1 Hz, 1H), 6.60-6.54 (m, 2H), 5.21 (q, J=7.1 Hz, 1H), 4.54-4.46 (m, 1H), 4.44 (s, 2H), 4.39-4.32 (m, 2H), 3.98-3.89 (m, 2H), 3.67-3.57 (m, 1H), 2.23-2.12 (m, 5H), 1.89-1.78 (m, 2H), 1.75-1.65 (m, 4H), 1.55 (d, J=7.1 Hz, 3H); ¹³C NMR (100 MHz, Methanol-d₄) δ 172.60, 148.25, 146.62, 145.50, 138.86, 135.16, 133.08, 132.80, 132.64, 130.46, 126.96, 125.71, 125.61, 124.94, 124.75, 115.56, 112.80, 59.87, 54.88, 50.55, 46.86, 45.41, 30.73, 25.04, 22.34, 18.62; HRMS (ESI) calcd for C₂₉H₃₇N₄OS [M+H]⁺ 489.2683, found 489.2663.

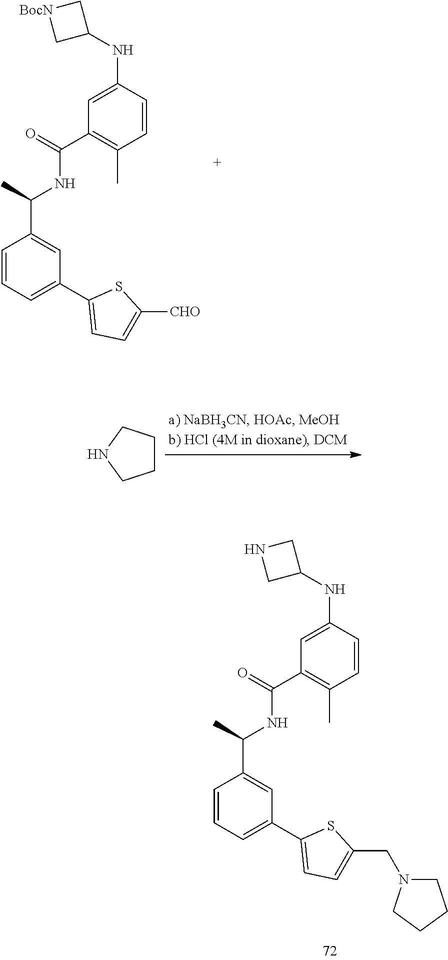 [SMILES: Cc1ccc(NC2CN(C(=O)OC(C)(C)C)C2)cc1C(=O)N[C@H](C)c1cccc(-c2ccc(C=O)s2)c1 ; C1CCNC1 ; Cc1ccc(NC2CNC2)cc1C(=O)N[C@H](C)c1cccc(-c2ccc(CN3CCCC3)s2)c1]

(R)-5-(azetidin-3-ylamino)-2-methyl-N-(1-(3-(5-(pyrrolidin-1-ylmethyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and pyrrolidine (7 mg, 0.09 mmol) was subjected to general reductive amination procedure with MeOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (15 mg, yield 54% for 2 steps) as a white solid: ¹H NMR (400 MHz, Methanol-d₄) δ 8.44 (s, 1H), 7.72-7.67 (m, 1H), 7.58-7.53 (m, 1H), 7.44-7.37 (m, 3H), 7.31-7.27 (m, 1H), 7.03 (d, J=7.9 Hz, 1H), 6.62-6.55 (m, 2H), 5.22 (q, J=7.0 Hz, 1H), 4.57 (s, 2H), 4.53-4.46 (m, 1H), 4.39-4.32 (m, 2H), 3.94 (dd, J=11.2, 6.5 Hz, 2H), 3.40-3.34 (m, 4H), 2.21 (s, 3H), 2.12-2.05 (m, 4H), 1.55 (d, J=7.1 Hz, 3H); ¹³C NMR (100 MHz, Methanol-d₄) δ 172.59, 148.64, 146.63, 145.49, 138.86, 135.11, 133.50, 132.72, 132.65, 130.47, 127.09, 125.73, 125.63, 124.91, 124.76, 115.56, 112.83, 54.90, 54.47, 53.16, 50.53, 46.86, 23.99, 22.36, 18.62; HRMS (ESI) calcd for C₂₈H₃₅N₄OS [M+H]⁺475.2526, found 475.2524.

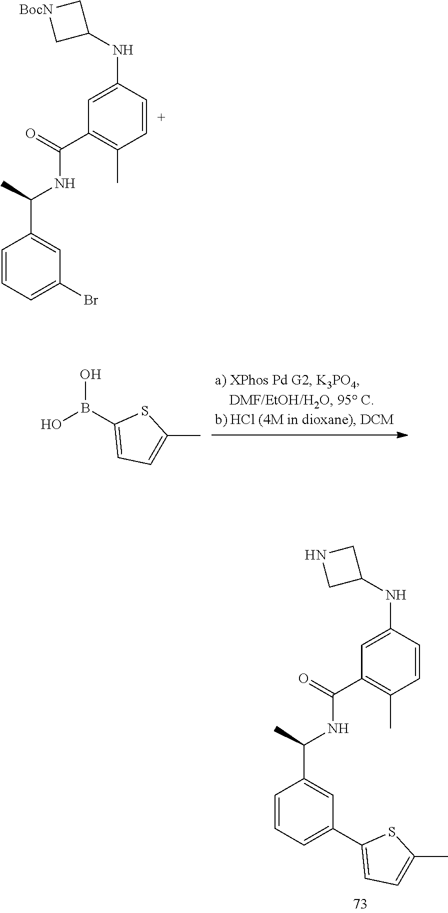

(R)-5-(azetidin-3-ylamino)-2-methyl-N-(1-(3-(5-methylthiophen-2-yl)phenyl)ethyl)benzamide. A flask fitted with a rubber septum was charged with tert-butyl (R)-3-((3-((1-(3-bromophenyl)ethyl)carbamoyl)-4-methylphenyl)amino)azetidine-1-carboxylate (49 mg, 0.10 mmol), (5-methylthiophen-2-yl)boronic acid (21 mg, 0.15 mmol), XPhos Pd G2 (8 mg, 0.01 mmol), K₃P04 (64 mg, 0.3 mmol), DMF/EtOH/H₂O (1 mL/1 mL/0.5 mL) and then purged with argon. The mixture was stirred at 95° C. overnight. The reaction mixture was then cooled to room temperature, diluted with ethyl acetate (20 mL), filtered through celite and concentrated in vacuo. After purification by Prep-HPLC, the product was subjected to general N-Boc deprotection procedure with HCl (4M in dioxane, 100 μL) and DCM (2 mL). The purification by Prep-HPLC afforded the product (19 mg, yield 47% for 2 steps) as a white solid: [α]₅₄₆²⁵=+14.7 (c 1.5, MeCOH); ¹H NMR (400 MHz, Methanol-d₄) δ 8.49 (s, 1H), 7.64-7.59 (m, 1H), 7.48-7.43 (m, 1H), 7.36-7.26 (m, 2H), 7.17 (d, J=3.7 Hz, 1H), 7.03 (d, J=8.2 Hz, 1H), 6.75 (dd, J=3.5, 1.2 Hz, 1H), 6.59-6.52 (m, 2H), 5.20 (q, J=7.0 Hz, 1H), 4.52-4.42 (m, 1H), 4.36-4.29 (m, 2H), 3.95-3.88 (m, 2H), 2.48 (d, J=1.0 Hz, 3H), 2.22 (s, 3H), 1.54 (d, J=7.1 Hz, 3H); ¹³C NMR (100 MHz, Methanol-d₄) δ 172.56, 146.15, 145.45, 143.05, 140.67, 138.93, 136.30, 132.63, 130.14, 127.43, 125.92, 125.71, 125.06, 124.17, 124.14, 115.62, 112.71, 54.84, 50.52, 46.84, 22.39, 18.63, 15.29; HRMS (ESI) calcd for C₂₄H₂₈N₃OS [M+H]⁺406.1948, found 406.1958.

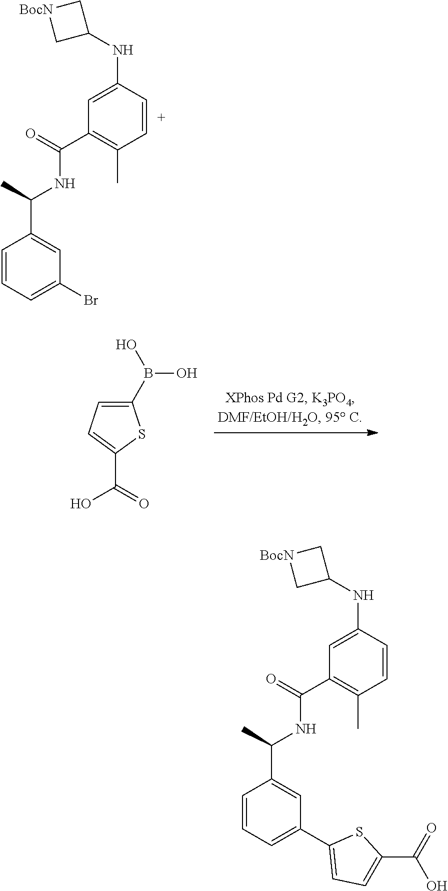 [SMILES: Cc1ccc(NC2CN(C(=O)OC(C)(C)C)C2)cc1C(=O)N[C@H](C)c1cccc(Br)c1 ; O=C(O)c1ccc(B(O)O)s1 ; Cc1ccc(NC2CN(C(=O)OC(C)(C)C)C2)cc1C(=O)N[C@H](C)c1cccc(-c2ccc(C(=O)O)s2)c1]

(R)-5-(3-(1-(5-((1-(tert-butoxycarbonyl)azetidin-3-yl)amino)-2-methylbenzamido)ethyl)Phenyl)thiophene-2-carboxylic acid. A flask fitted with a rubber septum was charged with tert-butyl (R)-3-((3-((1-(3-bromophenyl)ethyl)carbamoyl)-4-methylphenyl)amino)azetidine-1-carboxylate (490 mg, 1.00 mmol), 5-boronothiophene-2-carboxylic acid (258 mg, 1.50 mmol), XPhos Pd G2 (40 mg, 0.05 mmol), K₃P04 (531 mg, 2.5 mmol), DMF/EtOH/H₂O (5 mL/5 mL/2.5 mL) and then purged with argon. The mixture was stirred at 95° C. overnight. The reaction mixture was then cooled to room temperature, diluted with ethyl acetate (50 mL), filtered through celite and concentrated in vacuo. The purification by Prep-HPLC afforded the product (150 mg, yield 28%) as a white solid: ¹H NMR (400 MHz, Methanol-d₄) δ 7.77-7.71 (m, 2H), 7.63-7.58 (m, 1H), 7.44-7.39 (m, 3H), 7.00 (d, J=8.0 Hz, 1H), 6.58-6.51 (m, 2H), 5.22 (q, J=7.0 Hz, 1H), 4.28-4.16 (m, 3H), 3.73-3.65 (m, 2H), 2.21 (s, 3H), 1.55 (d, J=7.1 Hz, 3H), 1.43 (s, 9H); ¹¹C NMR (100 MHz, Methanol-d₄) δ 172.83, 158.16, 146.73, 146.29, 138.69, 135.32, 135.12, 132.48, 130.44, 127.77, 125.78, 125.06, 124.79, 115.71, 112.60, 81.05, 50.37, 44.18, 28.64, 22.42, 18.58; LRMS (ESI) calcd 536.22, found 536.27.

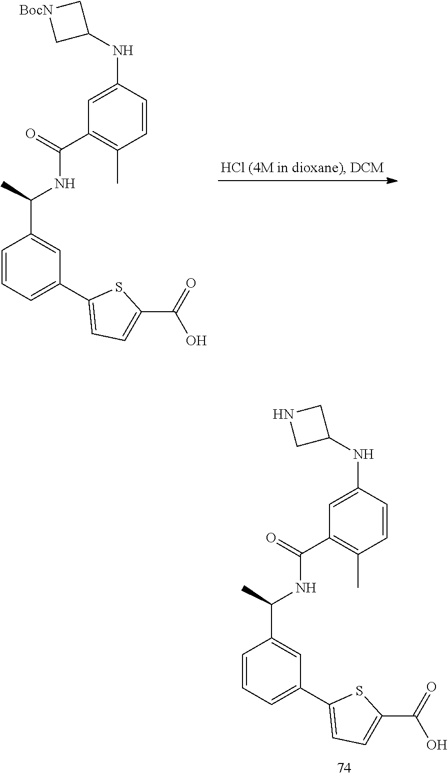 [SMILES: Cc1ccc(NC2CN(C(=O)OC(C)(C)C)C2)cc1C(=O)N[C@H](C)c1cccc(-c2ccc(C(=O)O)s2)c1 ; Cc1ccc(NC2CNC2)cc1C(=O)N[C@H](C)c1cccc(-c2ccc(C(=O)O)s2)c1]

(R)-5-(3-(1-(5-(azetidin-3-ylamino)-2-methylbenzamido)ethyl)phenyl)thiophene-2-carboxylic acid. (R)-5-(3-(1-(5-((1-(tert-butoxycarbonyl)azetidin-3-yl)amino)-2-methylbenzamido)ethyl)phenyl)thiophene-2-carboxylic acid (20 mg, 0.04 mmol) was subjected to general N-Boc deprotection procedure with HCl (4M in dioxane, 100 μL) and DCM (2 mL). The purification by Prep-HPLC afforded the product (14 mg, yield 86%) as a white solid: [α]₅₄₆²⁵=+61.7 (c 0.3, MeOH); ¹H NMR (400 MHz, Methanol-d₄) δ 7.75-7.72 (m, 1H), 7.65-7.62 (m, 1H), 7.58 (d, J=3.8 Hz, 1H), 7.44-7.32 (m, 3H), 7.05 (d, J=8.3 Hz, 1H), 6.68-6.61 (m, 2H), 5.21 (q, J=7.0 Hz, 1H), 4.67 (p, J=7.3 Hz, 1H), 4.50-4.37 (m, 2H), 4.05-3.93 (m, 2H), 2.27 (s, 3H), 1.54 (d, J=7.1 Hz, 3H); ¹³C NMR (100 MHz, Methanol-d₄) b 172.77, 149.48, 146.65, 145.45, 138.95, 135.71, 132.75, 132.68, 130.30, 127.54, 125.77, 124.96, 124.73, 123.55, 116.74, 111.35, 55.54, 55.33, 50.12, 46.78, 22.65, 18.47; HRMS (ESI) calcd for C₂₄H₂₆N₃O₃S [M+H]⁺436.1689, found 436.1697.

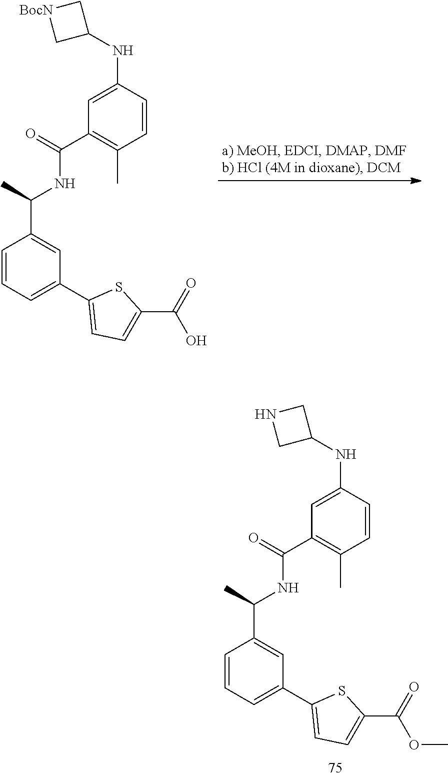 [SMILES: Cc1ccc(NC2CN(C(=O)OC(C)(C)C)C2)cc1C(=O)N[C@H](C)c1cccc(-c2ccc(C(=O)O)s2)c1 ; COC(=O)c1ccc(-c2cccc([C@@H](C)NC(=O)c3cc(NC4CNC4)ccc3C)c2)s1]

Methyl-(R)-5-(3-(1-(5-(azetidin-3-ylamino)-2-methylbenzamido)ethyl)phenyl)thiophene-2-carboxylate. (R)-5-(3-(1-(5-((1-(tert-butoxycarbonyl)azetidin-3-yl)amino)-2-methylbenzamido)ethyl)phenyl)thiophene-2-carboxylic acid (16 mg, 0.03 mmol), MeCOH (5 mg, 0.15 mmol), EDCl (10 mg, 0.05 mmol) and DMAP (11 mg, 0.09 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (11 mg, yield 82% for 2 steps) as a white solid: [α]₅₄₆²⁵=+14.5 (c 0.4, MeCOH); ¹H NMR (400 MHz, Acetone-d₆) δ 8.21 (s, 1H), 7.89-7.86 (m, 1H), 7.79 (d, J=3.9 Hz, 1H), 7.67-7.61 (m, 1H), 7.56-7.42 (m, 3H), 6.95 (d, J=8.4 Hz, 1H), 6.62 (d, J=2.6 Hz, 1H), 6.55 (dd, J=8.2, 2.6 Hz, 1H), 5.38-5.24 (m, 1H), 4.08 (p, J=6.7 Hz, 1H), 3.87 (s, 3H), 3.80-3.73 (m, 2H), 3.26-3.19 (m, 2H), 2.20 (s, 3H), 1.59 (d, J=7.0 Hz, 3H); ¹³C NMR (100 MHz, Acetone-d₆) δ 169.76, 162.83, 151.97, 147.31, 146.22, 138.76, 135.33, 134.21, 132.90, 132.08, 130.22, 127.91, 125.32, 125.01, 124.76, 124.44, 114.67, 112.51, 54.53, 54.49, 52.46, 49.35, 43.41, 22.72, 18.81; HRMS (ESI) calcd for C₂₅H₂₈N₃O₃S [M+H]⁺450.1846, found 450.1842.

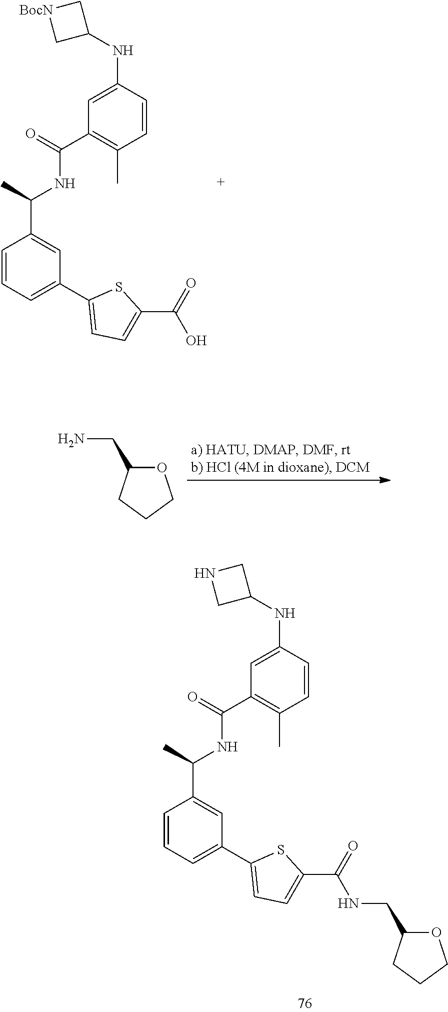

5-(3-((R)-1-(5-(azetidin-3-ylamino)-2-methylbenzamido)ethyl)phenyl)-N—(((R)-tetrahydrofuran-2-yl)methyl)thiophene-2-carboxamide. (R)-5-(3-(1-(5-((1-(tert-butoxycarbonyl)azetidin-3-yl)amino)-2-methylbenzamido)ethyl)phenyl)thiophene-2-carboxylic acid (16 mg, 0.03 mmol), (R)-(tetrahydrofuran-2-yl)methanamine (6 mg, 0.06 mmol), HATU (15 mg, 0.04 mmol) and DMAP (11 mg, 0.09 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (13 mg, yield 84% for 2 steps) as a white solid: [α]56=+56.7 (c 1.2, MeOH); ¹H NMR (400 MHz, Methanol-d₄) δ 8.41 (s, 1H), 7.74-7.72 (m, 1H), 7.69 (d, J=3.9 Hz, 1H), 7.63-7.59 (m, 1H), 7.45-7.37 (m, 3H), 7.04 (d, J=8.2 Hz, 1H), 6.64-6.56 (m, 2H), 5.22 (q, J=7.1 Hz, 1H), 4.61-4.52 (m, 1H), 4.46-4.36 (m, 2H), 4.09 (qd, J=6.9, 4.6 Hz, 1H), 4.01-3.93 (m, 2H), 3.92-3.84 (m, 1H), 3.80-3.72 (m, 1H), 3.52-3.38 (m, 2H), 2.24 (s, 3H), 2.09-1.86 (m, 3H), 1.72-1.61 (m, 1H), 1.55 (d, J=7.1 Hz, 3H); ¹³C NMR (100 MHz, Methanol-d₄) δ 172.68, 164.48, 150.42, 146.71, 145.47, 139.09, 138.89, 135.06, 132.68, 130.59, 130.45, 127.72, 125.72, 125.44, 125.04, 124.34, 116.18, 112.02, 79.18, 69.09, 55.11, 50.32, 46.81, 44.85, 29.93, 26.57, 22.49, 18.57; HRMS (ESI) calcd for C₂₉H₃₅N₄O₃S [M+H]⁺519.2424, found 519.2424.

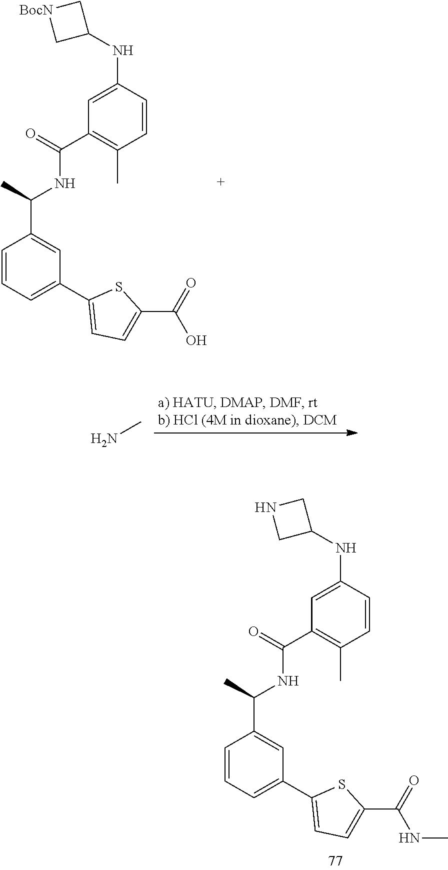

(R)-5-(3-(1-(5-(azetidin-3-ylamino)-2-methylbenzamido)ethyl)phenyl)-N-methylthiophene-2-carboxamide. (R)-5-(3-(1-(5-((1-(tert-butoxycarbonyl)azetidin-3-yl)amino)-2-methylbenzamido)ethyl)phenyl)thiophene-2-carboxylic acid (16 mg, 0.03 mmol), Methylamine (2 M in THF, 75 μL), HATU (15 mg, 0.04 mmol) and DMAP (11 mg, 0.09 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (12 mg, yield 89% for 2 steps) as a white solid: [α]₅₄₆²⁵=+41.5 (c 0.8, MeOH); ¹H NMR (400 MHz, Methanol-d₄) δ 8.45 (s, 1H), 7.75-7.71 (m, 1H), 7.63-7.59 (m, 2H), 7.45-7.37 (m, 3H), 7.04 (d, J=8.2 Hz, 1H), 6.65-6.55 (m, 2H), 5.22 (q, J=7.0 Hz, 1H), 4.60-4.52 (m, 1H), 4.46-4.36 (m, 2H), 4.01-3.92 (m, 2H), 2.92 (s, 3H), 2.23 (s, 3H), 1.55 (d, J=7.1 Hz, 3H); ¹³C NMR (100 MHz, Methanol-d₄) δ 172.68, 164.91, 150.17, 146.69, 145.46, 139.09, 138.90, 135.10, 132.69, 130.44, 130.34, 127.68, 125.76, 125.48, 125.01, 124.40, 116.12, 112.10, 55.13, 50.35, 46.85, 26.77, 22.47, 18.56; HRMS (ESI) calcd for C₂₅H₂₉N₄O₂S [M+H]⁺449.2006, found 449.2015.

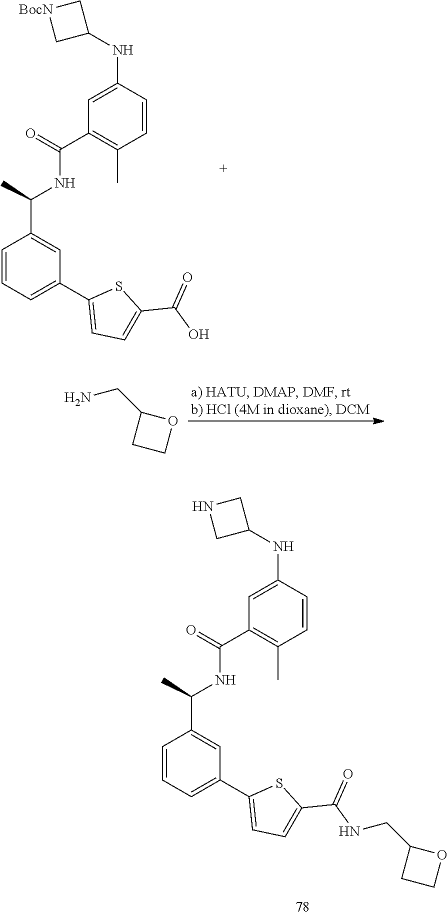 [SMILES: Cc1ccc(NC2CN(C(=O)OC(C)(C)C)C2)cc1C(=O)N[C@H](C)c1cccc(-c2ccc(C(=O)O)s2)c1 ; NCC1CCO1 ; Cc1ccc(NC2CNC2)cc1C(=O)N[C@H](C)c1cccc(-c2ccc(C(=O)NCC3CCO3)s2)c1]

5-(3-((R)-1-(5-(azetidin-3-ylamino)-2-methylbenzamido)ethyl)phenyl)-N-(oxetan-2-ylmethyl)thiophene-2-carboxamide. (R)-5-(3-(1-(5-((1-(tert-butoxycarbonyl)azetidin-3-yl)amino)-2-methylbenzamido)ethyl)phenyl)thiophene-2-carboxylic acid (16 mg, 0.03 mmol), oxetan-2-ylmethanamine (5 mg, 0.06 mmol), HATU (15 mg, 0.04 mmol) and DMAP (11 mg, 0.09 mmol) was subjected to general amine coupling procedure with DMF (1 mL). After purification by Prep-HPLC, the product was subjected to general N-Boc deprotection procedure with HCl (4M in dioxane, 100 μL) and DCM (2 mL). The purification by Prep-HPLC afforded the product (13 mg, yield 86% for 2 steps) as a white solid: ¹H NMR (400 MHz, Methanol-d₄) δ 8.36 (s, 1H), 7.76-7.70 (m, 1H), 7.64-7.58 (m, 2H), 7.46-7.37 (m, 3H), 7.04 (d, J=8.2 Hz, 1H), 6.64-6.52 (m, 2H), 5.23 (q, J=7.3 Hz, 1H), 4.99-4.92 (m, 1H), 4.55-4.47 (m, 1H), 4.42-4.32 (m, 2H), 4.17-4.09 (m, 1H), 3.98-3.90 (m, 2H), 3.79-3.65 (m, 3H), 2.23 (s, 3H), 2.05-1.84 (m, 2H), 1.55 (d, J=7.2 Hz, 3H); ¹¹C NMR (100 MHz, Methanol-d₄)δ 172.60, 161.81, 150.50, 146.72, 145.44, 138.92, 134.94, 133.18, 132.68, 130.50, 129.71, 127.79, 125.78, 125.08, 124.73, 115.75, 112.52, 79.84, 60.38, 59.15, 55.04, 54.99, 50.47, 46.88, 39.16, 22.44, 18.60; HRMS (ESI) calcd for C₂₈H₃₃N₄O₃S [M+H]⁺505.2268, found 505.2260.

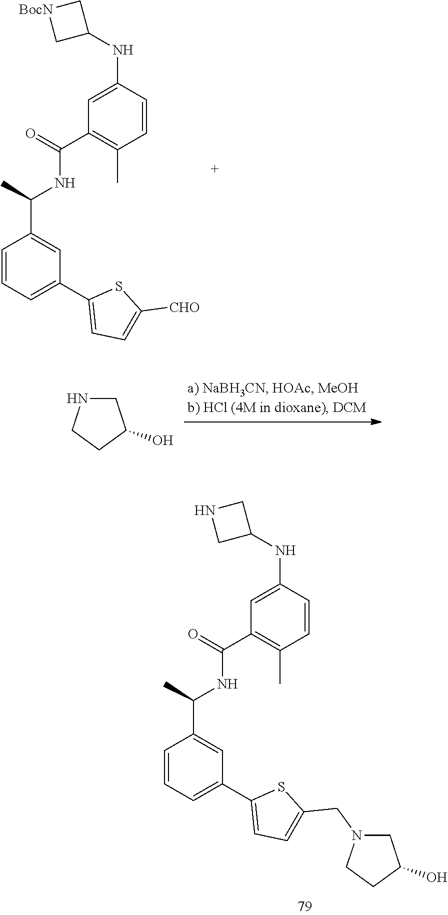 [SMILES: Cc1ccc(NC2CN(C(=O)OC(C)(C)C)C2)cc1C(=O)N[C@H](C)c1cccc(-c2ccc(C=O)s2)c1 ; O[C@@H]1CCNC1 ; Cc1ccc(NC2CNC2)cc1C(=O)N[C@H](C)c1cccc(-c2ccc(CN3CC[C@@H](O)C3)s2)c1]

5-(azetidin-3-ylamino)-N—((R)-1-(3-(5-(((R)-3-hydroxypyrrolidin-1-yl)methyl)thiophen-2-yl)phenyl)ethyl)-2-methylbenzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and (R)-pyrrolidin-3-ol (8 mg, 0.09 mmol) was subjected to general reductive amination procedure with MeOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (19 mg, yield 63% for 2 steps) as a white solid: [α]₅₄₆²⁵=+10.0 (c 0.3, MeOH); ¹H NMR (400 MHz, Methanol-d₄) δ 8.49 (s, 1H), 7.70-7.65 (m, 1H), 7.56-7.51 (m, 1H), 7.43-7.31 (m, 3H), 7.15 (d, J=3.7 Hz, 1H), 7.04 (d, J=8.3 Hz, 1H), 6.58 (dd, J=8.1, 2.6 Hz, 1H), 6.54 (d, J=2.7 Hz, 1H), 5.22 (q, J=7.2 Hz, 1H), 4.55-4.44 (m, 2H), 4.38-4.32 (m, 2H), 4.29-4.20 (m, 2H), 3.96-3.90 (m, 2H), 3.28-3.19 (m, 1H), 3.17-3.10 (m, 1H), 3.06-2.94 (m, 2H), 2.27-2.16 (m, 4H), 1.95-1.86 (m, 1H), 1.55 (d, J=7.1 Hz, 3H); ¹³C NMR (100 MHz, Methanol-d₄) δ 172.59, 147.10, 146.50, 145.46, 138.93, 135.57, 132.66, 131.36, 130.36, 126.77, 125.76, 125.46, 124.67, 124.36, 115.66, 112.64, 70.90, 62.56, 55.00, 54.86, 53.38, 50.53, 46.90, 34.65, 22.37, 18.61; HRMS (ESI) calcd for C₂₈H₃₅N₄O₂S [M+H]⁺491.2475, found 491.2483.

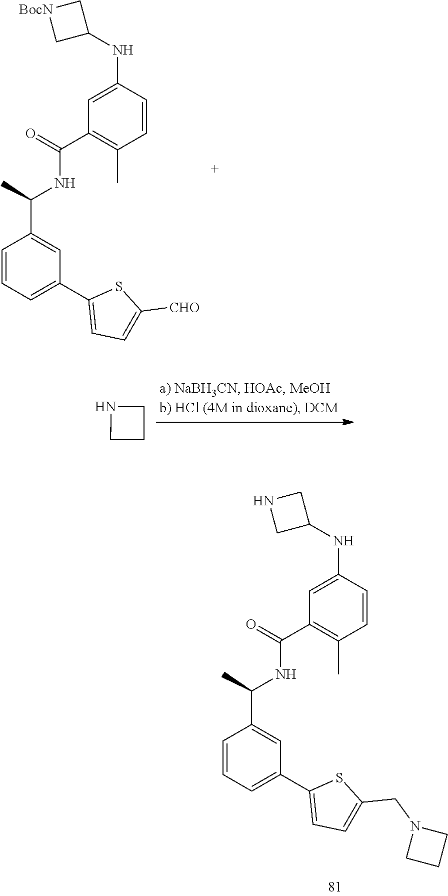

(R)—N-(1-(3-(5-(azetidin-1-ylmethyl)thiophen-2-yl)phenyl)ethyl)-5-(azetidin-3-ylamino)-2-methylbenzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and azetidine hydrochloride salt (8 mg, 0.09 mmol) was subjected to general reductive amination procedure with MeCOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (14 mg, yield 49% for 2 steps) as a white solid: ¹H NMR (400 MHz, Methanol-d₄) b 8.51 (s, 1H), 7.69-7.65 (m, 1H), 7.55-7.51 (m, 1H), 7.43-7.33 (m, 3H), 7.17 (d, J=3.8 Hz, 1H), 7.04 (d, J=8.2 Hz, 1H), 6.60-6.53 (m, 2H), 5.21 (q, J=7.0 Hz, 1H), 4.50 (p, J=6.5 Hz, 1H), 4.39-4.29 (m, 4H), 3.97-3.87 (m, 6H), 2.39 (p, J=7.8 Hz, 2H), 2.21 (s, 3H), 1.55 (d, J=7.1 Hz, 3H); ¹³C NMR (100 MHz, Methanol-d₄) δ 172.60, 146.56, 145.47, 138.91, 135.34, 132.65, 131.71, 130.41, 126.87, 125.73, 125.53, 124.81, 124.61, 115.58, 112.73, 55.19, 55.03, 54.96, 50.55, 46.89, 22.37, 18.62, 17.39; HRMS (ESI) calcd for C₂₇H₃₃N₄OS [M+H]⁺ 461.2370, found 461.2370.

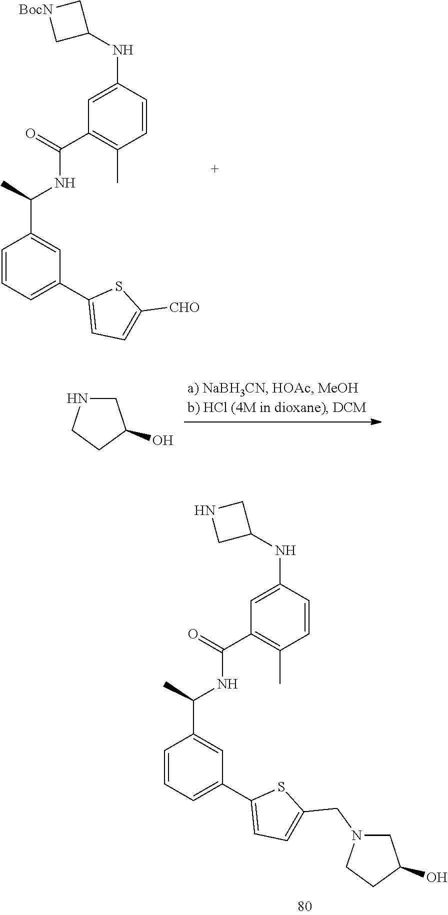 [SMILES: Cc1ccc(NC2CN(C(=O)OC(C)(C)C)C2)cc1C(=O)N[C@H](C)c1cccc(-c2ccc(C=O)s2)c1 ; O[C@H]1CCNC1 ; Cc1ccc(NC2CNC2)cc1C(=O)N[C@H](C)c1cccc(-c2ccc(CN3CC[C@H](O)C3)s2)c1]

5-(azetidin-3-ylamino)-N—((R)-1-(3-(5-(((S)-3-hydroxypyrrolidin-1-yl)methyl)thiophen-2-yl)phenyl)ethyl)-2-methylbenzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and (S)-pyrrolidin-3-ol (8 mg, 0.09 mmol) was subjected to general reductive amination procedure with MeCOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (16 mg, yield 55% for 2 steps) as a white solid: ¹H NMR (400 MHz, Methanol-d₄) δ 8.37 (s, 1H), 7.72-7.66 (m, 1H), 7.58-7.53 (m, 1H), 7.44-7.37 (m, 3H), 7.28 (d, J=3.7 Hz, 1H), 7.04 (d, J=8.2 Hz, 1H), 6.61-6.54 (m, 2H), 5.22 (q, J=7.0 Hz, 1H), 4.61-4.46 (m, 4H), 4.40-4.33 (m, 2H), 3.98-3.91 (m, 2H), 3.53 (dt, J=11.4, 8.0 Hz, 1H), 3.41-3.31 (m, 2H), 3.28-3.22 (m, 1H), 2.34-2.22 (m, 1H), 2.21 (s, 3H), 2.09-1.99 (m, 1H), 1.55 (d, J=7.1 Hz, 3H); ¹³C NMR (100 MHz, Methanol-d₄) δ 172.60, 148.48, 146.61, 145.47, 138.87, 135.16, 133.36, 133.28, 132.65, 130.46, 127.02, 125.74, 125.61, 124.92, 124.70, 115.57, 112.77, 70.41, 62.07, 54.99, 54.37, 53.34, 50.55, 46.86, 34.22, 22.36, 18.62; HRMS (ESI) calcd for C₂₈H₃₅N₄O₂S [M+H]⁺491.2475, found 491.2483.

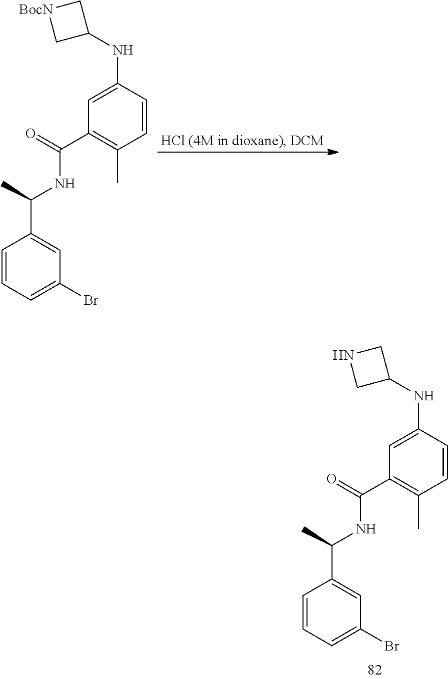 [SMILES: Cc1ccc(NC2CN(C(=O)OC(C)(C)C)C2)cc1C(=O)N[C@H](C)c1cccc(Br)c1 ; Cc1ccc(NC2CNC2)cc1C(=O)N[C@H](C)c1cccc(Br)c1]

(R)-5-(azetidin-3-ylamino)-N-(1-(3-bromophenyl)ethyl)-2-methylbenzamide. White solid (yield 86%): [α]₅₄₆²⁵=+16.4 (c 0.9, MeOH); ¹H NMR (400 MHz, Methanol-d₄) δ 7.62-7.54 (m, 1H), 7.43-7.35 (m, 2H), 7.30-7.23 (m, 1H), 7.03 (d, J=8.2 Hz, 1H), 6.60-6.51 (m, 2H), 5.15 (q, J=7.1 Hz, 1H), 4.50 (p, J=6.8 Hz, 1H), 4.36 (t, J=9.1 Hz, 2H), 3.97-3.88 (m, 2H), 2.20 (s, 3H), 1.50 (d, J=7.1 Hz, 3H); 13C NMR (100 MHz, Methanol-d₄) δ 172.58, 148.13, 145.47, 138.75, 132.66, 131.41, 131.16, 130.33, 126.19, 125.71, 123.47, 115.63, 112.68, 54.89, 50.15, 46.84, 22.21, 18.53.HRMS (ESI) calcd for C₁₉H₂₃BrN₃O [M+H]⁺388.1019, found 388.1022.

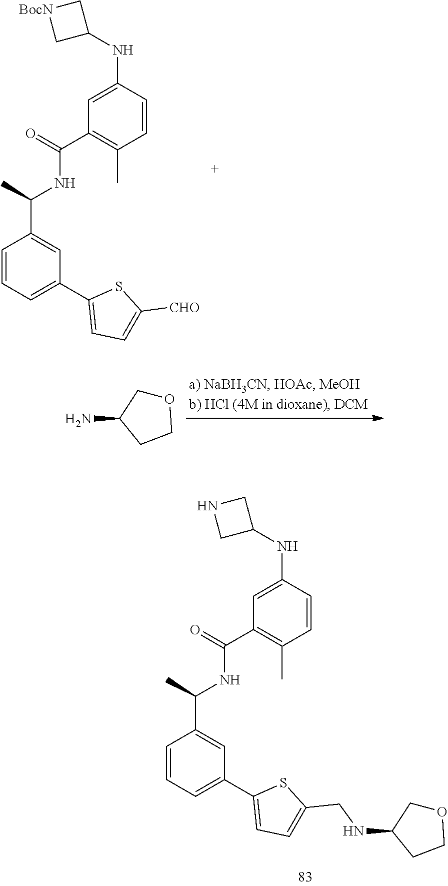 [SMILES: Cc1ccc(NC2CN(C(=O)OC(C)(C)C)C2)cc1C(=O)N[C@H](C)c1cccc(-c2ccc(C=O)s2)c1 ; N[C@@H]1CCOC1 ; Cc1ccc(NC2CNC2)cc1C(=O)N[C@H](C)c1cccc(-c2ccc(CN[C@@H]3CCOC3)s2)c1]

5-(azetidin-3-ylamino)-2-methyl-N—((R)-1-(3-(5-((((R)-tetrahydrofuran-3-yl)amino)methyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and (R)-tetrahydrofuran-3-amine (8 mg, 0.09 mmol) was subjected to general reductive amination procedure with MeOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (21 mg, yield 71% for 2 steps) as a white solid: [α]₅₄₆²⁵=+1.5 (c 0.4, MeOH); ¹H NMR (400 MHz, Methanol-d₄) δ 8.36 (s, 1H), 7.71-7.66 (m, 1H), 7.57-7.52 (m, 1H), 7.44-7.34 (m, 3H), 7.22 (d, J=3.7 Hz, 1H), 7.03 (d, J=8.2 Hz, 1H), 6.61-6.52 (m, 2H), 5.21 (q, J=7.0 Hz, 1H), 4.50 (p, J=6.9 Hz, 1H), 4.40-4.30 (m, 4H), 4.03 (td, J=8.4, 5.4 Hz, 1H), 3.97-3.81 (m, 5H), 3.75 (td, J=8.4, 6.8 Hz, 1H), 2.41-2.28 (m, 1H), 2.20 (s, 3H), 2.08-1.97 (m, 1H), 1.55 (d, J=7.1 Hz, 3H); ¹³C NMR (100 MHz, Methanol-d₄) δ 172.59, 147.63, 146.56, 145.47, 138.89, 135.36, 135.25, 132.65, 131.92, 130.41, 126.84, 125.74, 125.54, 124.83, 124.62, 115.59, 112.76, 71.32, 68.06, 59.04, 54.94, 50.55, 46.88, 45.74, 31.17, 22.34, 18.61; HRMS (ESI) calcd for C₂₈H₃₅N₄O₂S [M+H]⁺491.2475, found 491.2483.

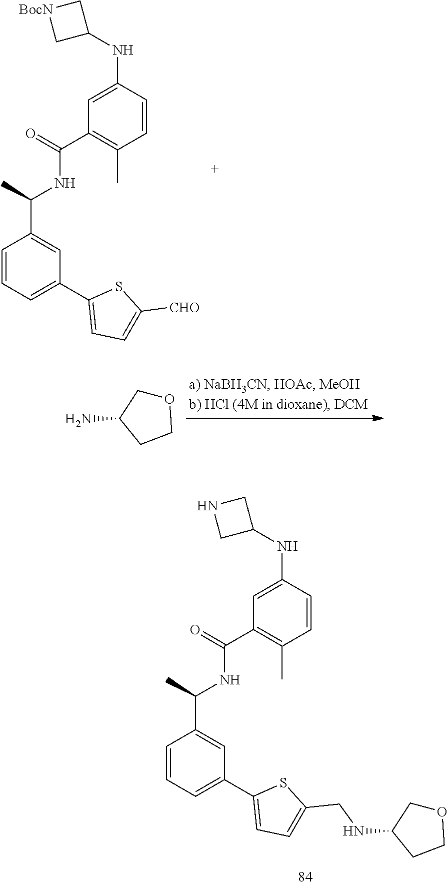 [SMILES: Cc1ccc(NC2CN(C(=O)OC(C)(C)C)C2)cc1C(=O)N[C@H](C)c1cccc(-c2ccc(C=O)s2)c1 ; N[C@H]1CCOC1 ; Cc1ccc(NC2CNC2)cc1C(=O)N[C@H](C)c1cccc(-c2ccc(CN[C@H]3CCOC3)s2)c1]

5-(azetidin-3-ylamino)-2-methyl-N—((R)-1-(3-(5-((((S)-tetrahydrofuran-3-yl)amino)methyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and (S)-tetrahydrofuran-3-amine (8 mg, 0.09 mmol) was subjected to general reductive amination procedure with MeOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (19 mg, yield 65% for 2 steps) as a white solid: [α]₅₄₆²⁵=+6.7 (c 0.9, MeCOH); ¹H NMR (400 MHz, Methanol-d₄) δ 8.43 (s, 1H), 7.67 (t, J=1.8 Hz, 1H), 7.56-7.50 (m, 1H), 7.42-7.30 (m, 3H), 7.12 (d, J=3.7 Hz, 1H), 7.04 (d, J=8.3 Hz, 1H), 6.58 (dd, J=8.2, 2.6 Hz, 1H), 6.53 (d, J=2.6 Hz, 1H), 5.21 (q, J=7.1 Hz, 1H), 4.54-4.45 (m, 1H), 4.39-4.31 (m, 2H), 4.22-4.12 (m, 2H), 4.03-3.89 (m, 3H), 3.86-3.64 (m, 4H), 2.30-2.23 (m, 1H), 2.21 (s, 3H), 1.98-1.87 (m, 1H), 1.55 (d, J=7.1 Hz, 3H); ¹³C NMR (100 MHz, Methanol-d₄) δ 171.18, 145.03, 144.04, 137.56, 134.30, 131.25, 128.92, 128.75, 125.20, 124.34, 124.02, 123.24, 122.95, 114.23, 111.24, 71.04, 66.72, 57.49, 53.59, 49.13, 45.50, 45.19, 30.79, 20.95, 17.20; HRMS (ESI) calcd for C₂₈H₃₅N₄O₂S [M+H]⁺491.2475, found 491.2479.

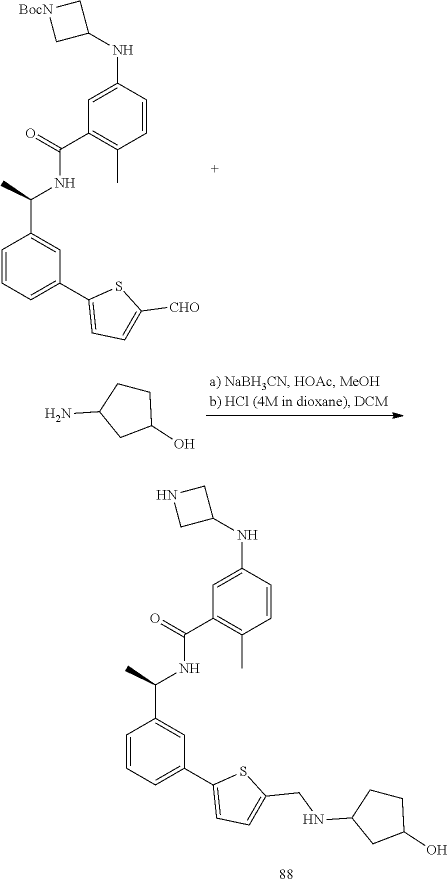

5-(azetidin-3-ylamino)-N-((1R)-1-(3-(5-(((3-hydroxycyclopentyl)amino)methyl)thiophen-2-yl)phenyl)ethyl)-2-methylbenzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and 3-aminocyclopentan-1-ol (9 mg, 0.09 mmol) was subjected to general reductive amination procedure with MeCOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (22 mg, yield 73% for 2 steps) as a white solid: ¹H NMR (400 MHz, Methanol-d₄) δ 8.44 (s, 1H), 7.69 (s, 1H), 7.55 (d, J=6.6 Hz, 1H), 7.44-7.37 (m, 3H), 7.27 (d, J=3.7 Hz, 1H), 7.03 (d, J=8.0 Hz, 1H), 6.62-6.54 (m, 2H), 5.26-5.15 (m, 1H), 4.54-4.26 (m, 6H), 3.98-3.90 (m, 2H), 3.73-3.63 (m, 1H), 2.30-2.12 (m, 5H), 2.04-1.92 (m, 1H), 1.88-1.79 (m, 3H), 1.55 (d, J=7.0 Hz, 3H); ¹³C NMR (100 MHz, Methanol-d₄) δ 172.60, 148.28, 146.62, 145.49, 138.86, 135.16, 133.09, 132.84, 132.64, 130.46, 126.94, 125.71, 125.60, 124.95, 124.76, 115.56, 112.79, 72.55, 58.48, 54.92, 50.55, 46.87, 45.19, 39.33, 34.42, 28.38, 22.34, 18.61; HRMS (ESI) calcd for C₂₉H₃₇N₄O₂S [M+H]⁺ 505.2632, found 505.2637.

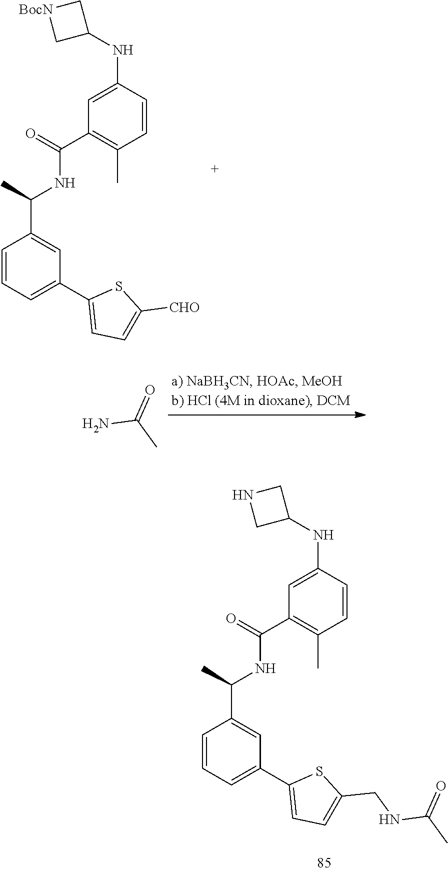

(R)—N-(1-(3-(5-(acetamidomethyl)thiophen-2-yl)phenyl)ethyl)-5-(azetidin-3-ylamino)-2-methylbenzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and acetamide (6 mg, 0.09 mmol) was subjected to general reductive amination procedure with MeOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (20 mg, yield 73% for 2 steps) as a white solid: ¹H NMR (400 MHz, Methanol-d₄) δ 8.55 (s, 2H), 7.65-7.61 (m, 1H), 7.52-7.48 (m, 1H), 7.39-7.29 (m, 2H), 7.23 (d, J=3.6 Hz, 1H), 7.03 (d, J=8.2 Hz, 1H), 6.96 (d, J=3.7 Hz, 1H), 6.58 (dd, J=8.2, 2.6 Hz, 1H), 6.53 (d, J=2.6 Hz, 1H), 5.23-5.17 (m, 1H), 4.52 (s, 2H), 4.49-4.42 (m, 1H), 4.31-4.23 (m, 2H), 3.91-3.84 (m, 2H), 2.22 (s, 3H), 1.98 (s, 3H), 1.54 (d, J=7.1 Hz, 3H); ¹³C NMR (100 MHz, Methanol-d₄) δ 172.63, 146.32, 145.58, 144.99, 142.64, 138.93, 136.03, 132.62, 130.21, 127.84, 126.46, 125.63, 125.20, 124.25, 123.87, 122.97, 115.72, 112.54, 55.04, 50.47, 47.24, 39.15, 22.51, 22.39, 18.60; HRMS (ESI) calcd for C₂₆H₃₁N₄O₂S [M+H]⁺463.2162, found 463.2164.

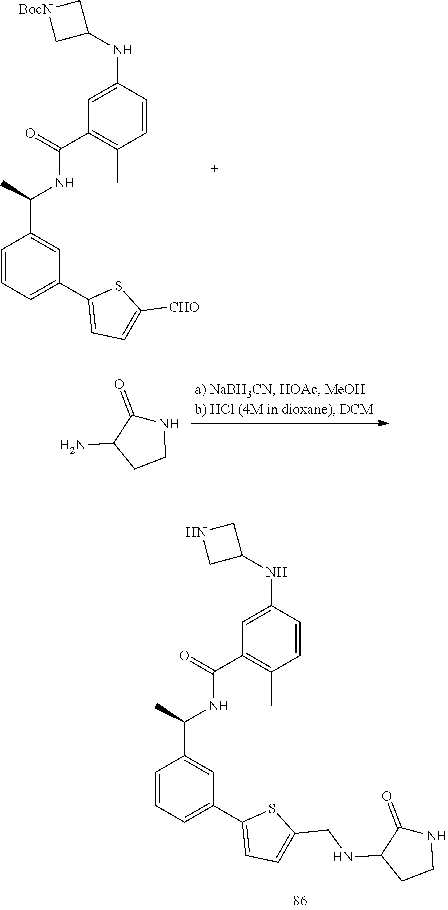

5-(azetidin-3-ylamino)-2-methyl-N-((1R)-1-(3-(5-(((2-oxopyrrolidin-3-yl)amino)methyl)thiophen-2-yl)phenyl)ethyl)benzamide. Tert-butyl (R)-3-((3-((1-(3-(5-formylthiophen-2-yl)phenyl)ethyl)carbamoyl)-4-methylphenyl)amino)azetidine-1-carboxylate (31 mg, 0.06 mmol) and 3-aminopyrrolidin-2-one (9 mg, 0.09 mmol) was subjected to general reductive amination procedure with MeCOH (2 mL), HOAc (500 μL) at 50° C., and then NaBH₃CN (12 mg, 0.18 mmol) was added. After purification by Prep-HPLC, the product was subjected to general N-Boc deprotection procedure with HCl (4M in dioxane, 100 μL) and DCM (2 mL). The purification by Prep-HPLC afforded the product (19 mg, yield 63% for 2 steps) as a white solid: ¹H NMR (400 MHz, Methanol-d₄) δ 8.28 (s, 2H), 7.71-7.65 (m, 1H), 7.56-7.51 (m, 1H), 7.42-7.30 (m, 3H), 7.14 (d, J=3.7 Hz, 1H), 7.03 (d, J=8.3 Hz, 1H), 6.59 (dd, J=8.2, 2.6 Hz, 1H), 6.55-6.51 (m, 1H), 5.21 (q, J=7.0 Hz, 1H), 4.49 (p, J=7.0 Hz, 1H), 4.39-4.27 (m, 4H), 3.98-3.90 (m, 2H), 3.79-3.72 (m, 1H), 3.44-3.33 (m, 2H), 2.55-2.46 (m, 1H), 2.22 (s, 3H), 2.11-1.99 (m, 1H), 1.55 (d, J=7.0 Hz, 3H); ¹³C NMR (100 MHz, Methanol-d₄) δ 176.76, 172.60, 146.50, 145.47, 138.85, 135.69, 132.66, 130.45, 130.32, 126.75, 125.77, 125.73, 125.37, 124.44, 124.39, 115.82, 112.49, 57.49, 54.86, 50.49, 46.84, 46.14, 40.23, 28.33, 22.42, 18.61; HRMS (ESI) calcd for C₂₈H₃₄N₅O₂S [M+H]⁺504.2428, found 504.2431.

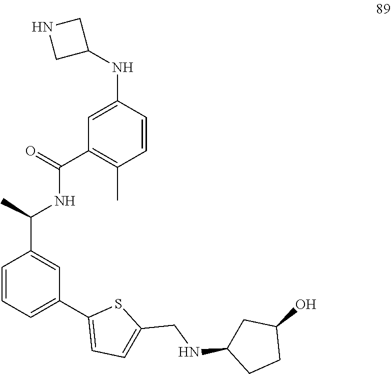 [SMILES: Cc1ccc(NC2CNC2)cc1C(=O)N[C@H](C)c1cccc(-c2ccc(CN[C@@H]3CC[C@H](O)C3)s2)c1]

5-(azetidin-3-ylamino)-N—((R)-1-(3-(5-((((1R,3S)-3-hydroxycyclopentyl)amino)methyl)thiophen-2-yl)phenyl)ethyl)-2-methylbenzamide. White solid (yield 67%): [α]₅₄₆²⁵=−6.8 (c 0.8, MeOH); ¹H NMR (400 MHz, Methanol-d₄) δ 8.40 (s, 1H), 7.73-7.67 (m, 1H), 7.57-7.52 (m, 1H), 7.40 (dd, J=6.8, 4.0 Hz, 3H), 7.28 (d, J=3.7 Hz, 1H), 7.03 (d, J=8.0 Hz, 1H), 6.61-6.55 (m, 2H), 5.21 (q, J=7.0 Hz, 1H), 4.55-4.43 (m, 3H), 4.39-4.30 (m, 3H), 3.97-3.89 (m, 2H), 3.68 (tt, J=8.0, 5.8 Hz, 1H), 2.30-2.12 (m, 5H), 2.05-1.93 (m, 1H), 1.89-1.82 (m, 3H), 1.55 (d, J=7.0 Hz, 3H); ¹³C NMR (100 MHz, Methanol-d₄) δ 172.60, 148.34, 146.62, 145.49, 138.84, 135.15, 132.95, 132.85, 132.64, 130.46, 126.98, 125.71, 125.60, 124.92, 124.77, 115.56, 112.81, 72.52, 58.47, 54.89, 50.55, 46.85, 45.14, 39.26, 34.40, 28.32, 22.35, 18.62; HRMS (ESI) calcd for C₂₉H₃₇N₄O₂S [M+H]⁺505.2632, found 505.2634.

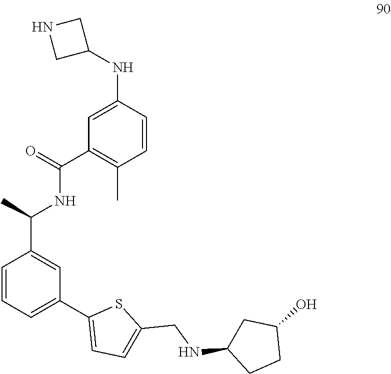 [SMILES: Cc1ccc(NC2CNC2)cc1C(=O)N[C@H](C)c1cccc(-c2ccc(CN[C@@H]3CC[C@@H](O)C3)s2)c1]

5-(azetidin-3-ylamino)-N—((R)-1-(3-(5-((((1R,3R)-3-hydroxycyclopentyl)amino)methyl)thiophen-2-yl)phenyl)ethyl)-2-methylbenzamide. White solid (yield 65%): [α]₅₄₆²⁵=−3.2 (c 2.3, MeOH); ¹H NMR (400 MHz, Methanol-d₄) δ 8.42 (s, 1H), 7.69 (d, J=1.9 Hz, 1H), 7.57-7.52 (m, 1H), 7.43-7.36 (m, 3H), 7.28 (d, J=3.7 Hz, 1H), 7.03 (d, J=8.0 Hz, 1H), 6.60-6.54 (m, 2H), 5.21 (q, J=7.0 Hz, 1H), 4.55-4.31 (m, 6H), 3.98-3.90 (m, 2H), 3.89-3.80 (m, 1H), 2.39-2.27 (m, 1H), 2.23-2.13 (m, 4H), 2.11-2.02 (m, 1H), 1.92-1.83 (m, 1H), 1.80-1.67 (m, 2H), 1.55 (d, J=7.0 Hz, 3H). ¹³C NMR (100 MHz, Methanol-d₄) δ 172.59, 148.27, 146.61, 145.49, 138.86, 135.17, 133.08, 132.80, 132.64, 130.45, 126.99, 125.73, 125.61, 124.91, 124.75, 115.57, 112.81, 72.45, 58.26, 54.90, 50.54, 46.86, 45.39, 39.88, 34.12, 28.21, 22.34, 18.62; HRMS (ESI) calcd for C₂₉H₃₇N₄O₂S [M+H]⁺505.2632, found 505.2636.

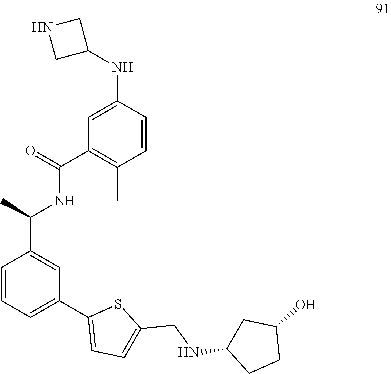 [SMILES: Cc1ccc(NC2CNC2)cc1C(=O)N[C@H](C)c1cccc(-c2ccc(CN[C@H]3CC[C@@H](O)C3)s2)c1]

5-(azetidin-3-ylamino)-N—((R)-1-(3-(5-((((1S,3R)-3-hydroxycyclopentyl)amino)methyl)thiophen-2-yl)phenyl)ethyl)-2-methylbenzamide. White solid (yield 72%): [α]₅₄₆²⁵=+12.9 (c 0.4, MeOH); ¹H NMR (400 MHz, Methanol-d₄) δ 8.45 (s, 1H), 7.72-7.67 (m, 1H), 7.58-7.51 (m, 1H), 7.44-7.38 (m, 3H), 7.26 (d, J=3.7 Hz, 1H), 7.03 (d, J=8.1 Hz, 1H), 6.61-6.54 (m, 2H), 5.21 (q, J=7.0 Hz, 1H), 4.50 (p, J=7.0 Hz, 1H), 4.43 (s, 2H), 4.35 (dt, J=8.8, 6.3 Hz, 3H), 3.93 (dd, J=11.1, 6.6 Hz, 2H), 3.71-3.62 (m, 1H), 2.29-2.12 (m, 5H), 2.04-1.92 (m, 1H), 1.89-1.80 (m, 3H), 1.55 (d, J=7.1 Hz, 3H); ¹³C NMR (100 MHz, Methanol-d₄) δ 172.60, 148.23, 146.61, 145.49, 138.89, 135.18, 133.26, 132.74, 132.64, 130.46, 126.92, 125.72, 125.60, 124.98, 124.76, 115.57, 112.79, 72.57, 58.51, 54.93, 50.55, 46.88, 45.24, 39.38, 34.43, 28.44, 22.33, 18.61; HRMS (ESI) calcd for C₂₉H₃₇N₄O₂S [M+H]⁺505.2632, found 505.2638.

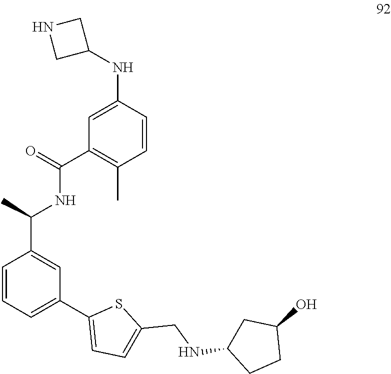 [SMILES: Cc1ccc(NC2CNC2)cc1C(=O)N[C@H](C)c1cccc(-c2ccc(CN[C@H]3CC[C@H](O)C3)s2)c1]

5-(azetidin-3-ylamino)-N—((R)-1-(3-(5-((((1S,3S)-3-hydroxycyclopentyl)amino)methyl)thiophen-2-yl)phenyl)ethyl)-2-methylbenzamide. White solid (yield 72%): [α]₅₄₆²⁵=−3.2 (c 1.4, MeOH); ¹H NMR (400 MHz, Methanol-d₄) δ 8.43 (s, 1H), 7.71-7.68 (m, 1H), 7.57-7.52 (m, 1H), 7.44-7.37 (m, 3H), 7.27 (d, J=3.7 Hz, 1H), 7.03 (d, J=8.0 Hz, 1H), 6.61-6.53 (m, 2H), 5.21 (q, J=7.0 Hz, 1H), 4.51 (p, J=7.0 Hz, 1H), 4.45 (s, 2H), 4.40-4.30 (m, 3H), 3.99-3.90 (m, 2H), 3.72-3.64 (m, 1H), 2.29-2.11 (m, 5H), 2.04-1.93 (m, 1H), 1.89-1.81 (m, 3H), 1.55 (d, J=7.1 Hz, 3H); ¹³C NMR (100 MHz, Methanol-d₄) δ 172.59, 148.31, 146.62, 145.49, 138.86, 135.16, 132.98, 132.89, 132.64, 130.46, 126.97, 125.73, 125.60, 124.95, 124.77, 115.57, 112.82, 72.55, 58.51, 54.93, 50.55, 46.87, 45.19, 39.31, 34.41, 28.36, 22.34, 18.62; HRMS (ESI) calcd for C₂₉H₃₇N₄O₂S [M+H]⁺505.2632, found 505.2636.

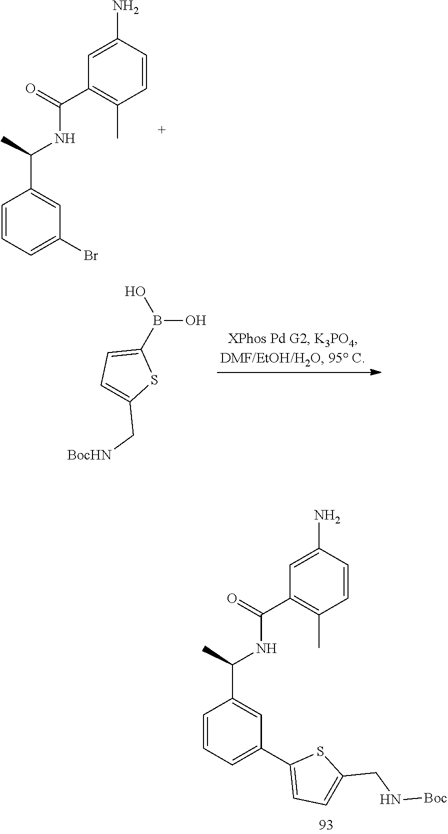 [SMILES: Cc1ccc(N)cc1C(=O)N[C@H](C)c1cccc(Br)c1 ; CC(C)(C)OC(=O)NCc1ccc(B(O)O)s1 ; Cc1ccc(N)cc1C(=O)N[C@H](C)c1cccc(-c2ccc(CNC(=O)OC(C)(C)C)s2)c1]

(R)-3-((1-(3-(5-(((tert-butoxycarbonyl)amino)methyl)thiophen-2-yl)phenyl)ethyl)carbamoyl)-4-methylbenzenaminium. A flask fitted with a rubber septum was charged with (R)-5-amino-N-(1-(3-bromophenyl)ethyl)-2-methylbenzamide (33 mg, 0.10 mmol), (5-(((tert-butoxycarbonyl)amino)methyl)thiophen-2-yl)boronic acid (39 mg, 0.15 mmol), XPhos Pd G2 (8 mg, 0.01 mmol), K₃PO₄ (64 mg, 0.3 mmol), DMF/EtOH/H₂O (1 mL/1 mL/0.5 mL) and then purged with argon. The mixture was stirred at 95° C. overnight. The reaction mixture was then cooled to room temperature, diluted with ethyl acetate (20 mL), filtered through celite and concentrated in vacuo. The purification by Prep-HPLC afforded the product (41 mg, yield 88%) as a white solid: [α]₅₄₆²⁵=+22.6 (c 1.5, MeCOH); ¹H NMR (400 MHz, CDCl₃) δ 7.57-7.54 (m, 1H), 7.49-7.45 (m, 1H), 7.37-7.33 (m, 1H), 7.29-7.27 (m, 1H), 7.13 (d, J=3.6 Hz, 1H), 6.97 (d, J=8.1 Hz, 1H), 6.90 (d, J=3.6 Hz, 1H), 6.70 (d, J=2.5 Hz, 1H), 6.63 (dd, J=8.1, 2.6 Hz, 1H), 6.00 (d, J=8.0 Hz, 1H), 5.36-5.27 (m, 1H), 4.46 (d, J=5.9 Hz, 2H), 2.30 (s, 3H), 1.59 (d, J=6.9 Hz, 3H), 1.47 (s, 9H); ¹³C NMR (100 MHz, CDCl₃) δ 169.44, 144.35, 144.05, 143.80, 141.83, 137.19, 134.94, 132.00, 129.43, 126.58, 125.39, 124.94, 123.72, 123.06, 116.83, 113.57, 49.03, 39.95, 28.54, 22.01, 18.90; HRMS (ESI) calcd for C₂₆H₃₂N₃O₃S [M+H]⁺466.2159, found 466.2157.

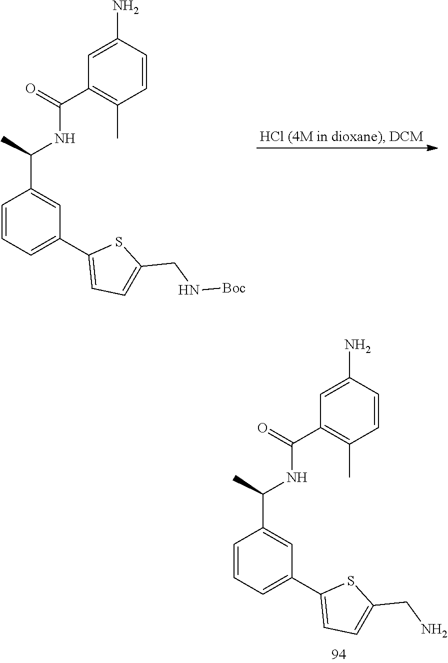 [SMILES: Cc1ccc(N)cc1C(=O)N[C@H](C)c1cccc(-c2ccc(CNC(=O)OC(C)(C)C)s2)c1 ; Cc1ccc(N)cc1C(=O)N[C@H](C)c1cccc(-c2ccc(CN)s2)c1]

(R)-5-amino-N-(1-(3-(5-(aminomethyl)thiophen-2-yl)phenyl)ethyl)-2-methylbenzamide. (R)-3-((1-(3-(5-(((tert-butoxycarbonyl)amino)methyl)thiophen-2-yl)phenyl)ethyl)carbamoyl)-4-methylbenzenaminium (20 mg, 0.04 mmol) was subjected to general N-Boc deprotection procedure with HCl (4M in dioxane, 100 μL) and DCM (2 mL). The purification by Prep-HPLC afforded the product (14 mg, yield 89%) as a white solid: [α]₅₄₆²⁵=−2.4 (c 2.0, MeOH); ¹H NMR (400 MHz, Methanol-d₄) δ 7.71 (q, J=1.5 Hz, 1H), 7.59-7.53 (m, 1H), 7.45-7.35 (m, 6H), 7.26-7.22 (m, 1H), 5.25 (q, J=7.0 Hz, 1H), 4.36 (s, 2H), 2.37 (s, 3H), 1.59 (d, J=7.1 Hz, 3H); ¹³C NMR (100 MHz, Methanol-d₄) δ 170.32, 147.64, 146.27, 139.89, 137.97, 135.35, 134.91, 133.53, 131.71, 130.52, 129.57, 126.86, 125.71, 125.18, 124.94, 124.73, 122.66, 50.78, 38.85, 22.27, 19.19; HRMS (ESI) calcd for C₂₁H₂₃N₃OS [M+H]⁺366.1635, found 366.1635.

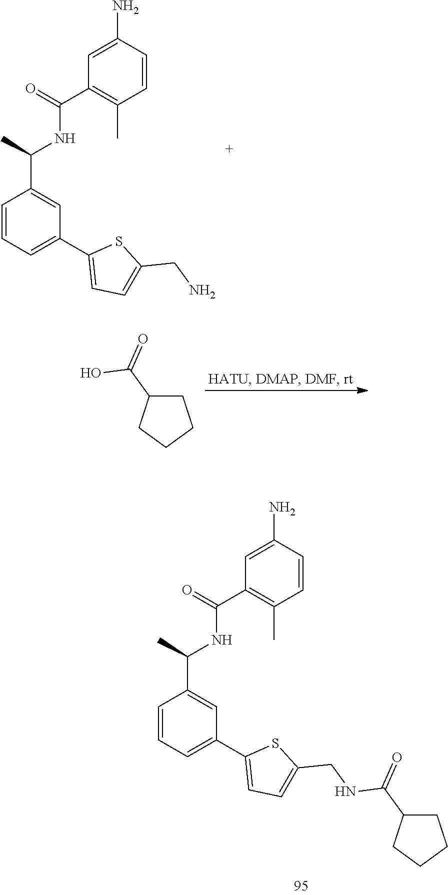 [SMILES: Cc1ccc(N)cc1C(=O)N[C@H](C)c1cccc(-c2ccc(CN)s2)c1 ; O=C(O)C1CCCC1 ; Cc1ccc(N)cc1C(=O)N[C@H](C)c1cccc(-c2ccc(CNC(=O)C3CCCC3)s2)c1]

(R)-5-amino-N-(1-(3-(5-(cyclopentanecarboxamidomethyl)thiophen-2-yl)phenyl)ethyl)-2-methylbenzamide. (R)-5-amino-N-(1-(3-(5-(aminomethyl)thiophen-2-yl)phenyl)ethyl)-2-methylbenzamide (20 mg, 0.05 mmol), cyclopentanecarboxylic acid (6 mg, 0.05 mmol), HATU (19 mg, 0.05 mmol), and DMAP (18 mg, 0.15 mmol) was subjected to general amine coupling procedure with DMF (2 mL). The purification by Prep-HPLC gave the product (20 mg, yield 87%) as a white solid: [α]₅₄₆²⁵=+23.5 (c 0.8, MeOH); ¹H NMR (400 MHz, Methanol-d₄) δ 7.66-7.61 (m, 1H), 7.50-7.45 (m, 1H), 7.38-7.28 (m, 2H), 7.22 (d, J=3.6 Hz, 1H), 6.98-6.91 (m, 2H), 6.74-6.66 (m, 2H), 5.24-5.14 (m, 1H), 4.53-4.49 (m, 2H), 2.65 (ddd, J=13.3, 10.5, 7.5 Hz, 1H), 2.20 (s, 3H), 1.91-1.82 (m, 2H), 1.80-1.69 (m, 4H), 1.65-1.56 (m, 2H), 1.53 (d, J=7.1 Hz, 3H); ¹³C NMR (100 MHz, Methanol-d₄) δ 178.96, 172.83, 146.46, 146.32, 144.88, 143.02, 138.53, 136.03, 132.29, 130.17, 127.60, 126.34, 125.44, 125.17, 124.28, 123.80, 118.01, 115.04, 50.39, 46.46, 39.13, 31.43, 27.02, 22.42, 18.67; HRMS (ESI) calcd for C₂₇H₃₂N₃O₂S [M+H]⁺462.2210, found 462.2214.

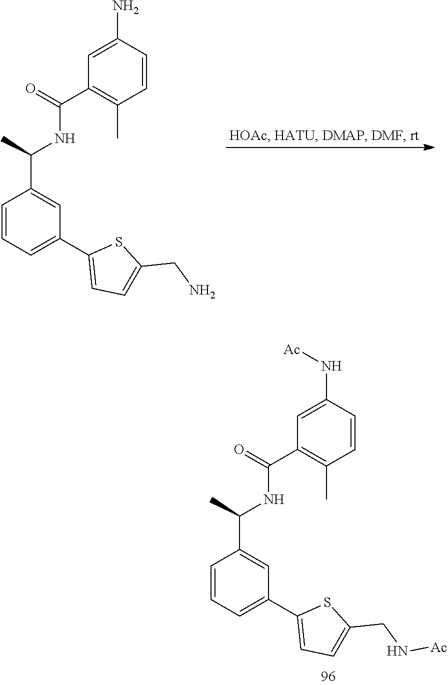 [SMILES: Cc1ccc(N)cc1C(=O)N[C@H](C)c1cccc(-c2ccc(CN)s2)c1 ; CC(=O)NCc1ccc(-c2cccc([C@@H](C)NC(=O)c3cc(NC(C)=O)ccc3C)c2)s1]

(R)-5-acetamido-N-(1-(3-(5-(acetamidomethyl)thiophen-2-yl)phenyl)ethyl)-2-methylbenzamide. (R)-5-amino-N-(1-(3-(5-(aminomethyl)thiophen-2-yl)phenyl)ethyl)-2-methylbenzamide (20 mg, 0.05 mmol), HOAc (6 mg, 0.10 mmol), HATU (19 mg, 0.05 mmol), and DMAP (18 mg, 0.15 mmol) was subjected to general amine coupling procedure with DMF (2 mL). The purification by Prep-HPLC gave the compound (17 mg, yield 76%) as a white solid: ¹H NMR (400 MHz, Methanol-d₄) δ 7.66-7.63 (m, 1H), 7.58 (d, J=2.3 Hz, 1H), 7.50-7.43 (m, 2H), 7.38-7.30 (m, 2H), 7.23 (d, J=3.6 Hz, 1H), 7.17 (d, J=8.3 Hz, 1H), 6.96-6.92 (m, 1H), 5.21 (q, J=7.0 Hz, 1H), 4.51 (d, J=0.9 Hz, 2H), 2.29 (s, 3H), 2.10 (s, 3H), 1.97 (s, 3H), 1.55 (d, J=7.1 Hz, 3H); ¹³C NMR (100 MHz, Methanol-d₄) δ 172.93, 171.89, 171.72, 146.21, 144.94, 142.48, 138.31, 137.56, 136.02, 132.27, 132.05, 130.24, 127.87, 126.32, 125.25, 124.32, 123.91, 122.43, 119.89, 50.50, 39.14, 23.72, 22.50, 22.40, 19.06; HRMS (ESI) calcd for C₂₅H₂₈N₃O₃S [M+H]⁺450.1846, found 450.1852.

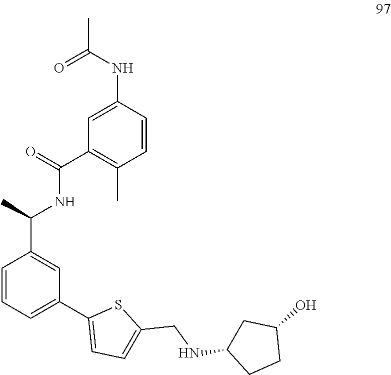 [SMILES: CC(=O)Nc1ccc(C)c(C(=O)N[C@H](C)c2cccc(-c3ccc(CN[C@H]4CC[C@@H](O)C4)s3)c2)c1]

5-acetamido-N—((R)-1-(3-(5-((((1S,3R)-3-hydroxycyclopentyl)amino)methyl)thiophen-2-yl)phenyl)ethyl)-2-methylbenzamide. [α]₅₄₆²⁵=−18.7 (c 0.5, MeCOH); ¹H NMR (400 MHz, Methanol-d₄) δ 8.36 (s, 1H), 7.73-7.66 (m, 2H), 7.58-7.53 (m, 1H), 7.44-7.36 (m, 4H), 7.27 (d, J=3.7 Hz, 1H), 7.18 (d, J=8.2 Hz, 1H), 5.22 (q, J=7.0 Hz, 1H), 4.45 (s, 2H), 4.33 (p, J=4.0 Hz, 1H), 3.68 (p, J=7.0 Hz, 1H), 2.30 (s, 3H), 2.27-2.14 (m, 2H), 2.12 (s, 3H), 2.03-1.92 (m, 1H), 1.89-1.80 (m, 3H), 1.56 (d, J=7.0 Hz, 3H); ¹³C NMR (100 MHz, Methanol-d₄) δ 171.96, 171.77, 148.41, 146.57, 138.25, 137.60, 135.14, 132.97, 132.74, 132.29, 132.08, 130.49, 126.96, 125.56, 124.83, 124.80, 122.41, 119.91, 72.52, 58.47, 50.53, 45.17, 39.27, 34.42, 28.32, 23.74, 22.35, 19.05; HRMS (ESI) calcd for C₂₈H₃₄N₃O₃S [M+H]⁺ 492.2315, found 492.2319.

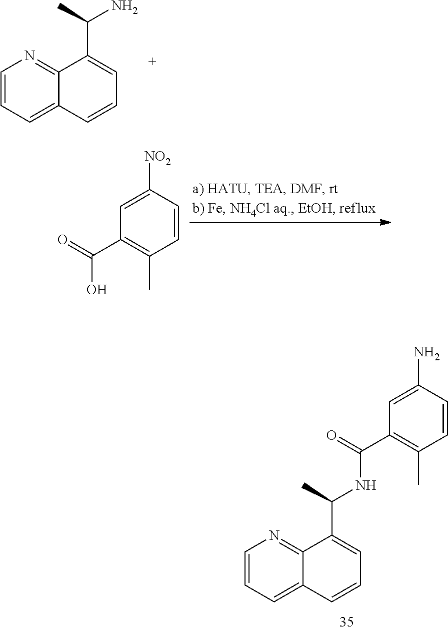 [SMILES: C[C@@H](N)c1cccc2cccnc12 ; Cc1ccc([N+](=O)[O-])cc1C(=O)O ; Cc1ccc(N)cc1C(=O)N[C@H](C)c1cccc2cccnc12]

(R)-5-amino-2-methyl-N-(1-(quinolin-8-yl)ethyl)benzamide. (R)-1-(quinolin-8-yl)ethan-1-amine (60 mg, 0.29 mmol), 2-methyl-5-nitrobenzoic acid (68 mg, 0.37 mmol), HATU (215 mg, 0.58 mmol), TEA (100 μL), and DMAP (5 mg, 0.04 mmol) was subjected to general amine coupling procedure with DMF (5 mL). After purification by Prep-HPLC, the product was applied to the general Aryl Nitro reduction procedure with ethanol/saturated aq. NH₄Cl (4 mL/1 mL) and Iron Powder (67 mg, 1.20 mmol). The purification by Prep-HPLC gave the product (55 mg, yield 63% for 2 steps) as a white solid: ¹H NMR (400 MHz, Methanol-d₄) δ 8.91 (dd, J=4.1, 2.0 Hz, 1H), 8.37-8.22 (m, 1H), 7.91 (d, J=2.4 Hz, 1H), 7.86-7.81 (m, 1H), 7.77 (d, J=7.1 Hz, 1H), 7.62-7.55 (m, 1H), 7.50 (dt, J=8.1, 4.0 Hz, 1H), 7.16 (d, J=2.5 Hz, 1H), 6.24 (q, J=7.9, 6.9 Hz, 1H), 2.38 (s, 3H), 1.66 (d, J=7.0 Hz, 3H); ¹³C NMR (100 MHz, Methanol-d₄) δ 162.01, 150.68, 150.62, 146.69, 144.42, 143.26, 142.50, 141.69, 138.06, 136.20, 136.09, 130.14, 128.57, 127.54, 127.19, 122.74, 122.43, 49.00, 47.98, 22.64, 20.47; HRMS (ESI) calcd for C₁₉H₂₀N₃O [M+H]⁺306.1601, found 306.1600.

### Example NMR Spectrum.

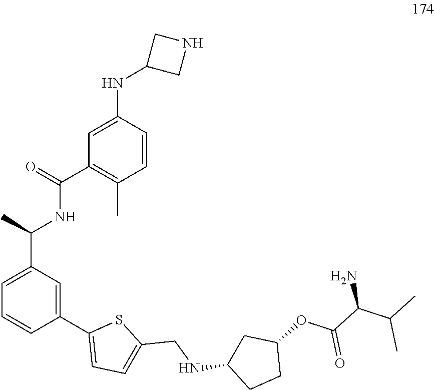 [SMILES: Cc1ccc(NC2CNC2)cc1C(=O)N[C@H](C)c1cccc(-c2ccc(CN[C@H]3CC[C@@H](OC(=O)[C@@H](N)C(C)C)C3)s2)c1]

¹H NMR (400 MHz, MeOD) δ 7.68 (s, 1H), 7.54 (d, J=6.7 Hz, 1H), 7.43-7.35 (m, 3H), 7.26 (s, 1H), 7.03 (d, J=8.1 Hz, 1H), 6.61-6.53 (m, 2H), 5.28 (s, 1H), 5.21 (q, J=7.0 Hz, 1H), 4.54-4.27 (m, 5H), 4.04-3.82 (m, 3H), 3.67 (s, 1H), 2.63 (s, 1H), 2.20 (s, 5H), 2.06-1.91 (m, 4H), 1.55 (d, J=7.0 Hz, 3H), 1.07 (d, J=6.9 Hz, 6H).

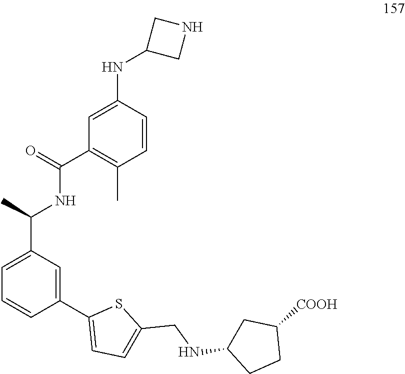 [SMILES: Cc1ccc(NC2CNC2)cc1C(=O)N[C@H](C)c1cccc(-c2ccc(CN[C@H]3CC[C@@H](C(=O)O)C3)s2)c1]

¹H NMR (500 MHz, MeOD) δ 7.73-7.67 (m, 1H), 7.58-7.52 (m, 1H), 7.43-7.34 (m, 3H), 7.28 (d, J=3.7 Hz, 1H), 7.04 (d, J=8.3 Hz, 1H), 6.61 (dd, J=8.3, 2.5 Hz, 1H), 6.46 (d, J=2.5 Hz, 1H), 5.22 (q, J=7.0 Hz, 1H), 4.49-4.26 (m, 5H), 3.95-3.87 (m, 1H), 3.77-3.67 (m, 3H), 2.28-2.18 (m, 4H), 2.16-2.09 (m, 2H), 2.07-1.97 (m, 3H), 1.55 (d, J=7.1 Hz, 3H).

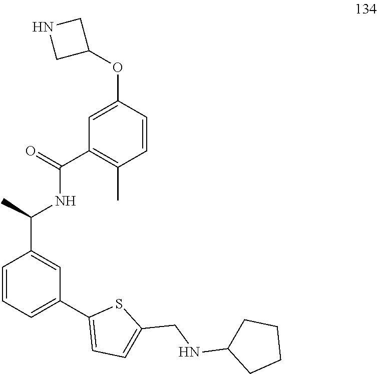 [SMILES: Cc1ccc(OC2CNC2)cc1C(=O)N[C@H](C)c1cccc(-c2ccc(CNC3CCCC3)s2)c1]

¹H NMR (500 MHz, MeOD) δ 7.69 (t, J=1.9 Hz, 1H), 7.55 (dt, J=7.3, 1.7 Hz, 1H), 7.44-7.37 (m, 3H), 7.25 (d, J=3.6 Hz, 1H), 7.19 (dd, J=8.4, 4.6 Hz, 1H), 6.86-6.76 (m, 2H), 5.22 (q, J=7.1 Hz, 1H), 5.13 (td, J=6.3, 2.8 Hz, 1H), 4.48-4.37 (m, 4H), 4.07 (dd, J=11.6, 4.7 Hz, 2H), 3.61-3.53 (m, 1H), 2.26 (s, 3H), 2.18-2.10 (m, 2H), 1.87-1.78 (m, 2H), 1.72-1.62 (m, 4H), 1.56 (d, J=7.1 Hz, 3H); ¹³C NMR (125 MHz, MeOD) δ 171.59, 155.36, 147.83, 146.49, 139.38, 135.30, 134.31, 133.18, 132.28, 130.48, 130.02, 126.88, 125.63, 124.88, 124.70, 117.15, 114.67, 69.39, 59.87, 54.44, 50.64, 45.66, 31.04, 25.04, 22.30, 18.72.

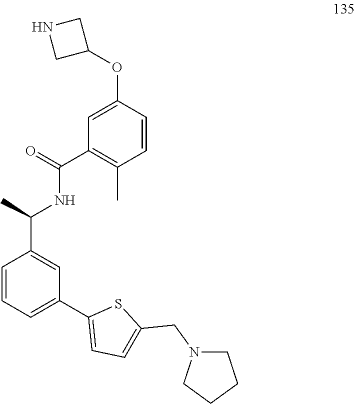 [SMILES: Cc1ccc(OC2CNC2)cc1C(=O)N[C@H](C)c1cccc(-c2ccc(CN3CCCC3)s2)c1]

¹H NMR (500 MHz, MeOD) δ 7.69 (t, J=1.8 Hz, 1H), 7.56 (dt, J=7.3, 1.8 Hz, 1H), 7.44-7.36 (m, 3H), 7.26 (d, J=3.7 Hz, 1H), 7.18 (t, J=7.6 Hz, 1H), 6.85-6.78 (m, 2H), 5.22 (q, J=7.1 Hz, 1H), 5.17-5.08 (m, 1H), 4.53-4.46 (m, 4H), 4.15-4.08 (m, 2H), 3.27 (dt, J=6.8, 4.0 Hz, 4H), 2.27 (s, 3H), 2.09-2.00 (m, 4H), 1.56 (dd, J=7.1, 1.9 Hz, 3H); ¹³C NMR (125 MHz, MeOD) δ 171.52, 164.60, 155.27, 148.16, 146.49, 139.37, 135.25, 133.19, 132.97, 130.47, 130.11, 127.02, 125.64, 125.62, 124.80, 124.67, 117.14, 114.72, 69.12, 54.42, 54.32, 53.33, 50.61, 24.02, 22.34, 18.75.

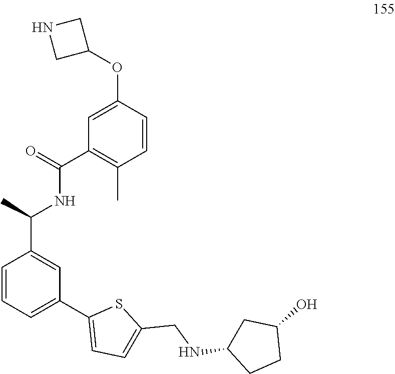 [SMILES: Cc1ccc(OC2CNC2)cc1C(=O)N[C@H](C)c1cccc(-c2ccc(CN[C@H]3CC[C@@H](O)C3)s2)c1]

¹H NMR (500 MHz, MeOD) δ 7.69 (t, J=1.9 Hz, 1H), 7.55 (dt, J=7.4, 1.7 Hz, 1H), 7.44-7.36 (m, 3H), 7.24 (d, J=3.7 Hz, 1H), 7.19 (d, J=8.3 Hz, 1H), 6.87-6.79 (m, 2H), 5.22 (q, J=7.1 Hz, 1H), 5.16-5.10 (m, 1H), 4.50-4.44 (m, 2H), 4.41-4.37 (m, 2H), 4.32 (dq, J=5.4, 4.0 Hz, 1H), 4.12-4.05 (m, 2H), 3.65-3.58 (m, 1H), 2.29-2.20 (m, 4H), 2.18-2.10 (m, 1H), 1.99-1.89 (m, 1H), 1.87-1.77 (m, 3H), 1.56 (d, J=7.0 Hz, 3H); ¹³C NMR (125 MHz, MeOD) δ 171.57, 155.32, 147.80, 146.48, 139.40, 135.32, 133.19, 132.25, 130.48, 130.07, 126.87, 125.62, 124.87, 124.70, 117.16, 114.67, 72.65, 69.29, 58.46, 54.43, 50.64, 45.46, 39.69, 34.46, 28.73, 22.30, 18.72.

### Biological Assays

The compounds of the present disclosure were then tested for their inhibition potency and affinity for PLpro from SARS-CoV-2. SARS-CoV-2 PLpro expression and purification was as described: pET11a vector containing SARS-CoV-2 PLpro protein (pp1ab aa 1564-1878) with N-terminal, TEV-cleavable His-tag was transformed into BL21(DE3) cells and maintained in media containing 100 ug/mL carbenicillin. Protein expression was induced using an auto-induction protocol modified from Studier et al.46 Briefly, 1 mL day cultures were used to inoculate a 2 L flask of 500 mL of Super LB containing 100 ug/mL carbenicillin. Cells were grown for 24 h at 25° C. and then harvested by centrifugation. All steps of SARS-CoV2 PLpro purification were performed at 4° C. Protein yield at each step was monitored by Bradford assay using BSA as a standard. Frozen cells pellets were lysed by sonication in Buffer A (50 mM HEPES, pH 8, 0.5 M NaCl) containing 10 ug/mL lysozyme. The lysate was clarified by centrifugation and loaded onto a 2-mL HiTrap Talon crude column equilibrated with Buffer A. Bound His6-PLpro was eluted with a linear gradient of 0-150 mM imidazole in Buffer A, and fractions containing His6-PLpro were pooled and exchanged into cleavage buffer (20 mM Tris-HCl pH 8.5, 5 mM DTT, 0.5 mM EDTA, 5% glycerol). A 1:100 molar ratio of TEV protease to PLpro was incubated at 4° C. overnight to cleave the His6-tag. To remove the tag and TEV protease, the reaction was loaded onto a UNO-Q column equilibrated with 20 mM Tris HCl, pH 8.5, 3 mM DTT. Cleaved PLpro eluted first in a gradient from 0-150 mM NaCl over 20 column volumes. Fractions containing cleaved PLpro were pooled and concentrated to 12 mg/mL, frozen in liquid nitrogen, and stored at −80° C.

The PLpro primary assay using PLpro as prepared above was conducted as described below and provided the data described in Table 7 in the column: “Enzymatic assay IC₅₀”. The PLpro primary assay, which measures protease activity with the short peptide substrate Z-RLRGG-AMC (SEQ ID NO. 4) (Bachem), was performed in black, flat-bottom 384-well plates containing a final reaction volume of 50 μL. The assays were assembled at room temperature as follows: 40 μL of 50 nM PLpro in Buffer B (50 mM HEPES, pH 7.5, 0.1 mg/mL BSA, 0.01% Triton-X 100, and 5 mM DTT) was dispensed into wells containing 0.1-1 μL of inhibitor in DMSO or appropriate controls. The enzyme was incubated with inhibitor for 10 min prior to substrate addition. Reactions were initiated with 10 μL of 62.5 μM RLRGG-AMC (SEQ ID NO. 4) in Buffer B. Plates were shaken vigorously for 30 s, and fluorescence from the release of AMC from peptide was monitored continuously for 15 min on a Tecan Infinite M200 Pro plate reader (λ_{excitation}=360 nm; λₑₘᵢₛₛᵢₒₙ=460 nm). Slopes from the linear portions of each progress curve were recorded and normalized to plate-based controls. Positive control wells, representing 100% inhibition, included 10 μM GRL0617; negative control wells, representing 0% inhibition, included vehicle.

The selectivity of the most potent inhibitors was tested against the human deubiquitinating enzymes USP7 and USP14 (Boston Biochem). Assay conditions were similar to the PLpro primary assay, with the following substitutions: USP7 assays contained 4 nM USP7 and 0.5 uM Ub-AMC (Boston Biochem); USP14 assays contained 1.7 uM USP14, 4 uM Ub-AMC, and the addition of 5% glycerol to Buffer B. PLpro activity with ISG15-AMC and Ub-AMC were assayed in a manner similar to the PLpro primary assay. PLpro and substrate concentrations were modified as follows: 80 nM PLpro was assayed with 0.5 uM Ub-AMC, and 4 nM PLpro was assayed with 0.5 uM ISG15-AMC.

Secondary analysis of PLpro interaction was performed by analysis of binding affinity using Surface Plasmon Resonance (SPR) providing data for Table 7 column: “SPR binding assay K_{D}”. The His-tagged SARS-CoV-2 PLpro enzyme was initially prepared in phosphate buffer and diluted to 50 pg/mL with 10 mM sodium acetate (pH 5.5) and immobilized on a CM5 sensor chip by standard amine-coupling with running buffer PBSP (10 mM phosphate, pH 7.4, 2.7 mM KCl, 137 mM NaCl, 0.05% Tween-20). The CM5 sensor chip surface was first activated by 1-ethyl-3-(3-dimethylaminopropyl) carbodiimide hydrochloride (EDC)/N-hydroxy succinimide (NHS) mixture using a Biacore 8K instrument (Cytiva). SARS-CoV-2 PLpro enzyme was immobilized to flow channels 1 through 4 followed by ethanolamine blocking on the unoccupied surface area, and immobilization levels for all four channels were similar at ˜12,000 RU. Each flow channel has its own reference channel, and blank immobilization using EDC/NHS and ethanolamine was done for all reference channels. Compound solutions with a series of increasing concentrations (0.049-30 μM at 2.5-fold dilution) were applied to all active and reference channels in SPR binding buffer (10 mM HEPES, pH 7.4, 150 mM NaCl, and 0.05% Tween-20, 0.5 mM TCEP, and 2% DMSO) at a 30 μL/min flow rate at 25° C. The data were double referenced with a reference channel and zero concentration (2% DMSO) responses, and reference subtracted sensorgrams were fitted with 1 to 1 Langmuir kinetic model using a Biacore Insight evaluation software, producing two rate constants (kₐ and k_{d}). The equilibrium dissociation constants (K_{D}) were determined from two rate constants (K_{D}=k_{d}/kₐ). For steady-state affinity fittings, response units at each concentration were measured during the equilibration phase, and the K_{D} values were determined by fitting the data to a single rectangular hyperbolic curve equation, where y is the response, yₘₐₓ is the maximum response and x is the compound concentration.

$$\\ y = \frac{{y}_{max} \cdot x}{{K}_{D} + x}$$

The data in Table 7 demonstrate the potency for inhibition of PL-pro, which generally correlates with and the binding affinity of the test compounds for PLpro from SARS-CoV-2.

TABLE 7
Biological Assays for Representative Compounds of the Present Disclosure

| | | | SPR |
|---|---|---|---|
| | | | binding |
| | | Enzymatic | assay |
| Ex- | | assay IC₅₀ | K_{D} |
| amples | Structure | (μM) | (μM) |
| GRL- 0167 | 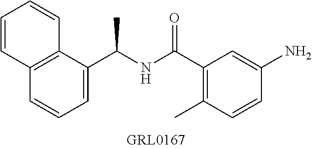 [SMILES: Cc1ccc(N)cc1C(=O)N[C@H](C)c1cccc2ccccc12] | 1.7 | 2.1 |
| 1 | 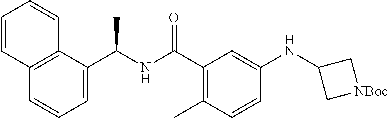 [SMILES: Cc1ccc(NC2CN(C(=O)OC(C)(C)C)C2)cc1C(=O)N[C@H](C)c1cccc2ccccc12] | 6.1 | 16.0 |
| 2 | 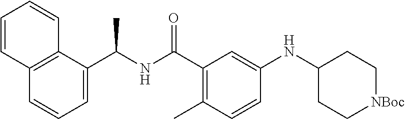 [SMILES: Cc1ccc(NC2CCN(C(=O)OC(C)(C)C)CC2)cc1C(=O)N[C@H](C)c1cccc2ccccc12] | 1.1 | 4.7 |
| 3 | 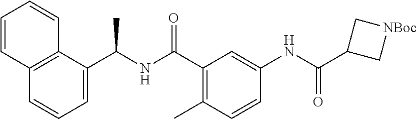 [SMILES: Cc1ccc(NC(=O)C2CN(C(=O)OC(C)(C)C)C2)cc1C(=O)N[C@H](C)c1cccc2ccccc12] | 5.5 | 32.6 |
| 4 | 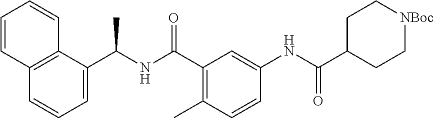 [SMILES: Cc1ccc(NC(=O)C2CCN(C(=O)OC(C)(C)C)CC2)cc1C(=O)N[C@H](C)c1cccc2ccccc12] | 6.0 | 31.1 |
| 5 | 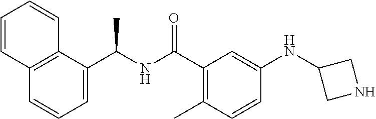 [SMILES: Cc1ccc(NC2CNC2)cc1C(=O)N[C@H](C)c1cccc2ccccc12] | 1.01 | 0.81 |
| 6 | 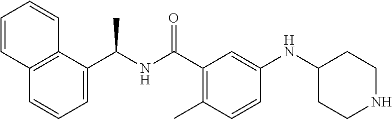 [SMILES: Cc1ccc(NC2CCNCC2)cc1C(=O)N[C@H](C)c1cccc2ccccc12] | 0.6 | 1.8 |
| 7 | 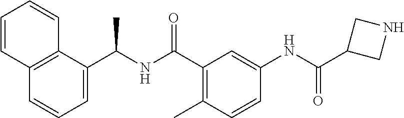 [SMILES: Cc1ccc(NC(=O)C2CNC2)cc1C(=O)N[C@H](C)c1cccc2ccccc12] | 1.2 | 3.1 |
| | 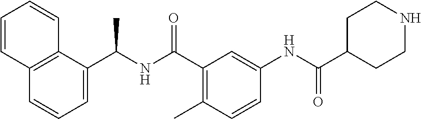 [SMILES: Cc1ccc(NC(=O)C2CCNCC2)cc1C(=O)N[C@H](C)c1cccc2ccccc12] | 0.8 | 5.4 |
| 8 | 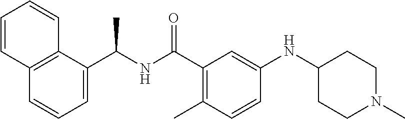 [SMILES: Cc1ccc(NC2CCN(C)CC2)cc1C(=O)N[C@H](C)c1cccc2ccccc12] | 0.7 | 1.3 |
| 10 | 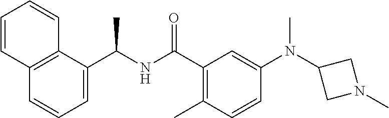 [SMILES: Cc1ccc(N(C)C2CN(C)C2)cc1C(=O)N[C@H](C)c1cccc2ccccc12] | 1.6 | 5.6 |
| 9 | 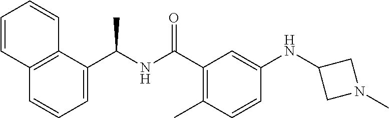 [SMILES: Cc1ccc(NC2CN(C)C2)cc1C(=O)N[C@H](C)c1cccc2ccccc12] | 4.3 | 3.4 |
| 11 | 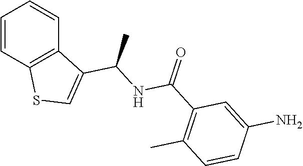 [SMILES: Cc1ccc(N)cc1C(=O)N[C@H](C)c1csc2ccccc12] | 3.3 | 6.7 |
| 12 | 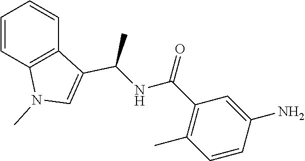 [SMILES: Cc1ccc(N)cc1C(=O)N[C@H](C)c1cn(C)c2ccccc12] | <10 | 29.7 |
| 17 | 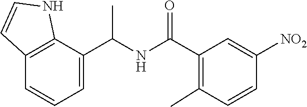 [SMILES: Cc1ccc([N+](=O)[O-])cc1C(=O)NC(C)c1cccc2cc[nH]c12] | >10 | >1000 |
| — | 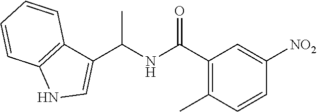 [SMILES: Cc1ccc([N+](=O)[O-])cc1C(=O)NC(C)c1c[nH]c2ccccc12] | >>100 | — |
| 18 | 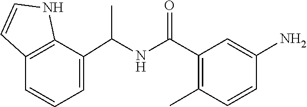 [SMILES: Cc1ccc(N)cc1C(=O)NC(C)c1cccc2cc[nH]c12] | 4.7 | 30.3 |
| 19 | 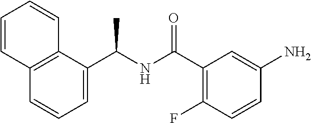 [SMILES: C[C@@H](NC(=O)c1cc(N)ccc1F)c1cccc2ccccc12] | >>100 | >1000 |
| 20 | 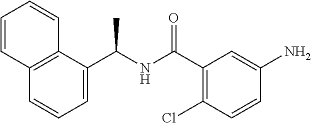 [SMILES: C[C@@H](NC(=O)c1cc(N)ccc1Cl)c1cccc2ccccc12] | 4.8 | 2.0 |
| 21 | 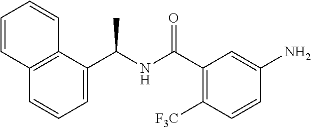 [SMILES: C[C@@H](NC(=O)c1cc(N)ccc1C(F)(F)F)c1cccc2ccccc12] | >10 | 454 |
| 13 | 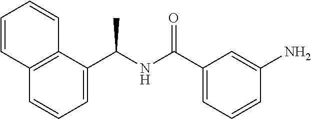 [SMILES: C[C@@H](NC(=O)c1cccc(N)c1)c1cccc2ccccc12] | >>100 | |
| 14 | 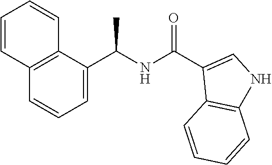 [SMILES: C[C@@H](NC(=O)c1c[nH]c2ccccc12)c1cccc2ccccc12] | >>100 | |
| 15 | 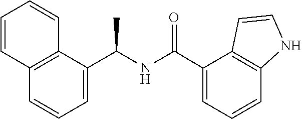 [SMILES: C[C@@H](NC(=O)c1cccc2[nH]ccc12)c1cccc2ccccc12] | >10 | 88.3 |
| 16 | 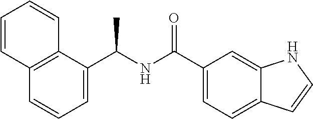 [SMILES: C[C@@H](NC(=O)c1ccc2cc[nH]c2c1)c1cccc2ccccc12] | >10 | 418 |
| 22 | 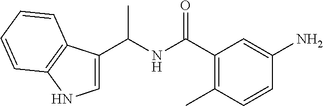 [SMILES: Cc1ccc(N)cc1C(=O)NC(C)c1c[nH]c2ccccc12] | ~100 | >1000 |
| 23 | 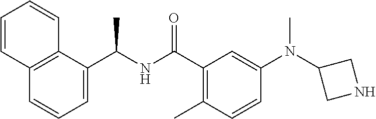 [SMILES: Cc1ccc(N(C)C2CNC2)cc1C(=O)N[C@H](C)c1cccc2ccccc12] | 2.4 | 2.8 |
| 24 | 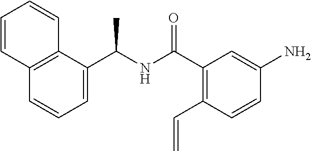 [SMILES: C=Cc1ccc(N)cc1C(=O)N[C@H](C)c1cccc2ccccc12] | >10 | 54.6 |
| 25 | 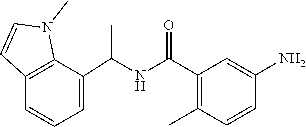 [SMILES: Cc1ccc(N)cc1C(=O)NC(C)c1cccc2ccn(C)c12] | >>100 | |
| 26 | 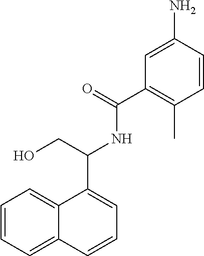 [SMILES: Cc1ccc(N)cc1C(=O)NC(CO)c1cccc2ccccc12] | ~100 | 39.1 |
| 27 | 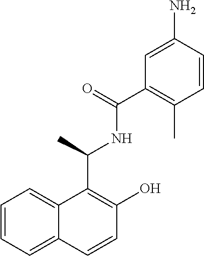 [SMILES: Cc1ccc(N)cc1C(=O)N[C@H](C)c1c(O)ccc2ccccc12] | >>100 | 184 |
| 28 | 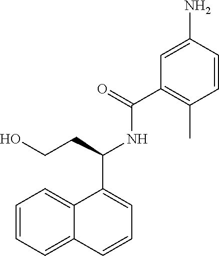 [SMILES: Cc1ccc(N)cc1C(=O)N[C@H](CCO)c1cccc2ccccc12] | ~100 | 721 |
| 29 | 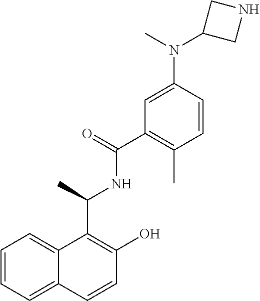 [SMILES: Cc1ccc(N(C)C2CNC2)cc1C(=O)N[C@H](C)c1c(O)ccc2ccccc12] | <100 | 240 |
| 30 | 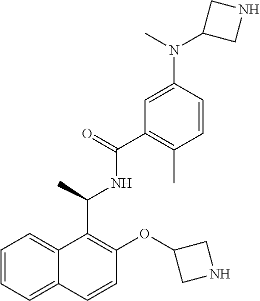 [SMILES: Cc1ccc(N(C)C2CNC2)cc1C(=O)N[C@H](C)c1c(OC2CNC2)ccc2ccccc12] | >>100 | |
| 31 | 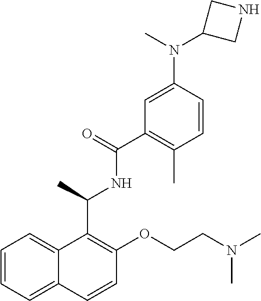 [SMILES: Cc1ccc(N(C)C2CNC2)cc1C(=O)N[C@H](C)c1c(OCCN(C)C)ccc2ccccc12] | NI | |
| 32 | 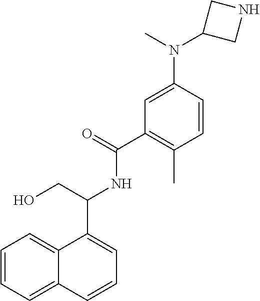 [SMILES: Cc1ccc(N(C)C2CNC2)cc1C(=O)NC(CO)c1cccc2ccccc12] | >100 | 186 |
| 33 | 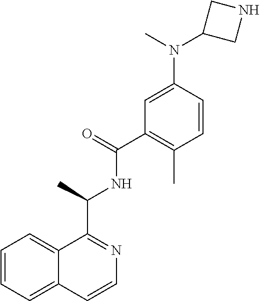 [SMILES: Cc1ccc(N(C)C2CNC2)cc1C(=O)N[C@H](C)c1nccc2ccccc12] | 56 | 19.6 |
| 34 | 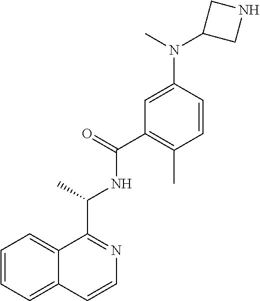 [SMILES: Cc1ccc(N(C)C2CNC2)cc1C(=O)N[C@@H](C)c1nccc2ccccc12] | NI | 889 |
| 35 | 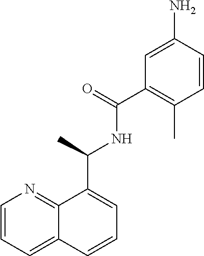 [SMILES: Cc1ccc(N)cc1C(=O)N[C@H](C)c1cccc2cccnc12] | NI | >1000 |
| 36 | 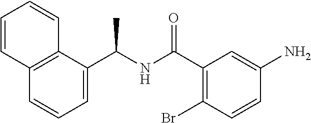 [SMILES: C[C@@H](NC(=O)c1cc(N)ccc1Br)c1cccc2ccccc12] | 21 | 83.0 |
| 37 | 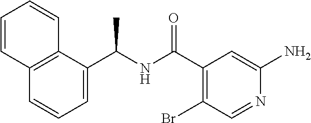 [SMILES: C[C@@H](NC(=O)c1cc(N)ncc1Br)c1cccc2ccccc12] | 7 | 29.6 |
| 38 | 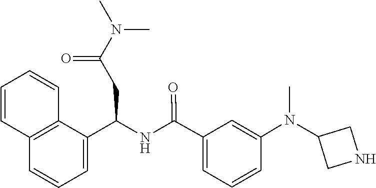 [SMILES: CN(C)C(=O)C[C@@H](NC(=O)c1cccc(N(C)C2CNC2)c1)c1cccc2ccccc12] | NI | >1000 |
| 39 | 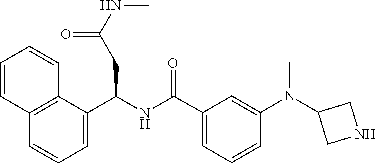 [SMILES: CNC(=O)C[C@@H](NC(=O)c1cccc(N(C)C2CNC2)c1)c1cccc2ccccc12] | NI | |
| 40 | 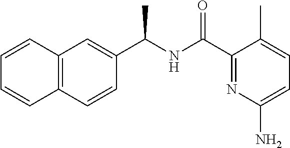 [SMILES: Cc1ccc(N)nc1C(=O)N[C@H](C)c1ccc2ccccc2c1] | 10-100 | >1000 |
| 41 | 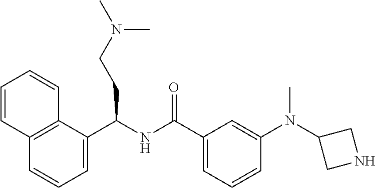 [SMILES: CN(C)CC[C@@H](NC(=O)c1cccc(N(C)C2CNC2)c1)c1cccc2ccccc12] | >>100 | |
| 42 | 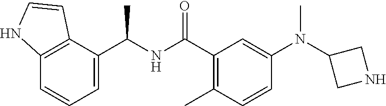 [SMILES: Cc1ccc(N(C)C2CNC2)cc1C(=O)N[C@H](C)c1cccc2[nH]ccc12] | 7 | 32.2 |
| 43 | 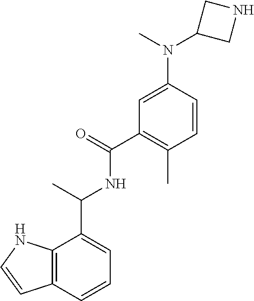 [SMILES: Cc1ccc(N(C)C2CNC2)cc1C(=O)NC(C)c1cccc2cc[nH]c12] | 5.7 | 18.8 |
| 44 | 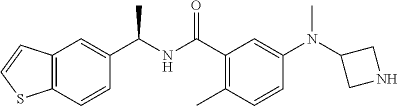 [SMILES: Cc1ccc(N(C)C2CNC2)cc1C(=O)N[C@H](C)c1ccc2sccc2c1] | 6.7 | 18.3 |
| 45 | 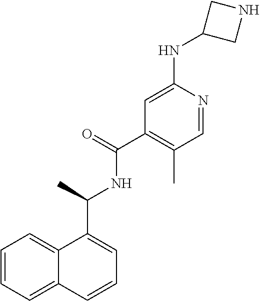 [SMILES: Cc1cnc(NC2CNC2)cc1C(=O)N[C@H](C)c1cccc2ccccc12] | 7.4 | 19.3 |
| 46 | 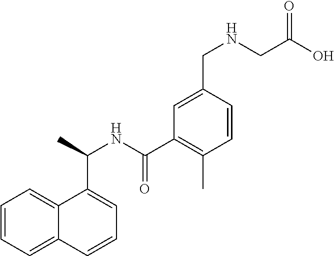 [SMILES: Cc1ccc(CNCC(=O)O)cc1C(=O)N[C@H](C)c1cccc2ccccc12] | 1.2 | |
| 47 | 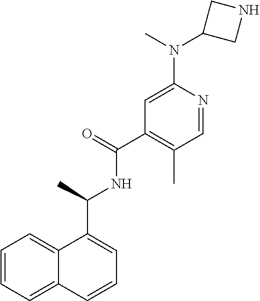 [SMILES: Cc1cnc(N(C)C2CNC2)cc1C(=O)N[C@H](C)c1cccc2ccccc12] | 10~100 | 245 |
| 48 | 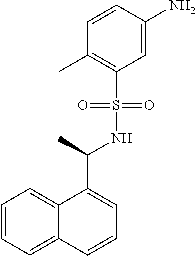 [SMILES: Cc1ccc(N)cc1S(=O)(=O)N[C@H](C)c1cccc2ccccc12] | >>100 | |
| 49 | 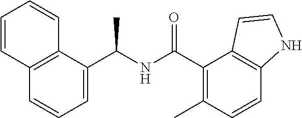 [SMILES: Cc1ccc2[nH]ccc2c1C(=O)N[C@H](C)c1cccc2ccccc12] | 6.3 | 43.2 |
| 50 | 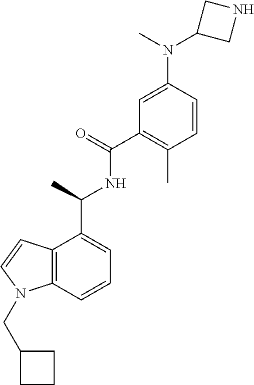 [SMILES: Cc1ccc(N(C)C2CNC2)cc1C(=O)N[C@H](C)c1cccc2c1ccn2CC1CCC1] | 3.3 | 14.1 |
| — | 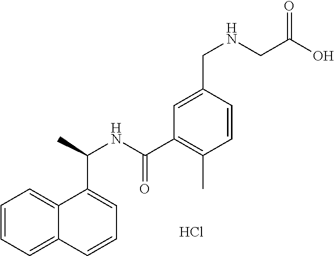 [SMILES: Cc1ccc(CNCC(=O)O)cc1C(=O)N[C@H](C)c1cccc2ccccc12 ; Cl] | 3.9 | 64.3 |
| 51 | 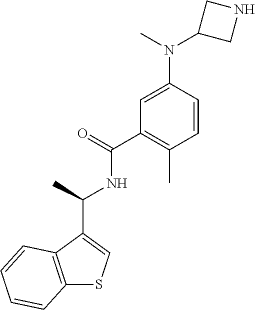 [SMILES: Cc1ccc(N(C)C2CNC2)cc1C(=O)N[C@H](C)c1csc2ccccc12] | 2.4 | 3.4 |
| 52 | 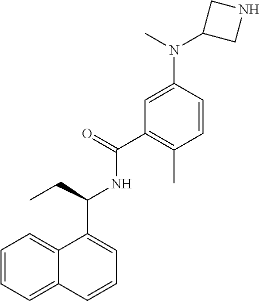 [SMILES: CC[C@@H](NC(=O)c1cc(N(C)C2CNC2)ccc1C)c1cccc2ccccc12] | >>10 | 282 |
| 53 | 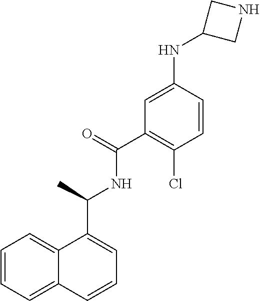 [SMILES: C[C@@H](NC(=O)c1cc(NC2CNC2)ccc1Cl)c1cccc2ccccc12] | 4.1 | 20.6 |
| 54 | 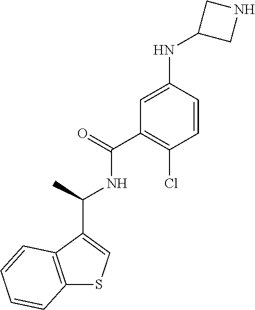 [SMILES: C[C@@H](NC(=O)c1cc(NC2CNC2)ccc1Cl)c1csc2ccccc12] | 8.5 | 33.0 |
| 55 | 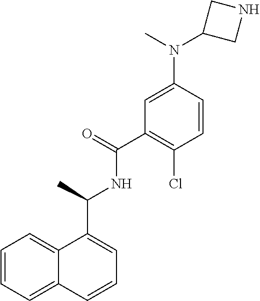 [SMILES: C[C@@H](NC(=O)c1cc(N(C)C2CNC2)ccc1Cl)c1cccc2ccccc12] | 10.7 | 41.9 |
| 56 | 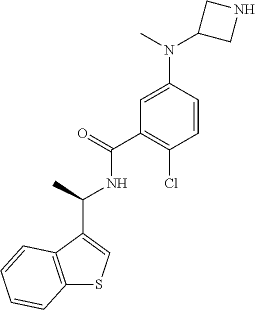 [SMILES: C[C@@H](NC(=O)c1cc(N(C)C2CNC2)ccc1Cl)c1csc2ccccc12] | 10.9 | 64.2 |
| 57 | 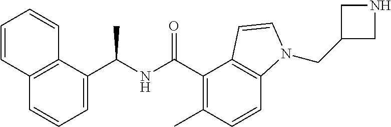 [SMILES: Cc1ccc2c(ccn2CC2CNC2)c1C(=O)N[C@H](C)c1cccc2ccccc12] | 6.4 | 47.4 |
| 58 | 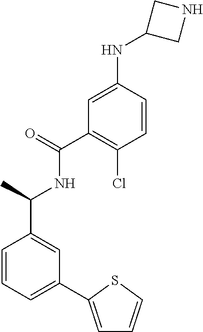 [SMILES: C[C@@H](NC(=O)c1cc(NC2CNC2)ccc1Cl)c1cccc(-c2cccs2)c1] | 2.8 | 35.9 |
| 59 | 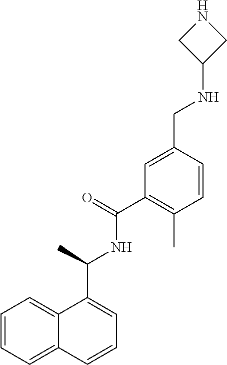 [SMILES: Cc1ccc(CNC2CNC2)cc1C(=O)N[C@H](C)c1cccc2ccccc12] | 1.3 | 6.0 |
| 60 | 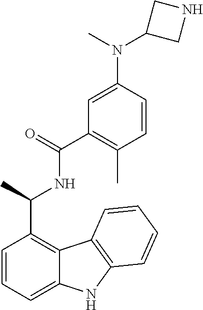 [SMILES: Cc1ccc(N(C)C2CNC2)cc1C(=O)N[C@H](C)c1cccc2[nH]c3ccccc3c12] | 1.6 | 8.4 |
| 61 | 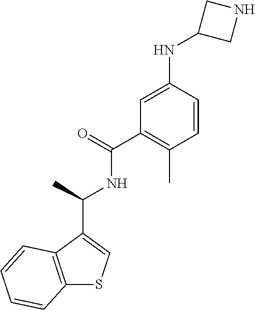 [SMILES: Cc1ccc(NC2CNC2)cc1C(=O)N[C@H](C)c1csc2ccccc12] | 1.9 | 8.4 |
| 62 | 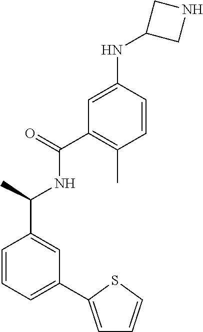 [SMILES: Cc1ccc(NC2CNC2)cc1C(=O)N[C@H](C)c1cccc(-c2cccs2)c1] | 0.65 | 2.7 |
| 63 | 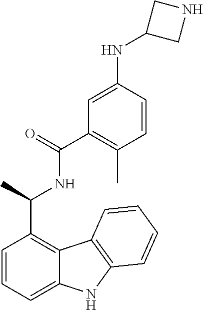 [SMILES: Cc1ccc(NC2CNC2)cc1C(=O)N[C@H](C)c1cccc2[nH]c3ccccc3c12] | 1.8 | 3.9 |
| 64 | 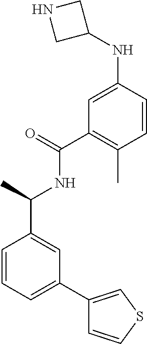 [SMILES: Cc1ccc(NC2CNC2)cc1C(=O)N[C@H](C)c1cccc(-c2ccsc2)c1] | 1.3 | 1.4 |
| 65 | 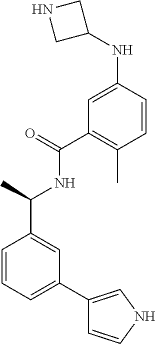 [SMILES: Cc1ccc(NC2CNC2)cc1C(=O)N[C@H](C)c1cccc(-c2cc[nH]c2)c1] | 1.8 | 2.9 |
| 67 | 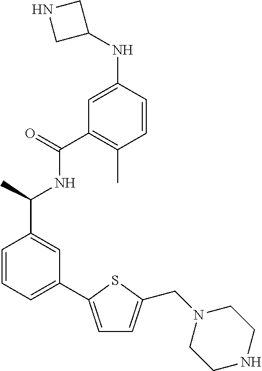 [SMILES: Cc1ccc(NC2CNC2)cc1C(=O)N[C@H](C)c1cccc(-c2ccc(CN3CCNCC3)s2)c1] | 1.2 | 0.8 |
| 68 | 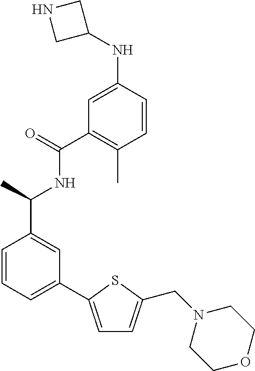 [SMILES: Cc1ccc(NC2CNC2)cc1C(=O)N[C@H](C)c1cccc(-c2ccc(CN3CCOCC3)s2)c1] | 0.93 | 1.6 |
| 69 | 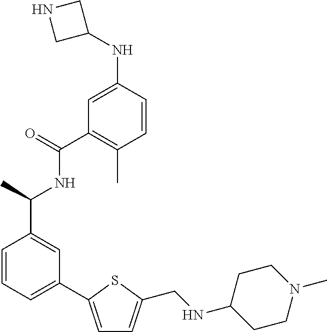 [SMILES: Cc1ccc(NC2CNC2)cc1C(=O)N[C@H](C)c1cccc(-c2ccc(CNC3CCN(C)CC3)s2)c1] | 1.6 | 0.43 |
| — | 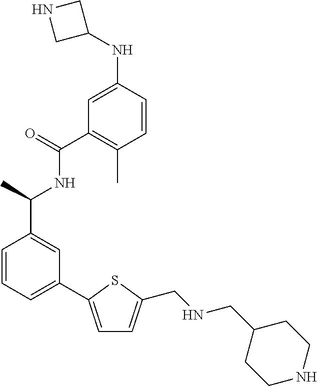 [SMILES: Cc1ccc(NC2CNC2)cc1C(=O)N[C@H](C)c1cccc(-c2ccc(CNCC3CCNCC3)s2)c1] | 2.7 | 0.78 |
| 71 | 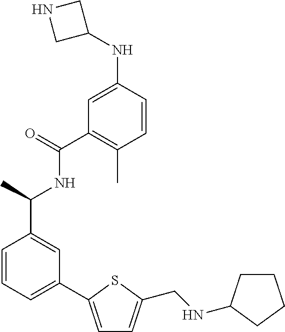 [SMILES: Cc1ccc(NC2CNC2)cc1C(=O)N[C@H](C)c1cccc(-c2ccc(CNC3CCCC3)s2)c1] | 0.38 | 0.11 |
| 72 | 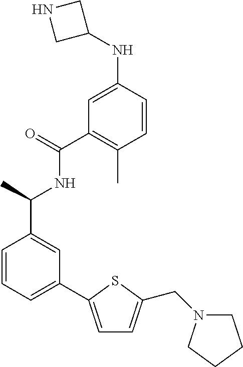 [SMILES: Cc1ccc(NC2CNC2)cc1C(=O)N[C@H](C)c1cccc(-c2ccc(CN3CCCC3)s2)c1] | 0.53 | 0.30 |
| 73 | 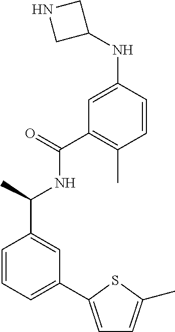 [SMILES: Cc1ccc(-c2cccc([C@@H](C)NC(=O)c3cc(NC4CNC4)ccc3C)c2)s1] | 0.75 | 1.15 |
| 85 | 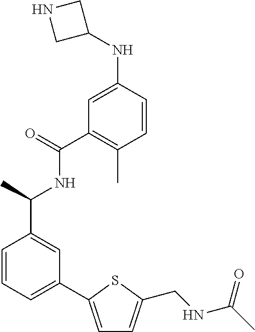 [SMILES: CC(=O)NCc1ccc(-c2cccc([C@@H](C)NC(=O)c3cc(NC4CNC4)ccc3C)c2)s1] | 0.80 | |
| 74 | 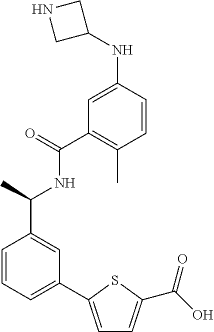 [SMILES: Cc1ccc(NC2CNC2)cc1C(=O)N[C@H](C)c1cccc(-c2ccc(C(=O)O)s2)c1] | 0.97 | 2.06 |
| 75 | 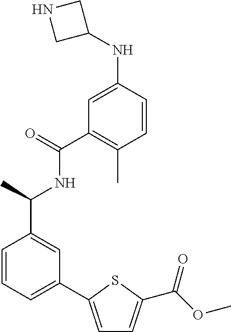 [SMILES: COC(=O)c1ccc(-c2cccc([C@@H](C)NC(=O)c3cc(NC4CNC4)ccc3C)c2)s1] | 0.81 | 2.40 |
| | 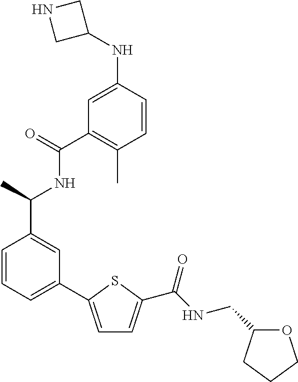 | 0.92 | 1.79 |
| 76 | 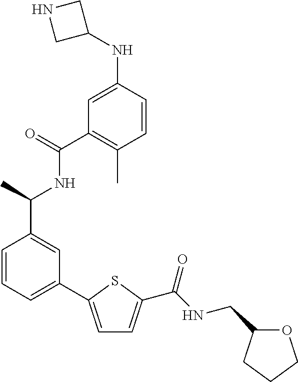 | 0.76 | 0.74 |
| 77 | 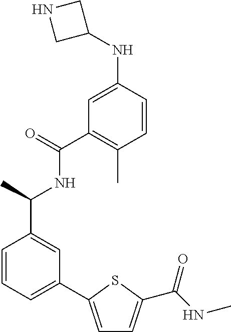 [SMILES: CNC(=O)c1ccc(-c2cccc([C@@H](C)NC(=O)c3cc(NC4CNC4)ccc3C)c2)s1] | 1.05 | 1.02 |
| 78 | 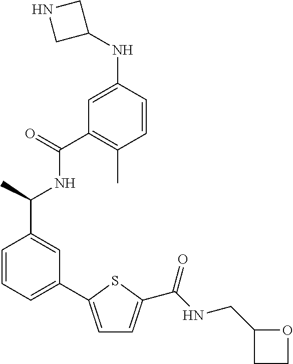 [SMILES: Cc1ccc(NC2CNC2)cc1C(=O)N[C@H](C)c1cccc(-c2ccc(C(=O)NCC3CCO3)s2)c1] | 0.82 | 1.42 |
| 79 | 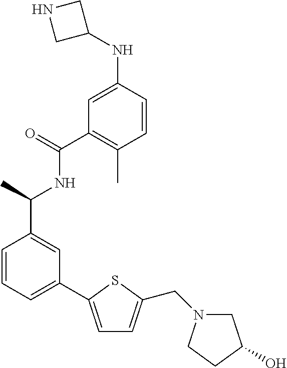 [SMILES: Cc1ccc(NC2CNC2)cc1C(=O)N[C@H](C)c1cccc(-c2ccc(CN3CC[C@@H](O)C3)s2)c1] | 0.64 | 0.76 |
| | 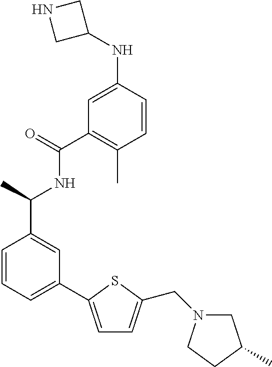 [SMILES: Cc1ccc(NC2CNC2)cc1C(=O)N[C@H](C)c1cccc(-c2ccc(CN3CC[C@@H](C)C3)s2)c1] | 1.11 | 0.78 |
| 81 | 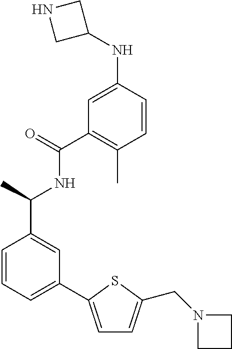 [SMILES: Cc1ccc(NC2CNC2)cc1C(=O)N[C@H](C)c1cccc(-c2ccc(CN3CCC3)s2)c1] | 2.15 | 1.79 |
| 80 | 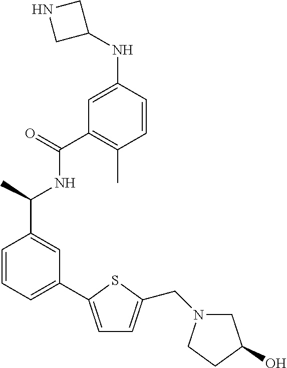 [SMILES: Cc1ccc(NC2CNC2)cc1C(=O)N[C@H](C)c1cccc(-c2ccc(CN3CC[C@H](O)C3)s2)c1] | 0.70 | 0.67 |
| 82 | 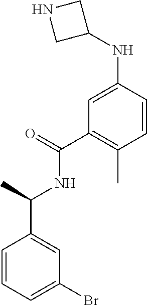 [SMILES: Cc1ccc(NC2CNC2)cc1C(=O)N[C@H](C)c1cccc(Br)c1] | 6.46 | 3.13 |
| 83 | 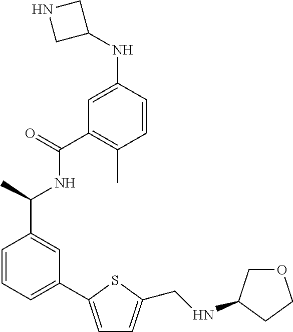 [SMILES: Cc1ccc(NC2CNC2)cc1C(=O)N[C@H](C)c1cccc(-c2ccc(CN[C@@H]3CCOC3)s2)c1] | 0.33 | 1.53 |
| 84 | 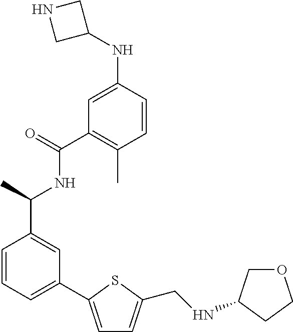 [SMILES: Cc1ccc(NC2CNC2)cc1C(=O)N[C@H](C)c1cccc(-c2ccc(CN[C@H]3CCOC3)s2)c1] | 0.62 | 0.84 |
| 88 | 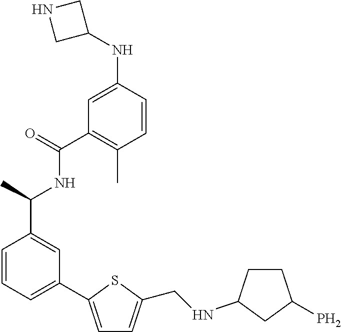 | 0.17 | 0.79 |
| 85 | 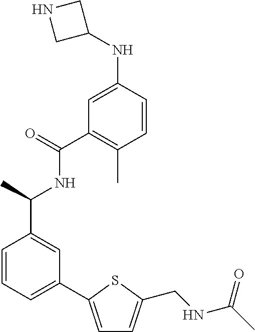 [SMILES: CC(=O)NCc1ccc(-c2cccc([C@@H](C)NC(=O)c3cc(NC4CNC4)ccc3C)c2)s1] | 0.37 | |
| 86 | 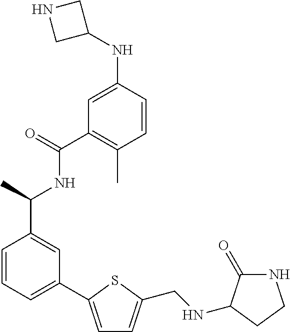 [SMILES: Cc1ccc(NC2CNC2)cc1C(=O)N[C@H](C)c1cccc(-c2ccc(CNC3CCNC3=O)s2)c1] | 0.64 | 1.78 |
| 87 | 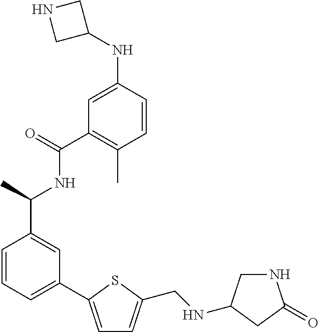 [SMILES: Cc1ccc(NC2CNC2)cc1C(=O)N[C@H](C)c1cccc(-c2ccc(CNC3CNC(=O)C3)s2)c1] | 0.41 | 0.72 |
| 89 | 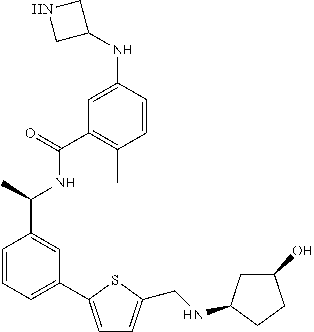 [SMILES: Cc1ccc(NC2CNC2)cc1C(=O)N[C@H](C)c1cccc(-c2ccc(CN[C@@H]3CC[C@H](O)C3)s2)c1] | 0.21 | 0.63 |
| 90 | 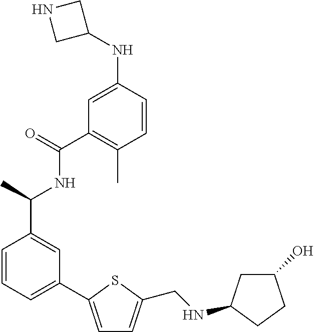 [SMILES: Cc1ccc(NC2CNC2)cc1C(=O)N[C@H](C)c1cccc(-c2ccc(CN[C@@H]3CC[C@@H](O)C3)s2)c1] | 0.43 | 0.53 |
| 91 | 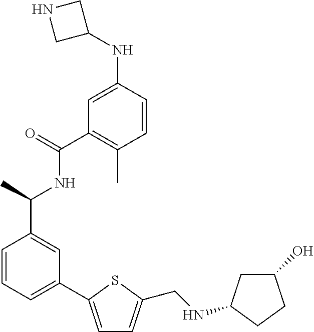 [SMILES: Cc1ccc(NC2CNC2)cc1C(=O)N[C@H](C)c1cccc(-c2ccc(CN[C@H]3CC[C@@H](O)C3)s2)c1] | 0.11 | 0.43 |
| 92 | 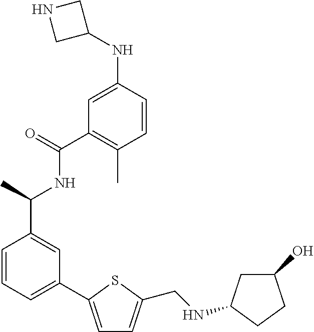 [SMILES: Cc1ccc(NC2CNC2)cc1C(=O)N[C@H](C)c1cccc(-c2ccc(CN[C@H]3CC[C@H](O)C3)s2)c1] | 0.25 | 0.4 |
| 93 | 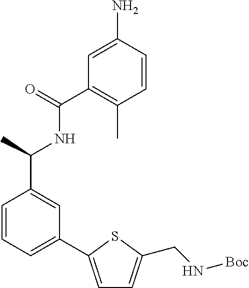 [SMILES: Cc1ccc(N)cc1C(=O)N[C@H](C)c1cccc(-c2ccc(CNC(=O)OC(C)(C)C)s2)c1] | 0.81 | 5.01 |
| 94 | 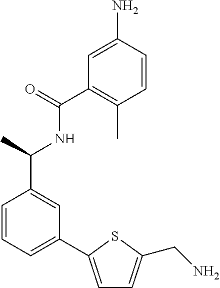 [SMILES: Cc1ccc(N)cc1C(=O)N[C@H](C)c1cccc(-c2ccc(CN)s2)c1] | 1.8 | 2.86 |
| 95 | 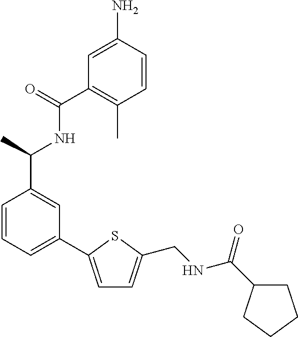 [SMILES: Cc1ccc(N)cc1C(=O)N[C@H](C)c1cccc(-c2ccc(CNC(=O)C3CCCC3)s2)c1] | 1.1 | 2.28 |
| 96 | 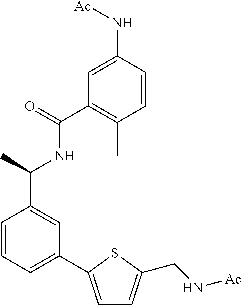 [SMILES: CC(=O)NCc1ccc(-c2cccc([C@@H](C)NC(=O)c3cc(NC(C)=O)ccc3C)c2)s1] | 2.3 | 3.07 |
| 97 | 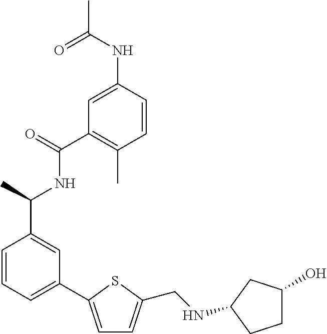 [SMILES: CC(=O)Nc1ccc(C)c(C(=O)N[C@H](C)c2cccc(-c3ccc(CN[C@H]4CC[C@@H](O)C4)s3)c2)c1] | 1.4 | 2.2 |
| | 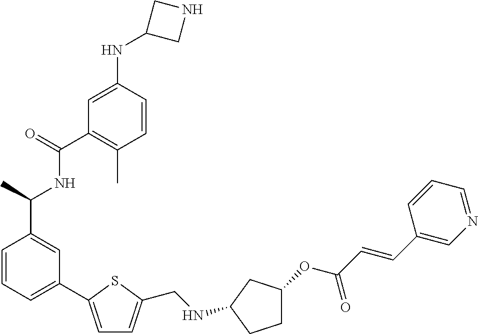 [SMILES: Cc1ccc(NC2CNC2)cc1C(=O)N[C@H](C)c1cccc(-c2ccc(CN[C@H]3CC[C@@H](OC(=O)/C=C/c4cccnc4)C3)s2)c1] | 0.3871 | |
| | 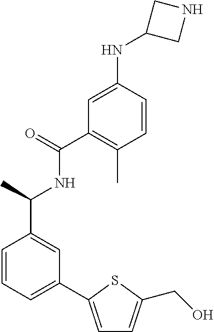 [SMILES: Cc1ccc(NC2CNC2)cc1C(=O)N[C@H](C)c1cccc(-c2ccc(CO)s2)c1] | 1.567 | |
| | 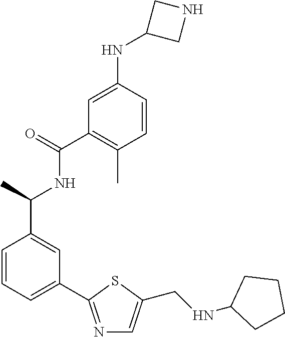 [SMILES: Cc1ccc(NC2CNC2)cc1C(=O)N[C@H](C)c1cccc(-c2ncc(CNC3CCCC3)s2)c1] | 1.560 | |
| | 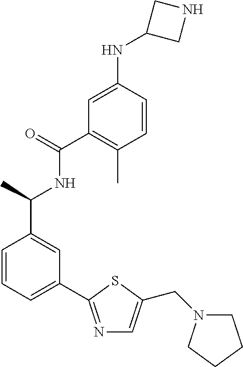 [SMILES: Cc1ccc(NC2CNC2)cc1C(=O)N[C@H](C)c1cccc(-c2ncc(CN3CCCC3)s2)c1] | 3.141 | |
| | 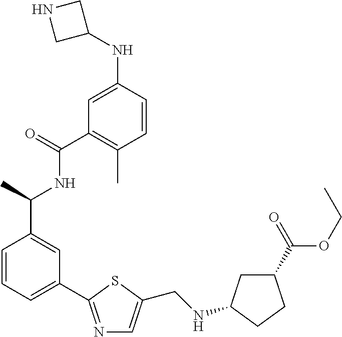 [SMILES: CCOC(=O)[C@@H]1CC[C@H](NCc2cnc(-c3cccc([C@@H](C)NC(=O)c4cc(NC5CNC5)ccc4C)c3)s2)C1] | 0.4956 | |
| | 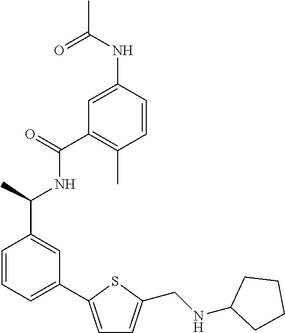 [SMILES: CC(=O)Nc1ccc(C)c(C(=O)N[C@H](C)c2cccc(-c3ccc(CNC4CCCC4)s3)c2)c1] | 1.792 | |
| | 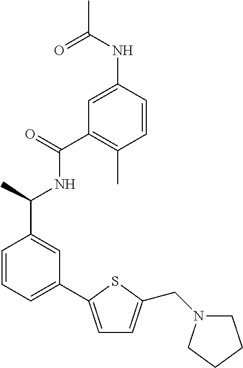 [SMILES: CC(=O)Nc1ccc(C)c(C(=O)N[C@H](C)c2cccc(-c3ccc(CN4CCCC4)s3)c2)c1] | 2.421 | |
| | 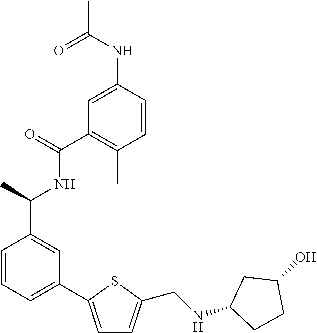 [SMILES: CC(=O)Nc1ccc(C)c(C(=O)N[C@H](C)c2cccc(-c3ccc(CN[C@H]4CC[C@@H](O)C4)s3)c2)c1] | 1.517 | |
| | 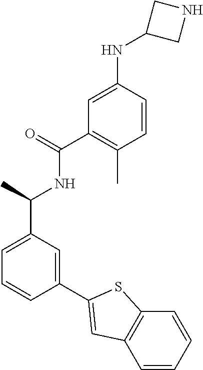 [SMILES: Cc1ccc(NC2CNC2)cc1C(=O)N[C@H](C)c1cccc(-c2cc3ccccc3s2)c1] | 1.131 | |
| | 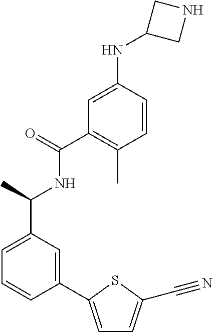 [SMILES: Cc1ccc(NC2CNC2)cc1C(=O)N[C@H](C)c1cccc(-c2ccc(C#N)s2)c1] | 5.526 | |
| | 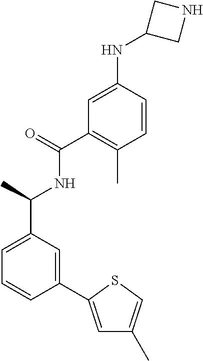 [SMILES: Cc1csc(-c2cccc([C@@H](C)NC(=O)c3cc(NC4CNC4)ccc3C)c2)c1] | 1.927 | |
| | 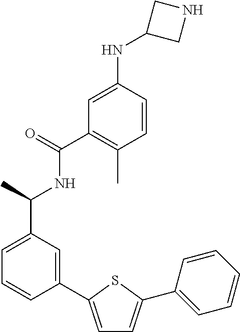 [SMILES: Cc1ccc(NC2CNC2)cc1C(=O)N[C@H](C)c1cccc(-c2ccc(-c3ccccc3)s2)c1] | 4.756 | |
| | 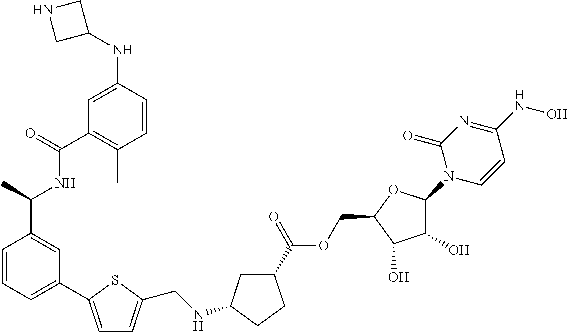 [SMILES: Cc1ccc(NC2CNC2)cc1C(=O)N[C@H](C)c1cccc(-c2ccc(CN[C@H]3CC[C@@H](C(=O)OC[C@H]4O[C@@H](n5ccc(NO)nc5=O)[C@H](O)[C@@H]4O)C3)s2)c1] | 0.6271 | |
| | 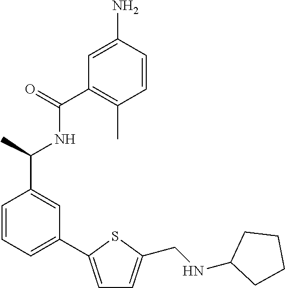 [SMILES: Cc1ccc(N)cc1C(=O)N[C@H](C)c1cccc(-c2ccc(CNC3CCCC3)s2)c1] | 1.394 | |
| | 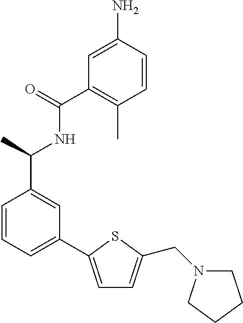 [SMILES: Cc1ccc(N)cc1C(=O)N[C@H](C)c1cccc(-c2ccc(CN3CCCC3)s2)c1] | 1.690 | |
| | 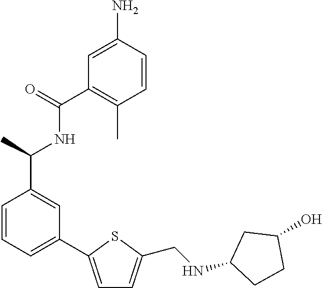 [SMILES: Cc1ccc(N)cc1C(=O)N[C@H](C)c1cccc(-c2ccc(CN[C@H]3CC[C@@H](O)C3)s2)c1] | 0.5967 | |
| | 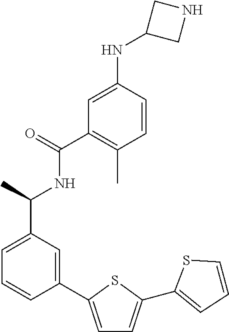 [SMILES: Cc1ccc(NC2CNC2)cc1C(=O)N[C@H](C)c1cccc(-c2ccc(-c3cccs3)s2)c1] | 4.435 | |
| | 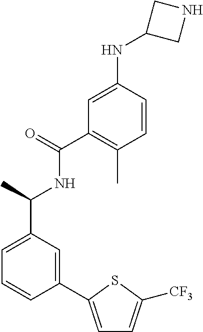 [SMILES: Cc1ccc(NC2CNC2)cc1C(=O)N[C@H](C)c1cccc(-c2ccc(C(F)(F)F)s2)c1] | 6.119 | |
| | 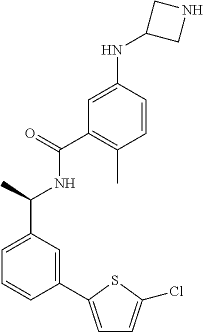 [SMILES: Cc1ccc(NC2CNC2)cc1C(=O)N[C@H](C)c1cccc(-c2ccc(Cl)s2)c1] | 1.506 | |
| | 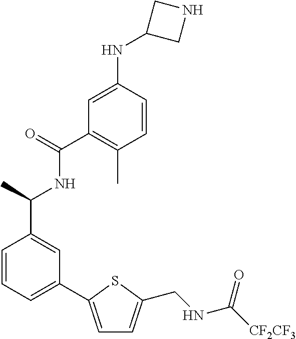 | 0.4416 | |
| | 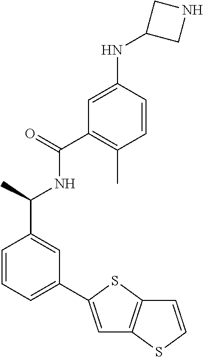 [SMILES: Cc1ccc(NC2CNC2)cc1C(=O)N[C@H](C)c1cccc(-c2cc3sccc3s2)c1] | 0.8239 | |
| | 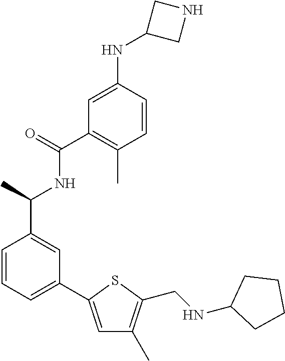 [SMILES: Cc1ccc(NC2CNC2)cc1C(=O)N[C@H](C)c1cccc(-c2cc(C)c(CNC3CCCC3)s2)c1] | 0.6519 | |
| | 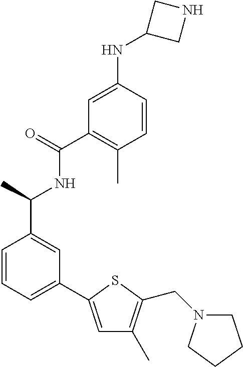 [SMILES: Cc1ccc(NC2CNC2)cc1C(=O)N[C@H](C)c1cccc(-c2cc(C)c(CN3CCCC3)s2)c1] | 0.9180 | |
| | 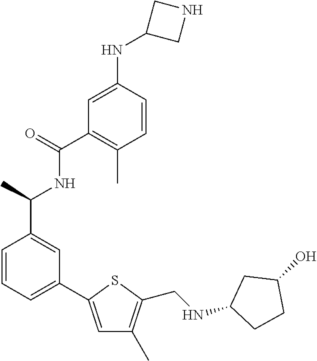 [SMILES: Cc1ccc(NC2CNC2)cc1C(=O)N[C@H](C)c1cccc(-c2cc(C)c(CN[C@H]3CC[C@@H](O)C3)s2)c1] | 0.3275 | |

The FRET enzymatic SARS-CoV-2 PLpro assay providing data in Table 8 was carried out in 50 mM HEPES, pH7.5, 0.01% triton-100 and 5 mM DTT. Briefly, the assay was performed in 96-well plates with 100 μl 200 nM PLPro protein, then 1 μl testing compound at various concentrations was added to each well and incubated at 30° C. for 30 min. The reaction was initiated by adding 1 μl of 1 mM FRET substrate (Dabcyl-FTLRGG/APTKV-Edans) (SEQ ID NO. 5) to 10 μM final substrate concentration. The reaction was monitored with filters for excitation at 360/40 nm and emission at 460/40 nm at 30° C. for 1 hr. The initial velocity of the enzymatic reaction with and without testing compounds was calculated by linear regression for the first 15 min of the kinetic progress curve.

The FlipGFP-PLpro assay data provided in Table 8 was obtained using plasmid pcDNA3-TEV-flipGFP-T2A-mCherry into which a SARS CoV-2 PLpro cleavage site LRGGAPTK (SEQ ID NO. 6) was introduced via overlapping PCRs to generate a fragment with SacI and HindIII sites at the ends. SARS CoV-2 PLpro expression plasmids was ordered from Genscript (Piscataway NJ) with codon optimization. For transfection, HEK-293T cells were treated with plasmids in the presence of transfection reagent TransIT-293 (Mirus). 3 hrs after transfection, 1 μl testing compound was added to each well at 100-fold dilution. Images were acquired 2 days after transfection and analysed and measured with GFP and mCherry channels. SARS CoV-2 PLpro protease activity was calculated by the ratio of GFP signal intensity over mCherry signal intensity.

The assay data provided in Table 8 provide further examples of test compounds' ability to inhibit PLpro enzymatic activity in a biochemical assay and in a cell-based assay in which PLpro is transiently transfected.

TABLE 8
Biological Assays for Representative Compounds of the Present
Disclosure

| | | | FlipGFP- |
|---|---|---|---|
| | | FRET | PLpro |
| | | Enzymatic | assay |
| | | assay | EC₅₀ |
| Examples | Structure | IC₅₀ (μM) | (μM) |
| GRL0167 | 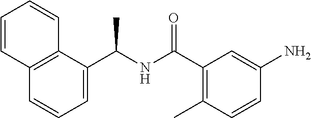 [SMILES: Cc1ccc(N)cc1C(=O)N[C@H](C)c1cccc2ccccc12] | 1.6 | 13.6 |
| | GRL0167 | | |
| 174 | 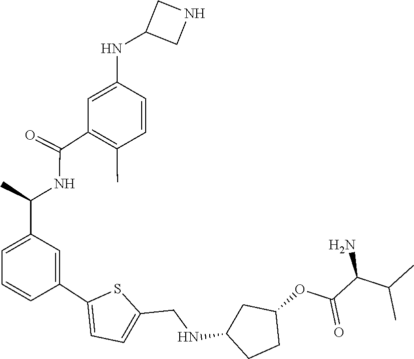 [SMILES: Cc1ccc(NC2CNC2)cc1C(=O)N[C@H](C)c1cccc(-c2ccc(CN[C@H]3CC[C@@H](OC(=O)[C@@H](N)C(C)C)C3)s2)c1] | 0.6 | >60 |
| 176 | 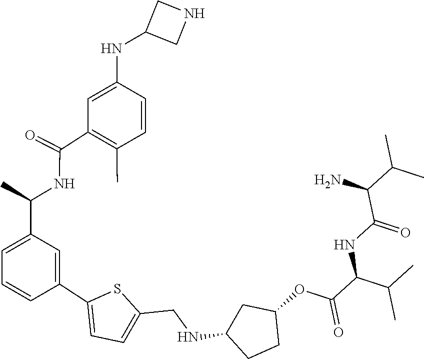 [SMILES: Cc1ccc(NC2CNC2)cc1C(=O)N[C@H](C)c1cccc(-c2ccc(CN[C@H]3CC[C@@H](OC(=O)[C@@H](NC(=O)[C@@H](N)C(C)C)C(C)C)C3)s2)c1] | 0.8 | >60 |
| 157 | 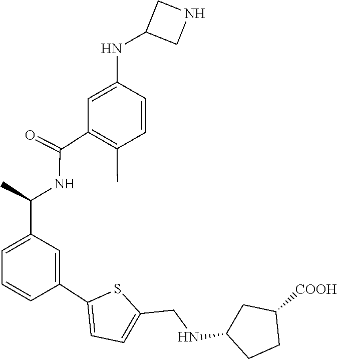 [SMILES: Cc1ccc(NC2CNC2)cc1C(=O)N[C@H](C)c1cccc(-c2ccc(CN[C@H]3CC[C@@H](C(=O)O)C3)s2)c1] | 0.6 | >60 |
| 134 | 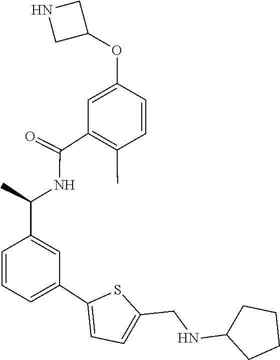 [SMILES: Cc1ccc(OC2CNC2)cc1C(=O)N[C@H](C)c1cccc(-c2ccc(CNC3CCCC3)s2)c1] | 1.3 | 21.3 |
| 135 | 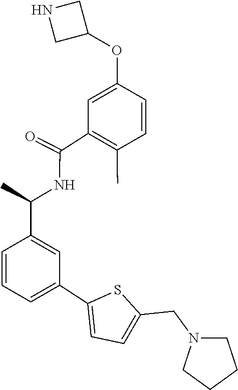 [SMILES: Cc1ccc(OC2CNC2)cc1C(=O)N[C@H](C)c1cccc(-c2ccc(CN3CCCC3)s2)c1] | 1.3 | 6.1 |
| 155 | 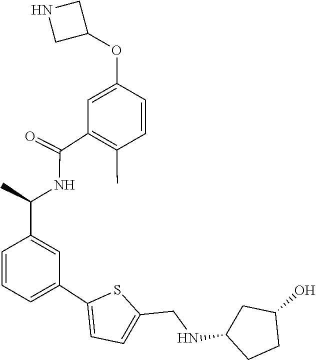 [SMILES: Cc1ccc(OC2CNC2)cc1C(=O)N[C@H](C)c1cccc(-c2ccc(CN[C@H]3CC[C@@H](O)C3)s2)c1] | 0.8 | 37.1 |
| | 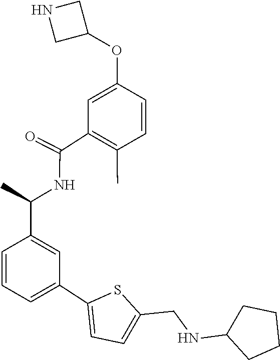 [SMILES: Cc1ccc(OC2CNC2)cc1C(=O)N[C@H](C)c1cccc(-c2ccc(CNC3CCCC3)s2)c1] | 1.33 | 21.33 ± 5.49 |
| | 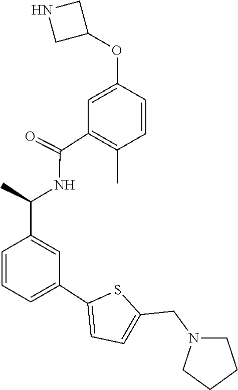 [SMILES: Cc1ccc(OC2CNC2)cc1C(=O)N[C@H](C)c1cccc(-c2ccc(CN3CCCC3)s2)c1] | 1.29 | 6.13 ± 0.95 |
| | 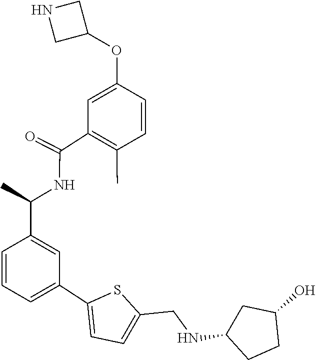 [SMILES: Cc1ccc(OC2CNC2)cc1C(=O)N[C@H](C)c1cccc(-c2ccc(CN[C@H]3CC[C@@H](O)C3)s2)c1] | 0.83 | 37.08 ± 3.58 |
| | 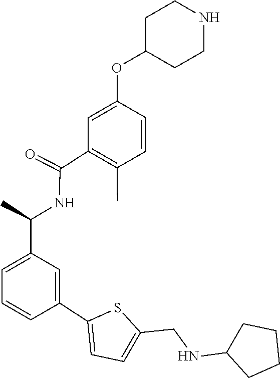 [SMILES: Cc1ccc(OC2CCNCC2)cc1C(=O)N[C@H](C)c1cccc(-c2ccc(CNC3CCCC3)s2)c1] | 1.77 ± 0.20 | >60 |
| | 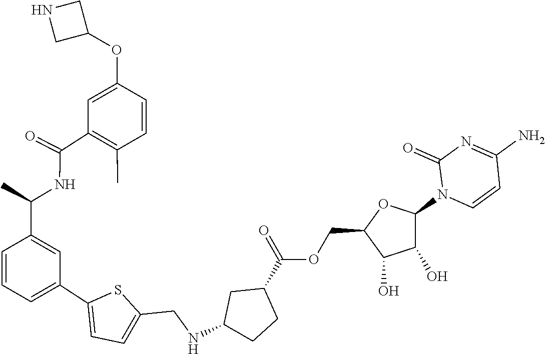 [SMILES: Cc1ccc(OC2CNC2)cc1C(=O)N[C@H](C)c1cccc(-c2ccc(CN[C@H]3CC[C@@H](C(=O)OC[C@H]4O[C@@H](n5ccc(N)nc5=O)[C@H](O)[C@@H]4O)C3)s2)c1] | 0.80 ± 0.08 | >20 (~50) |
| | 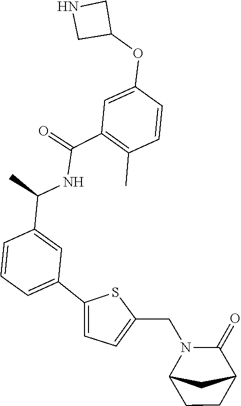 [SMILES: Cc1ccc(OC2CNC2)cc1C(=O)N[C@H](C)c1cccc(-c2ccc(CN3C(=O)[C@@H]4CC[C@H]3C4)s2)c1] | 1.91 ± 0.11 | Toxic (>10) |
| | 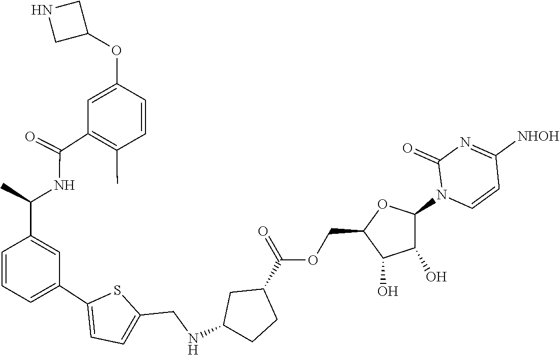 | 0.75 ± 0.05 | >20 |
| | 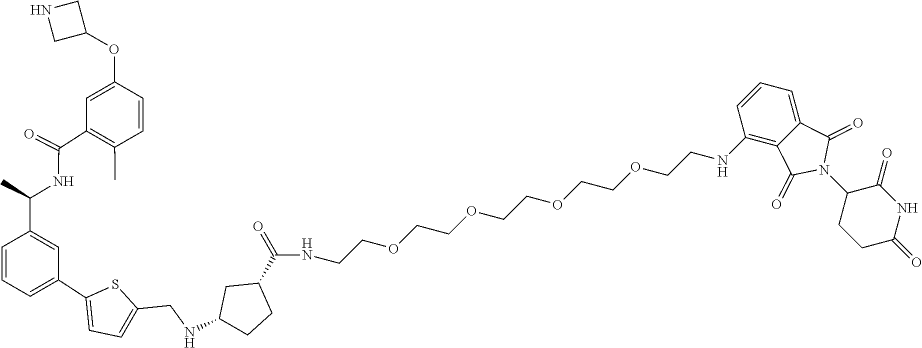 [SMILES: Cc1ccc(OC2CNC2)cc1C(=O)N[C@H](C)c1cccc(-c2ccc(CN[C@H]3CC[C@@H](C(=O)NCCOCCOCCOCCOCCNc4cccc5c4C(=O)N(C4CCC(=O)NC4=O)C5=O)C3)s2)c1] | 1.67 ± 0.33 | >20 |
| | 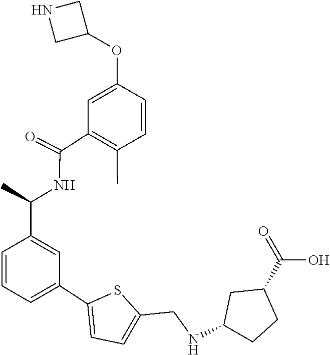 [SMILES: Cc1ccc(OC2CNC2)cc1C(=O)N[C@H](C)c1cccc(-c2ccc(CN[C@H]3CC[C@@H](C(=O)O)C3)s2)c1] | 0.58 ± 0.07 | >60 |
| | 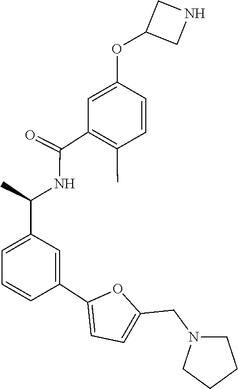 [SMILES: Cc1ccc(OC2CNC2)cc1C(=O)N[C@H](C)c1cccc(-c2ccc(CN3CCCC3)o2)c1] | 4.58 ± 0.92 | >60 |
| | 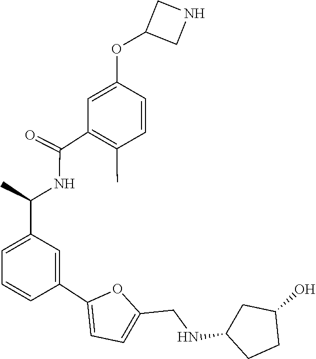 [SMILES: Cc1ccc(OC2CNC2)cc1C(=O)N[C@H](C)c1cccc(-c2ccc(CN[C@H]3CC[C@@H](O)C3)o2)c1] | 3.02 ± 0.58 | >60 |
| | 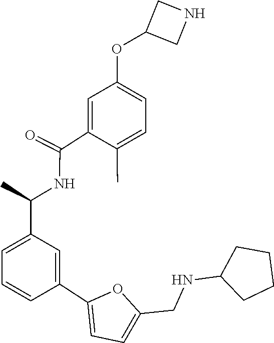 [SMILES: Cc1ccc(OC2CNC2)cc1C(=O)N[C@H](C)c1cccc(-c2ccc(CNC3CCCC3)o2)c1] | 3.49 ± 0.35 | >20 |
| | 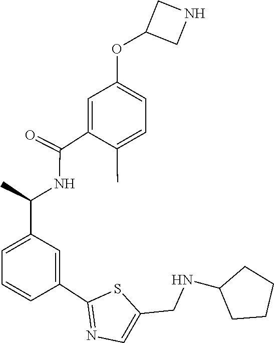 [SMILES: Cc1ccc(OC2CNC2)cc1C(=O)N[C@H](C)c1cccc(-c2ncc(CNC3CCCC3)s2)c1] | 1.88 ± 0.41 | 23.72 ± 2.77 |

### Example 2: SARS-CoV-2 PLpro Inhibitors Block Viral Replication

Antiviral agents blocking SARS-CoV-2 viral replication are needed to complement vaccination to end the COVID-19 pandemic. Viral replication and assembly are entirely dependent on two viral cysteine proteases: 3C-like protease (3CLpro) and the papain-like protease (PLpro). PLpro also has deubiquitinase (DUB) activity, removing ubiquitin (Ub) and Ub-like modifications from host proteins, disrupting the host immune response. 3CLpro is inhibited by many known cysteine protease inhibitors, whereas PLpro is a relatively unusual cysteine protease, being resistant to blockade by such inhibitors. A high-throughput screen of biased and unbiased libraries gave a low hit rate, identifying only CPI-169 and the positive control, GRL0617, as inhibitors with good potency (IC50<10 μM). Analogues of both inhibitors were designed to develop structure-activity relationships; however, without a co-crystal structure of the CPI-169 series, the following study focused on GRL0617 as a starting point for structure-based drug design, obtaining several co-crystal structures to guide optimization. A series of novel 2-phenylthiophene-based non-covalent SARS-CoV-2 PLpro inhibitors were obtained, culminating in low nanomolar potency. The high potency and slow inhibitor off-rate were rationalized by newly identified ligand interactions with a “BL2 groove” that is distal from the active site cysteine. Trapping of the conformationally flexible BL2 loop by these inhibitors blocks binding of viral and host protein substrates; however, until now it has not been demonstrated that this mechanism can induce potent and efficacious antiviral activity. In this study, PLpro inhibitors were identified with excellent antiviral efficacy and potency against infectious SARS-CoV-2 replication in cell cultures. Together, the data provide structural insights into the design of potent PLpro inhibitors and the first validation that non-covalent inhibitors of SARS-CoV-2 PLpro can block infection of human cells with low micromolar potency. Further details of this study are included in “Novel, Potent SARS-CoV-2 PLpro Inhibitors Block Replication in Monkey and Human Cell Cultures,” Zhengnan Shen, et al., bioRxiv, 2021 (doi.org/10.1101/2021.02.13.431008), incorporated herein by reference and attached as Appendix A. Appendix A in its entirety including figures and supplementary material is included as part of this disclosure.

### Materials and Methods

SARS-CoV-2 PLpro expression and purification: pET11a vector containing SARS-CoV-2 PL proprotein (pp1ab aa 1564-1878) with N-terminal, TEV-cleavable His-tag was transformed into BL21(DE3) cells and maintained in media containing 100 ug/mL carbenicillin. Protein expression was induced using an auto-induction protocol modified from Studier et al 2005 [Studier, 2005]. Briefly, 1 mL day cultures were used to inoculate a 2 L flask of 500 mL of SuperLB containing 100 ug/mL carbenicillin. Cells were grown for 24 h at 25° C. and then harvested by centrifugation. All steps of SARS-CoV2 PLpro purification were performed at 4° C. Protein yield at each step was monitored by Bradford assay using BSA as a standard. Frozen cells pellets were lysed by sonication in Buffer A (50 mM HEPES, pH 8, 0.5 M NaCl) containing 10 ug/mL lysozyme. The lysate was clarified by centrifugation and loaded onto a 2-mL HiTrap Talon crude column equilibrated with Buffer A. Bound His₆-PLpro was eluted with a linear gradient of 0-150 mM imidazole in Buffer A, and fractions containing His₆-PLpro were pooled and exchanged into cleavage buffer (20 mM Tris-HCl pH 8.5, 5 mM DTT, 0.5 mM EDTA, 5% glycerol). A 1:100 molar ratio of TEV protease to PLpro was incubated at 4° C. overnight to cleave the His₆-tag. To remove the tag and TEV protease, the reaction was loaded onto a UNO-Q column equilibrated with 20 mM Tris HCl, pH 8.5, 3 mM DTT. Cleaved PLpro eluted first in a gradient from 0-150 mM NaCl over 20 column volumes. Fractions containing cleaved PLpro were pooled and concentrated to 12 mg/mL, frozen in liquid nitrogen, and stored at −80° C.

PLpro primary assay: The PLpro primary assay, which measures protease activity with the short peptide substrate Z-RLRGG-AMC (SEQ ID NO. 4) (Bachem), was performed in black, flat-bottom 384-well plates containing a final reaction volume of 50 μL. The assays were assembled at room temperature as follows: 40 μL of 50 nM PLpro in Buffer B (50 mM HEPES, pH 7.5, 0.1 mg/mL BSA, 0.01% Triton-X 100, and 5 mM DTT) was dispensed into wells containing 0.1-1 μL of inhibitor in DMSO or appropriate controls. The enzyme was incubated with inhibitor for 10 min prior to substrate addition. Reactions were initiated with 10 μL of 62.5 μM RLRGG-AMC (SEQ ID NO. 4) in Buffer B. Plates were shaken vigorously for 30 s, and fluorescence from the release of AMC from peptide was monitored continuously for 15 min on a Tecan Infinite M200 Pro plate reader (λ_{excitation}=360 nm; λₑₘᵢₛₛᵢₒₙ=460 nm). Slopes from the linear portions of each progress curve were recorded and normalized to plate-based controls. Positive control wells, representing 100% inhibition, included 10 μM GRL0617; negative control wells, representing 0% inhibition, included vehicle.

PLpro high-throughput screening: High-throughput screening for inhibitors of PLpro was performed using the primary assay above. Test compounds (20 μM final concentration) and controls were delivered via 100 nL pin tool (V&P Scientific). The libraries included in the screenwere purchased from TargetMol (Bioactive Library) and ChemDiv (a 10,000-compound SMART library subset). Each 384-well plate contains 32 positive control wells and 32 negative control wells. Average Z′ values for this assay ranged from 0.85-0.90. Compounds producing >40% inhibition of PLpro activity were selected for follow-up analysis. To eliminate compounds that interfered with AMC fluorescence and thus produced false positives, the fluorescence of 10 μM free AMC was measured in the presence of 20 μM compound in Buffer B. Inhibitors that produced a >25% decrease in AMC fluorescence signal were eliminated from further analysis. Similarly, compounds that were frequent hitters in in-house screens, or that were documented redox-cycling compounds, were eliminated from follow-up studies.

The dose responses of remaining hit compounds were tested in the primary assay over 10 compound concentrations. Percent inhibition (% I) of each data point was calculated using Equation 1:

$$\\ \begin{matrix}\% I = 100 ( 1 - \frac{{v}_{i}}{{v}_{c}} ) & ( 1 )\end{matrix}$$

where vᵢ is the reaction rate in the presence of inhibitor and v_{c} is the reaction rate in the absence of inhibitor (DMSO control). Data were fit to Equation 2 using GraphPad Prism to establish an IC₅₀:

$$\\ \begin{matrix}\% I = \frac{100}{1 + \frac{x}{{IC}_{50}^{n}}} & ( 2 )\end{matrix}$$

where x is the concentration of inhibitor and n is the Hill coefficient.

The selectivity of the most potent inhibitors was tested against the human deubiquitinating enzymes USP7 and USP14 (Boston Biochem). Assay conditions were similar to the PLpro primary assay, with the following substitutions: USP7 assays contained 4 nM USP7 and 0.5 uM Ub-AMC (Boston Biochem); USP14 assays contained 1.7 uM USP14, 4 uM Ub-AMC, and the addition of 5% glycerol to Buffer B.PLpro activity with ISG15-AMC and Ub-AMC were assayed in a manner similar to the PLpro primary assay. PLpro and substrate concentrations were modified as follows: 80 nM PLpro was assayed with 0.5 uM Ub-AMC, and 4 nM PLpro was assayed with 0.5 μM ISG15-AMC.

Crystallization: Crystals of SARS-CoV-2 PLpro complexed with XR compounds were grown by hanging drop vapor diffusion at 16° C. Prior to crystallization, 12 mg/mL PLpro protein was incubated with 2 mM XR824 (or XR865, XR869, XR883, XR889) for 30 min on ice. Crystals of the complexes were grown by mixing 1-2 μL of PLpro:inhibitor complex with 2 μL of reservoir solution containing 0.1 M MIB buffer, pH 7.2, 0.2 M (NH₄)₂SO₄, and 24-28% PEG 4000 or 0.1 MIB buffer, pH 6.0-6.8, 0.2 M (NH₄)₂SO₄, 13-16% PEG 3350, and 20% glycerol. Crystals grew overnight from the PEG 4000 conditions and were used to streak seed drops of PLpro:inhibitor equilibrating against the PEG 3350 conditions.

Data Collection and Structure Refinement: The glycerol present in the crystallization solution was sufficient to cryo-protect crystals, which were flash-cooled in liquid nitrogen. Data were collected at the Life Sciences Collaborative Access Team beamlines 21-ID-D, 21-ID-G, and 21-ID-F at the Advanced Photon Source, Argonne National Laboratory. Data indexing and integration were performed using XDS.¹² Ellipsoidal truncation and anisotropic scaling were performed by the UCLA-DOE lab's Diffraction Anisotropy Server for the XR824 complex.¹³ Phases were determined by molecular replacement using Molrep¹⁴ and a SARS-CoV-2 PLpro: GRL0617 complex (PDB entry: 7JRN) as search model. Rigid body refinement followed by iterative rounds of restrained refinement and model building were performed with CCP4i modules Refmac5¹⁵ and Coot¹⁶. The coordinates and structure factors have been deposited with PDB accession codes 7LBR (XR8-89 complex), 7LBS (XR8-24 complex), and 7LLF (XR-883 complex).

Secondary binding analysis by Surface Plasmon Resonance (SPR). The His-tagged SARS-CoV-2 PLpro enzyme was initially prepared in phosphate buffer and diluted to 50 pg/mL with 10 mM sodium acetate (pH 5.5) and immobilized on a CM5 sensor chip by standard amine-coupling with running buffer PBSP (10 mM phosphate, pH 7.4, 2.7 mM KCl, 137 mM NaCl, 0.05% Tween-20). The CM5 sensor chip surface was first activated by 1-ethyl-3-(3-dimethylaminopropyl) carbodiimide hydrochloride (EDC)/N-hydroxy succinimide (NHS) mixture using a Biacore 8K instrument (Cytiva). SARS-CoV-2 PLpro enzyme was immobilized to flow channels 1 through 4 followed by ethanolamine blocking on the unoccupied surface area, and immobilization levels for all four channels were similar at -12,000 RU. Each flow channel has its own reference channel, and blank immobilization using EDC/NHS and ethanolamine was done for all reference channels. Compound solutions with a series of increasing concentrations (0.049-30 μM at 2.5-fold dilution) were applied to all active and reference channels in SPR binding buffer (10 mM HEPES, pH 7.4, 150 mM NaCl, and 0.05% Tween-20, 0.5 mM TCEP, and 2% DMSO) at a 30 μL/min flow rate at 25° C. The data were double referenced with a reference channel and zero concentration (2% DMSO) responses, and reference subtracted sensorgrams were fitted with 1 to 1 Langmuir kinetic model using a Biacore Insight evaluation software, producing two rate constants (ka and kd). The equilibrium dissociation constants (K_{D}) were determined from two rate constants (K_{D}=k_{d}/kₐ). For steady-state affinity fittings, response units at each concentration were measured during the equilibration phase, and the K_{D} values were determined by fitting the data to a single rectangular hyperbolic curve equation (3), where y is the response, yₘₐₓ is the maximum response and x is the compound concentration.

$$\\ \begin{matrix}y = \frac{{y}_{max} \cdot x}{{K}_{D} + x} & ( 3 )\end{matrix}$$

Data collection and structure refinement: The glycerol present in the crystallization solution was sufficient to cryo-protect crystals, which were flash-cooled in liquid nitrogen. Data were collected at the Life Sciences Collaborative Access Team beamlines 21-ID-D, 21-ID-G, and 21-ID-F at the Advanced Photon Source, Argonne National Laboratory. Data indexing and integration were performed using XDS.⁵⁶ Ellipsoidal truncation and anisotropic scaling were performed by the UCLA-DOE lab's Diffraction Anisotropy Server for the XR824 complex.⁵⁷ Phases were determined by molecular replacement using Molrep⁵⁸ and a SARS-CoV-2 PLpro: GRL0617 complex (PDB entry: 7JRN) as search model. Rigid body refinement followed by iterative rounds of restrained refinement and model building were performed with CCP4i modules Refmac5⁵⁹ and Coot.⁶⁰ The coordinates and structure factors have been deposited with PDB accession codes 7LBR (XR8-89 complex), 7LBS (XR8-24 complex), 7LLF (XR8-83 complex), 7LLZ (XR8-69 complex), and 7LOS (XR8-65 complex).

Cell Culture and Cytotoxicity: African green monkey kidney epithelial cells Vero E6 (ATCC #CRL-1586) were cultured in DMEM supplemented with 10% fetal bovine serum (Gibco), 100 units of penicillin and 100 pg/mL streptomycin (Invitrogen). Human alveolar epithelial cell line (A549) that stably express hACE2 are from BEI Resources (NR-53821). They were grown DMEM supplemented with 10% fetal bovine serum (Gibco), 100 units of penicillin and 100 pg/mL streptomycin (Invitrogen), 1% nonessential amino acids (NEAA) with 100 μg/mL Blasticidin S. HCl for selection. All cells were grown at 37° C. and 5% CO₂. Low passage vero E6 and A549 cells (5000 cells/well) were seeded in 96-well plates and incubated at 37° C. and 5% CO₂ for 24 hours prior to treatment. All compounds were dissolved in DMSO and final DMSO concentrations never exceeded 1%. The cytotoxicity of compounds (100 μM to 1 μM, 3-fold dilution) was examined using the CellTiter-Glo Luminescent Cell Viability Assay (Promega). Cell cytotoxicity data was normalized to DMSO control as 0% cell death.

Antiviral Activity Assays: Vero E6 cells were seeded 5×10⁵ cells/well in DMEM complete into 12-well plates (1 mL/well). Cells were pretreated with 1.5 μM CP-100356 for 30 min and with both compound+1.5 μM CP-100356 for 1-hour prior to infection. The plaque reduction assay was performed using a clinical isolate of SARS-CoV-2 (SARS-CoV-2, Isolate USA-WA1/2020) from BEI Resources. 2-fold serial dilutions of compound+CP-100356 were added to the same volume of SARS-CoV-2 (final MOI=0.0001), the mixture was added to the monolayer of Vero E6 cells and incubated for 1 hour at 37° C. and 5% CO₂. The mixture was removed, 1 mL of 1.25% (w/v) Avicel-591 in 2×MEM supplied with 4% (v/v) FBS was added onto infected cells with 10× compound+CP-100356. Plates were incubated 48 hours at 37° C. and 5% CO₂. After the 48-hour incubation, the plates were fixed with 10% (v/v) formaldehyde and stained with 1% (w/v) crystal violet to visualize the plaques. All experiments were performed in a Biosafety level 3 facility. IC₅₀ values were determined by fitting the dose-response curves with four-parameter logistic regression in Prism GraphPad (version 8.1.2). All data was normalized to virus alone. All error bars represent S.D. from three replicates. A549-hACE2 cells were seeded 1.5×10⁵ cells/well in DMEM complete into 24-well plates (0.5 mL/well) then incubated for 16 hours at 37° C. and 5% CO₂. Cells were pretreated with compound for 1-hour prior to infection. 2-fold serial dilutions of compound added to the same volume of SARS-CoV-2 (final MOI=0.01), the mixture was added to the monolayer cells and incubated for 1 hour at 37° C. and 5% CO₂. After, the mixture was removed and replaced with 0.5 mL of infection media and incubated at 37° C. and 5% CO₂. After 48 hours, supernatants were harvested and processed for RT-qPCR.

RNA Extraction and RT-qPCR: 250 μL of culture fluids were mixed with 750 μL of TRIzol™ LS Reagent (Thermo Fisher Scientific). RNA was purified following phase separation by chloroform as recommended by the manufacturer. RNA in the aqueous phase was collected and further purified using PureLink RNA Mini Kits (Invitrogen) according to manufacturer's protocol. Viral RNA was quantified by reverse-transcription quantitative PCR (RT-qPCR) using a 7500 Real-Time PCR System (Applied Biosystems) using TaqMan Fast Virus 1-Step Master Mix chemistry (Applied Bio-systems). SARS-CoV-2 N1 gene RNA was amplified using forward (5′-GACCCCAAAATCAGCGAAAT) (SEQ ID NO. 1) and reverse (5′-TCTGGTTACTGCCAGTTGAATCTG) (SEQ ID NO. 2) primers and probe (5′-FAM-ACCCCGCATTACGTTTGGTGGACC-BHQ1) (SEQ ID NO. 3) designed by the United States Centers for Disease Control and Prevention (oligonucleotides produced by IDT, cat #10006713). RNA copy numbers were determined from a standard curve produced with serial 10-fold dilutions of RNA standard material of the amplicon region from BEI Resources (NR-52358). All data was normalized to virus alone. All error bars represent S.D. from three replicates.

### Results

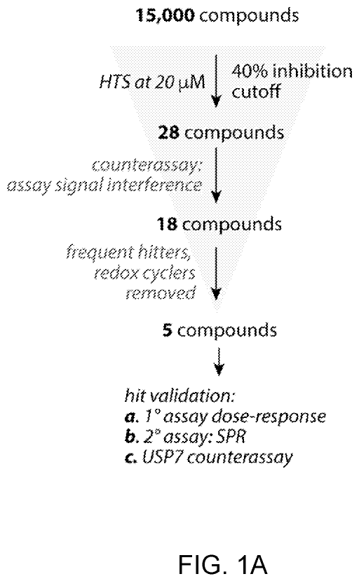

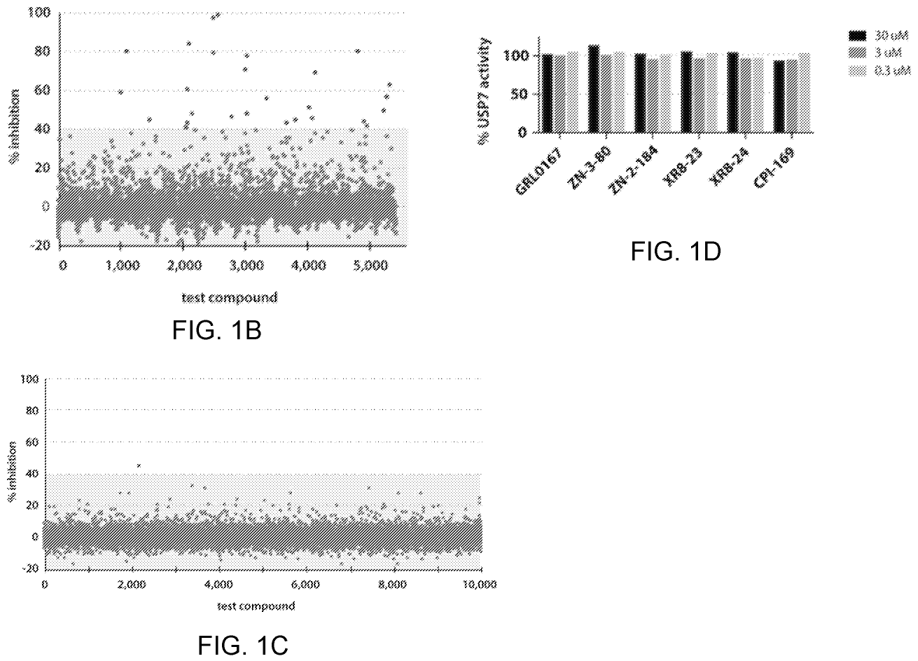

High-throughput screening to identify inhibitors of SARS-CoV-2 PLpro. To discover novel chemical scaffolds that inhibit SARS-CoV-2 PLpro, a HTS assay was performed to measure PLpro protease activity with the short peptide substrate Z-RLRGG-AMC (SEQ ID NO. 4). Assay performance was excellent, with plate Z′ values ranging from 0.85-0.90. An unbiased ChemDiv library (10,000-compound SMART library subset excluding PAINS compounds) and a biased, annotated TargetMol Bioactive library (5,370 compounds) were screened. The Bioactive library contains 1,283 FDA-approved drugs, 761 drugs approved by regulatory bodies in other countries such as European Medicines Agency (EMA), and 3,326 advanced-stage developmental candidates. Compounds were screened against PLpro at a final compound concentration of 20 μM, which is a more stringent threshold than other contemporary screens of PLpro.38 Assay of PLpro in the presence of 5 mM DTT, as reducing agent and electrophile trap, strongly biases against reactive (electrophilic and redox) hits. FIG. 1A is a schematic of the HTS assay for SARS-CoV-2 PLpro inhibitors including the hit triage and validation workflow.

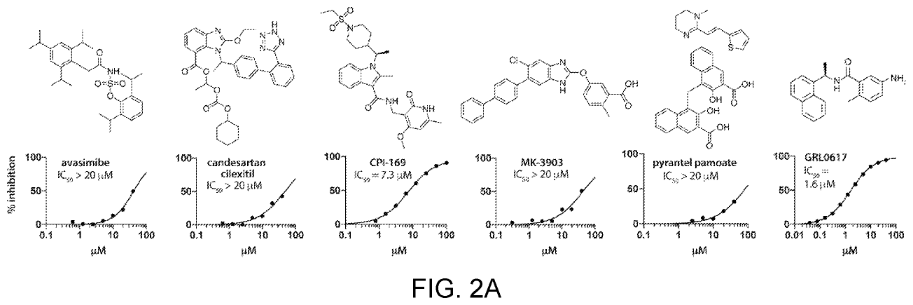

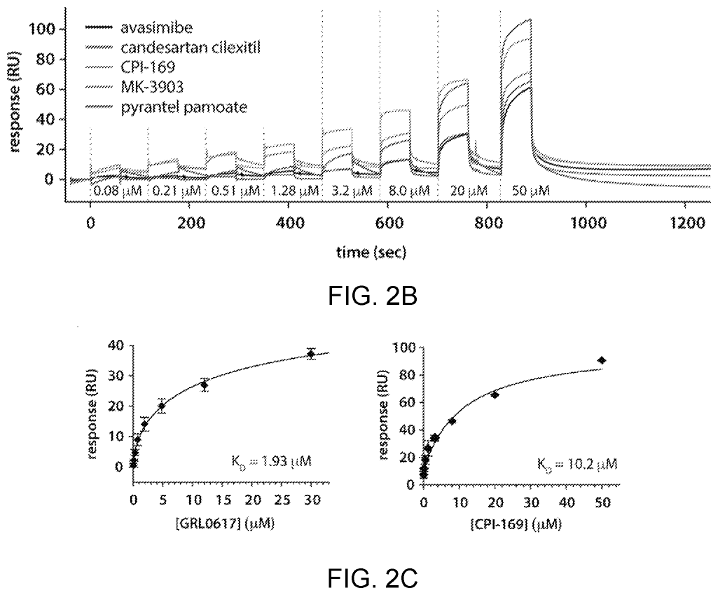

The 28 hit compounds from HTS were counter-assayed to remove false positives associated with signal interference and then further pruned to remove frequent hitters and known redox cyclers (FIG. 1A-1C). A set of five compounds producing >40% inhibition of SARS-CoV-2 PLpro activity along with the SARS-CoV PLpro inhibitor GRL0617 were selected for follow-up 8-point dose-response assays (FIG. 2A). All six active compounds were also tested in an orthogonal binding assay using surface plasmon resonance (SPR) (8-point titration) (FIG. 2B). Only GRL0617 and CPI-169 inhibited PLpro with IC50<10 μM in the primary enzyme inhibition assay (IC50 values of 1.6 μM and 7.3 μM, respectively (FIG. 2A). These values compare well with the binding affinities measured by SPR: GRL0617 KD=1.9 μM, CPI-169 KD=10.2 μM (FIG. 2C).

PLpro has DUB enzyme activity, therefore, human DUBs represent off-targets for any PLpro inhibitor. To confirm that hits were selective for SARS-CoV-2 PLpro over host cell DUBs, the catalytic domain of human USP7, the closest structural homolog of PLpro, was used as an additional counter-assay. Consistent with the initial findings, GRL0617 did not inhibit USP7-catalyzed Ub-AMC hydrolysis.²⁹ Similarly, CPI-169 was not able to inhibit USP7 up to a concentration of 30 μM (FIG. 1D). This data confirmed and validated GRL0617 and CPI-169 as selective SARS-CoV-2 PLpro inhibitors.

Structure-based design of PLpro inhibitors. With these two inhibitors in hand, the next steps were to embark on a medicinal chemistry campaign to optimize both GRL0617 and CPI-169 (SAR for CPI-169 will be reported in a separate paper). As shown above, GRL0617 displayed only modest potency against SARS-CoV-2 PLpro.

Furthermore, this family of compounds was reported to be unstable to metabolism by liver cytochrome P450s, probably due to the presence of aniline and naphthalene groups, which are known culprits for rapid metabolism, precluding use as in vivo probe compounds.^{32,39} Any modifications to the GRL0617 series would need to incorporate improved potency and metabolic stability.

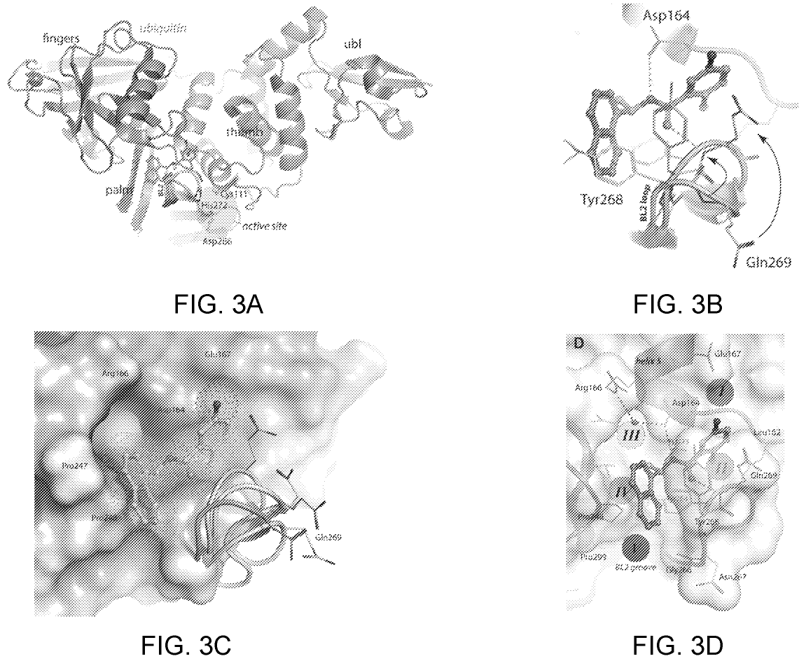

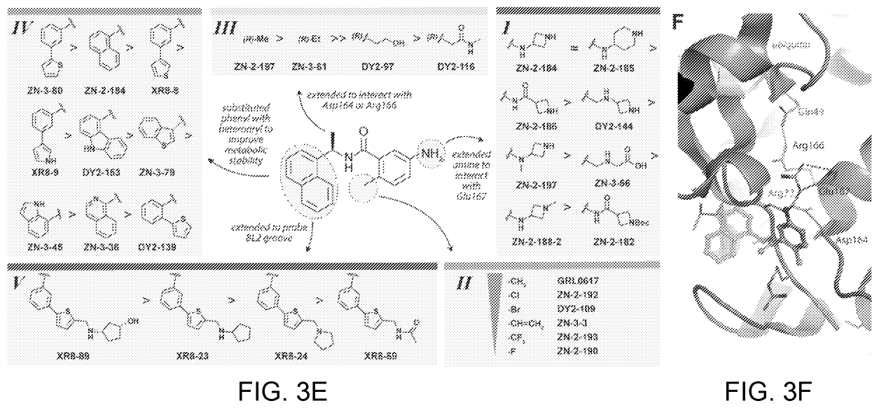

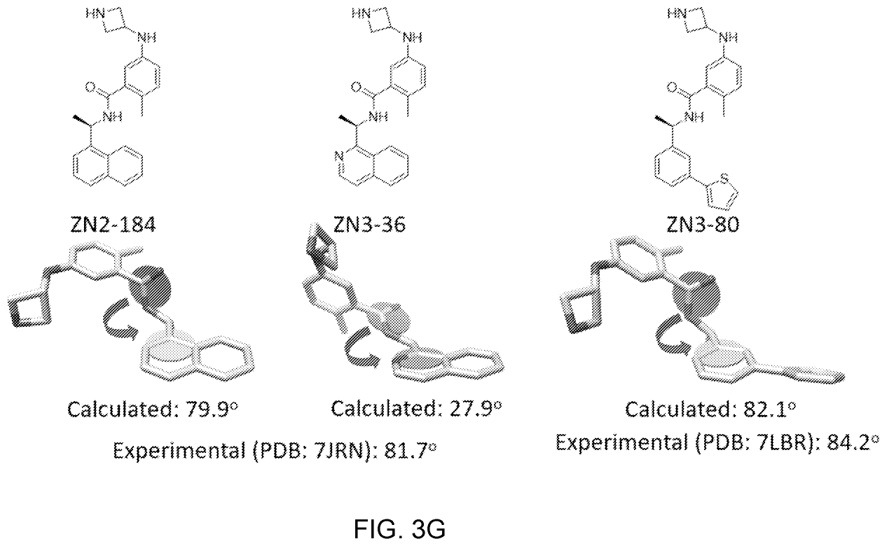

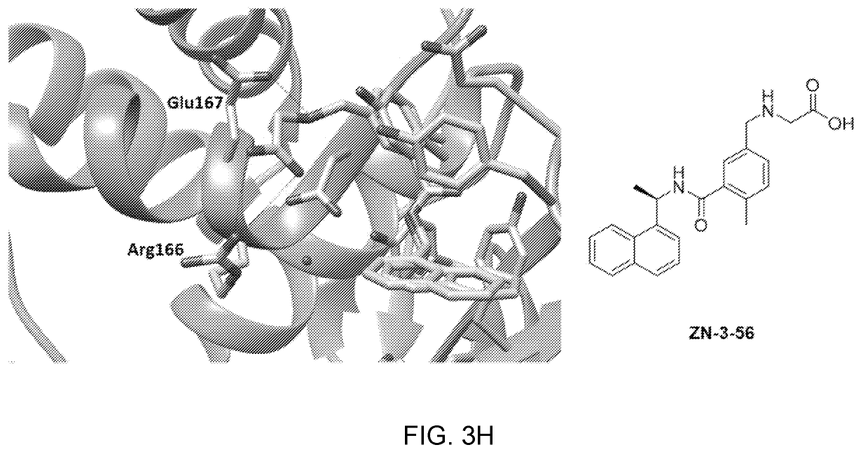

SARS-CoV-2 PLpro has 83% sequence identity to SARS-CoV PLpro and 100% identity at the active site, therefore, the GRL0617:PLpro (SARS-CoV) co-crystal structure (PDB: 3E9S) can be used to guide initial structure-based optimization of this series. The PLpro monomer is comprised of four distinct domains, including an N-terminal ubiquitin-like (Ubl) domain (first 62 residues) and an extended right-hand architecture with distinct palm, thumb, and finger domains (FIG. 3A). The active site formed by the catalytic triad, Cys111, His272, and Asp286 (SARS-CoV-2 PLpro numbering, PDB: 7JRN), sits in a solvent-exposed cleft at the interface of the thumb and palm domains. Binding of host and viral protein substrates is controlled by the flexible β-hairpin BL2 loop, which contains an unusual beta-turn formed by Tyr268 and Gln269 (FIGS. 3A-3B), controlling access to the active site.

Superposition of crystal structures of the SARS-CoV-2 PLpro apoenzyme (PDBs _{6WZU, 7D47, 7JCD}) highlights the flexibility of the BL2 loop (FIG. 3C). The binding of GRL0617, presumably by induced-fit, requires reorganization of the PLpro secondary structure, and thus the association rate of the ligand is anticipated to be slower than the rate of diffusion. In SPR experiments, the association rate was measured to be 1.8×10⁵ M⁻¹s⁻¹, which is significantly slower than the expected 1×10⁹ M⁻¹s⁻¹ rate of diffusion-controlled association. The unique structural reorganization of the BL2 loop, in part, explains the low hit rate from the HTS campaign (as also reported by others⁴⁰) and represents a challenge and an opportunity for developing potent, selective small molecule PLpro inhibitors.

In the GRL0617:PLpro (SARS-CoV) co-crystal structure, the ligand stabilizes the closed BL2 loop, blocking access to the active site; thus, unusually for a cysteine protease inhibitor, GRL0617 does not interact with the active site cysteine and the closest point of contact is 7.6 Å distant. It was hypothesized that CPI-169 also stabilizes the BL2 loop; however, co-crystal structures of CPI-169 or its analogues with SARS-CoV-2 PLpro to guide optimization was not obtained.

Optimization of novel benzamide PLpro inhibitors. GRL0617 forms two key hydrogen bonding interactions with the mainchain nitrogen of Gln269 and sidechain of Asp164 in PLpro (FIG. 31B), causing the BL2 loop to adopt a twisted conformation that narrows the solvent-exposed surface and exposes a hydrophobic binding site. To test the importance of this hydrogen bonding interaction in regulating the BL2 loop conformation, two tool compounds were synthesized by reducing the amide to amine (DY2-64) or replacing the amide with a sulfonamide bioisostere (DY3-63). Both modifications led to a sharp decline in potency, therefore the benzamide was conserved moving forward (Table 9).

TABLE 9

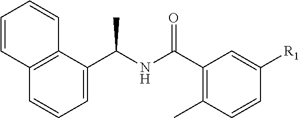

| | | | SPR |
|---|---|---|---|
| | | Enzyme | binding |
| | | inhibition | assays |
| Compound | R₁ | IC₅₀ (μM) | K_{D} (μM) |
| GRL0167 | 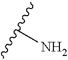 [SMILES: *N] | 1.61 | 2.70 |
| ZN-2-180 | 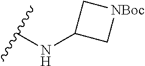 [SMILES: *NC1CN(C(=O)OC(C)(C)C)C1] | 6.08 | 16.0 |
| ZN-2-181 | 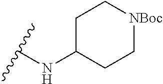 [SMILES: *NC1CCN(C(=O)OC(C)(C)C)CC1] | 1.1 | 4.7 |
| ZN-2-182 | 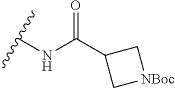 [SMILES: *NC(=O)C1CN(C(=O)OC(C)(C)C)C1] | 5.5 | 32.6 |
| ZN-2-183 | 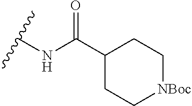 [SMILES: *NC(=O)C1CCN(C(=O)OC(C)(C)C)CC1] | 6.0 | 31.1 |
| ZN-2-184 | 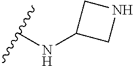 [SMILES: *NC1CNC1] | 1.01 | 1.03 |
| ZN-2-185 | 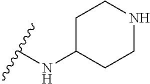 [SMILES: *NC1CCNCC1] | 0.6 | 1.8 |
| ZN-2-186 | 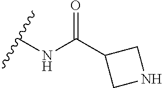 [SMILES: *NC(=O)C1CNC1] | 1.2 | 3.1 |
| ZN-2-187 | 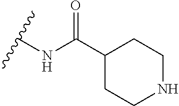 [SMILES: *NC(=O)C1CCNCC1] | 0.8 | 5.4 |
| ZN-2-189 | 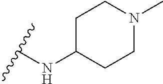 [SMILES: *NC1CCN(C)CC1] | 0.7 | 1.3 |
| ZN-2-188-1 | 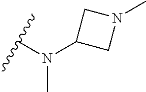 [SMILES: *N(C)C1CN(C)C1] | 1.6 | 5.6 |
| ZN-2-188-2 | 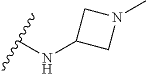 [SMILES: *NC1CN(C)C1] | 4.3 | 3.4 |
| ZN-2-197 | 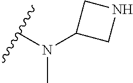 [SMILES: *N(C)C1CNC1] | 2.4 | 2.8 |
| ZN-3-56 | 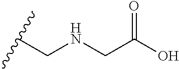 [SMILES: *CNCC(=O)O] | 3.9 | 26.5 |
| DY2-144 | 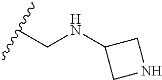 [SMILES: *CNC1CNC1] | 1.3 | 6.0 |
| DY2-137 | 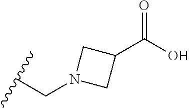 [SMILES: *CN1CC(C(=O)O)C1] | 3.3 | 8.16 |
| Dy2-138-2 | 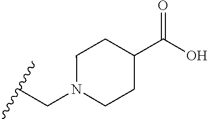 [SMILES: *CN1CCC(C(=O)O)CC1] | 6.1 | NA |

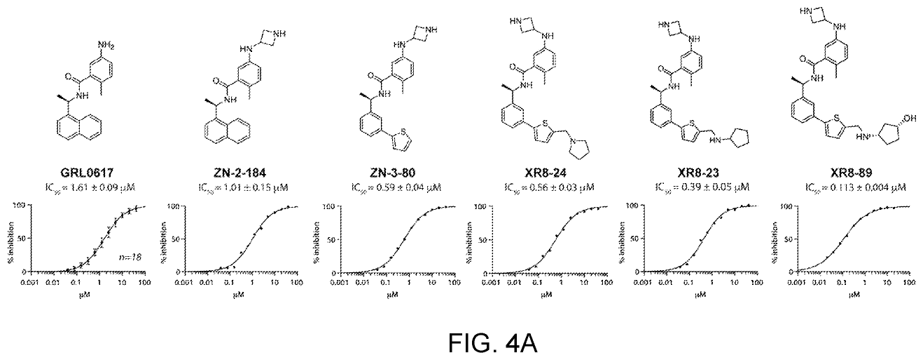

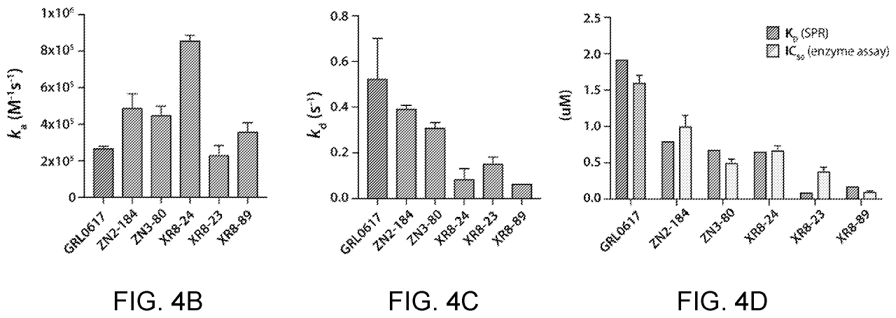

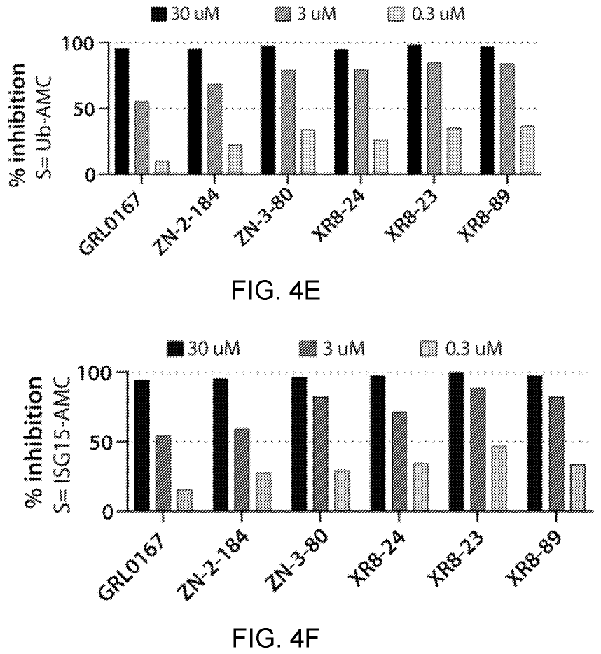

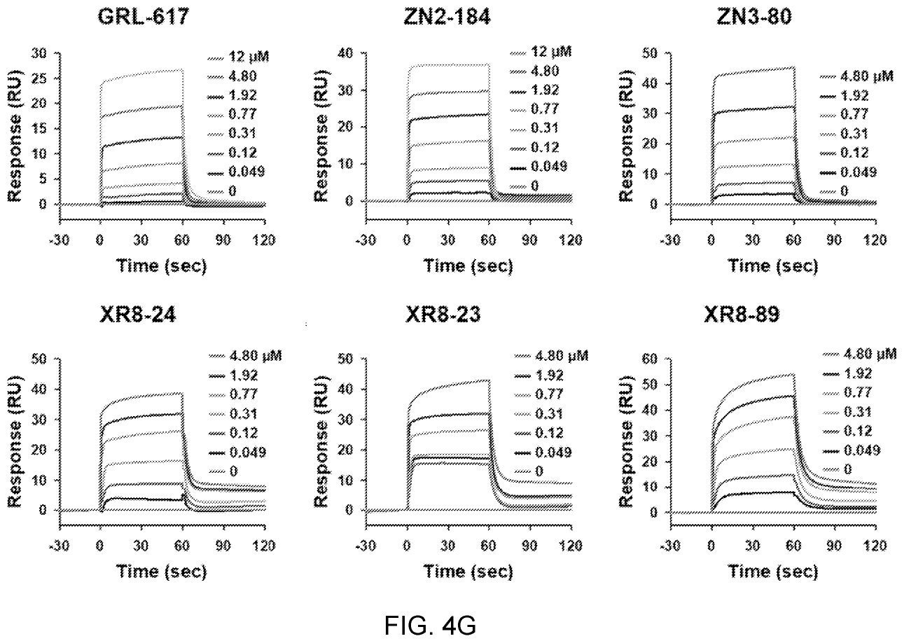

A detailed analysis of residues within the GRL0617 binding site of PLpro revealed five potential regions that were hypothesized could be targeted to increase affinity and potency for 1BL2-binding ligands (I-V, FIGS. 3D-3E). Corn-pounds extended into Site I explore potential interactions with Glu 167, which forms electrostatic contacts with the Arg72 of ubiquitin in the Ub-PLpro SARS-CoV co-crystal structure (PDB-: 4MM3) (FIG. 3F).¹⁹ It was contemplated that a basic amine appended to the aniline group would capture this interaction and improve binding affinity. A library of 16 compounds was synthesized to identify suitable basic side chains (FIG. 3E). The azetidine-substituted ZN2-184 was the most potent analogue targeting Site I, with a two-fold improvement relative to GRL0617, which correlated with affinity measured by SPR. The increase in affinity and potency was also accompanied by a twofold increase in rate of association (FIGS. 4A-4D).

Site II is located at the 53 site of the substrate-binding channel, which is formed by the BL12 loop, helix 5, and neighboring hydrophobic residues Tyr264, Tyr273, and LeuR62 (FIG. 3D). Small hydrophobic moieties such as a halogen or trifluoromethyl group were synthesized to probe the hydrophobic interaction at this site (FIG. 3E, Table 10).

TABLE 10

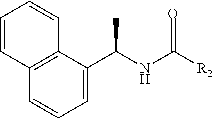

| | | Enzyme | |
|---|---|---|---|
| | | inhi- | SPR |
| | | bition | binding |
| Com- | | IC₅₀ | assays |
| pound | R₂ | (μM) | K_{D} (μM) |
| ZN-2-190 | 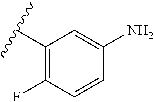 [SMILES: *c1cc(N)ccc1F] | >>100 | >1000 |
| ZN-2-192 | 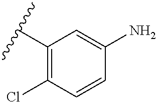 [SMILES: *c1cc(N)ccc1Cl] | 4.8 | 2.0 |
| ZN-2-193 | 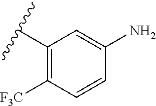 [SMILES: *c1cc(N)ccc1C(F)(F)F] | >10 | 454 |
| ZN-3-3 | 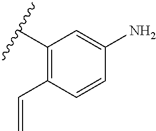 [SMILES: *c1cc(N)ccc1C=C] | >10 | 54.6 |
| DY2-109 | 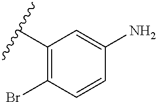 [SMILES: *c1cc(N)ccc1Br] | 21 | 83.0 |
| DY2-115 | 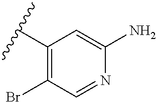 [SMILES: *c1cc(N)ncc1Br] | 7 | 29.6 |
| DY-3-8 | 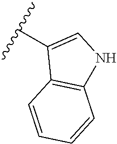 [SMILES: *c1c[nH]c2ccccc12] | >>100 | NA |
| DY-3-14 | 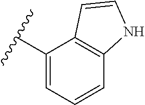 [SMILES: *c1cccc2[nH]ccc12] | >10 | 88.3 |
| DY-3-15 | 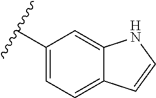 [SMILES: *c1ccc2cc[nH]c2c1] | 0.8 | 418.0 |
| DY-3-65 | 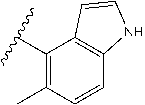 [SMILES: *c1c(C)ccc2[nH]ccc12] | 6.3 | 43.2 |
| DY-3-70 | 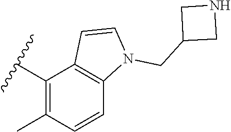 [SMILES: *c1c(C)ccc2c1ccn2CC1CNC1] | 6.4 | 43.4 |
| ZN-3-41 | 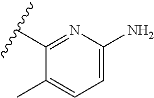 [SMILES: *c1nc(N)ccc1C] | 10-100 | >1000 |
| ZN-3-55 | 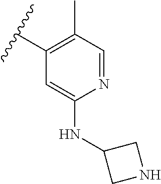 [SMILES: *c1cc(NC2CNC2)ncc1C] | 7.4 | 19.3 |
| ZN-3-57 | 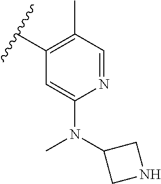 [SMILES: *c1cc(N(C)C2CNC2)ncc1C] | 10-100 | 245.5 |
| ZN-3-66 | 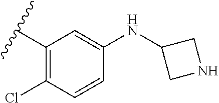 [SMILES: *c1cc(NC2CNC2)ccc1Cl] | 4.1 | 20.6 |
| ZN-3-70 | 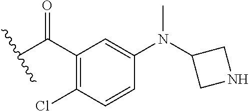 [SMILES: *C(=O)c1cc(N(C)C2CNC2)ccc1Cl] | 10.7 | 41.9 |

Interestingly, small substitutions such as methyl to fluorine at Site II led to a dramatic decrease in potency. Only bromo and chloro substituents did not significantly right-shift potency. Attempts to make fused-ring indole analogs to replace aniline also did not lead to any improvement in potency.

Site III is positioned next to the charged side chains of Arg166 and Asp164 (FIG. 3D). Arg166 forms an electrostatic interaction with Asp164 via its charged guanidino group, leaving the other guanidine nitrogens available for hydrogen bonding interactions. In the Ub-PLpro complex, this interaction is captured by hydrogen bonding to the Gln49 of ubiquitin (FIG. 3F). To exploit hydrogen bonding with Arg166 at Site III, analogues modified at the: 1) 2-napthelene position; 2) α-methyl position; and 3) aniline nitrogen, all without success (Tables 10-12) were synthesized.

TABLE 11

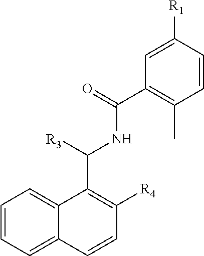

| | | | | | SPR |
|---|---|---|---|---|---|
| | | | | Enzyme | binding |
| | | | | inhibition | assays |
| Compound | R₁ | R₃ | R₄ | IC₅₀ (μM) | K_{D} (μM) |
| ZN-3-13 | NH₂ | 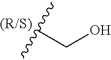 [SMILES: *CO] | H | ~100 | 39.1 |
| ZN-3-19 | NH₂ | (R)—CH₃ | OH | >100 | 184.0 |
| DY2-97 | NH₂ | 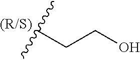 [SMILES: *CCO] | H | ~100 | 721.5 |
| ZN-3-61 | 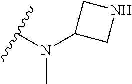 [SMILES: *N(C)C1CNC1] | (R)—CH₂CH₃ | H | >>10 | 281.5 |
| ZN-3-32 | 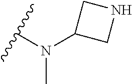 [SMILES: *N(C)C1CNC1] | (R)—CH₃ | OH | <100 | 240.2 |
| ZN-3-33 | 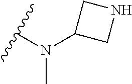 [SMILES: *N(C)C1CNC1] | (R)—CH₃ | 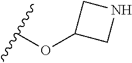 [SMILES: *OC1CNC1] | >10 | 54.6 |
| ZN-3-34 | 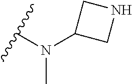 [SMILES: *N(C)C1CNC1] | (R)—CH₃ | 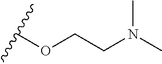 [SMILES: *OCCN(C)C] | NI | NA |
| ZN-3-35 | 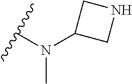 [SMILES: *N(C)C1CNC1] | 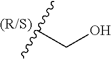 [SMILES: *CO] | H | >100 | 186.0 |
| DY2-116 | 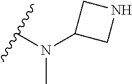 [SMILES: *N(C)C1CNC1] | 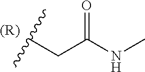 | H | NI | >1000 |
| DY2-117 | 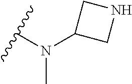 [SMILES: *N(C)C1CNC1] | 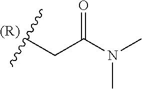 | H | NI | NA |

TABLE 12

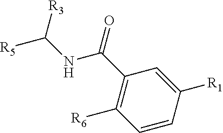

| | | | | | IC₅₀ | K_{D} |
|---|---|---|---|---|---|---|
| Compound | R₁ | R₃ | R₅ | R₆ | (μM) | (μM) |
| XDY2-62 | NH₂ | (R)—CH₃ | 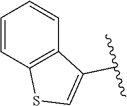 [SMILES: *c1csc2ccccc12] | CH₃ | 3.3 | 6.7 |
| XDY2-58 | NH₂ | (R)—CH₃ | 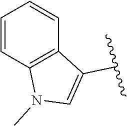 [SMILES: *c1cn(C)c2ccccc12] | CH₃ | <10 | 29.7 |
| YF4-134 | NO₂ | (R/S)—CH₃ | 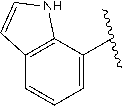 [SMILES: *c1cccc2cc[nH]c12] | CH₃ | >10 | >1000 |
| YF4-136 | NH₂ | (R/S)—CH₃ | 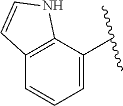 [SMILES: *c1cccc2cc[nH]c12] | CH₃ | 4.7 | 30.3 |
| YF4-137 | NH₂ | (R/S)—CH₃ | 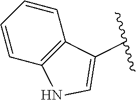 [SMILES: *c1c[nH]c2ccccc12] | CH₃ | ~100 | >1000 |
| YF4-145 | NH₂ | (R/S)—CH₃ | 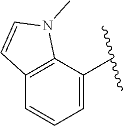 [SMILES: *c1cccc2ccn(C)c12] | CH₃ | >>100 | NA |
| DY2-125 | 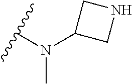 [SMILES: *N(C)C1CNC1] | (R)—CH₃ | 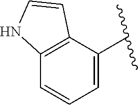 [SMILES: *c1cccc2[nH]ccc12] | CH₃ | 7 | 32.2 |
| ZN-3-45 | 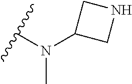 [SMILES: *N(C)C1CNC1] | (R/S)—CH₃ | 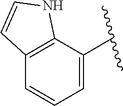 [SMILES: *c1cccc2cc[nH]c12] | CH₃ | 5.7 | 18.8 |
| DY-3-59 | 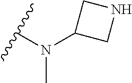 [SMILES: *N(C)C1CNC1] | (R)—CH₃ | 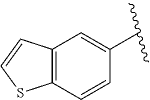 [SMILES: *c1ccc2sccc2c1] | CH₃ | 6.7 | 18.3 |
| DY-3-66 | 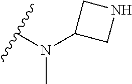 [SMILES: *N(C)C1CNC1] | (R)—CH₃ | 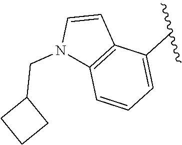 [SMILES: *c1cccc2c1ccn2CC1CCC1] | CH₃ | 3.3 | 14.1 |
| ZN-3-59 | 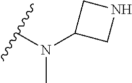 [SMILES: *N(C)C1CNC1] | (R)—CH₃ | 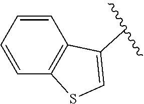 [SMILES: *c1csc2ccccc12] | CH₃ | 2.4 | 3.4 |
| ZN-3-67 | 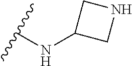 [SMILES: *NC1CNC1] | (R)—CH₃ | 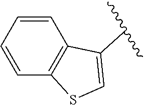 [SMILES: *c1csc2ccccc12] | Cl | 8.5 | 33.0 |
| ZN-3-71 | 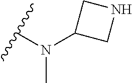 [SMILES: *N(C)C1CNC1] | (R)—CH₃ | 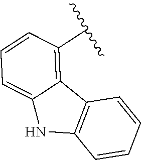 [SMILES: *c1cccc2[nH]c3ccccc3c12] | Cl | 10.9 | 64.2 |
| DY2-149 | 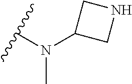 [SMILES: *N(C)C1CNC1] | (R)—CH₃ | 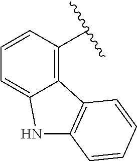 [SMILES: *c1cccc2[nH]c3ccccc3c12] | CH₃ | 1.6 | 8.4 |
| ZN-3-79 | 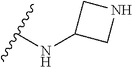 [SMILES: *NC1CNC1] | (R)—CH₃ | 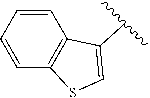 [SMILES: *c1csc2ccccc12] | CH₃ | 1.9 | 8.4 |
| DY-2-153 | 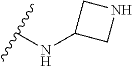 [SMILES: *NC1CNC1] | (R)—CH₃ | 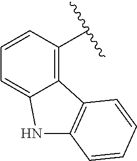 [SMILES: *c1cccc2[nH]c3ccccc3c12] | CH₃ | 1.8 | 3.9 |
| ZN-3-36 | 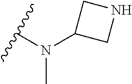 [SMILES: *N(C)C1CNC1] | (R)—CH₃ | 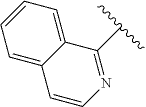 [SMILES: *c1nccc2ccccc12] | CH₃ | 56 | 19.6 |
| ZN-3-40 | 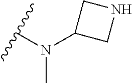 [SMILES: *N(C)C1CNC1] | (S)—CH₃ | 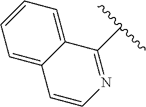 [SMILES: *c1nccc2ccccc12] | CH₃ | NI | NA |
| DY2-139 | 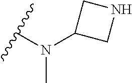 [SMILES: *N(C)C1CNC1] | (R)—CH₃ | 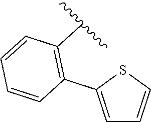 [SMILES: *c1ccccc1-c1cccs1] | CH₃ | >40 | NA |

Indeed, minor modifications, such as ZN3-36 with a 2-isoquinoline, designed to engage with a structurally conserved water molecule at Site III, significantly lost activity against PLpro (IC50=56 μM, FIG. 3D, Table 12). Conformational minimization (B3LYP/6-31G* with a polarizable continuum model for aqueous solvation) indicated a dihedral angle of 27.9° between the amide and isoquinoline planes of ZN3-36 (FIG. 3G). This angle is significantly different from that seen in the crystal structure of GRL-0617 (81.7°, PDB-7JRN), which may highlight the importance of maintaining a dihedral angle ˜90° for optimal hydrogen bonding with the 1BL2 loop (FIG. 3G).

Extending from the α-methyl position also proved to be futile. A minor ethyl modification led to a significant decrease in potency (ZN3-61); further expansion from this position resulted in almost completely inactive compounds such as DY2-97 and DY2-116 (Table 11). Only ZN3-56 with a glycine tail extended from the aniline site could engage with Site III and led to a slight improvement of potency over GRL0617. The proposed binding model of ZN3-56 predicts an electrostatic interaction with Arg 166 (FIG. 3H); however, this incremental improvement in potency suggests that electrostatic stabilization at this highly solvent-exposed region is counterbalanced by a solvation penalty. No further exploration of Site III interactions was attempted.

Scaffold hopping: naphthalene ring replacement. Although the active sites of PLpro from SARS-CoV and SARS CoV-2 are identical, there are several amino acids that differ between the two enzymes, which are within approximately 10 Å of the binding site of BL2-stabilizing ligands GRL-0617 (e.g., E251/Q250 and N263/S262). It is reasonable to assume that these substitutions might subtly alter ligand binding to the BL2 loop in SARS-CoV-2 PLpro versus SARS-CoV. This observation alone supports exploration of scaffolds to replace the naphthalene of GRL0617. Retaining the essential geometry between the benzamide and naphthalene rings should be possible using heteroaryl or bi-aryl group replacements. Replacement of the naphthalene ring is also anticipated to reduce metabolic instability.³⁰ Modifications at this site should also allow favorable interactions with Site IV (FIG. 3D). Fused heteroaryls such as benzothiophene, indole, and carbazole with various linkages were prepared and tested (Table 12); however, most modifications weakened or lost activity. Only the 3-benzothiophene (ZN3-79) and the carbazole-based (DY2-153) analogues showed comparable potency to GRL0617 (IC₅₀=1.9 μM and 1.8 μM, respectively, Table 12). In contrast, the biaryl analogues not only retained the activity of GRL0617 but also gained potency. Both 2-phenylthiophene (ZN-3-80; IC50=0.59 μM) and 3-phenylthiophene (XR8-8; IC50=1.3 μM) demonstrated enhanced potency. Importantly, ZN3-80, the most potent analog in this subset, was found to be more stable than GRL0617 in human liver microsome stability assays (Table 13), which encouraged us to explore further optimization.

TABLE 13
Metabolic Stability of Test Compounds
in Pooled Human Liver Microsomes

| | | | In vitro CIᵢₙₜ | |
|---|---|---|---|---|
| | | In vitro T_{1/2} | (μL/min/mg | Scale-up CIᵢₙₜ |
| Compound ID | Species | (min) | protein) | (mL/min/kg) |
| Verapamil | Human | 13.91 | 99.66 | 124.99 |
| XR8-23 | Human | 235.50 | 5.89 | 7.38 |
| XR8-24 | Human | 144.38 | 9.60 | 12.04 |
| XR8-57 | Human | 5125.53 | 0.27 | 0.34 |
| GRL0617 | Human | 105.56 | 13.13 | 16.47 |
| ZN3-80 | Human | 245.82 | 5.64 | 7.07 |

Engaging the BL2 groove decreases inhibitor off-rate and improves potency. Examination of crystal structures identified a ligand binding site, coined the “BL2 groove” (Site V, FIG. 3D), which is positioned at the N-terminal side of the BL12 loop and features hydrophobic residues such as Pro248 and Pro299 and potential hydrogen bonding partners such as the backbone amide of Gly266. With an optimized 2-phenylthiophene (ZN3-80) in hand, the next steps included exploration of derivatization to exploit further interactions with the BL12 groove, synthesizing 22 compounds. Interestingly, nine compounds in this family significantly impacted potency, improving IC₅₀ to below 500 nM (FIG. 4A, Table 14).

TABLE 14

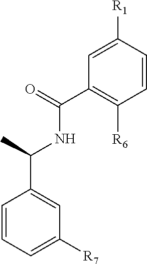

| | | | | IC₅₀ | K_{D} |
|---|---|---|---|---|---|
| Compound | R₁ | R₆ | R₇ | (μM) | (μM) |
| ZN-3-74 | 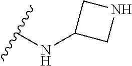 [SMILES: *NC1CNC1] | Cl | 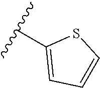 [SMILES: *c1cccs1] | 2.8 | NA |
| ZN-3-80 | 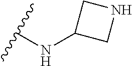 [SMILES: *NC1CNC1] | CH₃ | 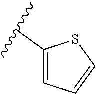 [SMILES: *c1cccs1] | 0.59 | 0.963 |
| XR8-8 | 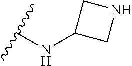 [SMILES: *NC1CNC1] | CH₃ | 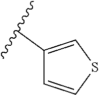 [SMILES: *c1ccsc1] | 1.3 | 1.39 |
| XR8-9 | 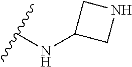 [SMILES: *NC1CNC1] | CH₃ | 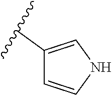 [SMILES: *c1cc[nH]c1] | 1.8 | 2.89 |
| XR8-14 | 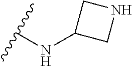 [SMILES: *NC1CNC1] | CH₃ | 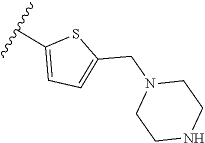 [SMILES: *c1ccc(CN2CCNCC2)s1] | 1.2 | 0.846 |
| XR8-15 | 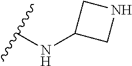 [SMILES: *NC1CNC1] | CH₃ | 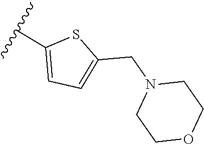 [SMILES: *c1ccc(CN2CCOCC2)s1] | 0.9 | 1.56 |
| XR8-16 | 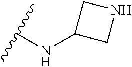 [SMILES: *NC1CNC1] | CH₃ | 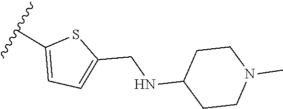 [SMILES: *c1ccc(CNC2CCN(C)CC2)s1] | 1.6 | 0.43 |
| XR8-17 | 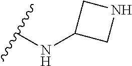 [SMILES: *NC1CNC1] | CH₃ | 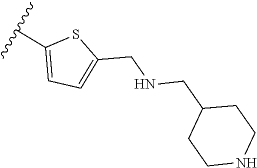 [SMILES: *c1ccc(CNCC2CCNCC2)s1] | 2.7 | 0.777 |
| XR8-23 | 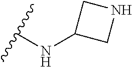 [SMILES: *NC1CNC1] | CH₃ | 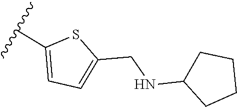 [SMILES: *c1ccc(CNC2CCCC2)s1] | 0.39 | 0.235 |
| XR8-24 | 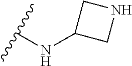 [SMILES: *NC1CNC1] | CH₃ | 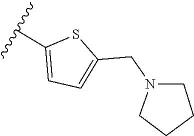 [SMILES: *c1ccc(CN2CCCC2)s1] | 0.56 | 0.372 |
| XR8-30 | 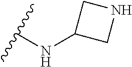 [SMILES: *NC1CNC1] | CH₃ | 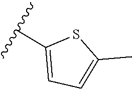 [SMILES: *c1ccc(C)s1] | 0.75 | 1.15 |
| XR8-32-1 | 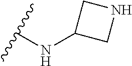 [SMILES: *NC1CNC1] | CH₃ | 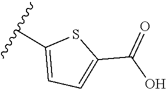 [SMILES: *c1ccc(C(=O)O)s1] | 0.97 | 2.06 |
| XR8-32-2 | 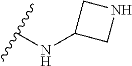 [SMILES: *NC1CNC1] | CH₃ | 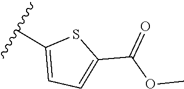 [SMILES: *c1ccc(C(=O)OC)s1] | 0.81 | 2.4 |
| XR8-35 | 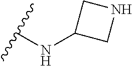 [SMILES: *NC1CNC1] | CH₃ | 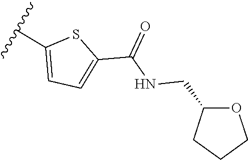 [SMILES: *c1ccc(C(=O)NC[C@H]2CCCO2)s1] | 0.92 | 1.79 |
| XR8-38 | 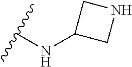 [SMILES: *NC1CNC1] | CH₃ | 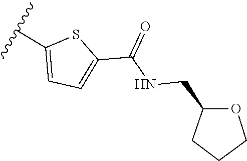 [SMILES: *c1ccc(C(=O)NC[C@@H]2CCCO2)s1] | 0.76 | 0.741 |
| XR8-39 | 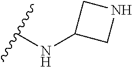 [SMILES: *NC1CNC1] | CH₃ | 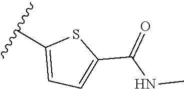 [SMILES: *c1ccc(C(=O)NC)s1] | 1.1 | 1.02 |
| XR8-40 | 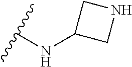 [SMILES: *NC1CNC1] | CH₃ | 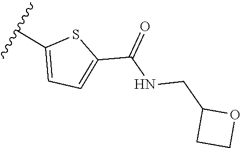 [SMILES: *c1ccc(C(=O)NCC2CCO2)s1] | 0.82 | 1.42 |
| XR8-49 | 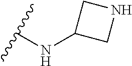 [SMILES: *NC1CNC1] | CH₃ | 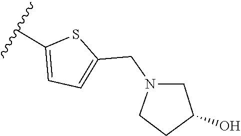 [SMILES: *c1ccc(CN2CC[C@@H](O)C2)s1] | 0.64 | 0.761 |
| XR8-51 | 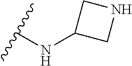 [SMILES: *NC1CNC1] | CH₃ | 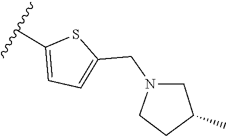 [SMILES: *c1ccc(CN2CC[C@@H](C)C2)s1] | 1.1 | 0.776 |
| XR8-56 | 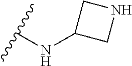 [SMILES: *NC1CNC1] | CH₃ | 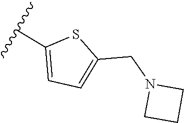 [SMILES: *c1ccc(CN2CCC2)s1] | 2.2 | 1.79 |
| XR8-57 | 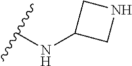 [SMILES: *NC1CNC1] | CH₃ | 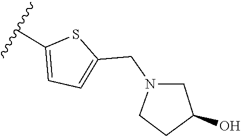 [SMILES: *c1ccc(CN2CC[C@H](O)C2)s1] | 0.70 | 0.668 |
| XR8-61 | 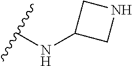 [SMILES: *NC1CNC1] | CH₃ | Br | 6.5 | 3.13 |
| XR8-65 | 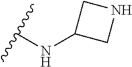 [SMILES: *NC1CNC1] | CH₃ | 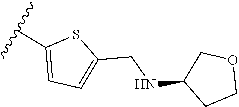 [SMILES: *c1ccc(CN[C@@H]2CCOC2)s1] | 0.33 | 1.53 |
| XR8-66 | 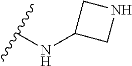 [SMILES: *NC1CNC1] | CH₃ | 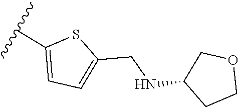 [SMILES: *c1ccc(CN[C@H]2CCOC2)s1] | 0.62 | 0.84 |
| XR8-67 | 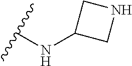 [SMILES: *NC1CNC1] | CH₃ | 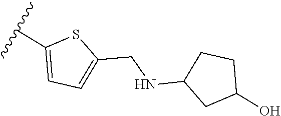 [SMILES: *c1ccc(CNC2CCC(O)C2)s1] | 0.17 | 0.79 |
| XR8-69 | 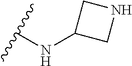 [SMILES: *NC1CNC1] | CH₃ | 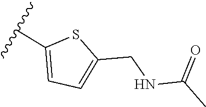 [SMILES: *c1ccc(CNC(C)=O)s1] | 0.37 | NA |
| XR8-77 | 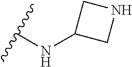 [SMILES: *NC1CNC1] | CH₃ | 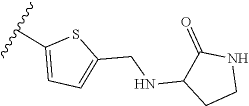 [SMILES: *c1ccc(CNC2CCNC2=O)s1] | 0.64 | 1.78 |
| XR8-79 | 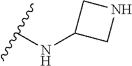 [SMILES: *NC1CNC1] | CH₃ | 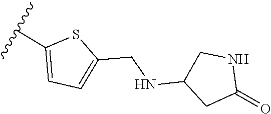 [SMILES: *c1ccc(CNC2CNC(=O)C2)s1] | 0.41 | 0.72 |
| XR8-83 | 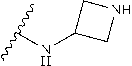 [SMILES: *NC1CNC1] | CH₃ | 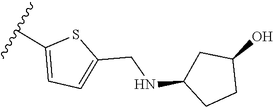 [SMILES: *c1ccc(CN[C@@H]2CC[C@H](O)C2)s1] | 0.21 | 0.63 |
| XR8-84 | 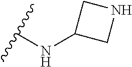 [SMILES: *NC1CNC1] | CH₃ | 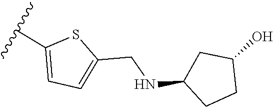 [SMILES: *c1ccc(CN[C@@H]2CC[C@@H](O)C2)s1] | 0.43 | 0.53 |
| XR8-89 | 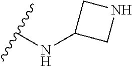 [SMILES: *NC1CNC1] | CH₃ | 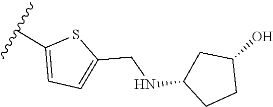 [SMILES: *c1ccc(CN[C@H]2CC[C@@H](O)C2)s1] | 0.113 | 0.113 |
| XR8-96 | 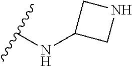 [SMILES: *NC1CNC1] | CH₃ | 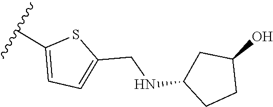 [SMILES: *c1ccc(CN[C@H]2CC[C@H](O)C2)s1] | 0.25 | 0.40 |
| XR8-98 | NH₂ | CH₃ | 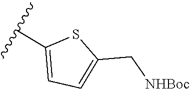 [SMILES: *c1ccc(CNC(=O)OC(C)(C)C)s1] | 0.81 | 5.01 |
| XR8-101 | NH₂ | CH₃ | 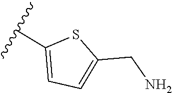 [SMILES: *c1ccc(CN)s1] | 1.8 | 2.86 |
| XR8-103 | NH₂ | CH₃ | 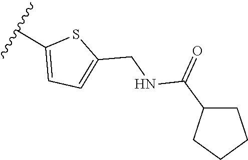 [SMILES: *c1ccc(CNC(=O)C2CCCC2)s1] | 1.1 | 2.28 |
| XR8-104 | NHAc | CH₃ | 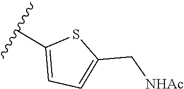 [SMILES: *c1ccc(CNC(C)=O)s1] | 2.3 | 3.07 |
| XR8-106 | NH₂ | CH₃ | 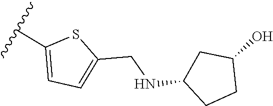 [SMILES: *c1ccc(CN[C@H]2CC[C@@H](O)C2)s1] | 1.4 | 2.20 |

It was hypothesized that once bound, these extended ligands interacting with Sites I-V might have slower off-rates because of the increased conformational reorganization of the BL2 loop required for ligand release (FIG. 4G, Table 15).

TABLE 15
SPR data from 4 replicates

| | kₐ1 | kₐ2 | kₐ3 | kₐ4 | Ave kₐ | STD kₐ |
|---|---|---|---|---|---|---|
| Compounds | (M⁻¹s⁻¹) | (M⁻¹s⁻¹) | (M⁻¹s⁻¹) | (M⁻¹s⁻¹) | (M⁻¹s⁻¹) | (M⁻¹s⁻¹) |
| GRL0617 | 2.02E+05 | 1.73E+05 | 1.45E+05 | 1.96E+05 | 1.79E+05 | 2.61E+04 |
| ZN2-184 | 4.26E+05 | 3.48E+05 | 2.31E+05 | 3.56E+05 | 3.40E+05 | 8.08E+04 |
| ZN3-80 | 2.91E+05 | 3.34E+05 | 3.03E+05 | 4.77E+05 | 3.51E+05 | 8.56E+04 |
| XR8-24 | 4.70E+05 | 2.71E+05 | 1.99E+05 | 4.17E+05 | 3.39E+05 | 1.26E+05 |
| XR8-23 | 5.42E+05 | 5.53E+05 | 4.14E+05 | 5.79E+05 | 5.22E+05 | 7.37E+04 |
| XR8-89 | 5.21E+05 | 4.27E+05 | | | 4.74E+05 | 6.61E+04 |
| | k_{d}1 | k_{d}2 | k_{d}3 | k_{d}4 | Ave k_{d} | STD k_{d} |
| Compounds | (s⁻¹) | (s⁻¹) | (s⁻¹) | (s⁻¹) | (s⁻¹) | (s⁻¹) |
| GRL0617 | 0.556 | 0.464 | 0.421 | 0.478 | 0.480 | 0.056 |
| ZN2-184 | 0.438 | 0.407 | 0.246 | 0.305 | 0.349 | 0.089 |
| ZN3-80 | 0.318 | 0.369 | 0.290 | 0.332 | 0.327 | 0.033 |
| XR8-24 | 0.157 | 0.110 | 0.066 | 0.175 | 0.127 | 0.049 |
| XR8-23 | 0.140 | 0.153 | 0.081 | 0.121 | 0.124 | 0.031 |
| XR8-89 | 0.0529 | 0.0531 | | | 0.0530 | 0.00017 |
| | K_{D}1 | K_{D}2 | K_{D}3 | K_{D}4 | Ave K_{D} | STD K_{D} |
| Compounds | (M) | (M) | (M) | (M) | (M) | (M) |
| GRL0617 | 2.75E−06 | 2.69E−06 | 2.91E−06 | 2.44E−06 | 2.70E−06 | 1.94E−07 |
| ZN2-184 | 1.03E−06 | 1.17E−06 | 1.06E−06 | 8.56E−07 | 1.03E−06 | 1.31E−07 |
| ZN3-80 | 1.09E−06 | 1.10E−06 | 9.56E−07 | 6.97E−07 | 9.63E−07 | 1.90E−07 |
| XR8-24 | 3.34E−07 | 4.04E−07 | 3.30E−07 | 4.20E−07 | 3.72E−07 | 4.70E−08 |
| XR8-23 | 2.59E−07 | 2.77E−07 | 1.96E−07 | 2.09E−07 | 2.35E−07 | 3.89E−08 |
| XR8-89 | 1.02E−07 | 1.24E−07 | | | 1.13E−07 | 1.61E−08 |

Association and dissociation rates were measured by SPR (FIG. 4B). The extended ligands, designed to engage the BL12 groove, showed a marked decrease in dissociation rates (FIG. 4C). For example, both XR8-23 and XR8-89, with basic amine side chains extending from the thiophene of ZN3-80, produced 4-fold and 9-fold reductions in off rates compared with GRL0617, respectively; suggesting that BL12 groove engagement is a novel strategy for development of potent PLpro inhibitors with slower off-rates.

To confirm that the improved analogs can inhibit the DUB activity of SARS-CoV-2 PLpro, both Ub-AMC (FIG. 4E) and ISG-15-AMC (FIG. 4F) were studied as substrates for the enzyme at three inhibitor concentrations. Complete ablation of DUB activity was observed with all tested inhibitors at 30 μM and at the approximate I050 concentration for GRL0617, all novel inhibitors produced greater inhibition of DUB activity. The data support the ability of these novel inhibitors to block SARS-CoV-2 PLpro-mediated deubiquitination and delSGylation of host proteins involved in host immune response.

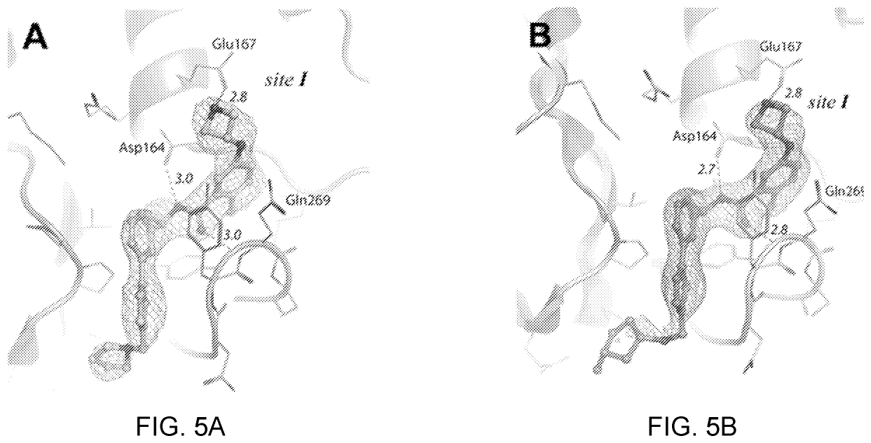

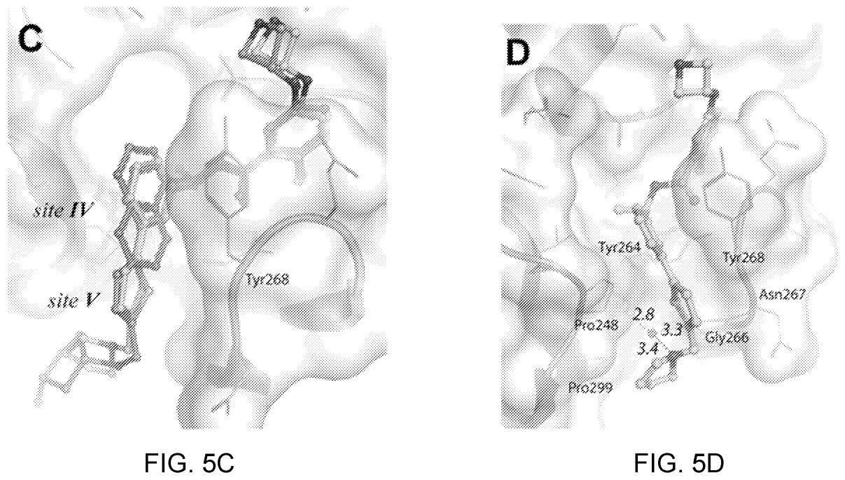

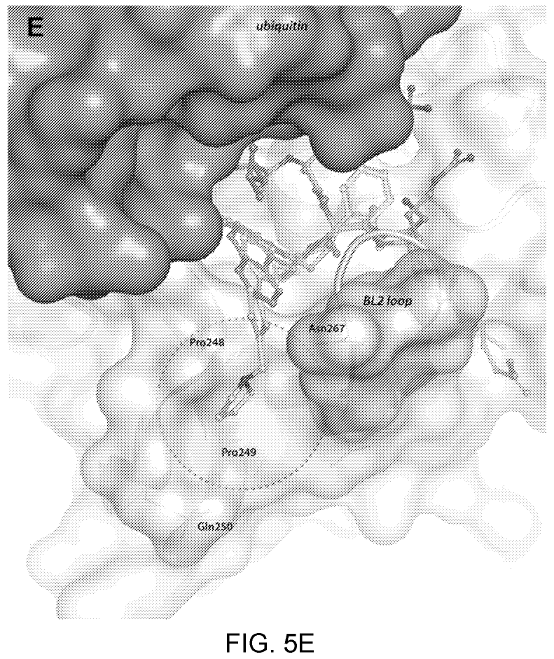

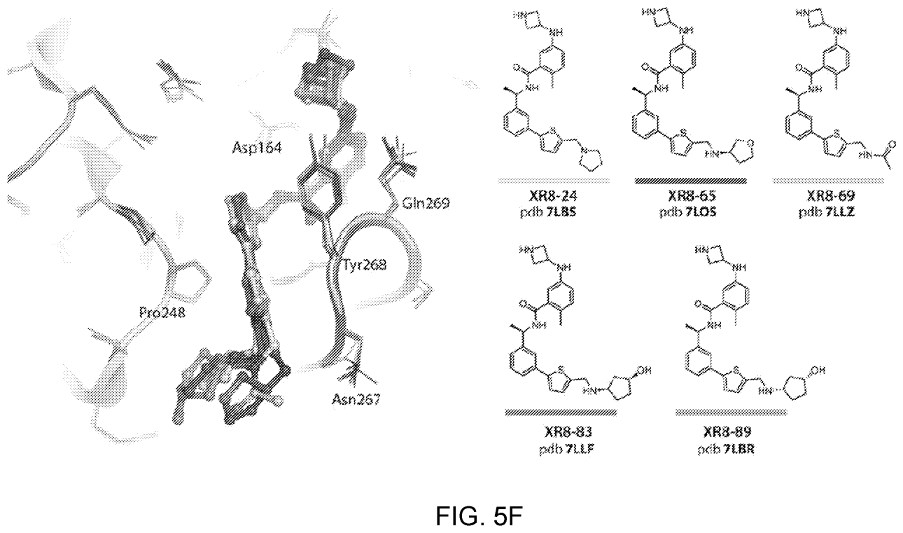

Co-crystal structures of XR8-23 and XR8-89 with SARS-CoV-2 PLpro. To examine the binding mode of the novel PLpro inhibitors and test the proposed binding site hypotheses, co-crystal structures of XR8-24, XR8-65, XR8-69, XR8-83, and XR8-89 complexed with SARS-CoV-2 PLpro were obtained. Superposition of the ligand-bound structures shows all inhibitors enforcing the same binding mode of the BL2 loop (FIG. 5F). Focusing on the co-crystal structures of XR8-24 and XR8-89, it was observe that the azetidine ring extends into Site/to within 3 Å of Glu168, likely engaging in the postulated electrostatic stabilizing interaction (FIGS. 5A-5B). The amide group of XR8-24 and XR8-89 is aligned closely with that of GRL0617 in SARS-CoV-2 PLpro (PDB: 7JRN) with the expected: i) carbonyl hydrogen bonding to the mainchain of Gln269 on the BL2 loop; and ii) amide nitrogen hydrogen bonding to Asp164 of helix 5. In Site IV, the 2-phenylthiophene of the ligand retains the T-shaped pi-interaction with Tyr268, as seen for the naphthalene ring of GRL0617; however, there is a shift in the biaryl ring of XR8-24 relative to the naphthalene of GRL0617, suggesting that the biaryl substituent maximizes interactions that may not be present in PLpro from SARS-CoV. These interactions place the thiophene firmly in the BL2 groove (Site V), where it takes part in van der Waals interactions with residues surrounding the cavity (Pro248, Tyr264, Tyr268; FIG. 5D).

The “tail” of both XR8-24 and XR8-89, which it was postulated to provide interactions with the BL2 groove in these co-crystal structures sits perpendicular to the thiophene and adjacent to the body of the protein near Pro248 and Pro299. The lack of electron density for the tail of XR8-89 (FIG. 5B), also observed for the co-crystal structures of XR8-65, XR8-69 and XR8-83 indicates that this region is largely disordered, which may be due in part both to high solvent-exposure and crystal packing forces induced by the proximity to a second symmetry-related monomer. However, the tail of XR8-24 is better defined, with the pyrrolidine ring forming a putative water-mediated hydrogen bond to the mainchain carbonyl oxygens of Tyr264 and Gly266 (FIG. 5D). The new co-crystal structures show that engagement of the BL2 groove contributes to binding affinity this binding interaction is unique and is not observed in structures of SARS CoV-2 PLpro in complex with ubiquitin or ISG15, nor with any known PLpro inhibitors (FIG. 5E).

Improved potency translates to improved antiviral activity. Of the potent, novel PLpro inhibitors, selection for antiviral testing was based on the following rationale: XR8-89 demonstrated highest potency for PLpro inhibition (IC50=113 nM); XR8-23 demonstrated a high association/dissociation rate ratio; and XR8-24 yielded a superior co-crystal structure. No toxicity was observed under assay conditions in Vero E6 cells for these compounds at <50 μM; therefore, all three compounds were evaluated and compared with GRL0617 in a plaque formation assay using the SARS-CoV-2 USA/WA1/2020 strain. Vero E6 cells are known to express high levels of efflux transporter proteins.⁴¹ In a report on the 3CLpro inhibitor, PF-00835231, currently in clinical trials for COVID-19, co-treatment with the dual P-gp/BCRP inhibitor, CP-100356, was required to elicit antiviral activity in Vero E6 cells. ⁴² In Caco-2 cells, XR8-24 demonstrated a high efflux ratio (Table 16), implying efficient P-gp mediated efflux; therefore CP-100356 co-treatment (1.5 μM) was used to test antiviral activity in Vero E6 cells.

TABLE 16
Permeability Results of Test Compounds in Caco-2 Cell Line

| | P_{app (A-B)} | P_{app (B-A)} | | Recovery | Recovery |
|---|---|---|---|---|---|
| Compound | (10⁻⁶, | (10⁻⁶, | Efflux | (%) | (%) |
| ID | cm/s) | cm/s) | Ratio | AP-BL | BL-AP |
| Propranolol | 17.92 | 18.26 | 1.02 | 58.60 | 84.44 |
| Digoxin | 0.40 | 10.45 | 26.32 | 85.20 | 92.39 |
| GRL0617 | 12.40 | 12.95 | 1.04 | 49.67 | 59.95 |
| XR8-23 | 0.16 | 1.56 | 9.94 | 68.60 | 80.29 |
| XR8-24 | 0.14 | 3.06 | 21.79 | 85.56 | 96.59 |

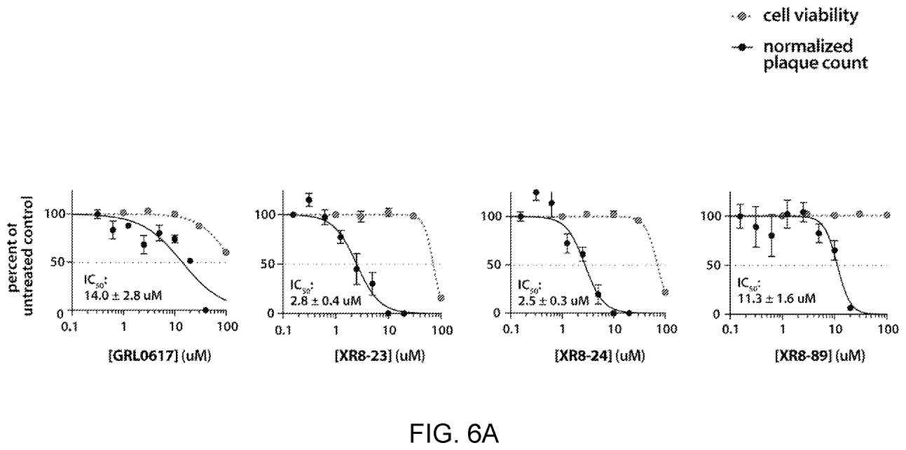

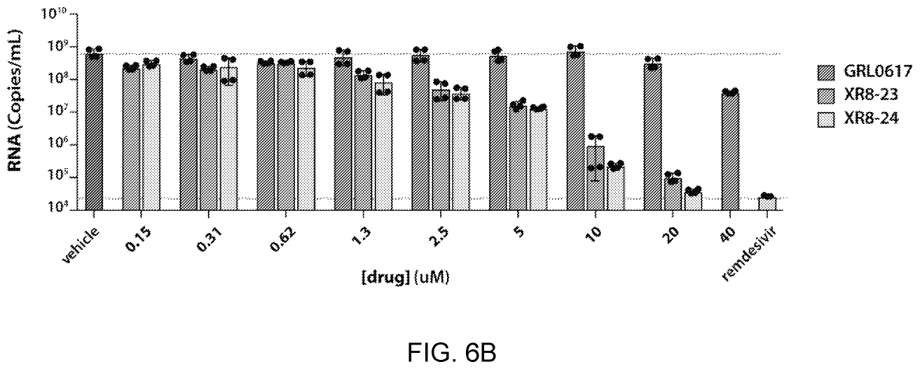

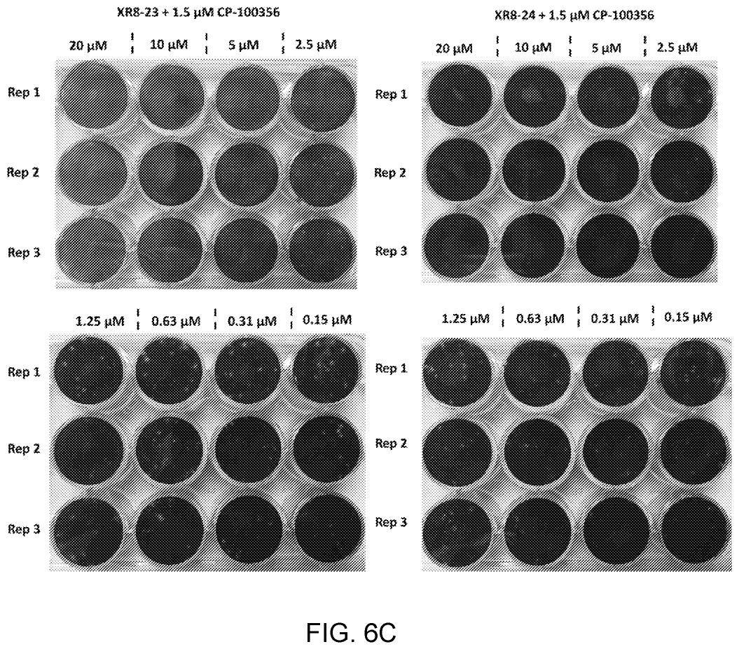

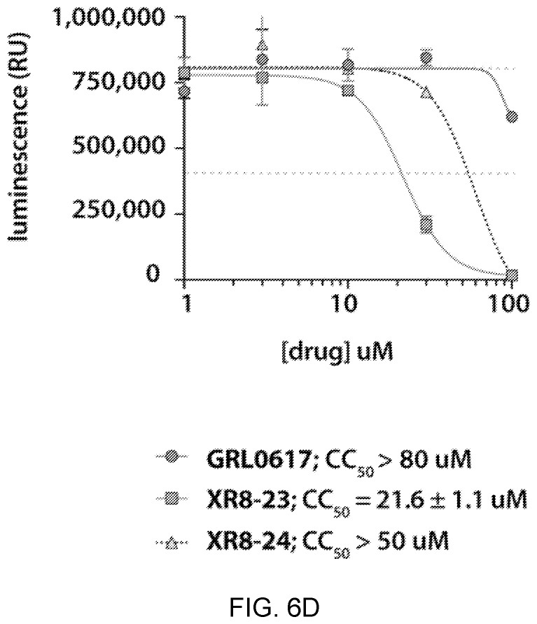

Consistent with reports on PF-00835231, CP-100356 itself had no antiviral activity and at the tested concentrations did not influence cytotoxicity alone or in combination with the PLpro inhibitors. Consistent with contemporary reports on GRL0617,²¹ an EC₅₀ of 21.7±1.6 μM was measured against infectious SARS-CoV-2 in an eight-point dose response assay. Both XR8-23 and XR8-24 were significantly more potent than GRL0617 with EC₅₀ measured at 2.8±0.4 μM and 2.5±1.9 μM, respectively (FIG. 6A). XR8-89 also demonstrated superior antiviral potency to GRL017; however, antiviral potency did not correlate with the superior potency of this inhibitor in biochemical assays. The lack of observable cytotoxicity for XR8-89 might indicate attenuated cell permeability as a cause of lower antiviral potency.

The two most potent antiviral agents in Vero E6 cells, XR8-23 and XR8-24, were tested and compared to GRL0617 and remdesivir, an FDA-approved COVID-19 antiviral agent, in the human lung epithelial A549 cell line stably overexpressing the human ACE2 receptor. Although monkey Vero E6 cells are a standard model for antiviral testing, a human cell line provides an orthogonal and more relevant model system. Viral RNA was assayed by RT-qPCR as a measure of replication of infectious SARS-CoV-2 USA/WA1/2020. The assay was conducted in the absence of CP-100356 and cytotoxicity was not observed under assay conditions at <50 μM for XR8-24 and <10 μM for XR8-23 (FIG. 6D). The antiviral activity of novel PLpro inhibitors was markedly superior to that of GRL0617 in this model system (FIG. 6B). Both XR8-23 and XR8-24 were again significantly more potent than GRL0617 (IC₅₀>20 μM) with IC₅₀ measured at 1.4±0.1 μM and 1.2±0.2 μM, respectively. By unpaired nonparametric t-test: 1) the effect of treatment with XR8-23 and XR8-24 (1.3 μM) was significantly different from vehicle control; and 2) the effect of treatment with XR8-24 (20 μM) was not significantly different from that of remdesivir (10 μM).

### Conclusion

The pathogenesis of the SARS-CoV-2 infection is characterized by a strong dysregulation of the innate immune and the type I interferon (IFN-I) responses.⁴³ The viral protein, PLpro, represents an excellent therapeutic target owing to multi-functional roles: i) in mediating viral replication via processing of the viral polyprotein; and ii) in reversing host-mediated post-translational modifications in response to viral infection via its actions as a DUB. The DUB enzyme activity of PLpro is responsible for removing ubiquitin chains and the ISG15 ubiquitin-like (Ubl) modification from host proteins. ISGylation of proteins is induced during viral infection as a host antiviral signaling mechanism.⁴⁴ Interestingly, despite the close homology of PLpro from SARS-CoV-2 and from the original SARS-CoV coronovirus, PLpro has been claimed differentially to modulate the host immune system: specifically, it is reported that SARS-CoV-2 PLpro preferentially cleaves ISG15, whereas PLpro from SARS-CoV predominantly targets ubiquitin chains.^{20,45} In addition to Ub- and Ubl-modified host proteins, the autophagy-activating kinase, ULK1, is also a substrate for PLpro, cleaving the N-terminal kinase domain from a C-terminal substrate recognition region to disrupt autophagy during early viral replication.⁴⁶ Pharmacological inhibitors of PLpro are needed to probe the effects of PLpro proteolytic activity on host cell immune response and autophagy; however, more urgent is the need for PLpro inhibitors that effectively block viral replication, since many SARS-CoV-2 gene products in addition to PLpro have immune modulatory effects.

Towards developing potent inhibitors, developed a robust high-throughput assay for SARS-CoV-2 PLpro using Z-RLRGG-AMC (SEQ ID NO. 4) to screen chemical libraries including FDA-approved drugs and molecules in clinical trials (15,370 molecules) was developed. Consistent with contemporary reports,^{38,40,47} an extremely low hit rate was observed, which makes repurposing of approved drugs as therapeutically useful PLpro inhibitors problematic. As recent reports have noted, the experimental data is not in accordance with in silico repurposing predictions:^{48,49} for example, isotretinoin reportedly in clinical trials for COVID-19 as a PLpro inhibitor, ⁴⁶ was inactive in the screen. Avasimibe and candesartan were identified as weak PLpro inhibitors; however, only GRL0617 and CPI-169 (initially developed as an EZH2 inhibitor) were validated as hits with potency/affinity <10 μM in both enzymatic assay and SPR binding assays. CPI-169 represents a novel chemical scaffold and a novel addition to the very limited PLpro inhibitor chemotypes identified in the literature.^{33,50}

Given the availability of co-crystal structures of GRL0617:PLpro (SARS-CoV-2), structure-guided drug design was pursued exploring this chemical scaffold. Five binding sites on PLpro were explored by modification of the benzamide scaffold to identify additional interactions to increase inhibitor potency. Site I contains Glu167, which forms a salt bridge with Arg72 of ubiquitin in the Ub:PLpro complex. This electrostatic interaction was captured with addition of a basic amine side chain in novel inhibitors to yield a significant increase in binding and potency. Additional modifications to engage with the S1 or S2 sites in the channel leading to the catalytic site were unsuccessful, consistent with recent findings profiling substrate specificity using a combinatorial library.⁵¹ Site III is defined by Arg166, which forms a hydrogen bonding interaction with Gln49 of ubiquitin; however, none of the modifications designed to mimic this interaction increased the affinity of inhibitors for PLpro. Site III, therefore, remains to be exploited in future work.

The BL2 groove is a new binding site identified in the process of inhibitor optimization, which was confirmed and validated by obtaining SARS-CoV-2 PLpro co-crystal structures. This BL2 groove is not involved in the binding of any PLpro substrates, such as Ubs and Ubls, by the enzyme. Novel inhibitors, such as XR8-23 and XR8-24, modified with BL2-interacting side chains, showed both improved binding affinity and slower off-rates, suggesting that BL2 groove interactions can yield more efficacious PLpro inhibitors. Gratifyingly, these enhanced biochemical properties translated to antiviral efficacy against infectious SARS-CoV-2 (USA/WA1/2020) in Vero E6 green monkey kidney epithelial cells and A549 human lung epithelial cells. The low micromolar potency observed in inhibition of viral plaque formation was superior to GRL0617 and suggests that optimization of PLpro inhibitors as therapeutic agents for SARS-CoV-2 is feasible. Vero E6 cells are highly susceptible to the cytopathic effects of SARS-CoV-2 infection in contrast to many human cell lines.⁵² The observations in a human lung epithelial cell line of inhibition of SARS-CoV-2 viral replication is therefore very promising. Novel PLpro inhibitors were markedly more efficacious than GRL0617, with significant suppression of viral RNA at low micromolar concentrations.

PLpro inhibitors such as XR8-23 and XR8-24 provide an opportunity to study combination therapy with FDA-approved RdRp inhibitors such as remdesivir, or 3CLPro inhibitors such as PF-00835231, now in Phase I/II clinical trials. Genotyping of SARS-CoV-2 virus strains circulating worldwide has identified multiple recurrent non-synonymous mutations in the receptor-binding domain (RBD) of the spike protein. For example, the SARS-CoV-2 B.1.1.7 strain identified in London contains a N501Y mutation in the RBD domain. Variants with multiple mutations in the spike protein pose a risk of resistance to current FDA-approved vaccines and therapeutic antibodies, mutations in the cysteine proteases 3CLpro and PLpro have not been reported.

In conclusion, this study included the identified a new drug-like PLpro inhibitor chemotype, CPI-169, adding to the very limited examples of PLpro inhibitor scaffolds. Guided by new SARS-CoV-2 PLpro co-crystal structures, this study included the design novel non-covalent PLpro inhibitors that exhibited superior nanomolar potency and inhibited PLpro DUB activity, without inhibiting human DUBs. Biochemical potency, affinity, and slow off-rates translated to low micromolar potency against infectious SARS-CoV-2 in primate and human cell lines. Non-covalent inhibitors that stabilize the BL2 loop by induced fit do not occupy the active site of PLpro and published data on SARS-CoV PLpro inhibitors showed relatively weak potency against infectious virus; therefore, the therapeutic relevance of this approach in SARS-CoV-2 antiviral therapy was problematic. This study shows shown that BL2-stabilizing PLpro inhibitors have therapeutically relevant activity against SARS-CoV-2.

We synthesized almost 100 analogues during structure-based optimization, identifying the novel BL2 groove as an important ligand binding site. The newly identified non-covalent inhibitors described herein are the most potent SARS-CoV-2 PLpro inhibitors reported to date, with improved potency and metabolic stability. The infectious virus data suggests that administration in combination with remdesivir or 3CLpro inhibitors may be therapeutically beneficial. Moreover, potent PLpro inhibitors such as XR8-23 and XR8-24 represent chemical probe tool compounds to study the details of PLpro-mediated disruption of host immune response and autophagy; and their contribution to COVID-19 infection and progression, including “long-COVID” and potential genetic bias.^{53,54}

### REFERENCES

1. Chen, N. et al. Epidemiological and clinical characteristics of 99 cases of 2019 novel coronavirus pneumonia in Wuhan, China: a descriptive study. The Lancet 395, 507-513, doi:https://doi.org/10.1016/S0140-6736(20)30211-7 (2020).
2. Zhou, P. et al. A pneumonia outbreak associated with a new coronavirus of probable bat origin. Nature 579, 270-273, doi:10.1038/s41586-020-2012-7 (2020).
3. Wu, F. et al. A new coronavirus associated with human respiratory disease in China. Nature 579, 265-269, doi:10.1038/s41586-020-2008-3 (2020).
4. Dong, E., Du, H. & Gardner, L. An interactive web-based dashboard to track COVID-19 in real time. The Lancet Infectious Diseases 20, 533-534, doi:10.1016/S1473-3099(20)30120-1 (2020).
5. Sabino, E. C. et al. Resurgence of COVID-19 in Manaus, Brazil, despite high seroprevalence. The Lancet, doi:10.1016/S0140-6736(21)00183-5.
6. Wan, Y., Shang, J., Graham, R., Baric, R. S. & Li, F. Receptor Recognition by the Novel Coronavirus from Wuhan: an Analysis Based on Decade-Long Structural Studies of SARS Coronavirus. J Virol 94, doi:10.1128/JVI.00127-20 (2020).
7. Kruse, R. L. Therapeutic strategies in an outbreak scenario to treat the novel coronavirus originating in Wuhan, China. F1000Res 9, 72, doi:10.12688/f1000research.22211.2 (2020).
8. Wrapp, D. et al. Cryo-EM structure of the 2019-nCoV spike in the prefusion conformation. Science 367, 1260-1263, doi:10.1126/science.abb2507 (2020).
9. Cui, J., Li, F. & Shi, Z.-L. Origin and evolution of pathogenic coronaviruses. Nature Reviews Microbiology 17, 181-192, doi:10.1038/s41579-018-0118-9 (2019).
10. V'kovski, P., Kratzel, A., Steiner, S., Stalder, H. & Thiel, V. Coronavirus biology and replication: implications for SARS-CoV-2. Nature Reviews Microbiology, doi:10.1038/s41579-020-00468-6 (2020).
11. Hoffmann, M. et al. SARS-CoV-2 Cell Entry Depends on ACE2 and TMPRSS2 and Is Blocked by a Clinically Proven Protease Inhibitor. Cell 181, 271-280.e278, doi:https://doi.org/10.1016/j.cell.2020.02.052 (2020).
12. Thiel, V. et al. Mechanisms and enzymes involved in SARS coronavirus genome expression. Journal of General Virology 84, 2305-2315, doi:https://doi.org/10.1099/vir.0.19424-0 (2003).
13. Beigel, J. H. et al. Remdesivir for the Treatment of Covid-19—Final Report. New England Journal of Medicine 383, 1813-1826, doi:10.1056/NEJMoa2007764 (2020).
14. Lei, J., Kusov, Y. & Hilgenfeld, R. Nsp3 of coronaviruses: Structures and functions of a large multi-domain protein. Antiviral Research 149, 58-74, doi:https://doi.org/10.1016/j.antiviral.2017.11.001 (2018).
15. Ratia, K. et al. Severe acute respiratory syndrome coronavirus papain-like protease: Structure of a viral deubiquitinating enzyme. Proceedings of the National Academy of Sciences 103, 5717-5722, doi:10.1073/pnas.0510851103 (2006).
16. Barretto, N. et al. The Papain-Like Protease of Severe Acute Respiratory Syndrome Coronavirus Has Deubiquitinating Activity. Journal of Virology 79, 15189-15198, doi:10.1128/jvi.79.24.15189-15198.2005 (2005).
17. Chen, X. et al. SARS coronavirus papain-like protease inhibits the type I interferon signaling pathway through interaction with the STING-TRAF3-TBK1 complex. Protein & Cell 5, 369-381, doi:10.1007/si3238-014-0026-3 (2014).
18. Frieman, M., Ratia, K., Johnston, R. E., Mesecar, A. D. & Baric, R. S. Severe Acute Respiratory Syndrome Coronavirus Papain-Like Protease Ubiquitin-Like Domain and Catalytic Domain Regulate Antagonism of IRF3 and NF-κB Signaling. Journal of Virology 83, 6689-6705, doi:10.1128/jvi.02220-08 (2009).
19. Ratia, K., Kilianski, A., Baez-Santos, Y. M., Baker, S. C. & Mesecar, A. Structural Basis for the Ubiquitin-Linkage Specificity and delSGylating Activity of SARS-CoV Papain-Like Protease. PLOS Pathogens 10, e1004113, doi:10.1371/journal.ppat.1004113 (2014).
20. Shin, D. et al. Papain-like protease regulates SARS-CoV-2 viral spread and innate immunity. Nature 587, 657-662, doi:10.1038/s41586-020-2601-5 (2020).
21. Freitas, B. T. et al. Characterization and Noncovalent Inhibition of the Deubiquitinase and delSGylase Activity of SARS-CoV-2 Papain-Like Protease. ACS Infectious Diseases 6, 2099-2109, doi:10.1021/acsinfecdis.0c00168 (2020).
22. Lindner, H. A. et al. The Papain-Like Protease from the Severe Acute Respiratory Syndrome Coronavirus Is a Deubiquitinating Enzyme. Journal of Virology 79, 15199-15208, doi:10.1128/jvi.79.24.15199-15208.2005 (2005).
23. Del Valle, D. M. et al. An inflammatory cytokine signature predicts COVID-19 severity and survival. Nature Medicine 26, 1636-1643, doi:10.1038/s41591-020-1051-9 (2020).
24. Dyall, J. et al. Repurposing of clinically developed drugs for treatment of Middle East respiratory syndrome coronavirus infection. Antimicrobial agents and chemotherapy 58, 4885-4893, doi:10.1128/AAC.03036-14 (2014).
25. Zhou, Y. et al. Network-based drug repurposing for novel coronavirus 2019-nCoV/SARS-CoV-2. Cell Discov 6, 14, doi:10.1038/s41421-020-0153-3 (2020).
26. Siklos, M., BenAissa, M. & Thatcher, G. R. Cysteine proteases as therapeutic targets: does selectivity matter? A systematic review of calpain and cathepsin inhibitors. Acta Pharm Sin B 5, 506-519, doi:10.1016/j.apsb.2015.08.001 (2015).
27. Sacco, M. D. et al. Structure and inhibition of the SARS-CoV-2 main protease reveals strategy for developing dual inhibitors against M(pro) and cathepsin L. bioRxiv, doi:10.1101/2020.07.27.223727 (2020).
28. Hoffman, R. L. et al. Discovery of Ketone-Based Covalent Inhibitors of Coronavirus 3CL Proteases for the Potential Therapeutic Treatment of COVID-19. J Med Chem 63, 12725-12747, doi:10.1021/acs.jmedchem.0c01063 (2020).
29. Ratia, K. et al. A noncovalent class of papain-like protease/deubiquitinase inhibitors blocks SARS virus replication. Proceedings of the National Academy of Sciences 105, 16119-16124, doi:10.1073/pnas.0805240105 (2008).
30. Ghosh, A. K. et al. Structure-Based Design, Synthesis, and Biological Evaluation of a Series of Novel and Reversible Inhibitors for the Severe Acute Respiratory Syndrome-Coronavirus Papain-Like Protease. Journal of Medicinal Chemistry 52, 5228-5240, doi:10.1021/jm900611 t (2009).
31. Ghosh, A. K. et al. Severe Acute Respiratory Syndrome Coronavirus Papain-like Novel Protease Inhibitors: Design, Synthesis, Protein-Ligand X-ray Structure and Biological Evaluation. Journal of Medicinal Chemistry 53, 4968-4979, doi:10.1021/jm1004489 (2010).
32. Báez-Santos, Y. M. et al. X-ray Structural and Biological Evaluation of a Series of Potent and Highly Selective Inhibitors of Human Coronavirus Papain-like Proteases. Journal of Medicinal Chemistry 57, 2393-2412, doi:10.1021/jm401712t (2014).
33. Ghosh, A. K., Brindisi, M., Shahabi, D., Chapman, M. E. & Mesecar, A. D.

Drug Development and Medicinal Chemistry Efforts toward SARS-Coronavirus and Covid-19 Therapeutics. Chem Med Chem 15, 907-932, doi:https://doi.org/10.1002/cmdc.202000223 (2020).
34. Ratia, K. et al. A noncovalent class of papain-like protease/deubiquitinase inhibitors blocks SARS virus replication. Proc Natl Acad Sci USA 105, 16119-16124, doi:10.1073/pnas.0805240105 (2008).
35. Ghosh, A. K. et al. Structure-based design, synthesis, and biological evaluation of a series of novel and reversible inhibitors for the severe acute respiratory syndrome-coronavirus papain-like protease. J Med Chem 52, 5228-5240, doi:10.1021/jm900611t (2009).
36. Ghosh, A. K. et al. Severe acute respiratory syndrome coronavirus papain-like novel protease inhibitors: design, synthesis, protein-ligand X-ray structure and biological evaluation. J Med Chem 53, 4968-4979, doi:10.1021/jm1004489 (2010).
37. Baez-Santos, Y. M., St John, S. E. & Mesecar, A. D. The SARS-coronavirus papain-like protease: structure, function and inhibition by designed antiviral compounds. Antiviral research 115, 21-38, doi:10.1016/j.antiviral.2014.12.015 (2015).
38. Fu, Z. et al. The complex structure of GRL0617 and SARS-CoV-2 PLpro reveals a hot spot for antiviral drug discovery. Nature communications 12, 488, doi:10.1038/s41467-020-20718-8 (2021).
39. Báez-Santos, Y. M., St. John, S. E. & Mesecar, A. D. The SARS-coronavirus papain-like protease: Structure, function and inhibition by designed antiviral compounds. Antiviral Research 115, 21-38, doi:https://doi.org/10.1016/j.antiviral.2014.12.015 (2015).
40. Klemm, T. et al. Mechanism and inhibition of the papain-like protease, PLpro, of SARS-CoV-2. The EMBO Journal 39, e106275, doi:https://doi.org/10.15252/embj.2020106275 (2020).
41. De Rosa, M. F., Sillence, D., Ackerley, C. & Lingwood, C. Role of multiple drug resistance protein 1 in neutral but not acidic glycosphingolipid biosynthesis. The Journal of biological chemistry 279, 7867-7876, doi:10.1074/jbc.M305645200 (2004).
42. Boras, B. et al. Discovery of a Novel Inhibitor of Coronavirus 3CL Protease as a Clinical Candidate for the Potential Treatment of COVID-19. bioRxiv, 2020.2009.2012.293498, doi:10.1101/2020.09.12.293498 (2020).
43. Vabret, N. et al. Immunology of COVID-19: Current State of the Science. Immunity 52, 910-941, doi:https://doi.org/10.1016/j.immuni.2020.05.002 (2020).
44. Ebner, P., Versteeg, G. A. & Ikeda, F. Ubiquitin enzymes in the regulation of immune responses. Crit Rev Biochem Mol Biol 52, 425-460, doi:10.1080/10409238.2017.1325829 (2017).
45. Klemm, T. et al. Mechanism and inhibition of the papain-like protease, PLpro, of SARS-CoV-2. EMBO J, e106275, doi:10.15252/embj.2020106275 (2020).
46. Mohamud, Y. et al. The papain-like protease of coronaviruses cleaves ULK1 to disrupt host autophagy. Biochemical and Biophysical Research Communications 540, 75-82, doi:https://doi.org/10.1016/j.bbrc.2020.12.091 (2021).
47. Smith, E. et al. High-Throughput Screening for Drugs That Inhibit Papain-Like Protease in SARS-CoV-2. SLAS DISCOVERY: Advancing the Science of Drug Discovery 25, 1152-1161, doi:10.1177/2472555220963667 (2020).
48. Kouznetsova, V. L., Zhang, A., Tatineni, M., Miller, M. A. & Tsigelny, I. F. Potential COVID-19 papain-like protease PLpro inhibitors: repurposing FDA-approved drugs. Peer J 8, e9965, doi:10.7717/peerj.9965 (2020).
49. Wu, C. et al. Analysis of therapeutic targets for SARS-CoV-2 and discovery of potential drugs by computational methods. Acta Pharm Sin B, doi:10.1016/j.apsb.2020.02.008 (2020).
50. Osipiuk, J. et al. Structure of papain-like protease from SARS-CoV-2 and its complexes with non-covalent inhibitors. Nature communications 12, 743, doi:10.1038/s41467-021-21060-3 (2021).
51. Rut, W. et al. Activity profiling and crystal structures of inhibitor-bound SARS-CoV-2 papain-like protease: A framework for anti-COVID-19 drug design. Science Advances 6, eabd4596, doi:10.1126/sciadv.abd4596 (2020).
52. Chu, H. et al. Comparative Replication and Immune Activation Profiles of SARS-CoV-2 and SARS-CoV in Human Lungs: An Ex Vivo Study With Implications for the Pathogenesis of COVID-19. Clin Infect Dis 71, 1400-1409, doi:10.1093/cid/ciaa410 (2020).
53. Del Ser, T. et al. Residence, Clinical Features, and Genetic Risk Factors Associated with Symptoms of COVID-19 in a Cohort of Older People in Madrid. Gerontology, 1-9, doi:10.1159/000513182 (2021).
54. Wang, C. et al. ApoE-Isoform-Dependent SARS-CoV-2 Neurotropism and Cellular Response. Cell Stem Cell, doi:10.1016/j.stem.2020.12.018 (2021).
55. Studier, F. W. Protein production by auto-induction in high density shaking cultures. Protein expression and purification 41, 207-234, doi:10.1016/j.pep.2005.01.016 (2005).
56. Kabsch, W. Integration, scaling, space-group assignment and post-refinement. Acta Crystallogr D Biol Crystallogr 66, 133-144, doi:10.1107/S0907444909047374 (2010).
57. Strong, M. et al. Toward the structural genomics of complexes: crystal structure of a PE/PPE protein complex from Mycobacterium tuberculosis. Proc Natl Acad Sci USA 103, 8060-8065, doi:10.1073/pnas.0602606103 (2006).
58. Vagin, A. & Teplyakov, A. Molecular replacement with MOLREP. Acta Crystallogr D Biol Crystallogr 66, 22-25, doi:10.1107/S0907444909042589 (2010).
59. Murshudov, G. N., Vagin, A. A. & Dodson, E. J. Refinement of macromolecular structures by the maximum-likelihood method. Acta Crystallogr D Biol Crystallogr 53, 240-255, doi:10.1107/s0907444996012255 (1997).
60. Emsley, P., Lohkamp, B., Scott, W. G. & Cowtan, K. Features and development of Coot. Acta Crystallogr D Biol Crystallogr 66, 486-501, doi:10.1107/s0907444910007493 (2010).

1. A compound having a Formula I: 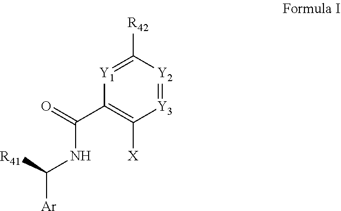
or a pharmaceutically acceptable salt thereof, wherein
X is -Me, -Et, —OMe, or —CH═CH₂;
Y₁-Y₃ are independently selected from —N and —CH;
Ar is selected from aryl, heteroaryl, heterocyclyl, cycloalkyl, and cycloalkenyl, wherein each Ar group can be substituted with 1, 2, 3, 4, or 5 R^{d} groups;
R₄₁ and R₄₂ are independently selected from —C₁-C₆ alkyl, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, —(C₁-C₆ alkylenyl)R^{c}R^{c′}, and —C₁-C₆ alkyl;
R^{a} and R^{b} are independently selected at each occurrence from —H, —C₁-C₆ alkenyl, —C₁-C₆ alkynyl, —C₁-C₆ haloalkyl, —R^{c}, and —C₁-C₆ alkyl, wherein the —C₁-C₆ alkyl can be substituted with a substituent selected from —OR^{e}, —NR^{e}R^{f}, —C(O)OR^{e}, —C(O)NR^{e}R^{f}, —S(O)₂R^{e}, —S(O)₂NR^{e}R^{f}, and
—R^{c}; R^{c} and R^{c′} are independently selected at each occurrence from aryl, heteroaryl, heterocyclyl, cycloalkyl, and cycloalkenyl, wherein each R^{c} group can be substituted with 1, 2, 3, 4, or 5 R^{d} groups; and R^{d} is independently selected at each occurrence from —C₁-C₆ alkyl, —C₂-C₆ alkenyl, —C₂-C₆ alkynyl halogen, C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f},
wherein R^{e} and R^{f}, are independently selected at each occurrence from —H, —C₁-C₆ alkyl, —C₁-C₆ cycloalkyl, aryl, heteroaryl and —C₁-C₆ haloalkyl.

2. The compound of claim 1, wherein X is -Me.

3. The compound of claim 1, wherein each of Y₁-Y₃ are —CH.

4. The compound of claim 1, wherein R₄₁ is a —C₁-C₆ alkyl.

5. The compound of claim 1, wherein Ar is an aryl.

6. The compound of claim 1, wherein said compound has the following Formula II: 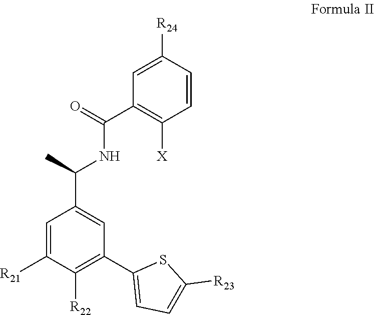
or a pharmaceutically acceptable salt thereof, wherein
X is -Me, -Et, —OMe, or —CH═CH₂;
R₂₁, R₂₂, R₂₃ and R₂₄ are independently selected from —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′}; and
R^{a} and R^{b} are independently selected at each occurrence from —H, —C₁-C₆ alkenyl, —C₁-C₆ alkynyl, —C₁-C₆ haloalkyl, R^{c}, and —C₁-C₆ alkyl, wherein the —C₁-C₆ alkyl can be substituted with —OR^{e}, —NR^{e}R^{f}, —C(O)OR^{e}, —C(O)NR^{e}R^{f}, —S(O)₂R^{e}, —S(O)₂NR^{e}R^{f}, or R^{c};
R^{c} and R^{c′} are independently selected at each occurrence from aryl, heteroaryl, heterocyclyl, cycloalkyl, or cycloalkenyl, wherein each R^{c} group can be substituted with 1, 2, 3, 4, or 5 R^{d} groups; and
R^{d} is independently selected at each occurrence from —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, wherein R^{e} and R^{f} are independently selected at each occurrence from —H, —C₁-C₆ alkyl, —C₁-C₆ cycloalkyl, aryl, heteroaryl and —C₁-C₆ haloalkyl.

7. The compound of claim 1, wherein said compound has the following Formula V: 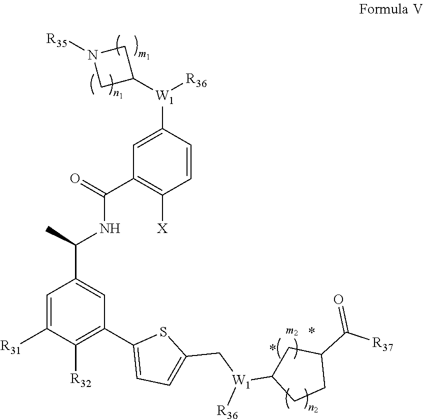
or a pharmaceutically acceptable salt thereof, wherein
X is -Me, -Et, —OMe, —CH═CH₂;
W₁ is N or O; wherein if W₁═O, R₃₆ does not exist;
R₃₁ and R₃₂ is independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₃ is selected from —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′}; PROTAC; hybridized compound, or prodrug;
R₃₄, R₃₅, R₃₆, and R₃₇ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, is independently selected from the group of: a C₁-C₆ alkyl, a C₂-C₆ alkenyl, a C₂-C₆ alkynyl, a halogen, a C₁-C₆ haloalkyl, —CN, NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, a —(C₁-C₆ alkylenyl)-C(O)NR^{e}R^{f}, a —(C₁-C₆ alkylenyl)-NR^{e}R^{f}, and a —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
m₁, m₂, n₁ and n₂=1-3.

8. The compound of claim 7, wherein W₁ is O.

9. The compound of claim 7, wherein W₁ is N.

10. The compound of claim 7, wherein m₁, m₂, n₁ and n₂ are 1.

11. The compound of claim 7, wherein said compound is:

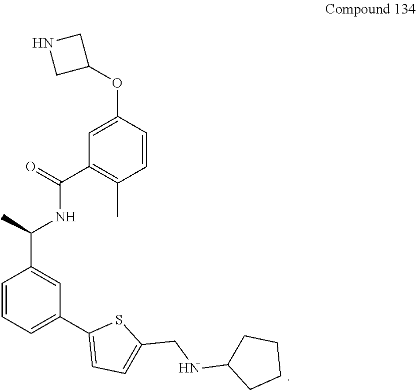 [SMILES: Cc1ccc(OC2CNC2)cc1C(=O)N[C@H](C)c1cccc(-c2ccc(CNC3CCCC3)s2)c1]

12. The compound of claim 1, wherein said compound has the following Formula XI: 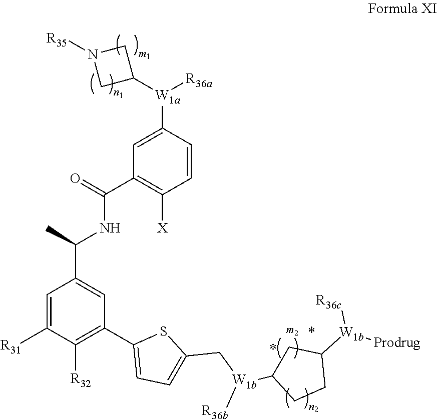
or a pharmaceutically acceptable salt thereof, wherein
X is -Me, -Et, —OMe, —CH═CH₂;
W₁ₐ and W_{1b} is —N, —O or —C; wherein if W₁ₐ═O, R₃₆ₐ does not exist and/or if W_{1b}═O, R_{36b} does not exist;
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

13. The compound of claim 12, wherein the prodrug is selected from the group consisting of hydroxyl, carboxyl, amine, phosphate, phosphonate, amidine, guanine and carbohydrate.

14. The compound of claim 1, wherein said compound has the following Formula XII: 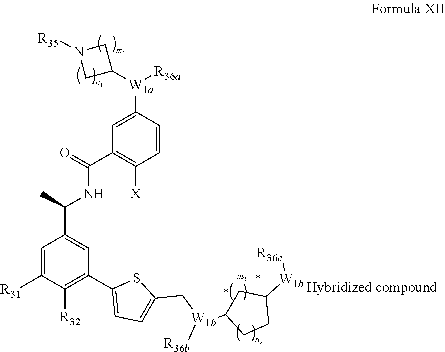
or a pharmaceutically acceptable salt thereof, wherein
X is -Me, -Et, —OMe, —CH═CH₂;
W₁ₐ and W_{1b} is —N, —O or —C; wherein if W₁═O, R₃₆ does not exist,
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₃, R₃₄, R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

15. The compound of claim 1, wherein said compound has the following Formula XIII: 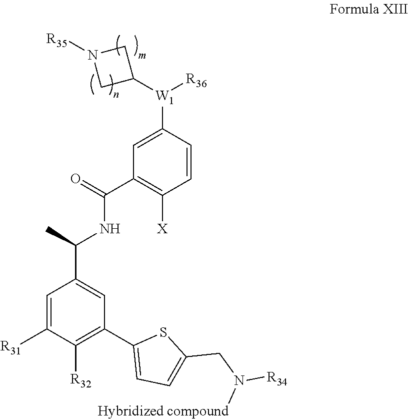
or a pharmaceutically acceptable salt thereof, wherein
X is -Me, -Et, —OMe, or —CH═CH₂;
W₁ is —N or —O; wherein if W₁═O, R₃₆ does not exist;
R₃₁ and R₃₂ are independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₄, R₃₅, and R₃₆ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
m and n=1-3.

16. The compound of claim 14, wherein the hybridized compound is selected from the group consisting of kinase inhibitors, NAMPT inhibitors, coronavirus inhibitors and/or virus inhibitors.

17. The compound of claim 14, wherein said compound is selected from:

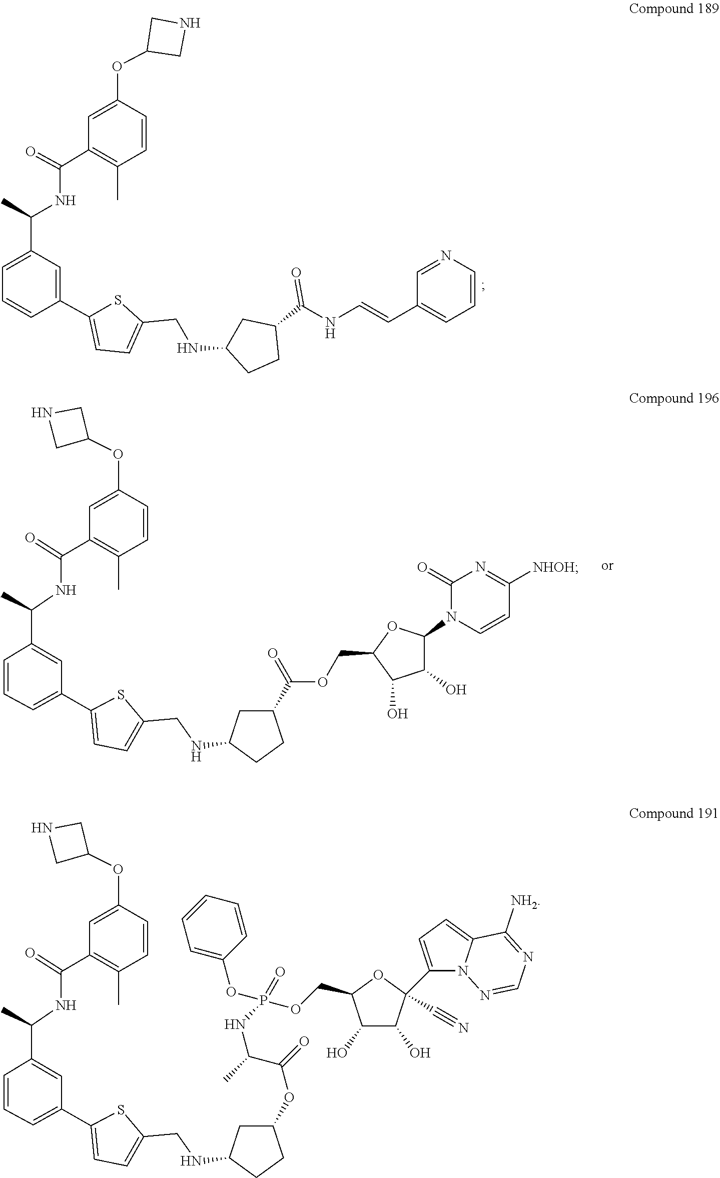

18. The compound of claim 1, wherein said compound has the following Formula XIV: 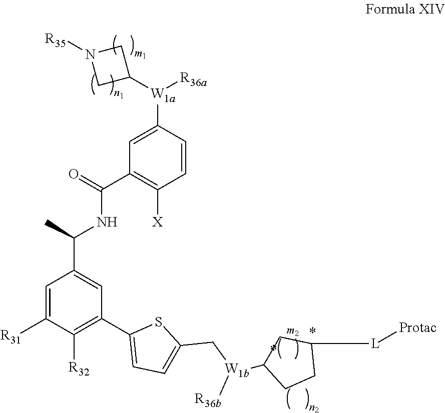
or a pharmaceutically acceptable salt thereof, wherein
X is -Me, -Et, —OMe, —CH═CH₂;
W₁ₐ and W_{1b} is —N, —O or —C; wherein if W₁═O, R₃₆ does not exist
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl;
m₁, m₂, n₁ and n₂=1-3; and
L is a (poly)ethyleneglycol or substituted alkyl groups optionally interdispersed with O, N, S, P or Si atoms.

19. The compound of claim 18, wherein the Protac is a hetero bifunctional molecule that connects a POI ligand to an E3 ubiquitin ligase (E3) (VHL, CRBN, IAPs, and MDM2) recruiting ligand selected from the group consisting of thalidomide, pomalidomide, lenalidomide, and VHL.

20. The compound of claim 18, wherein said compound is:

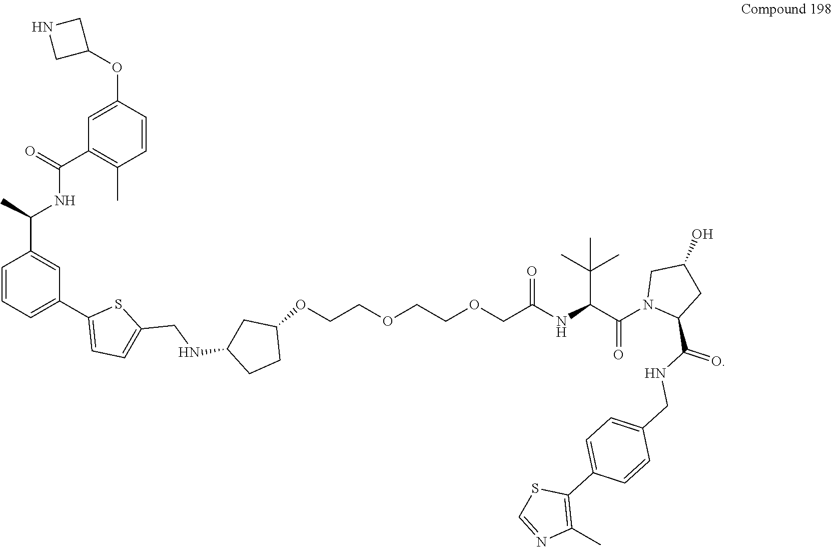 [SMILES: Cc1ccc(OC2CNC2)cc1C(=O)N[C@H](C)c1cccc(-c2ccc(CN[C@H]3CC[C@@H](OCCOCCOCC(=O)N[C@H](C(=O)N4C[C@H](O)C[C@H]4C(=O)NCc4ccc(-c5scnc5C)cc4)C(C)(C)C)C3)s2)c1]

21. The compound of claim 1, wherein said compound has the following Formula XII(a): 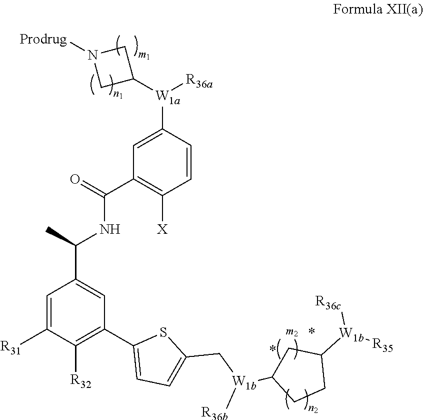
or a pharmaceutically acceptable salt thereof, wherein
X is -Me, -Et, —OMe, halogen or —CH═CH₂;
W₁ₐ and W_{1b} is —N, —O or —C; wherein if W₁═O, R₃₆ does not exist,
R₃₁ and R₃₂ is independently selected from the group of H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′};
R₃₃, R₃₄, R₃₅, R₃₆ₐ, R_{36b} and R_{36c} are independently selected from the group of H, —═O, —═S, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′};
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
—R^{c} and —R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups;
R^{d}, at each occurrence, are each independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: —H, —C₁-C₆ alkyl, —C₁-C₆ cycloalkyl, aryl, heteroaryl and —C₁-C₆ haloalkyl; and
m₁, m₂, n₁ and n₂=1-3.

22. The compound of claim 1, wherein said compound has the following Formula XIII(a): 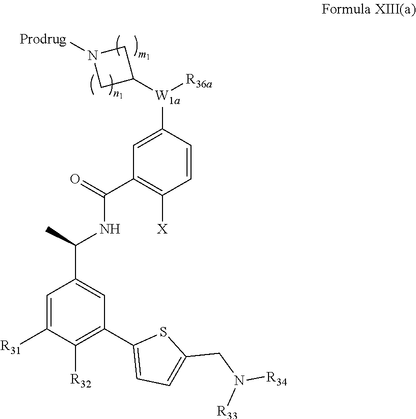
or a pharmaceutically acceptable salt thereof, wherein
X is -Me, -Et, —OMe, halogen or —CH═CH₂;
W₁ is —N or —O; wherein if W₁═O, R₃₆ does not exist;
R₃₁ and R₃₂ are independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₄ and R₃₆ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, and —(C₁-C₆ alkylenyl)R^{c}R^{c′}; R₃₃ is selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, —(C₁-C₆ alkylenyl)R^{c}R^{c′}, PROTACs, hybrid compounds, and Prodrugs;
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
m and n=1-3.

23. The compound of claim 1, wherein said compound has the following Formula XIV(a): 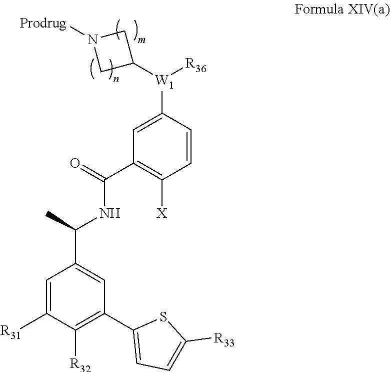
or a pharmaceutically acceptable salt thereof, wherein
X is -Me, -Et, —OMe, halogen or —CH═CH₂;
W₁ is —N or —O; wherein if W₁═O, R₃₆ does not exist;
R₃₁, R₃₂ and R₃₃ are independently selected from the group of —H, halogen, —(C₁-C₆ alkylenyl)NR^{a}R^{b}, —OR^{a}, —(C₁-C₆ alkylenyl)NC(O)R^{a}, —(C₁-C₆ alkylenyl) C(O)NR^{a}, —N(R^{a})S(O)₂R^{b}, —S(O)₂NR^{a}R^{b}, —C(O)NR^{a}R^{b}, —N(R^{a})C(O)R^{b}, —NR^{a}R^{b}, —(C₁-C₆ alkylenyl)R^{c}, —(C₁-C₃ cycloalkylenyl)R^{c}, and —(C₁-C₆ alkylenyl)R^{c}R^{c′},
R₃₄ and R₃₆ are independently selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃cycloalkylenyl)R^{c}, and —(C₁-C₆ alkylenyl)R^{c}R^{c′}; R₃₃ is selected from the group of —H, —(C₁-C₆ alkylenyl)NR^{a}R^{b}, —(C₁-C₆ alkylenyl)NC(O)R^{a}, —(C₁-C₆ alkylenyl), C(O)R^{a}, —S(O)₂R^{b}, —(C₁-C₆ alkylenyl)R^{c}, —(C₁-C₃ cycloalkylenyl)R^{c}, —(C₁-C₆ alkylenyl)R^{c}R^{c′}, PROTACs, hybrid compounds, and Prodrugs:
R^{a} and R^{b}, at each occurrence, are each independently selected from the group of: —H, —C₁-C₆ alkenyl, —C₁-C₆ alkynyl, —C₁-C₆ haloalkyl, —R^{c}, or —C₁-C₆ alkyl, where the —C₁-C₆ alkyl can be substituted with one substituent selected from the group of: —OR^{e}, —NR^{e}R^{f}, —C(O)OR^{e}, —C(O)NR^{e}R^{f}, —S(O)₂R^{e}, —S(O)₂NR^{e}R^{f}, and —R^{c};
R^{c} and R^{c′}, at each occurrence, are each independently selected from the group of: an aryl, a heteroaryl, a heterocycle, a cycloalkyl, or a cycloalkenyl, and where each R^{c} group can be substituted with 1, 2, 3, 4, or 5 R^{d} groups; where R^{d}, at each occurrence, can be independently selected from the group of: —C₁-C₆ alkyl, —C₂-C₆ alkenyl, —C₂-C₆ alkynyl, halogen, —C₁-C₆ haloalkyl, —CN, —NO₂, —OR^{e}, —S(O)₂NR^{e}R^{f}, —C(O)R^{e}, —C(O)NR^{e}R^{f}, —NR^{e}R^{f}, —N(R^{e})C(O)R^{f}, a —(C₁-C₆ alkylenyl)-OR^{e}, —(C₁-C₆ alkylenyl)-C(O)NR^{e}R^{f}, —(C₁-C₆ alkylenyl)-NR^{e}R^{f}, and —(C₁-C₆ alkylenyl)-N(R^{e})C(O)R^{f}, and where R^{e} and R^{f}, at each occurrence, can each be independently selected from the group of: H, a C₁-C₆ alkyl, a C₁-C₆ cycloalkyl, a aryl, a heteroaryl and a C₁-C₆ haloalkyl; and
m and n=1-3.

24. The compound of claim 21, wherein the prodrug is selected from the group consisting of hydroxyl, carboxyl, amine, phosphate, phosphonate, amidine, guanine, and carbohydrate.

25. A pharmaceutical composition comprising the compound of claim 1.

26. The pharmaceutical composition of claim 25, being packaged in a packaging material and identified in print, in or on said packaging material, for use in treating and/or preventing an infection caused by a coronavirus.

27. A method of treating and/or preventing an infection caused by a coronavirus in a subject in need thereof, the method comprising administering to the subject the pharmaceutical composition of claim 25.

28. The method of claim 27, wherein the coronavirus is SARS-CoV-2.

29. The method of claim 27, wherein the subject is 65 years or older.

30. The method of claim 27, wherein the subject has one or more underlying medical conditions selected from the group consisting of cancer, chronic kidney disease, chronic obstructive pulmonary disease (COPD), immunocompromised state, obesity, serious heart conditions, sickle cell disease, Type 2 diabetes mellitus, asthma, cerebrovascular disease, cystic fibrosis, hypertension or high blood pressure, neurologic conditions, liver disease, pregnancy, pulmonary fibrosis, smoking, thalassemia, and Type 1 diabetes mellitus.

31. The method of claim 27, further comprising administering to the subject one or more antiviral agents.

32. The method of claim 31, wherein the one or more antiviral agents is selected from the group consisting of remdesivir, favipiravir, lopinavir, ritonavir, nitazoxanide, danoprevir, ASC-09, umifenovir, nafamostat, brequinar, AT-527, ABX464, merimepodib, molnupiravir, opaganib, ivermectin, and hydroxychloroquine.

33. The method of claim 31, wherein the one or more antiviral agents is a vaccine.

34. The method of claim 33, wherein the vaccine is selected from the group consisting of BNT162b2 (Pfizer/BioNTech), mRNA-1273 (Moderna), AZD1222/ChAdOxl (AstraZeneca/Oxford Univ), Ad5-vectored COVID-19 vaccine (CanSino Biologies), CoronaVac (Sinovac), and NVX-CoV2373 (Novavax).


SEQUENCE LISTING (6 sequences)

SEQ ID NO: 1 - DNA, 20 bp, Artificial Sequence
  Primer
gaccccaaaatcagcgaaat

SEQ ID NO: 2 - DNA, 24 bp, Artificial Sequence
  Primer
tctggttactgccagttgaatctg

SEQ ID NO: 3 - DNA, 24 bp, Artificial Sequence
  Probe
accccgcattacgtttggtggacc

SEQ ID NO: 4 - protein, 8 aa, Artificial Sequence
  Synthetic
RLRGGAMC

SEQ ID NO: 5 - protein, 11 aa, Artificial Sequence
  Synthetic
FTLRGGAPTKV

SEQ ID NO: 6 - protein, 8 aa, Artificial Sequence
  Synthetic
LRGGAPTK